(19) United States
(12) Patent Application Publication
(10) Pub. No.: US 2023/0265192 A1
(43) Pub. Date: Aug. 24, 2023

(54) ANTI-CTLA-4 BINDING PROTEINS AND METHODS OF USE THEREOF

(71) Applicant: GigaGen, Inc., San Carlos, CA (US)

(72) Inventors: David Scott JOHNSON, San Francisco, CA (US); Adam Shultz ADLER, Belmont, CA (US); Rena Aviva MIZRAHI, Pacifica, CA (US); Yoong Wearn LIM, San Carlos, CA (US); Michael ASENSIO, San Carlos, CA (US); Erica Lyn STONE, San Carlos, CA (US)

(73) Assignee: GigaGen, Inc., San Carlos, CA (US)

(21) Appl. No.: 18/003,076

(22) PCT Filed: Jul. 2, 2021
(86) PCT No.: PCT/US2021/040379

§ 371 Date: Dec. 22, 2022

Related U.S. Application Data
(60) Provisional application No. 63/047,785, filed on Jul. 2, 2020.
(60) Provisional application No. 63/107,376, filed on Oct. 29, 2020.

(51) Int. Cl. C07K 16/28; A61P 35/00

(52) U.S. Cl. CPC C07K 16/2818; A61P 35/00; C07K 2317/565; C07K 2317/92; C07K 2317/41; C07K 2317/52

(57) ABSTRACT

Provided herein are antigen-binding proteins (ABPs) that selectively bind to CTLA-4 and its isoforms and homologs, and compositions comprising the ABPs. Also provided are methods of using the ABPs, such as therapeutic and diagnostic methods.

### 1. CROSS REFERENCE TO RELATED APPLICATIONS

This application claims priority benefit to U.S. provisional application Ser. No. 63/047,785 filed on Jul. 2, 2020, and 63/107,376 filed Oct. 29, 2020, which applications are incorporated herein by reference in their entireties.

### 2. SEQUENCE LISTING

The instant application contains a Sequence Listing with 12088 sequences which has been submitted via EFS-Web and is hereby incorporated by reference in its entirety. Said ASCII copy, created on Jul. 2, 2021, is named “49228WO Sequence_Listing”, and is 1.86 megabytes in size.

### 3. FIELD

Provided herein are antigen-binding proteins (ABPs) with binding specificity for CTLA-4 and compositions comprising such ABPs, including pharmaceutical compositions, diagnostic compositions, and kits. Also provided are methods of making CTLA-4 ABPs, and methods of using CTLA-4 ABPs, for example, for therapeutic purposes, diagnostic purposes, and research purposes.

### 4. BACKGROUND

CTLA-4, also known as cytotoxic T-lymphocyte associated protein 4 and CD152 (cluster of differentiation 152), is a cell surface receptor that suppresses T cell inflammatory activity. CTLA-4 is constitutively expressed by regulatory T cells (Tregs) and upregulated in stimulated T cells. CD80 and CD86, also expressed in antigen presenting cells (APCs) such as dendritic cells (DCs), are the primary ligands of CTLA-4. The interaction between CTLA-4 and its ligands is vitally important for downregulating the immune responses and promoting self-tolerance by suppressing T cell inflammatory activity. This activity prevents autoimmune diseases, as well as prevents the immune system from killing cancer cells.

CTLA-4 is a member of the immunoglobulin superfamily that is expressed by activated T cells and transmits an inhibitory signal to T cells. CTLA-4 binds CD80 and CD86 with greater affinity and avidity than CD28 thus enabling it to outcompete CD28 for its ligands. CTLA-4 transmits an inhibitory signal to T cells, whereas CD28 transmits a stimulatory signal. CTLA-4 is also found in regulatory T cells (Tregs) and contributes to their inhibitory function. T cell activation through the T cell receptor and CD28 leads to increased expression of CTLA-4. The mechanism by which CTLA-4 acts in T cells remains somewhat controversial. Biochemical evidence suggested that CTLA-4 recruits a phosphatase to the T cell receptor (TCR), thus attenuating the signal. This work remains unconfirmed in the literature since its first publication. More recent work has suggested that CTLA-4 may function in vivo by capturing and removing B7-1 and B7-2 from the membranes of antigen-presenting cells, thus making these unavailable for triggering of CD28.

Variants in CTLA-4 have been associated with insulin-dependent diabetes mellitus, Graves' disease, Hashimoto's thyroiditis, celiac disease, systemic lupus erythematosus, thyroid-associated orbitopathy, primary biliary cirrhosis and other autoimmune diseases. The comparatively high binding affinity of CTLA-4 for CD80 and CD86 has made it a potential therapy for autoimmune diseases. Soluble fusion proteins of CTLA-4 and antibodies (CTLA-4-Ig) have been used in clinical trials for rheumatoid arthritis.

Recently, CTLA-4 antibodies have been used with varying success to treat some types of cancer. CTLA-4 inhibitors have been shown to antagonize binding of CTLA-4 to its ligands, thereby activating the immune system to attack tumors. The current mechanism of action of known anti-CTLA-4 therapies is to block the interaction between CTLA-4 and its ligands for checkpoint inhibition. For example, CTLA-4 monoclonal antibodies (mAbs) such as ipilimumab were originally intended to block the binding of CTLA-4 to its ligands, the B7 proteins CD80 and CD86, i.e., “checkpoint inhibition”. Blocking CTLA-4 binding to B7 proteins frees B7 proteins to bind to CD28, inducing T cell co-stimulation and activation. CTLA-4 antibodies have also been used to induce antibody-dependent cell-mediated cytotoxicity (ADCC) of Tregs specific to the tumor microenvironment, thus reducing immune tolerance to the tumor. Thus, in addition to blocking the interaction of CTLA-4 with its B7 ligands, anti-CTLA-4 mAbs are also able to induce antibody-dependent cell-mediated cytotoxicity (ADCC) and antibody-dependent cellular phagocytosis (ADCP) of intratumoral FOXP3⁺ regulatory T cells (Tregs), which express comparatively high levels of surface CTLA-4.

Thus, inhibition of CTLA-4 function is currently one of the most promising systemic therapeutic approach for various diseases. There is a need for developing CTLA-4 ABPs that can be used for treatment, diagnosis, and research of various diseases, including cancer and autoimmune disease.

PCT Application PCT/US2019/068820, filed on Dec. 27, 2019 and published as Publication No. WO2020140084A1, describes CTLA-4 ABPs, which application is incorporated by reference in its entirety herein.

### 5. SUMMARY

Provided herein are novel ABPs with binding specificity for CTLA-4 and methods of using such ABPs. The ABPs specifically bind a human CTLA-4 (SEQ ID: 7001) or a fragment of the human CTLA-4.

In particular, in one aspect, the present disclosure provides a novel CTLA-4 monoclonal antibody with minimal ability to block CTLA-4 binding to its CD80/CD86 ligands but has superior anti-tumor activity with reduced toxicity. The anti-CTLA4 antibody was demonstrated to induce less peripheral Treg proliferation, and more efficient intratumoral Treg depletion, in murine models expressing human CTLA-4.

The present disclosure also provides that the anti-CTLA-4 monoclonal antibodies bind to CTLA-4 at an epitope that differs from other, known anti-CTLA-4 antibodies (e.g., Ipilimumab), and has limited checkpoint inhibitor activity and thus is a weak checkpoint inhibitor. Surprisingly, efficacy of the anti-CTLA-4 antibodies presented herein was found to be associated with FcR-mediated Treg depletion in the tumor microenvironment. The anti-CTLA-4 antibodies also induce less Treg proliferation and has increased ability to induce in vitro FcR signaling and in vivo depletion of intratumoral Tregs. Experimental results described herein suggest that the enhanced FcR activity of the weak checkpoint inhibitor likely contributes to its enhanced anti-tumor activity. They also show that weak checkpoint inhibition was associated with lower toxicity in murine models.

In some embodiments, when bound to CTLA-4, the ABP contacts amino acids K130, Y139, L141, I143 but does not contact amino acid R70 of the CTLA-4, or R70 is not energetically a major contributor to the interaction between CTLA-4 and the ABP; and/or the CTLA-4 can associate with CD80/CD86 when bound to the ABP; and/or an interaction between the ABP and amino acid L74A and/or E68 of the CTLA-4 is greater than an interaction between Ipilimumab and amino acid L74A of CTLA-4.

In some embodiments, the ABP comprises a CDR1-L consisting of SEQ ID NO:12078 or SEQ ID NO: 1014, a CDR2-L consisting of SEQ ID NO:12079 or SEQ ID NO: 2014, a CDR3-L consisting of SEQ ID NO:12080 or SEQ ID NO: 3014, a CDR1-H consisting of SEQ ID NO:12075 or SEQ ID NO: 4014, a CDR2-H consisting of SEQ ID NO:12076 or SEQ ID NO: 5014 and a CDR3-H consisting of SEQ ID NO:12077 or SEQ ID NO: 6014. In some embodiments, the ABP comprises a CDR1-L consisting of SEQ ID NO: 12004, a CDR2-L consisting of SEQ ID NO: 12014, a CDR3-L consisting of SEQ ID NO: 12024, a CDR1-H consisting of SEQ ID NO: 12039, a CDR2-H consisting of SEQ ID NO: 12049, and a CDR3-H consisting of SEQ ID NO: 12059. In some embodiments, the ABP comprises a CDR1-L consisting of SEQ ID NO: 12005, a CDR2-L consisting of SEQ ID NO: 12015, a CDR3-L consisting of SEQ ID NO: 12025, a CDR1-H consisting of SEQ ID NO: 12040, a CDR2-H consisting of SEQ ID NO: 12050, and a CDR3-H consisting of SEQ ID NO: 12060. In some embodiments, the ABP comprises a CDR1-L consisting of SEQ ID NO: 12006, a CDR2-L consisting of SEQ ID NO: 12016, a CDR3-L consisting of SEQ ID NO: 12026, a CDR1-H consisting of SEQ ID NO: 12041, a CDR2-H consisting of SEQ ID NO: 12051, and a CDR3-H consisting of SEQ ID NO: 12061. In some embodiments, the ABP comprises a CDR1-L consisting of SEQ ID NO: 12007, a CDR2-L consisting of SEQ ID NO: 12017, a CDR3-L consisting of SEQ ID NO: 12027, a CDR1-H consisting of SEQ ID NO: 12042, a CDR2-H consisting of SEQ ID NO: 12052, and a CDR3-H consisting of SEQ ID NO: 12062. In some embodiments, the ABP comprises a CDR1-L consisting of SEQ ID NO: 12008, a CDR2-L consisting of SEQ ID NO: 12018, a CDR3-L consisting of SEQ ID NO: 12028, a CDR1-H consisting of SEQ ID NO: 12043, a CDR2-H consisting of SEQ ID NO: 12053, and a CDR3-H consisting of SEQ ID NO: 12063.

In some embodiments, the ABP comprises a variable light chain (V_{L}) comprising a sequence at least 97% identical to SEQ ID NO:14 and a variable heavy chain (V_{H}) comprising a sequence at least 97% identical to SEQ ID NO:114.

In some embodiments, the ABP comprises an scFv or a full length monoclonal antibody. In some embodiments, the ABP comprises an immunoglobulin constant region.

In some embodiments, the ABP binds human CTLA-4 with a K_{D} of less than 500 nM, as measured by surface plasmon resonance; or the ABP binds human CTLA-4 with a K_{D} of less than 200 nM, as measured by surface plasmon resonance; or the ABP binds human CTLA-4 with a K_{D} of less than 25 nM, as measured by surface plasmon resonance; or the ABP binds to human CTLA-4 on a cell surface with a K_{D} of less than 25 nM.

In some embodiments, the ABP is a IgG1 ABP. In some embodiments, the ABP comprises an IGHG1*01 human heavy chain constant region gene segment. In some embodiments, the ABP comprises a lysine at amino acid position 97 (R97) according to IMGT exon numbering. In some embodiments, the ABP comprises a lysine at amino acid position 97 (R214) according to EU numbering.

In some embodiments, the ABP comprises an afucosylated Fc region.

In some embodiments, the ABP is produced from a cell comprising a bacterial protein RMD (GDP-6-deoxy-D-lyxo-4-hexulose reductase) or a modification thereof. In some embodiments, the cell is cultured in the absence of fucose.

In some embodiments, the ABP is produced from a cell lacking or reduced expression of Fut8. In some embodiments, the ABP is produced from a cell cultured in the presence of a fucosylation inhibitor, 2-Fluorfucose (2FF). In some embodiments, the ABP is produced from a cell overexpressing glycosyltransferase (GnTIII). In some embodiments, the ABP has been isolated based on its fucosylation status.

In some embodiments, the ABP comprises an Fc region lacking core fucosylation of the N-glycan of the Fc portion. In some embodiments, the ABP is an afucosylated monoclonal antibody.

Aspects of the present disclosure also include a pharmaceutical composition comprising the ABP of the present disclosure and a pharmaceutically acceptable excipient.

In some embodiments, less than 50% of the ABP is fucosylated. In some embodiments, less than 40% of the ABP is fucosylated. In some embodiments, less than 30% of the ABP is fucosylated. In some embodiments, less than 20% of the ABP is fucosylated. In some embodiments, less than 10% of the ABP is fucosylated. In some embodiments, more than 30% of the ABP is fucosylated. In some embodiments, more than 40% of the ABP is fucosylated. In some embodiments, more than 50% of the ABP is fucosylated. In some embodiments, more than 60% of the ABP is fucosylated. In some embodiments, more than 70% of the ABP is fucosylated. In some embodiments, more than 80% of the ABP is fucosylated. In some embodiments, more than 90% of the ABP is fucosylated.

In some embodiments, the pharmaceutical composition has a pH from 5.0 to 6.5. In some embodiments, the pharmaceutical composition comprises 20 mM of histidine or citrate buffer. In some embodiments, the pharmaceutical composition comprises 50 mM of NaCl. In some embodiments, the pharmaceutical composition comprises sucrose at a concentration from 170 mM to 270 mM. In some embodiments, the pharmaceutical composition comprises 170 mM or 270 mM of sucrose. In some embodiments, the pharmaceutical composition comprises 5 mg/mL to 20 mg/mL of the ABP. In some embodiments, the pharmaceutical composition comprises 20 mg/mL of the ABP. In some embodiments, the pharmaceutical composition comprises 5 mg/mL of the ABP.

Aspects of the present disclosure provide a method of treating a disease comprising the step of: administering to a subject in need thereof an effective amount of the ABP of any of the ABPs of the present disclosure or the pharmaceutical composition thereof.

In some embodiments, the disease is selected from the group consisting of cancer, AIDS, Alzheimer's disease and viral or bacterial infection.

In some embodiments, further comprising the step of administering one or more additional therapeutic agents to the subject. In some embodiments, additional therapeutic agent is selected from an anti-PD-L1, an anti-PD1, a LAG-3 inhibitor, aCD47 inhibitor, a TIGIT inhibitor, a chemotherapy agent, an immune-stimulatory agent, radiation, a BRAF inhibitor, a MEK inhibitor, a PI3K inhibitor, a cytokine, a polynucleotide encoding a cytokine, an oncolytic virus encoding a cytokine, and a combination thereof.

Aspects of the present disclosure include an isolated polynucleotide encoding the ABP. Aspects of the present disclosure include a vector comprising the isolated polynucleotide. Aspects of the present disclosure provide a host cell comprising the isolated polynucleotide or the vector of the present disclosure. In some embodiments, the host cell further comprises a bacterial protein RMD (GDP-6-deoxy-D-lyxo-4-hexulose reductase). In some embodiments, the host cell is cultured in the absence of fucose. In some embodiments, the host cell is lacking or having reduced expression of Fut8. In some embodiments, the host cell is cultured in the presence of a fucosylation inhibitor 2-Fluorfucose (2FF). In some embodiments, the host cell is overexpressing glycosyltransferase (GnTIII).

Aspects of the present disclosure provide a method of producing an isolated antigen binding protein (ABP) that specifically binds human CTLA-4, comprising inducing expression of the ABP in the host cell of the present disclosure, and isolating the ABP.

In some embodiments, further comprising the step of isolating the ABP based on its fucosylation status. In some embodiments, the host cell is cultured in a cultured medium comprising a fucosylation inhibitor. In some embodiments, the fucosylation inhibitor is 2-Fluorfucose (2FF).

A method of reducing CTLA-4^{HI} Tregs in a subject with limited proliferation of remaining Tregs comprising administering an effective dose of the ABP or the pharmaceutical composition described in the present disclosure.

In some embodiments, the subject is a human subject, optionally, a human subject with RCC (renal cell cancer), NSCLC (non-small cell lung cancer), Merkel cell carcinoma, cSCC, mesothelioma, MSI colorectal cancer, ovarian cancer, or cervical cancer.

In some embodiments, further comprising the step of administering one or more additional therapeutic agents to the subject. In some embodiments, the additional therapeutic agent is an anti-PD-L1 or an anti-PD1 or a combination thereof.

In another aspect, the present disclosure provides an isolated antigen binding protein (ABP) that specifically binds a human cytotoxic T-lymphocyte associated protein 4 (CTLA-4), wherein the ABP comprises: (a) a CDR1-L consisting of SEQ ID NO:12078, a CDR2-L consisting of SEQ ID NO: 12079, a CDR3-L consisting of SEQ ID NO:12080, a CDR1-H consisting of SEQ ID NO:12075, a CDR2-H consisting of SEQ ID NO:12076 and a CDR3-H consisting of SEQ ID NO:12077; (b) a CDR1-L consisting of SEQ ID NO: 1014, a CDR2-L consisting of SEQ ID NO: 2014, a CDR3-L consisting of SEQ ID NO: 3014, a CDR1-H consisting of SEQ ID NO: 4014, a CDR2-H consisting of SEQ ID NO: 5014 and a CDR3-H consisting of SEQ ID NO: 6014; (c) a CDR1-L consisting of SEQ ID NO: 12004, a CDR2-L consisting of SEQ ID NO: 12014, a CDR3-L consisting of SEQ ID NO: 12024, a CDR1-H consisting of SEQ ID NO: 12039, a CDR2-H consisting of SEQ ID NO: 12049, and a CDR3-H consisting of SEQ ID NO: 12059; (d) a CDR1-L consisting of SEQ ID NO: 12005, a CDR2-L consisting of SEQ ID NO: 12015, a CDR3-L consisting of SEQ ID NO: 12025, a CDR1-H consisting of SEQ ID NO: 12040, a CDR2-H consisting of SEQ ID NO: 12050, and a CDR3-H consisting of SEQ ID NO: 12060; (e) a CDR1-L consisting of SEQ ID NO: 12006, a CDR2-L consisting of SEQ ID NO: 12016, a CDR3-L consisting of SEQ ID NO: 12026, a CDR1-H consisting of SEQ ID NO: 12041, a CDR2-H consisting of SEQ ID NO: 12051, and a CDR3-H consisting of SEQ ID NO: 12061; (f) a CDR1-L consisting of SEQ ID NO: 12007, a CDR2-L consisting of SEQ ID NO: 12017, a CDR3-L consisting of SEQ ID NO: 12027, a CDR1-H consisting of SEQ ID NO: 12042, a CDR2-H consisting of SEQ ID NO: 12052, and a CDR3-H consisting of SEQ ID NO: 12062; or (g) a CDR1-L consisting of SEQ ID NO: 12008, a CDR2-L consisting of SEQ ID NO: 12018, a CDR3-L consisting of SEQ ID NO: 12028, a CDR1-H consisting of SEQ ID NO: 12043, a CDR2-H consisting of SEQ ID NO: 12053, and a CDR3-H consisting of SEQ ID NO: 12063.

In some embodiments, the ABP comprises a variable light chain (V_{L}) comprising a sequence at least 97% identical to SEQ ID NO:14 and a variable heavy chain (V_{H}) comprising a sequence at least 97% identical to SEQ ID NO: 114.

In some embodiments, the ABP comprises an scFv or a full length monoclonal antibody. In some embodiments, the ABP comprises an immunoglobulin constant region.

In some embodiments, the ABP binds human CTLA-4 with a K_{D} of less than 500 nM, as measured by surface plasmon resonance; or the ABP binds human CTLA-4 with a K_{D} of less than 200 nM, as measured by surface plasmon resonance; or the ABP binds human CTLA-4 with a K_{D} of less than 25 nM, as measured by surface plasmon resonance; or the ABP binds to human CTLA-4 on a cell surface with a K_{D} of less than 25 nM.

In some embodiments, the ABP is a IgG1 ABP. In some embodiments, the ABP comprises an IGHG1*01 human heavy chain constant region gene segment. In some embodiments, the ABP comprises a lysine at amino acid position 97 (R97) according to IMGT exon numbering. In some embodiments, the ABP comprises a lysine at amino acid position 97 (R214) according to EU numbering.

In some embodiments, the ABP comprises an afucosylated Fc region.

In some embodiments, the ABP is produced from a cell comprising a bacterial protein RMD (GDP-6-deoxy-D-lyxo-4-hexulose reductase) or a modification thereof. In some embodiments, the cell is cultured in the absence of fucose. In some embodiments, the ABP is produced from a cell lacking or reduced expression of Fut8. In some embodiments, the ABP is produced from a cell cultured in the presence of a fucosylation inhibitor, 2-Fluorfucose (2FF). In some embodiments, the ABP is produced from a cell overexpressing glycosyltransferase (GnTIII). In some embodiments, the ABP has been isolated based on its fucosylation status.

In some embodiments, the ABP comprising an Fc region lacking core fucosylation of the N-glycan of the Fc portion. In some embodiments, the ABP is an afucosylated monoclonal antibody. In some embodiments, the afucosylated Fc region has less than 30% fucosylation, and wherein less than 30% fucosylation enhances FcgRIII (Fc Gamma Receptor III) signaling. In some embodiments, the afucosylated Fc region has less than 30% fucosylation, and wherein less than 30% fucosylation enhances FcgRIIIa (Fc Gamma Receptor IIIa) signaling. In some embodiments, the afucosylated Fc region has less than 30% fucosylation, and wherein less than 30% fucosylation enhances a protein encoded by protein encoded by FcgR3a.

Aspects of the present disclosure include a pharmaceutical composition comprising the ABP of the present disclosure, and a pharmaceutically acceptable excipient.

In some embodiments, less than 50% of the ABP is fucosylated. In some embodiments, less than 40% of the ABP is fucosylated. In some embodiments, less than 30% of the ABP is fucosylated. In some embodiments, less than 20% of the ABP is fucosylated. In some embodiments, less than 10% of the ABP is fucosylated. In some embodiments, more than 30% of the ABP is fucosylated. In some embodiments, more than 40% of the ABP is fucosylated. In some embodiments, more than 50% of the ABP is fucosylated. In some embodiments, more than 60% of the ABP is fucosylated. In some embodiments, more than 70% of the ABP is fucosylated. In some embodiments, more than 80% of the ABP is fucosylated. In some embodiments, more than 90% of the ABP is fucosylated.

In some embodiments, the pharmaceutical composition has a pH from 5.0 to 6.5. In some embodiments, the pharmaceutical composition comprises 20 mM of histidine or citrate buffer. In some embodiments, the pharmaceutical composition comprises 50 mM of NaCl. In some embodiments, the pharmaceutical composition comprises sucrose at a concentration from 170 mM to 270 mM. In some embodiments, the pharmaceutical composition comprises 170 mM or 270 mM of sucrose. In some embodiments, the pharmaceutical composition comprises 5 mg/mL to 20 mg/mL of the ABP. In some embodiments, the pharmaceutical composition comprises 20 mg/mL of the ABP. In some embodiments, the pharmaceutical composition comprises 5 mg/mL of the ABP.

Aspects of the present disclosure provide a method of treating a disease comprising the step of: administering to a subject in need thereof an effective amount of the ABP of any of the ABPs of the present disclosure or the pharmaceutical composition thereof.

In some embodiments, the disease is selected from the group consisting of cancer, AIDS, Alzheimer's disease and viral or bacterial infection.

In some embodiments, further comprising the step of administering one or more additional therapeutic agents to the subject. In some embodiments, additional therapeutic agent is selected from an anti-PD-L1, an anti-PD1, a LAG-3 inhibitor, aCD47 inhibitor, a TIGIT inhibitor, a chemotherapy agent, an immune-stimulatory agent, radiation, a BRAF inhibitor, a MEK inhibitor, a PI3K inhibitor, a cytokine, a polynucleotide encoding a cytokine, an oncolytic virus encoding a cytokine, and a combination thereof.

Aspects of the present disclosure include an isolated polynucleotide encoding the ABP. Aspects of the present disclosure include a vector comprising the isolated polynucleotide. Aspects of the present disclosure provide a host cell comprising the isolated polynucleotide or the vector of the present disclosure. In some embodiments, the host cell further comprises a bacterial protein RMD (GDP-6-deoxy-D-lyxo-4-hexulose reductase). In some embodiments, the host cell is cultured in the absence of fucose. In some embodiments, the host cell is lacking or having reduced expression of Fut8. In some embodiments, the host cell is cultured in the presence of a fucosylation inhibitor 2-Fluorfucose (2FF). In some embodiments, the host cell is overexpressing glycosyltransferase (GnTIII).

Aspects of the present disclosure provide a method of treating cancer comprising the step of administering to a subject in need thereof an effective amount of the ABP or the pharmaceutical composition of the present disclosure. In some embodiments, the subject has a malignant tumor. In some embodiments, when administered, comprises increased Fc receptor (FcR) signaling as compared to ipilimumab, and wherein said administering reduces the amount of CTLA-4^{HI} Tregs in the subject. In some embodiments, said administering reduces proliferation of peripheral Tregs in the subject as compared to ipilimumab.

In some embodiments, further comprising the step of administering one or more additional therapeutic agents to the subject. In some embodiments, the additional therapeutic agent is selected from an anti-PD-L1, an anti-PDT, a TIGIT inhibitor, a LAG-3 inhibitor, a CD47 inhibitor, a BRAF inhibitor, a MEK inhibitor, a PI3K inhibitor, a chemotherapy agent, an immune-stimulatory agent, radiation, a cytokine, a polynucleotide encoding a cytokine, an oncolytic virus encoding a cytokine, and a combination thereof.

In some embodiments, the ABP comprises an afucosylated Fc region that has less than 30% fucosylation, and wherein less than 30% fucosylation enhances FcgRIII signaling. In some embodiments, the ABP comprises an afucosylated Fc region that has less than 30% fucosylation, and wherein less than 30% fucosylation enhances FcgRIIIa signaling. In some embodiments, the ABP comprises a fucosylated Fc region that has more than 70% fucosylation. In some embodiments, the afucosylated Fc region has less than 30% fucosylation, and wherein less than 30% fucosylation enhances a protein encoded by protein encoded by FcgR3a.

Aspects of the present disclosure include an isolated polynucleotide encoding the ABP. Aspects of the present disclosure include a vector comprising the isolated polynucleotide. Aspects of the present disclosure provide a host cell comprising the isolated polynucleotide or the vector of the present disclosure. In some embodiments, the host cell further comprises a bacterial protein RMD (GDP-6-deoxy-D-lyxo-4-hexulose reductase). In some embodiments, the host cell is cultured in the absence of fucose. In some embodiments, the host cell is lacking or having reduced expression of Fut8. In some embodiments, the host cell is cultured in the presence of a fucosylation inhibitor 2-Fluorfucose (2FF). In some embodiments, the host cell is overexpressing glycosyltransferase (GnTIII).

Aspects of the present disclosure provide a method of producing an isolated antigen binding protein (ABP) that specifically binds human CTLA-4, comprising inducing expression of the ABP in the host cell of the present disclosure, and isolating the ABP, wherein the ABP comprises an afucosylated Fc.

In some embodiments, further comprising the step of isolating the ABP based on its fucosylation status. In some embodiments, the host cell is cultured in a cultured medium comprising a fucosylation inhibitor. In some embodiments, the fucosylation inhibitor is 2-Fluorfucose (2FF).

In another aspect, the present disclosure provides an isolated antigen binding protein (ABP) that specifically binds a human cytotoxic T-lymphocyte associated protein 4 (CTLA-4), comprising an IGHG1*01 human heavy chain constant region gene segment.

In some embodiments, the ABP comprises: (a) a CDR1-L consisting of SEQ ID NO:12078, a CDR2-L consisting of SEQ ID NO: 12079, a CDR3-L consisting of SEQ ID NO:12080, a CDR1-H consisting of SEQ ID NO:12075, a CDR2-H consisting of SEQ ID NO:12076 and a CDR3-H consisting of SEQ ID NO: 12077; (b) a CDR1-L consisting of SEQ ID NO: 1014, a CDR2-L consisting of SEQ ID NO: 2014, a CDR3-L consisting of SEQ ID NO: 3014, a CDR1-H consisting of SEQ ID NO: 4014, a CDR2-H consisting of SEQ ID NO: 5014 and a CDR3-H consisting of SEQ ID NO: 6014; (c) a CDR1-L consisting of SEQ ID NO: 12004, a CDR2-L consisting of SEQ ID NO: 12014, a CDR3-L consisting of SEQ ID NO: 12024, a CDR1-H consisting of SEQ ID NO: 12039, a CDR2-H consisting of SEQ ID NO: 12049, and a CDR3-H consisting of SEQ ID NO: 12059; (d) a CDR1-L consisting of SEQ ID NO: 12005, a CDR2-L consisting of SEQ ID NO: 12015, a CDR3-L consisting of SEQ ID NO: 12025, a CDR1-H consisting of SEQ ID NO: 12040, a CDR2-H consisting of SEQ ID NO: 12050, and a CDR3-H consisting of SEQ ID NO: 12060; (e) a CDR1-L consisting of SEQ ID NO: 12006, a CDR2-L consisting of SEQ ID NO: 12016, a CDR3-L consisting of SEQ ID NO: 12026, a CDR1-H consisting of SEQ ID NO: 12041, a CDR2-H consisting of SEQ ID NO: 12051, and a CDR3-H consisting of SEQ ID NO: 12061; (f) a CDR1-L consisting of SEQ ID NO: 12007, a CDR2-L consisting of SEQ ID NO: 12017, a CDR3-L consisting of SEQ ID NO: 12027, a CDR1-H consisting of SEQ ID NO: 12042, a CDR2-H consisting of SEQ ID NO: 12052, and a CDR3-H consisting of SEQ ID NO: 12062; or (g) a CDR1-L consisting of SEQ ID NO: 12008, a CDR2-L consisting of SEQ ID NO: 12018, a CDR3-L consisting of SEQ ID NO: 12028, a CDR1-H consisting of SEQ ID NO: 12043, a CDR2-H consisting of SEQ ID NO: 12053, and a CDR3-H consisting of SEQ ID NO: 12063. In some embodiments, the ABP comprises a variable light chain (V_{L}) comprising a sequence at least 97% identical to SEQ ID NO:14 and a variable heavy chain (V_{H}) comprising a sequence at least 97% identical to SEQ ID NO:114.

In some embodiments, the ABP comprises: (a) a CDR1-L consisting of any one of SEQ ID NOs:, a CDR2-L consisting of any one of SEQ ID NOs: 1001-1028, a CDR3-L consisting of any one of SEQ ID NOs: 2001-2028, a CDR1-H consisting of any one of SEQ ID NOs:, a CDR2-H consisting of consisting of any one of SEQ ID NOs: 3001-3028. In some embodiments, the ABP comprises a variable light chain (V_{L}) comprising a sequence at least 97% identical to any one of SEQ ID NOs:1-28 and a variable heavy chain (V_{H}) comprising a sequence at least 97% identical to SEQ ID NO:1-128.

In some embodiments, the ABP comprises a lysine at amino acid position 97 (R97) according to IMGT exon numbering. In some embodiments, the ABP comprises a lysine at amino acid position 97 (R214) according to EU numbering. In some embodiments, comprising an afucosylated Fc region. In some embodiments, produced from a cell comprising a bacterial protein RMD (GDP-6-deoxy-D-lyxo-4-hexulose reductase) or a modification thereof. In some embodiments, the cell is cultured in the absence of fucose. In some embodiments, produced from a cell lacking or reduced expression of Fut8. In some embodiments, produced from a cell cultured in the presence of a fucosylation inhibitor, 2-Fluorfucose (2FF). In some embodiments, produced from a cell overexpressing glycosyltransferase (GnTIII). In some embodiments, having been isolated based on its fucosylation status. In some embodiments, comprising an Fc region lacking core fucosylation of the N-glycan of the Fc portion. In some embodiments, the ABP is an afucosylated monoclonal antibody.

Aspects of the present disclosure include a pharmaceutical composition comprising the ABP of the present disclosure, and a pharmaceutically acceptable excipient.

In some embodiments, less than 50% of the ABP is fucosylated. In some embodiments, less than 40% of the ABP is fucosylated. In some embodiments, less than 30% of the ABP is fucosylated. In some embodiments, less than 20% of the ABP is fucosylated. In some embodiments, less than 10% of the ABP is fucosylated. In some embodiments, more than 30% of the ABP is fucosylated. In some embodiments, more than 40% of the ABP is fucosylated. In some embodiments, more than 50% of the ABP is fucosylated. In some embodiments, more than 60% of the ABP is fucosylated. In some embodiments, more than 70% of the ABP is fucosylated. In some embodiments, more than 80% of the ABP is fucosylated. In some embodiments, more than 90% of the ABP is fucosylated.

In some embodiments, the pharmaceutical composition has a pH from 5.0 to 6.5. In some embodiments, the pharmaceutical composition comprises 20 mM of histidine or citrate buffer. In some embodiments, the pharmaceutical composition comprises 50 mM of NaCl. In some embodiments, the pharmaceutical composition comprises sucrose at a concentration from 170 mM to 270 mM. In some embodiments, the pharmaceutical composition comprises 170 mM or 270 mM of sucrose. In some embodiments, the pharmaceutical composition comprises 5 mg/mL to 20 mg/mL of the ABP. In some embodiments, the pharmaceutical composition comprises 20 mg/mL of the ABP. In some embodiments, the pharmaceutical composition comprises 5 mg/mL of the ABP.

Aspects of the present disclosure provide a method of treating a disease comprising the step of administering to a subject in need thereof an effective amount of the ABP or the pharmaceutical composition.

In some embodiments, the disease is selected from the group consisting of cancer, AIDS, Alzheimer's disease and viral or bacterial infection.

In some embodiments, further comprising the step of administering one or more additional therapeutic agents to the subject. In some embodiments, the additional therapeutic agent is selected from an anti-PD-L1, an anti-PD1, a TIGIT inhibitor, a LAG-3 inhibitor, a CD47 inhibitor, a BRAF inhibitor, a MEK inhibitor, a PI3K inhibitor, a chemotherapy agent, an immune-stimulatory agent, radiation, a cytokine, a polynucleotide encoding a cytokine, an oncolytic virus encoding a cytokine, and a combination thereof.

Aspects of the present disclosure include an isolated polynucleotide encoding the ABP. Aspects of the present disclosure include a vector comprising the isolated polynucleotide. Aspects of the present disclosure provide a host cell comprising the isolated polynucleotide or the vector of the present disclosure. In some embodiments, the host cell further comprises a bacterial protein RMD (GDP-6-deoxy-D-lyxo-4-hexulose reductase). In some embodiments, the host cell is cultured in the absence of fucose. In some embodiments, the host cell is lacking or having reduced expression of Fut8. In some embodiments, the host cell is cultured in the presence of a fucosylation inhibitor 2-Fluorfucose (2FF). In some embodiments, the host cell is overexpressing glycosyltransferase (GnTIII).

Aspects of the present disclosure provide a method of producing an isolated antigen binding protein (ABP) that specifically binds human CTLA-4, comprising inducing expression of the ABP in the host cell, and isolating the ABP.

In some embodiments, further comprising the step of isolating the ABP based on its fucosylation status. In some embodiments, the host cell is cultured in a cultured medium comprising a fucosylation inhibitor. In some embodiments, the fucosylation inhibitor is 2-Fluorfucose (2FF).

Aspects of the present disclosure provide a method of reducing CTLA-4^{HI} Tregs in a subject with limited proliferation of remaining Tregs comprising administering an effective dose of the ABP or the pharmaceutical composition.

In some embodiments, the subject is a human subject, optionally, a human subject with melanoma, RCC (renal cell cancer), NSCLC (non-small cell lung cancer), Merkel cell carcinoma, cSCC, mesothelioma, MSI colorectal cancer, ovarian cancer, or cervical cancer. In some embodiments, further comprising the step of administering one or more additional therapeutic agents to the subject. In some embodiments, the additional therapeutic agent is an anti-PD-L1 or an anti-PD1 or a combination thereof. In some embodiments, the subject has a tumor with high levels of Tregs, high levels of CTLA-4, high levels of NK cells, or high levels of activating FcRs.

In another aspect, the present disclosure provides an isolated antigen binding protein (ABP) that specifically binds to an antigen, comprising an IGHG1*01 human heavy chain constant region gene segment.

In some embodiments, the ABP comprises a lysine at amino acid position 97 (R97) according to IMGT exon numbering. In some embodiments, the ABP comprises a lysine at amino acid position 97 (R214) according to EU numbering.

In some embodiments, comprising an afucosylated Fc region. In some embodiments, produced from a cell comprising a bacterial protein RMD (GDP-6-deoxy-D-lyxo-4-hexulose reductase) or a modification thereof. In some embodiments, the cell is cultured in the absence of fucose. In some embodiments, produced from a cell lacking or reduced expression of Fut8. In some embodiments, produced from a cell cultured in the presence of a fucosylation inhibitor, 2-Fluorfucose (2FF).

In some embodiments, produced from a cell overexpressing glycosyltransferase (GnTIII). In some embodiments, having been isolated based on its fucosylation status. In some embodiments, comprising an Fc region lacking core fucosylation of the N-glycan of the Fc portion. In some embodiments, the ABP is an afucosylated monoclonal antibody.

In some embodiments, the ABP is selected from an anti-CTLA-4 antibody or antigen-binding fragment thereof, anti-PD-L1 antibody or antigen-binding fragment thereof, an anti-PD1 antibody or antigen-binding fragment thereof, a TIGIT antibody or antigen-binding fragment thereof, a LAG-3 antibody or antigen-binding fragment thereof, a CD47 antibody or antigen-binding fragment thereof, a BRAF antibody or antigen-binding fragment thereof, a MEK antibody or antigen-binding fragment thereof, and a PI3K antibody or antigen-binding fragment thereof.

In some embodiments, the ABP comprises: (a) a CDR1-L consisting of SEQ ID NO:12078, a CDR2-L consisting of SEQ ID NO: 12079, a CDR3-L consisting of SEQ ID NO:12080, a CDR1-H consisting of SEQ ID NO:12075, a CDR2-H consisting of SEQ ID NO:12076 and a CDR3-H consisting of SEQ ID NO: 12077; (b) a CDR1-L consisting of SEQ ID NO: 1014, a CDR2-L consisting of SEQ ID NO: 2014, a CDR3-L consisting of SEQ ID NO: 3014, a CDR1-H consisting of SEQ ID NO: 4014, a CDR2-H consisting of SEQ ID NO: 5014 and a CDR3-H consisting of SEQ ID NO: 6014; (c) a CDR1-L consisting of SEQ ID NO: 12004, a CDR2-L consisting of SEQ ID NO: 12014, a CDR3-L consisting of SEQ ID NO: 12024, a CDR1-H consisting of SEQ ID NO: 12039, a CDR2-H consisting of SEQ ID NO: 12049, and a CDR3-H consisting of SEQ ID NO: 12059; (d) a CDR1-L consisting of SEQ ID NO: 12005, a CDR2-L consisting of SEQ ID NO: 12015, a CDR3-L consisting of SEQ ID NO: 12025, a CDR1-H consisting of SEQ ID NO: 12040, a CDR2-H consisting of SEQ ID NO: 12050, and a CDR3-H consisting of SEQ ID NO: 12060; (e) a CDR1-L consisting of SEQ ID NO: 12006, a CDR2-L consisting of SEQ ID NO: 12016, a CDR3-L consisting of SEQ ID NO: 12026, a CDR1-H consisting of SEQ ID NO: 12041, a CDR2-H consisting of SEQ ID NO: 12051, and a CDR3-H consisting of SEQ ID NO: 12061; (f) a CDR1-L consisting of SEQ ID NO: 12007, a CDR2-L consisting of SEQ ID NO: 12017, a CDR3-L consisting of SEQ ID NO: 12027, a CDR1-H consisting of SEQ ID NO: 12042, a CDR2-H consisting of SEQ ID NO: 12052, and a CDR3-H consisting of SEQ ID NO: 12062; or (g) a CDR1-L consisting of SEQ ID NO: 12008, a CDR2-L consisting of SEQ ID NO: 12018, a CDR3-L consisting of SEQ ID NO: 12028, a CDR1-H consisting of SEQ ID NO: 12043, a CDR2-H consisting of SEQ ID NO: 12053, and a CDR3-H consisting of SEQ ID NO: 12063.

In some embodiments, the ABP comprises a variable light chain (V_{L}) comprising a sequence at least 97% identical to SEQ ID NO:14 and a variable heavy chain (V_{H}) comprising a sequence at least 97% identical to SEQ ID NO:114.

In some embodiments, the ABP comprises: (a) a CDR1-L consisting of any one of SEQ ID NO: 12081:, a CDR2-L consisting of SEQ ID NO: 12082, a CDR3-L consisting of SEQ ID NO: 12083, a CDR1-H consisting of SEQ ID NO: 12084, a CDR2-H consisting of consisting of SEQ ID NO: 12085, and a CDR3-H consisting of SEQ ID NO: 12086.

In some embodiments, the ABP comprises a variable light chain (V_{L}) comprising a sequence at least 97% identical to SEQ ID NO: 12088 and a variable heavy chain (V_{H}) comprising a sequence at least 97% identical to SEQ ID NO: 12087.

In some embodiments, the pharmaceutical composition has a pH from 5.0 to 6.5. In some embodiments, the pharmaceutical composition comprises 20 mM of histidine or citrate buffer. In some embodiments, the pharmaceutical composition comprises 50 mM of NaCl. In some embodiments, the pharmaceutical composition comprises sucrose at a concentration from 170 mM to 270 mM. In some embodiments, the pharmaceutical composition comprises 170 mM or 270 mM of sucrose. In some embodiments, the pharmaceutical composition comprises 5 mg/mL to 20 mg/mL of the ABP. In some embodiments, the pharmaceutical composition comprises 20 mg/mL of the ABP. In some embodiments, the pharmaceutical composition comprises 5 mg/mL of the ABP.

Aspects of the present disclosure include a method of treating a disease comprising the step of: administering to a subject in need thereof an effective amount of the ABP or the pharmaceutical composition.

In some embodiments, the disease is selected from the group consisting of cancer, AIDS, Alzheimer's disease and viral or bacterial infection. In some embodiments, further comprising the step of administering one or more additional therapeutic agents to the subject.

In some embodiments, the additional therapeutic agent is selected from a chemotherapy agent, an immune-stimulatory agent, radiation, a cytokine, a polynucleotide encoding a cytokine, an oncolytic virus encoding a cytokine, and a combination thereof.

Aspects of the present disclosure include an isolated polynucleotide encoding the ABP. Aspects of the present disclosure include a vector comprising the isolated polynucleotide. Aspects of the present disclosure provide a host cell comprising the isolated polynucleotide or the vector of the present disclosure. In some embodiments, the host cell further comprises a bacterial protein RMD (GDP-6-deoxy-D-lyxo-4-hexulose reductase). In some embodiments, the host cell is cultured in the absence of fucose. In some embodiments, the host cell is lacking or having reduced expression of Fut8. In some embodiments, the host cell is cultured in the presence of a fucosylation inhibitor 2-Fluorfucose (2FF). In some embodiments, the host cell is overexpressing glycosyltransferase (GnTIII).

Aspects of the present disclosure provide a method of producing an isolated antigen binding protein (ABP) that specifically binds human CTLA-4, comprising inducing expression of the ABP in the host cell, and isolating the ABP.

In some embodiments, further comprising the step of isolating the ABP based on its fucosylation status. In some embodiments, the host cell is cultured in a cultured medium comprising a fucosylation inhibitor. In some embodiments, the fucosylation inhibitor is 2-Fluorfucose (2FF).

Aspects of the present disclosure provide a method of reducing CTLA-4^{HI} Tregs in a subject with limited proliferation of remaining Tregs comprising administering an effective dose of the ABP or the pharmaceutical composition.

In some embodiments, the subject is a human subject, optionally, a human subject with melanoma, RCC (renal cell cancer), NSCLC (non-small cell lung cancer), Merkel cell carcinoma, cSCC, mesothelioma, MSI colorectal cancer, ovarian cancer, or cervical cancer.

In some embodiments, further comprising the step of administering one or more additional therapeutic agents to the subject. In some embodiments, the subject has a tumor with high levels of Tregs, high levels of CTLA-4, high levels of NK cells, or high levels of activating FcRs.

Aspects of the present disclosure provide a method of reducing CTLA-4^{HI} Tregs in a subject with limited proliferation of remaining Tregs comprising administering to the subject an effective dose of an antigen binding protein (ABP) that specifically binds a human cytotoxic T-lymphocyte associated protein 4 (CTLA-4).

In some embodiments, the subject is a human subject, optionally, a human subject with melanoma, RCC (renal cell cancer), NSCLC (non-small cell lung cancer), Merkel cell carcinoma, cSCC, mesothelioma, MSI colorectal cancer, ovarian cancer, or cervical cancer.

In some embodiments, further comprising the step of administering one or more additional therapeutic agents to the subject.

In some embodiments, the ABP comprises a variable light chain (V_{L}) comprising a sequence at least 97% identical to SEQ ID NO:14 and a variable heavy chain (V_{H}) comprising a sequence at least 97% identical to SEQ ID NO:114.

In some embodiments, the ABP comprises: (a) a CDR1-L consisting of any one of SEQ ID NOs:, a CDR2-L consisting of any one of SEQ ID NOs: 1001-1028, a CDR3-L consisting of any one of SEQ ID NOs: 3001-3028, a CDR1-H consisting of any one of SEQ ID NOs:, a CDR2-H consisting of consisting of any one of SEQ ID NOs: 3001-3028.

In some embodiments, the ABP comprises a variable light chain (V_{L}) comprising a sequence at least 97% identical to any one of SEQ ID NOs:1-28 and a variable heavy chain (V_{H}) comprising a sequence at least 97% identical to SEQ ID NO: 101-128.

### 6. BRIEF DESCRIPTION OF THE DRAWINGS

FIG. 1 summarizes the method of generating scFv libraries from B cells isolated from fully human mice and selecting for a yeast expressing an scFv having affinity to the antigen derived from a B cell expressing an antibody having affinity to the antigen. FIG. 1 discloses SEQ ID NOS 11971-11998, respectively, in order of appearance.

FIG. 2 illustrates scFv amplification procedure. First, a mixture of primers directed against the IgK C region, the IgG C region, and all V regions is used to separately amplify IgK and IgH. Second, the V-H and C-K primers contain a region of complementarity that results in the formation of an overlap extension amplicon that is a fusion product between IgK and IgH. The region of complementarity comprises a DNA sequence that encodes a Gly-Ser rich scFv linker sequence. Third, semi-nested PCR is performed to add adapters for Illumina sequencing or yeast display.

FIG. 3 includes a schematic for the monoclonal antibodies sorted in their epitope bins.

FIG. 4A-4E include plots from the histopathological staining of hCTLA-4 KI mice bearing MC38 tumors treated with PBS or an anti-CTLA-1 ABP. The plots show scoring of H&E (FIG. 4A), immunoglobulin (Ig) (FIG. 4B and FIG. 4C), and C3 stains (FIG. 4D and FIG. 4E) from the right kidney. ipi is Ipilimumab, and CTLA4. A14.2a is antibody A14 cloned onto a mouse IgG2a backbone.

FIG. 5 includes a plot showing the alkaline phosphatase levels in treated hCTLA4 KI mice bearing MC38 tumors. IPI is Ipilimumab, and CTLA4. A14.2a is antibody A14 cloned onto a mouse IgG2a backbone. U/L is units per liter.

FIG. 6 includes plots for the percentages of intratumoral regulatory T cells (Treg) cells and intratumoral natural killer (NK) cells after the indicated treatments.

FIG. 7 includes a plot showing the changes in body weight of the hCTLA4 mice receiving the indicated treatments. Ipi is Ipilimumab, and CTLA4. A14.2a is antibody A14 cloned onto a mouse IgG2a backbone. Error bars represent+/−standard error of the mean.

FIG. 8A-8F include plots showing the influence of the control, Ipi and the anti-CTLA4 treatments on percentage of the indicated cell populations, including CD3+ cells (FIG. 8A), CD4+ cells (FIG. 8B), CD69+ cells (FIG. 8C), ICOS+ cells (FIG. 8D), PD1+ cells (FIG. 8E), and FOXP3+ cells (FIG. 8F). Ipi is Ipilimumab, and CTLA4. A14.2a is antibody A14 cloned onto a mouse IgG2a backbone.

FIG. 9A-9D include plots showing the influence of the control, Ipi and the anti-CTLA4 treatments on percentage of the indicated cell populations, including CD8+ cells (FIG. 9A), CD69+ cells (FIG. 9B), ICOS+ cells (FIG. 9C) and PD1+ cells (FIG. 9D). Ipi is Ipilimumab, and CTLA4. A14.2a is antibody A14 cloned onto a mouse IgG2a backbone.

FIG. 10 includes plots showing the influence of the control, Ipi and the anti-CTLA4 treatments on percentage of dendritic cells (DCs) and activated dendritic cells (CD86+). Ipi is Ipilimumab, and CTLA4. A14.2a is antibody A14 cloned onto a mouse IgG2a backbone.

FIG. 11 includes a plot showing the mean tumor volume after treatment with 0.3 mg/kg of the indicated anti-CTLA4s.

FIG. 12A-12B shows that FcR effector function is required for the anti-tumor efficacy of ipilimumab in a murine model. FIG. 12A. The plot shows the frequency of CD4+FOXP3− T cells from the LN of individual control (C57BL/6) or hCTLA4-KI mice that are CD44LoCD62L+ as determined by flow cytometry (mean+/−SEM). FIG. 12B. hCTLA-4 KI mice bearing MC38 tumors were randomized when tumors were 50-151 mm3 (day 0) and treated bi-weekly with the indicated antibody at 5 mg/kg for 5 doses. The plot shows the tumor volume (mean+SEM) of MC38 tumors over time that were treated with the indicated antibody. Tumor volume from mice euthanized due to tumor burden above 3000 mm³ was carried forward. Thin vertical lines indicate censored data: Thin gray vertical lines indicate animals likely lost post dosing due to anti-drug antibody (ADA)-induced hypersensitivity, thin black vertical lines refer to animals euthanized due to tumor burden and for which the data was carried forward. Ipi analog indicates an ipilimumab mAb produced and purified by Applicant; N297Q indicates that the antibody contains an N297Q mutation in the Fc domain of the antibody to abrogate Fc effector function. n=8 for isotype, aCTLA-4.28, Ipi-N297Q; n=7 for ipi analog and aCTLA-4.28. p=0.0004 when comparing Ipi analog to isotype and p=0.0006 when comparing aCTLA-4.28 to isotype for change in tumor volume between groups (linear mixed effects model).

FIG. 13A-13D show that GIGA-564 has little ability to block the interaction of CTLA-4 and CD80/CD86 in vitro. FIG. 13A. The ability of CTLA-4 mAbs to block binding of CD80 or CD86 was measured using a plate-based ELISA method. CTLA-4 was used to coat the plate, then after the antibody samples were incubated, His-tagged CD80 or CD86 was added, and the amount of ligand able to bind to CTLA-4 was measured. Blocking mAbs that prevent CD80 or CD86 from binding to CTLA-4 reduce the absorbance signal due to lack of CD80 or CD86 binding to CTLA-4. Weak-blocking mAbs still allow CD80 or CD86 to bind, preventing the loss of all absorbance signal. FIG. 13B. Plots show the ability of GIGA-564 to block the interaction between CTLA-4 and the B7 ligands CD80 and CD86 compared to ipilimumab and CTLA-4.28, as assessed by ELISA as described in (FIG. 13A). Absorbance values were normalized to an anti-PD-1 control (pembrolizumab) and displayed as the average of two technical replicates. FIG. 13C-13D. The key residues mediating CTLA-4 binding were identified for GIGA-564 and ipilimumab by shotgun mutagenesis of CTLA-4, followed by staining and flow cytometry assessment of binding. FIG. 13C. Shown is the crystal structure (Protein Database [PDB] 1I8L) of the complex between CD80 (blue) and CTLA-4 (gray) on which the CTLA-4 epitope residues shared between ipilimumab and GIGA-564 were colored orange and the key differentiating residue R70 was colored red (visualized with Pymol). Additionally, G142 was identified as a secondary residue for the epitope of ipilimumab but not GIGA-564. FIG. 13D. Table showing key amino acids on CTLA-4 of interest for these epitopes; those found by mutational analysis to be important for binding of CTLA-4 to CD80 or CD86 in a cell-based assay are marked in gray to indicate the epitope residues for those proteins.

FIG. 14A-14C show that GIGA-564 inhibits tumor growth in murine models. FIG. 14A. hCTLA-4 KI mice bearing MC38 tumors were randomized when tumors were 50-150 mm³ (day 0) and treated bi-weekly with the indicated antibody at 5 mg/kg for 5 doses. Plots show the tumor volume (mean+SEM) of MC38 tumors over time that were treated with the indicated antibody. Tumor volume from mice euthanized due to tumor burden above 3000 mm³ was carried forward. Thin gray vertical lines indicate censored data (animals likely lost post dosing due to ADA-induced hypersensitivity). This experiment is also described in FIG. 12B. n=7 for GIGA-564 and n=6 for vehicle and commercial ipilimumab. FIG. 14B. hCTLA-4 KI mice bearing RM-1 tumors were randomized when tumors were 40-125 mm³ (day 0) and treated on day 0, 3, and 6 with 5 mg/kg of the indicated antibody. Plots show the tumor volume (mean±SEM) of RM-1 tumors over time that were treated with the indicated antibody. Tumor volume from mice euthanized due to tumor burden above 3000 mm³ was carried forward until no mice from that group were alive (thin black vertical lines). Thin vertical lines indicate censored data. The thin gray vertical line indicates one mouse from the ipilimumab treated group was euthanized due to tumor ulceration. n=11 for ipilimumab and GIGA-564 treatment and n=7 for isotype treatment. FIG. 14C (as is also shown in FIG. 11). hCTLA-4 KI mice bearing MC38 tumors were randomized when tumors were 65-125 mm³ (day 0) and treated on days 0, 3, and 6 with 0.3 mg/kg of the indicated antibody. Plots show the tumor volume (mean±SEM) of MC38 tumors over time that were treated with the indicated antibody. Tumor volume from mice euthanized due to tumor burden above 3000 mm³ was carried forward until no mice from that group remained alive (thin black vertical lines). n=13 for ipilimumab and GIGA-564 and n=8 for isotype treated animals. P-values are shown for statistically significant differences in changes in tumor volume measured longitudinally (linear mixed effects model).

FIG. 15A-15E shows that GIGA-564 induces less peripheral Treg proliferation but potently mediates depletion of intratumoral Tregs. FIG. 15A. hCTLA-4 KI mice (n=12) were treated with 5 mg/kg hIgG1 isotype control, ipilimumab, or GIGA-564 on days 0, 3, and 6, and euthanized for flow cytometry analysis on day 7. Two samples in the GIGA-564 group were excluded from analysis due to low cell count. The percent of CD8 T cells (Live, CD45⁺TCRβ⁺CD8⁺), CD4 Tconv (Live, CD45⁺TCRβ⁺CD4⁺FOXP3⁻), and Tregs (Live, CD45⁺TCRβ⁺CD4⁺FOXP3⁺) within the non-draining (left anterior axillary) lymph node (LN) expressing Ki67 was determined by flow cytometry (first 3 panels). The right panel shows the fold-change in the percent of cells of each subtype expressing Ki67 in ipilimumab or GIGA-564 treated mice relative to the mean frequency of that cell type expressing in the isotype control treated group. In this right panel, p-values were calculated using the Mann-Whitney (Wilcoxon) test without adjustment for multiple pairwise comparison. Fewer proliferating Tregs may further enhance efficacy as compared to ipilimumab in patients. FIG. 15B-15E. hCTLA-4 KI mice bearing established MC38 tumors were randomized (n=6) and treated once with 5 mg/kg hIgG1 isotype control, ipilimumab, or GIGA-564 and cells from the non-draining LN and tumor were analyzed the following day by flow cytometry. FIG. 15B. Frequency of Tregs (as percentage of CD45⁺ cells) in LN (left) and tumor (right) in each treatment group. FIG. 15C. CTLA-4 geometric mean fluorescence intensity (MFI) in Tregs in LN (left) and tumor (right). FIG. 15D. Representative contour plots of CD4 T cells in each treatment group. X-axis and Y-axis correspond to FOXP3 and CTLA-4, respectively. FIG. 15E. Ratio of CD8 T cells relative to Tregs in LN (left) and tumor (right). Unless otherwise indicated, p-values were calculated using Wilcoxon rank sum test and horizontal lines indicate means. Flow cytometry showed that GIGA-564 is more efficient than ipilimumab at depleting CTLA-4⁺ Tregs in the tumor microenvironment. Data from FIG. 15D is reshown in FIG. 32.

FIGS. 16A-16D shows that GIGA-564 induces more FcR signaling than ipilimumab. FIGS. 16A-16D. Target CHO cells expressing human CTLA-4 with the Y201G mutation to enhance surface expression were incubated with a titration series of ipilimumab (black squares), GIGA-564 (black stars), or a variant of GIGA-564 with LALA-PG mutations to disrupt FcγR binding (GIGA-564_LALA-PG, gray circles). Jurkat/NFAT-Luc effector cells with (FIG. 16A) mouse FcγRIV or FcγRIII, (FIG. 16B) human FcγRIIIa (high-affinity V158 or low-affinity F158 variant), (FIG. 16C) human FcγRIIa (high-affinity H131 or low-affinity R131 variant), or (FIG. 16D) human FcγRIIb were then added. Cells were incubated for 6 hours at 37° C. then luciferase activity was measured. Data shown are relative luminescence units (RLU) emitting from the effectors cells and are plotted as the average of two technical replicates.

FIGS. 17A-17B shows GIGA-564 provides protection in a murine tumor re-challenge model. FIG. 17A. hCTLA-4 KI mice bearing MC38 tumors were randomized when tumors were 60-120 mm³ (day 8) and then treated every 3 days for 3 doses with the indicated antibody at 1 mg/kg. Plots show the tumor volume (mean±SEM) of MC38 tumors over time that were treated with the indicated antibody. Tumor volume from mice euthanized due to tumor burden above 3000 mm³ was carried forward (thin vertical black lines). n=8 for isotype and commercial ipilimumab, n=9 for GIGA-564. FIG. 17B. MC38 cells were implanted on the opposite flank of naïve C57BL/6 mice and mice from (FIG. 17A) that were previously treated with ipilimumab or GIGA-564 and had a durable complete response on day 43 (0 mm³ tumor volume). Plots show the tumor volume (mean SEM) of MC38 tumors over time that were previously treated with the indicated antibody or naïve mice (n=5). Tumor volume (in mm³) was analyzed using a linear mixed effects model including treatment group and day as fixed effects and animal identifier (ID) as a random effect to account for repeated measures. Statistical comparisons were made using the Wald test against the isotype control group in the initial challenge model and against the naïve control group in the re-challenge model. Compared to the naïve group, previous treatment with ipilimumab (p=0.0089) or GIGA-564 (p=0.0088) limited tumor growth.

FIGS. 18A-18C shows that GIGA-564 plus pembrolizumab induces less toxicity than ipilimumab plus pembrolizumab in a murine model. hCTLA-4/hPD-1 double KI mice on the BALB/c background between 4-5 weeks of age were treated with vehicle, pembrolizumab, pembrolizumab plus ipilimumab (Ipi), or pembrolizumab plus GIGA-564 every 3 days for 9 doses. One week after the last dose mice were euthanized and tissues collected for pathology analysis. FIG. 18A. Plot shows percent change in body weight over time in mice treated with the indicated therapy (mean±SEM, n=10). Four mice from the pembrolizumab plus ipilimumab group died on day 12 and from the pembrolizumab plus GIGA-564 treated group three mice died on day 12 and one mouse died on day 15, all likely due to post dosing ADA-induced hypersensitivity. A mixed effect model found no statistical differences in the percent body weight change between groups. FIGS. 18B-18C. Plots show skin inflammation (FIG. 18B) or colonic epithelial damage (colitis; FIG. 18C) scores (mean+/−SEM) induced by each treatment regimen. Horizontal lines indicate the median. Adjusted p-values were calculated using the Benjamini-Hochberg step-down procedure to account for multiple comparisons.

FIG. 19 shows a Model depicting ipilimumab and GIGA-564 mechanisms of action. Top panel: Ipilimumab blocks CTLA-4 interaction with CD80/CD86, which allows antigen presenting cells (APCs) to co-stimulate peripheral Tregs enhancing their proliferation. GIGA-564 weakly blocks CTLA-4 interaction with CD80/CD86 and thus induces less Treg proliferation. Bottom panel: Ipilimumab and GIGA-564 bind CTLA-4 on intratumoral Tregs to induce Treg killing via interactions with Fc receptor (FcR) on effector cells. GIGA-564 induces stronger FcR signaling and thus more efficiently depletes intratumoral Tregs than ipilimumab.

FIGS. 20A-20E shows in vitro characterization of scFvs reformatted as full-length antibodies. FIG. 20A. Clonal cluster analysis for the FACS-enriched anti-CTLA-4 scFv clones. Each node represents an scFv clone (full-length IgK+IgH). The total number of amino acid differences were computed between each pairwise alignment of scFv sequences. Edges indicate pairwise alignments with ≤9 amino acid differences (Clustergram modified from FIG. 7 of Asensio et al., 2019). The scFv sequence for ipilimumab (ipi) was included for comparison. scFv clones for full-length antibodies described in this study are labeled with ID numbers. FIG. 20B. The affinity of the indicated antibody to soluble CTLA-4 was determined by SPR (Carterra). Plots show association and dissociation signals with a 5-fold dilution series of antigen starting at 500 nM. FIG. 20C. A 50:50 mixture of CTLA-4⁺ and CD27⁺ (CTLA-4⁻) CHO cells were stained with 10 μg/ml of the indicated antibody. An anti-human IgG secondary antibody conjugated to PE was used to detect cells labeled with the indicated anti-CTLA-4 antibody, while anti-CD27-FITC identified CD27⁺ CHO cells. Histograms show staining of CTLA-4⁺ (CD27-FITC⁻) or CTLA-4⁻ (CD27-FITC⁺) cells by the indicated antibody as determine by flow cytometry. FIG. 20D. The cell-based CTLA-4 Blockade Bioassay (Promega) involved co-culturing CTLA-4-expressing Jurkat cells with Raji cells, which naturally express CD80 and CD86, in the presence of the indicated mAbs. mAbs that bind to CTLA-4 and block the ability of CTLA-4 to interact with CD80/CD86 lead to CD28 pathway-activated luciferase expression. Plot shows the amount of luciferase expression induced (relative luciferase units; RLU) when cells were cultured with a titration series of the indicated antibody. Due to constraints on sample size in each Promega bioassay kit, this set of aCTLA-4 mAbs was analyzed using multiple plates. To control for plate-to-plate variability in maximum signal, the ipilimumab analog was run on each plate, and a representative sample was used to calculate the EC50 and maximum signal for ipilimumab in Table 23. The plate in which each antibody was tested is indicated in Table 23. Plate A and B were run at the same time, while Plate C and Plate D were run separately at later times. E. Correlation between antibody affinity and cell-based assay activity. Antibody affinity (KD) for CTLA-4 was compared to the blocking EC50 and the RLU maximum signal; no correlation was found for either when the data was fit by linear regression. Each data point represents a single antibody, and the following antibodies are distinguished as follows: ipilimumab analog (red square), GIGA-564 (inverted triangle), and aCTLA-4.28 (star). Data is from (FIG. 20D) and Table 23.

FIG. 21 provides a validation of N297Q mutants binding to cell-surface CTLA-4. CHO cells with and without human CTLA-4 expression were incubated with 10 μg/mL of the indicated antibody, then an anti-human IgG secondary antibody conjugated to FITC was used to detect cells bound by the indicated CTLA-4 antibody. Histograms show staining of CTLA-4⁺ or CTLA-4⁻ cells by the indicated antibody as determine by flow cytometry.

FIGS. 22A-22D shows that co-stimulation enhances Treg proliferation. Histograms show CellTrace Violet signal (gated on Live, CD3⁺CD4⁺ cells) of FIG. 22A. Treg or FIG. 22B. Tconv cells cultured in the presence of M-450 Tosyl activated beads coated with anti-CD3 antibody with and without CD80, or FIG. 22C. Treg or FIG. 22D. Tconv cells activated with M-450 Tosyl activated beads coated with anti-CD3 antibody plus CD80 in the presence of rhCTLA-4 (Abatacept) and/or anti-CTLA-4 mAb (aCTLA-4.28).

FIGS. 23A-23D show anti-CTLA-4s deplete intratumoral Tregs in hCTLA-4 KI mice. Flow cytometry analysis of cells from MC38 tumor bearing hCTLA-4 KI mice receiving CTLA-4 mAbs. FIGS. 23A and 23C. 6 mice per group were treated with 5 mg/kg hIgG1 isotype control, ipilimumab, or GIGA-564 on day 0 and 3, and cells were analyzed on day 4. FIGS. 23B and 23D. 12 mice per group were treated with 5 mg/kg hIgG1 isotype control, ipilimumab, or GIGA-564 on day 0, 3, and 6, and cells were analyzed on day 7. Two lymph node samples in the GIGA-564 group were excluded from analysis due to low cell count. FIGS. 23A-23B. Frequency of Tregs (Live, CD45⁺TCRβ-CD4⁺FOXP3⁺, as percentage of CD45⁺ cells) in lymph nodes (left) and tumor (right) in each treatment group. FIGS. 23C-23D. Geometric mean fluorescence intensity (MFI) of intracellular CTLA-4 in Tregs in LN (left) and tumor (right). P-values were calculated using Wilcoxon rank sum test. Lines depict mean+/−SEM.

FIG. 24 shows GIGA-564 induces more FcR signaling than ipilimumab. FIG. 24A. Purified CTLA-4 antibodies were diluted to a starting concentration of 5 μg/mL for an 8-point, 5-fold titration series in different pH buffers, then added to wells coated with rhCTLA-4-Fc. Bound antibodies were detected with anti-constant kappa-HRP and measured for absorbance at 450 nm. FIG. 24B. CHO cells expressing wildtype hCTLA-4 incubated with titrations of ipilimumab or GIGA-564 at 37° C. to allow for internalization. The amount of antibody remaining on the surface was then determined by staining with anti-human IgG Fc. FIGS. 24C-24D. GIGA-564 from three different productions was tested for fucosylation levels (bar graphs each depict a single data point) (FIG. 24C) and human FcγRIIIA signaling (FIG. 24D). PN-2758.01 and PN-4088.01 were generated from transient transfection of ExpiCHO cells, while PN-4261.01 was from a stably-expressing pool of CHOZN clones (EP-1, enriched pool 1). FIG. 24C. The fucosylation level, as determined by UPLC analysis, is shown for each sample. FIG. 24D-24E. Graph shows human FcγRIIIA (V variant) signaling as determined in a cell-based assay for the indicated three preparations of GIGA-564 (FIG. 24D) or GIGA-564 and ipilimumab with the indicated amount of fucosylation (FIG. 24E). A negative control protein with mutations to disrupt Fc receptor binding (GIGA-564_LALAPG) was also tested in the reporter bioassays. Data shown are RLU emitting from effectors cells.

FIGS. 25A-25E shows GIGA-564 results in less toxicity than ipilimumab in murine models. FIGS. 25A-25D. hCTLA-4/hPD-1 double KI mice on the BALB/c background between 4 to 5 weeks of age were treated with vehicle, pembrolizumab, pembrolizumab plus ipilimumab, or pembrolizumab plus GIGA-564 every 3 days for 9 doses. One week after the last dose mice were euthanized and tissues collected for pathology analysis. Graphs show colon length (FIG. 25A) or spleen weight (FIG. 2511) from mice of each group at time of euthanasia. The number of CD45⁺ cells/mm² counted on randomly selected CD45 stained heart FFPE sections (FIG. 25C) or the heart pathology score (FIG. 25D) as determined by a pathologist are shown. FIG. 25E. hCTLA-4 KI mice bearing MC38 tumors were treated with vehicle (PBS) or 5 mg/kg ipilimumab or GIGA-564 twice a week for 5 doses (FIG. 14A). On day 20 post initiation of treatment, mice were euthanized and kidneys were processed into formalin fixed paraffin embedded blocks (FFPE). FFPE sections were stained for anti-mouse immunoglobin or C3 and scored by a board-certified veterinary pathologist blinded from the study. Positive staining in glomeruli was along the capillary basement membranes. Graphs show percent of glomeruli positive for anti-murine IgG or C3 and the relative intensity of positive glomeruli; at least 5 glomeruli were examined in each section on 40×/high power. Data from FIG. 25E is also shown in FIGS. 4A-4E. Lines indicate mean+/−SEM. Adjusted p-value were calculated using the Benjamini-Hochberg step-down procedure to account for multiple comparisons.

FIG. 26 shows that checkpoint inhibitor Ipilimumab increases the percentage of proliferating Tregs in mice expressing humanized CTLA-4. Data from FIG. 26 is from the same experiment as FIG. 15A.

FIG. 27 shows that intratumoral Treg depletion is the mechanism of action for anti-CTLA-4, not checkpoint inhibition. FIG. 27 (left) shows that Fc effector function in anti-CTLA-4 is necessary for robust anti-tumor responses. As shown, tumor volume decreased with ipilimumab and GIGA-577 as compared to ADCC-deficient ipilimumab and ADCC-deficient GIGA-577. Data from FIG. 27 includes data as shown in FIGS. 12B and 14A.

FIG. 28 shows that GIGA-564 anti-CTLA-4 antibody has weak checkpoint inhibitor activity, but has a strong affinity for CTLA-4. A schematic showing the conventional mechanism versus the current mechanism of action as described in the present application is compared. Depletion of Tregs in the tumor after treatment with GIGA-564 is through ADCC/ADCP binding, rather than the interaction of CTLA-4 and its ligands.

FIG. 29 shows that GIGA-564 weakly blocks CD80/CD86 binding interactions to CTLA-4 as compared to Ipilimumab. FIG. 29 may reshow data shown in FIG. 20D (plate A).

FIG. 30 shows that GIGA-564 has superior anti-tumor activity compared to Ipilimumab. Humanized CTLA-4 knock-in mice bearing MC38 tumors (n=8-13) were dosed with GIGA-564 or Ipilimumab at 0.3 mpk on days 0, 3, and 6 of the study. Following administration of GIGA-564, CTLA-4 knock-in mice having MC38 tumors showed reduced tumor volume and an increased probability of survival, as compared to Ipilimumab and the isotype. The superior efficacy of GIGA-564 confirms that weakness of GIGA-564 is its inability for checkpoint inhibition. Experiments described in FIG. 30 is also described in FIGS. 11 and 14C. Tumor growth data from FIG. 30 includes data as shown in FIGS. 11 and 14C.

FIG. 31 shows that GIGA-564 induces less Treg proliferation than Ipilimumab. Human CTLA-4 knock-in mice bearing MC38 tumors were treated with 5 mpk of ipilimumab or GIGA-564 on days 0, 3, and 6 and sacrified on Day 7. GIGA-564 treatment resulted in fewer proliferating Tregs in the periphery. Experiments described here was also described in FIG. 15A. FIG. 31 includes data previously shown in FIG. 15A.

FIG. 32 shows representative contour plots of CD4 T cells in each treatment group. X-axis and Y-axis correspond to FOXP3 and CTLA-4, respectively. FIG. 32 reshows results from FIG. 15D.

FIG. 33 shows that GIGA-564 induces more FcR signaling than Ipilimumab when co-cultured with hCTLA-4+ cells. Cellular signaling via CtLA-4-Fc-FcR interaction was assessed in vitro with human CTLA-4+ cells. GIGA-564 showed increased FcR signaling over ipilimumab. Further experiments ruled out that the difference is due to Fc-FcR affinity or CTLA-4 cells surface cycling. FIG. 33 may reshow some data from FIG. 16B.

FIG. 34 provides the development and validation of CHO cells expressing cynomolgus (cyno) CTLA-4 on the cell surface. Flow histograms show that the cyno CTLA-4 and hCTLA-4 CHO cell lines express antigen on the cell surface bound by anti-CTLA-4 clone L3D10 or BNL3, this validates expression of cyno or human CTLA-4 on the surface of these cell lines, respectively.

FIG. 35 shows that GIGA-564 has reduced ability to bind surface expressed cyno CTLA-4 compared to Ipilimumab. FIG. 35 shows flow cytometric analysis of binding of mAbs to cyno or human CTLA-4+ CHO cells. The data shows that while GIGA-564 and Ipi have relatively similar ability to bind hCTLA-4; compared to Ipi, GIGA-564 has a greatly reduced ability to bind to cyno CTLA-4 expressed on the cell surface. Data from atezo binding (negative control) is reshown on multiple plots for reference.

FIG. 36A-36E show that GIGA-564 has reduced binding to some presentations of cyno CTLA-4 compared to Ipilimumab as tested using an ELISA assay. In these ELISAs binding of GIGA-564 to various CTLA-4 proteins sourced from multiple manufacturers, including Fc chimera and his-tagged formats, was tested. To test the binding of GIGA-564 to Fc chimera CTLA-4 proteins, rcmCTLA-4-Fc from R&D systems (9336-CT-200, FIG. 36A), Sino Biological (90213-C02H, FIG. 36B), or ACROBiosystems (CT4-C5256, FIG. 36C) were coated at 1 ug/mL on one half of separate ELISA plates. Recombinant human CTLA-4-Fc (rhCTLA-4) from R&D Systems (7268-CT-100. FIGS. 36A-36C) was coated at 1 ug/mL, on the other half of each of the plates. Similarly, to test the binding of GIGA-564 to his-tagged CTLA-4, rcmCTLA-4-His from Sino Biological (90213-C08H, FIG. 36D) or ACROBiosystems (CT4-C5227, FIG. 36E) were coated at 1 ug/mL on one half of separate ELISA plates. RhCTLA-4-Fc (ACROBiosystems, CT4-H5229, FIGS. 36D-E) was coated at 1 ug/mL, on the other half of each of these plates. After coating, the plates were incubated overnight at 4° C. The following day, the plates were blocked with 5% milk in PBST for 1 hour on a plate shaker at room temperature. Titration series of Ipilimumab, Atezolizumab (negative control), and GG-564 (starting at 5 ug/mL) were added to the plates and incubated on a plate shaker for 1 hour at room temperature to allow mAb binding. Excess, unbound mAbs were removed by washing with PBST. Bound mAbs were then detected with an HRP-conjugated anti-kappa light chain antibody (0.5 ug/ml, Southern Biotech 2060-50). After incubation on a plate shaker for 1 hour at room temperature and washing, the plates were developed with TMB substrate. After sufficient signal was achieved, 1 N hydrochloric acid was added to stop development. Absorbance at 450 nm was read using a Spectramax i3x plate reader (Molecular Devices). EC50 values were calculated by plotting absorbance vs. the log of concentration using Prism (GraphPad). As Ipi has similar binding to human and cyno CTLA-4 but GIGA-564 in most cases has reduced ability to bind cyno CTLA-4 compared to human CTLA-4 the results suggest that the Ipi and GIGA-564 epitopes are different in practice.

FIG. 37 provides response plots generated to determine an optimized formulation for the GIGA-564. Response plots showed the impact of pH and sucrose concentration when NaCl and buffer concentration are fixed at 100 mM and 30 mM, respectively

FIG. 38 shows theoretical response plots generated to determine the buffer concentration and NaCl amount expected to result in the highest Tm for the formulation of GIGA-564 antibody as shown.

FIG. 39 shows a pareto analysis that provided which variables had the most impact on the formulation for GIGA-564.

FIG. 40 provides a conformational analysis of a formulation for GIGA-564. The plots show that high NaCl is the most important for conformation, pH has some effect, with lower pH being better, and sucrose has some effect, with higher being better.

FIG. 41 shows that GIGA-2328 is designed to have low fucosylation for the purpose of enhancing FcγRIIIa signaling. An ADCC reporter bioassay for human FcγRIIIa, V variant (G7011) from Promega Corporation was carried out following the manufacturer's instructions. Briefly, CHO target cells stably expressing human CTLA-4 with the Y201G mutation to enhance cell surface expression were suspended in RPMI 1640+4% FBS media with the indicated antibody and incubated at 37° C. for 30 minutes. Jurkat/NFAT-Luc effector cells expressing human FcγRIIIa, V variant were added to each well at an effector:target ratio of 5:1 and incubated at 37° C. for 6 hours. Luciferase activity was measured by using the included Bio-Glo Luciferase Assay Reagent with the SpectraMax i3x reader. Luciferase activity measured in relative luminescence units (RLU) were plotted against the concentration of antibody.

FIGS. 42A-42B shows that GIGA-564 induces antibody-dependent cellular cytotoxicity (ADCC) and/or antibody-dependent cellular phagocytosis (ADCP) by human PBMCs against a target cell line expressing CTLA-4. Cryopreserved human PBMCs from two donors were thawed and recovered overnight in RMPI media containing 100 U/mL IL-2 and 10% FBS. CHO target cells stably expressing human CTLA-4 with the Y201G mutation to enhance cell surface expression were stained with CellTrace Violet (Thermo Fisher), then incubated with either GIGA-564 or a human IgG1 isotype control at the indicated concentrations at 37° C. for 30 minutes. PMBC effector cells were then added at a 20:1 effector to target ratio and the samples were incubated at 37° C. for 4 hours to allow for ADCC. The samples were then washed with MACS buffer, stained with the viability dye 7-Aminoactinomycin D (7-AAD) to label dead cells, and analyzed by flow cytometry. (FIG. 42A) Representative gating strategy to determine ADCC. Samples were first gated for target cells (CellTrace Violet+), then single cells, then dead (7-AAD+) cells. (FIG. 42B) Plots of percent dead cells at each concentration of GIGA-564 and human IgG1 isotype. GIGA-564 leads to higher percent dead CTLA-4+ target cells than the isotype control.

FIGS. 43A-43B show that IgG1 allotype may impact signaling via the FcγRIIIa receptor. FIG. 43A shows sequence information of IgG1 allotype IGHG1*08 and IGHG1*01, which differ by one amino acid in the CH1 domain. The differing residue between these two allotypes is highlighted, and surrounding residues are provided for reference. Position given in both IMGT and EU numbering. FIG. 43B shows clinical ipilimumab (Yervoy) uses the IGHG1*08 allotype, and results in lower FcγRIIIa signaling than Ipilimumab and GIGA-564 produced by GigaGen, which use the IGHG1*01. These results suggest that allotype may impact FcγRIIIa signaling. ADCC reporter bioassays for human FcγRIIIa, V variant (G7011) were purchased from Promega Corporation. The assays were carried out following the manufacturer's instructions. Briefly, CHO target cells stably expressing human CTLA-4 with the Y201G mutation to enhance cell surface expression were suspended in RPMI 1640+4% FBS media with the indicated antibody and incubated at 37° C. for 30 minutes. Jurkat/NFAT-Luc effector cells expressing human FcγRIIIa, V variant were added to each well at an effector:target ratio of 5:1 and incubated at 37° C. for 6 hours. Luciferase activity was measured by using the included Bio-Glo Luciferase Assay Reagent with the SpectraMax i3x reader. Luciferase activity measured in relative luminescence units (RLU) were plotted against the concentration of antibody.

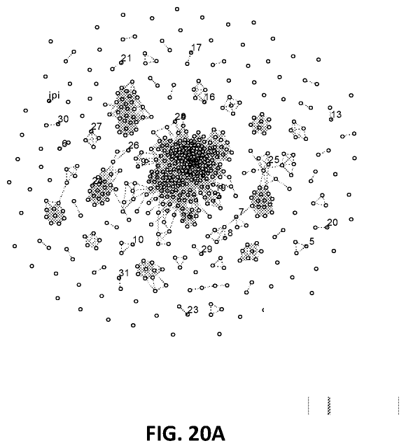

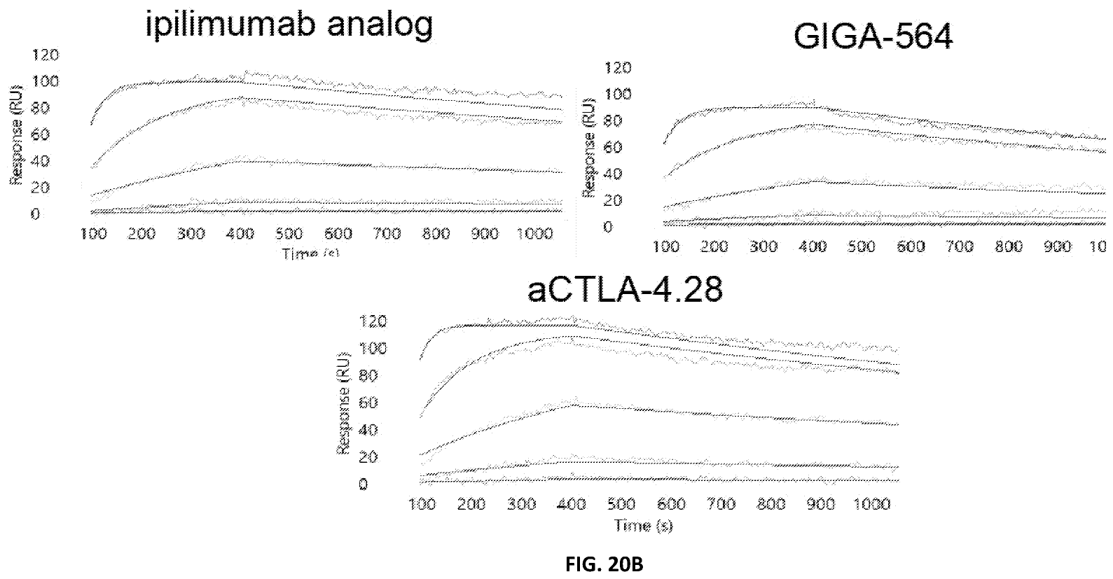

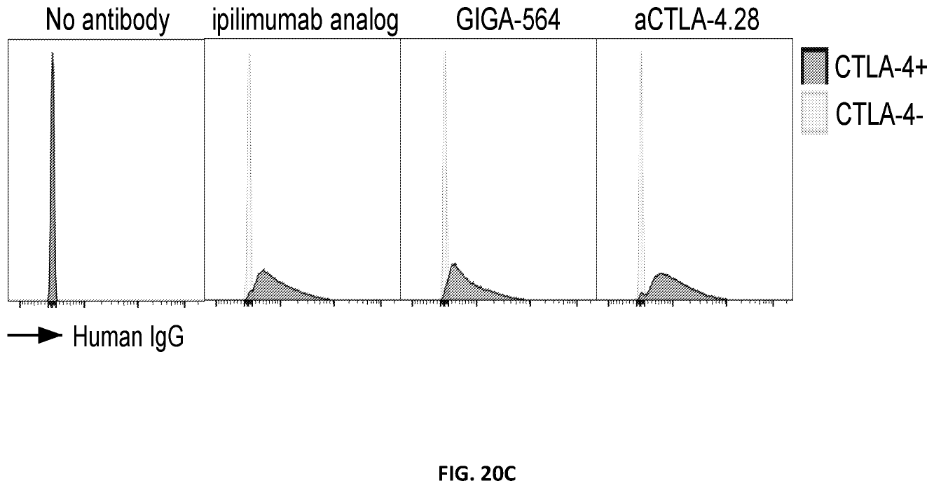

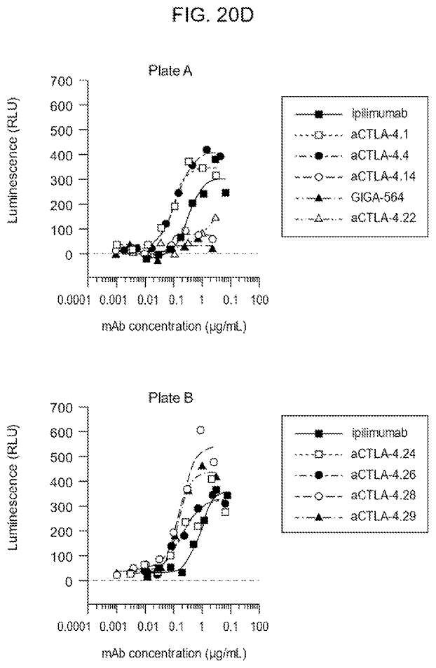

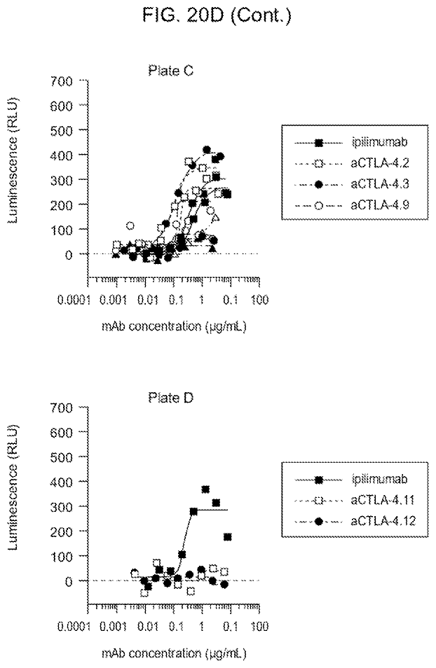

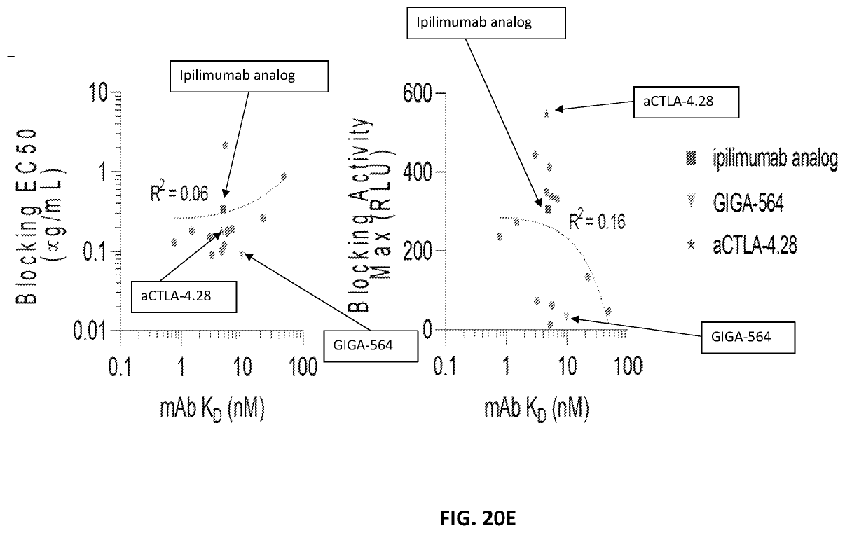

Table 23. In vitro characterization of scFvs reformatted as full-length antibodies. kon, koff, and KD of the indicated CTLA-4 mAbs for soluble human CTLA-4 was determined by SPR (Carterra). Geometric MFI of the indicated CTLA-4 mAbs bound to CTLA-4⁺ or CD27⁺ CHO cells as detected by a secondary human IgG antibody and flow cytometry. Affinity (KD) of the indicated CTLA-4 mAbs for soluble cynomolgus CTLA-4 was determined by a single antigen concentration BLI measurement (Octet). The ability of each aCTLA-4 mAb to block CTLA-4 binding to CD80/CD86 was determined by a cell-based assay (Promega). Due to constraints on sample size in each Promega bioassay kit, this set of aCTLA-4 mAbs was analyzed using multiple plates (plate letters correspond to Plate A-D in FIG. 20D). To control for plate-to-plate variability in maximum signal, the ipilimumab analog was run on each plate; a representative sample was used for this table.

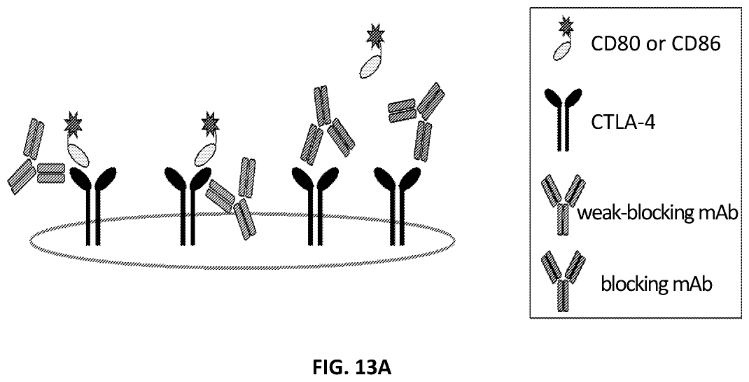

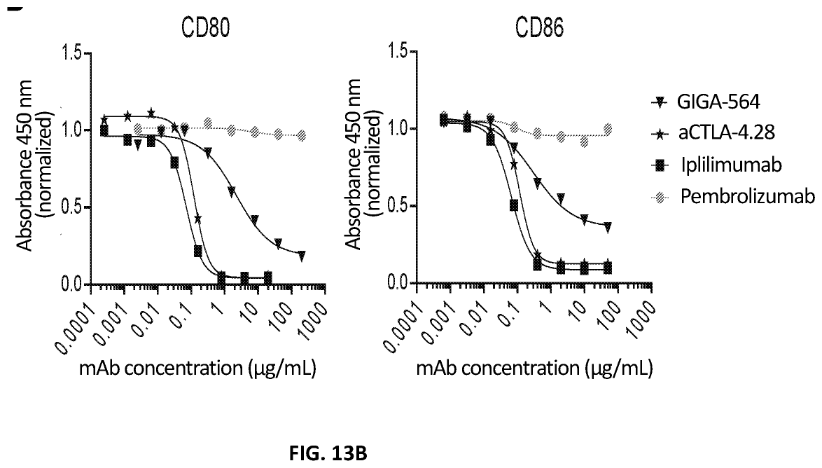

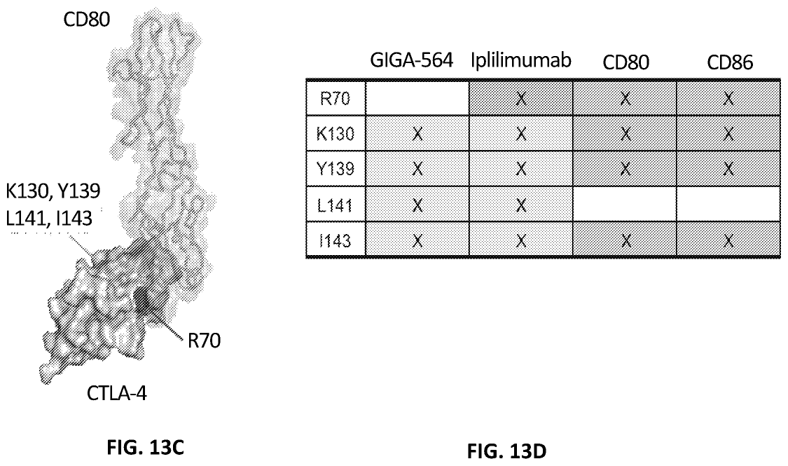

Table 24. GIGA-564 has minimal ability to block CD80 or CD86 from binding CTLA-4. Summary of blocking ELISA data from FIG. 13B. Absorbance values from the detected CD80 or CD86 were normalized to signal from ELISA plates incubated with pembrolizumab. The data were then analyzed and fit by nonlinear regression. The normalized maximum and minimum signals and the IC50 for blocking are provided.

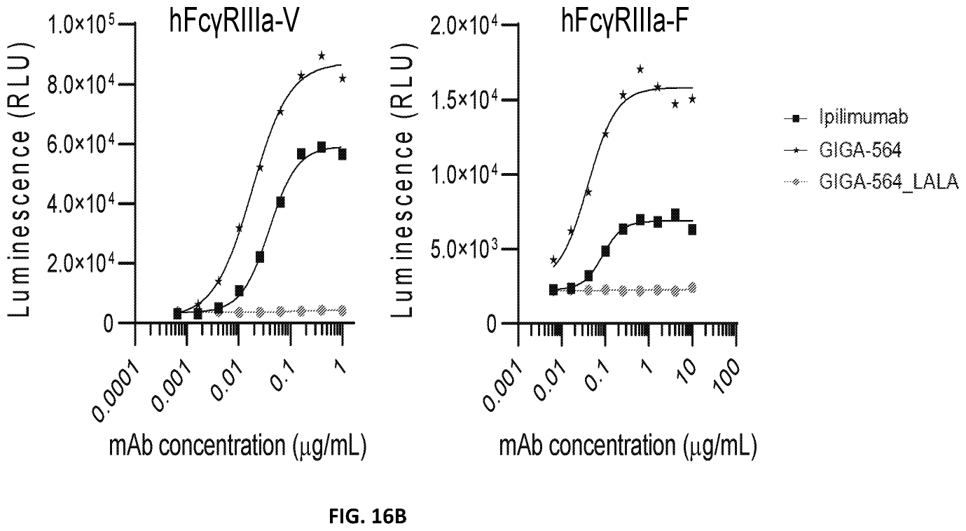

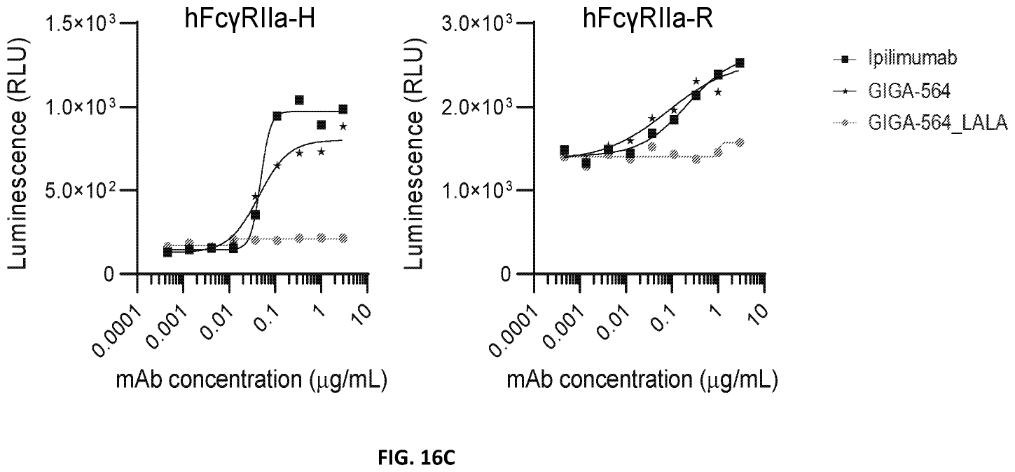

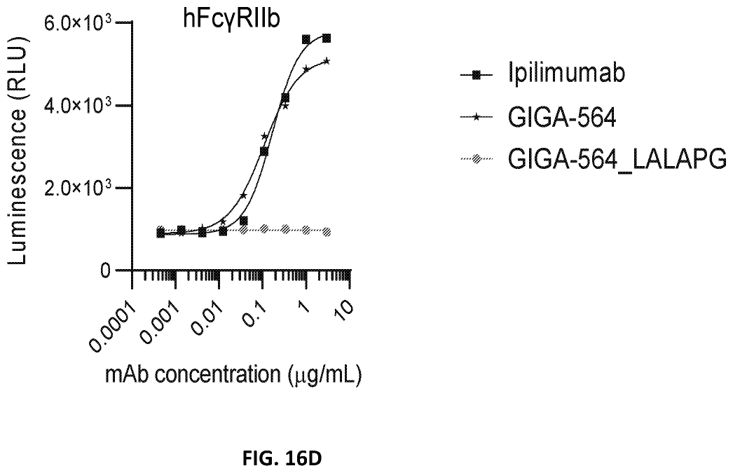

Table 25. Fc receptor signaling of GIGA-564 and ipilimumab. Summary of FcR Bioassay results from FIG. 16. Luminescence (RLU) data from the Bioassay were fit by nonlinear regression. The maximum signal and EC50 for FcR signaling are provided.

Table 26. CTLA-4 antibody sequences. Amino acid sequences are provided of the IgK and IgG variable regions for the 14 characterized full-length CTLA-4 antibodies.

Table 27. Reagent antibodies for immunophenotyping. Antibody clone details used for immunophenotyping are listed.

Table 32-33. A14 Light Chain CDR sequences and A14 Heavy Chain CDR sequences for GIGA-564 identified by different algorithms (Antibody A14):

### 7. DETAILED DESCRIPTION

### 7.1. Definitions

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well-known and commonly used in the art. The methods and techniques of the present disclosure are generally performed according to conventional methods well known in the art and as described in various general and more specific references that are cited and discussed throughout the present specification unless otherwise indicated. See, e.g., Sambrook et al. Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992), and Harlow and Lane Antibodies: A Laboratory Manual Cold Spring Harbor Laboratory Press, Cold Spring Harbor, N.Y. (1990), which are incorporated herein by reference. Enzymatic reactions and purification techniques are performed according to manufacturer's specifications, as commonly accomplished in the art or as described herein. The terminology used in connection with, and the laboratory procedures and techniques of, analytical chemistry, synthetic organic chemistry, and medicinal and pharmaceutical chemistry described herein are those well-known and commonly used in the art. Standard techniques can be used for chemical syntheses, chemical analyses, pharmaceutical preparation, formulation, and delivery, and treatment of patients.

The following terms, unless otherwise indicated, shall be understood to have the following meanings:

The terms “CTLA-4,” “CTLA-4 protein,” and “CTLA-4 antigen” are used interchangeably herein to refer to human CTLA-4, or any variants (e.g., splice variants and allelic variants), isoforms, and species homologs of human CTL A-4 that are naturally expressed by cells, or that are expressed by cells transfected with a ctla4 gene. In some embodiments, the CTLA-4 protein is a CTLA-4 protein naturally expressed by a primate (e.g., a monkey or a human), a rodent (e.g., a mouse or a rat), a dog, a camel, a cat, a cow, a goat, a horse, or a sheep. In some embodiments, the CTLA-4 protein is human CTLA-4 (hCTLA-4; SEQ ID NO: 7001).

The term “immunoglobulin” refers to a class of structurally related proteins generally comprising two pairs of polypeptide chains: one pair of light (L) chains and one pair of heavy (H) chains. In an “intact immunoglobulin,” all four of these chains are interconnected by disulfide bonds. The structure of immunoglobulins has been well characterized. See, e.g., Paul, Fundamental Immunology 7th ed., Ch. 5 (2013) Lippincott Williams & Wilkins, Philadelphia, Pa. Briefly, each heavy chain typically comprises a heavy chain variable region (V_{H}) and a heavy chain constant region (CH). The heavy chain constant region typically comprises three domains, abbreviated C_{H1}, C_{H2}, and C_{H3}. Each light chain typically comprises a light chain variable region (V_{L}) and a light chain constant region. The light chain constant region typically comprises one domain, abbreviated CL.

The term “antigen-binding protein” (ABP) refers to a protein comprising one or more antigen-binding domains that specifically bind to an antigen or epitope. In some embodiments, the antigen-binding domain binds the antigen or epitope with specificity and affinity similar to that of naturally occurring antibodies. In some embodiments, the ABP comprises an antibody. In some embodiments, the ABP consists of an antibody. In some embodiments, the ABP consists essentially of an antibody. In some embodiments, the ABP comprises an alternative scaffold. In some embodiments, the ABP consists of an alternative scaffold. In some embodiments, the ABP consists essentially of an alternative scaffold. In some embodiments, the ABP comprises an antibody fragment. In some embodiments, the ABP consists of an antibody fragment. In some embodiments, the ABP consists essentially of an antibody fragment. A “CTLA-4 ABP,” “anti-CTLA-4 ABP,” or “CTLA-4-specific ABP” is an ABP, as provided herein, which specifically binds to the antigen CTLA-4. In some embodiments, the ABP binds the extracellular domain of CTLA-4. In certain embodiments, a CTLA-4 ABP provided herein binds to an epitope of CTLA-4 that is conserved between or among CTLA-4 proteins from different species.

The term “antibody” is used herein in its broadest sense and includes certain types of immunoglobulin molecules comprising one or more antigen-binding domains that specifically bind to an antigen or epitope. An antibody specifically includes intact antibodies (e.g., intact immunoglobulins), antibody fragments, and multi-specific antibodies. One example of an antigen-binding domain is an antigen-binding domain formed by a V_{H}-V_{L} dimer. An antibody is one type of ABP.

The term “afucosylation” or “afucosylated” in the context of an Fc refers to a substantial lack of core fucosylation of the N-glycan covalently attached, directly or indirectly, to the N-glycosylation site, e.g., amino acid residue position 297 of the human IgG1 Fc region, numbered according to the EU index (Kabat et al., Sequences of Proteins of Immunological Interest, 5th Ed. Public Health Service, National Institutes of Health, Bethesda, Md. (1991)), or the corresponding residue in non-IgG1 or non-human IgG1 immunoglobulins.

When the fucosylation rate is indicated in the context of a composition comprising antibodies, the rate indicates the proportion of focusylated antibodies among the total antibodies in the composition. For example, 70% of fucosylation indicates that 70% of the antibodies in the composition are fucosylated and 30% of the antibodies in the composition are afucosylated.

The term “alternative scaffold” refers to a molecule in which one or more regions may be diversified to produce one or more antigen-binding domains that specifically bind to an antigen or epitope. In some embodiments, the antigen-binding domain binds the antigen or epitope with specificity and affinity similar to that of naturally occurring antibodies. Exemplary alternative scaffolds include those derived from fibronectin (e.g., Adnectins™), the β-sandwich (e.g., iMab), lipocalin (e.g., Anticalins®), EETI-II/AGRP, BPTI/LACI-D1/ITI-D2 (e.g., Kunitz domains), thioredoxin peptide aptamers, protein A (e.g., Affibody®), ankyrin repeats (e.g., DARPins), gamma-B-crystallin/ubiquitin (e.g., Affilins), CTLD3 (e.g., Tetranectins), Fynomers, and (LDLR-A module) (e.g., Avimers). Additional information on alternative scaffolds is provided in Binz et al., Nat. Biotechnol., 2005 23:1257-1268; Skerra, Current Opin. in Biotech., 2007 18:295-304; and Silacci et al., J. Biol. Chem., 2014, 289:14392-14398; each of which is incorporated by reference in its entirety. An alternative scaffold is one type of ABP.

The term “antigen-binding domain” means the portion of an ABP that is capable of specifically binding to an antigen or epitope.

The terms “full length antibody,” “intact antibody,” and “whole antibody” are used herein interchangeably to refer to an antibody having a structure substantially similar to a naturally occurring antibody structure and having heavy chains that comprise an Fc region.

The term “Fc region” means the C-terminal region of an immunoglobulin heavy chain that, in naturally occurring antibodies, interacts with Fc receptors and certain proteins of the complement system. The structures of the Fc regions of various immunoglobulins, and the glycosylation sites contained therein, are known in the art. See Schroeder and Cavacini, J Allergy Clin. Immunol., 2010, 125:S41-52, incorporated by reference in its entirety. The Fc region may be a naturally occurring Fc region, or an Fc region modified as described elsewhere in this disclosure.

The V_{H} and V_{L} regions may be further subdivided into regions of hypervariability (“hypervariable regions (HVRs);” also called “complementarity determining regions” (CDRs)) interspersed with regions that are more conserved. The more conserved regions are called framework regions (FRs). Each V_{H} and V_{L} generally comprises three CDRs and four FRs, arranged in the following order (from N-terminus to C-terminus): FR1-CDR1-FR2-CDR2-FR3-CDR3-FR4. The CDRs are involved in antigen binding, and influence antigen specificity and binding affinity of the antibody. See Kabat et al., Sequences of Proteins of Immunological Interest 5th ed. (1991) Public Health Service, National Institutes of Health, Bethesda, Md., incorporated by reference in its entirety.

The light chain from any vertebrate species can be assigned to one of two types, called kappa (κ) and lambda (λ), based on the sequence of its constant domain.

The heavy chain from any vertebrate species can be assigned to one of five different classes (or isotypes): IgA, IgD, IgE. IgG, and IgM. These classes are also designated a, δ, ε, γ, and μ, respectively. The IgG and IgA classes are further divided into subclasses on the basis of differences in sequence and function. Humans express the following subclasses: IgG1, IgG2, IgG3, IgG4, IgA1, and IgA2.

The amino acid sequence boundaries of a CDR can be determined by one of skill in the art using any of a number of known numbering schemes, including those described by Kabat et al., supra (“Kabat” numbering scheme); Al-Lazikani et al., 1997, J. Mol. Biol., 273:927-948 (“Chothia” numbering scheme); MacCallum et al., 1996, J Mol. Biol. 262:732-745 (“Contact” numbering scheme); Lefranc et al., Dev. Comp. Immunol., 2003, 27:55-77 (“IMGT” numbering scheme); and Honegge and Plückthun, J. Mol. Biol, 2001, 309:657-70 (“AHo” numbering scheme); each of which is incorporated by reference in its entirety.

Table 1 provides the positions of CDR1-L (CDR1 of V_{L}), CDR2-L (CDR2 of V_{L}), CDR3-L (CDR3 of V_{L}), CDR1-H (CDR1 of V_{H}), CDR2-H (CDR2 of V_{H}), and CDR3-H (CDR3 of V_{H}), as identified by the Kabat and Chothia schemes. For CDR1-H, residue numbering is provided using both the Kabat and Chothia numbering schemes.

CDRs may be assigned, for example, using antibody numbering software, such as Abnum, available at www.bioinf.org.uk/abs/abnum/, and described in Abhinandan and Martin, Immunology, 2008, 45:3832-3839, incorporated by reference in its entirety.

TABLE 1
Residues in CDRs according to Kabat
and Chothia numbering schemes.

| CDR | Kabat | Chothia |
|---|---|---|
| CDR1-L | 24-34 | 24-34 |
| CDR2-L | 50-56 | 50-56 |
| CDR3-L | 89-97 | 89-97 |
| CDR1-H (Kabat Numbering) | 31-35B | 26-32 or 34* |
| CDR1-H (Chothia Numbering) | 31-35 | 26-32 |
| CDR2-H | 50-65 | 52-56 |
| CDR3-H | 95-102 | 95-102 |

*The C-terminus of CDR1-H, when numbered using the Kabat numbering convention, varies between 32 and 34, depending on the length of the CDR.

The “EU numbering scheme” is generally used when referring to a residue in an antibody heavy chain constant region (e.g., as reported in Kabat et al., supra).

An “antibody fragment” comprises a portion of an intact antibody, such as the antigen-binding or variable region of an intact antibody. Antibody fragments include, for example, Fv fragments, Fab fragments, F(ab′)₂ fragments, Fab′ fragments, scFv (sFv) fragments, and scFv-Fc fragments.

“Fv” fragments comprise a non-covalently-linked dimer of one heavy chain variable domain and one light chain variable domain.

“Fab” fragments comprise, in addition to the heavy and light chain variable domains, the constant domain of the light chain and the first constant domain (C_{H1}) of the heavy chain. Fab fragments may be generated, for example, by recombinant methods or by papain digestion of a full-length antibody.

“F(ab′)₂” fragments contain two Fab′ fragments joined, near the hinge region, by disulfide bonds. F(ab′)₂ fragments may be generated, for example, by recombinant methods or by pepsin digestion of an intact antibody. The F(ab′) fragments can be dissociated, for example, by treatment with ß-mercaptoethanol.

“Single-chain Fv” or “sFv” or “scFv” antibody fragments comprise a V_{H} domain and a V_{L} domain in a single polypeptide chain. The V_{H} and V_{L} are generally linked by a peptide linker. See Pluckthun A. (1994). In some embodiments, the linker is a (GGGGS)ₙ(SEQ ID NO: 11968). In some embodiments, n=1, 2, 3, 4, 5, or 6. See Antibodies from Escherichia coli. In Rosenberg M. & Moore G. P. (Eds.), The Pharmacology of Monoclonal Antibodies vol. 113 (pp. 269-315). Springer-Verlag, New York, incorporated by reference in its entirety.

“scFv-Fc” fragments comprise an scFv attached to an Fc domain. For example, an Fc domain may be attached to the C-terminal of the scFv. The Fc domain may follow the V_{H} or V_{L}, depending on the orientation of the variable domains in the scFv (i.e., V_{H}-V_{L} or V_{L}-V_{H}). Any suitable Fc domain known in the art or described herein may be used. In some cases, the Fc domain comprises an IgG4 Fc domain.

The term “single domain antibody” refers to a molecule in which one variable domain of an antibody specifically binds to an antigen without the presence of the other variable domain. Single domain antibodies, and fragments thereof, are described in Arabi Ghahroudi et al., FEBS Letters, 1998, 414:521-526 and Muyldermans et al., Trends in Biochem. Sci., 2001, 26:230-245, each of which is incorporated by reference in its entirety.

A “monospecific ABP” is an ABP that comprises a binding site that specifically binds to a single epitope. An example of a monospecific ABP is a naturally occurring IgG molecule which, while divalent, recognizes the same epitope at each antigen-binding domain. The binding specificity may be present in any suitable valency.

The term “monoclonal antibody” refers to an antibody from a population of substantially homogeneous antibodies. A population of substantially homogeneous antibodies comprises antibodies that are substantially similar and that bind the same epitope(s), except for variants that may normally arise during production of the monoclonal antibody. Such variants are generally present in only minor amounts. A monoclonal antibody is typically obtained by a process that includes the selection of a single antibody from a plurality of antibodies. For example, the selection process can be the selection of a unique clone from a plurality of clones, such as a pool of hybridoma clones, phage clones, yeast clones, bacterial clones, or other recombinant DNA clones. The selected antibody can be further altered, for example, to improve affinity for the target (“affinity maturation”), to humanize the antibody, to improve its production in cell culture, and/or to reduce its immunogenicity in a subject.

The term “chimeric antibody” refers to an antibody in which a portion of the heavy and/or light chain is derived from a particular source or species, while the remainder of the heavy and/or light chain is derived from a different source or species.

“Humanized” forms of non-human antibodies are chimeric antibodies that contain minimal sequence derived from the non-human antibody. A humanized antibody is generally a human antibody (recipient antibody) in which residues from one or more CDRs are replaced by residues from one or more CDRs of a non-human antibody (donor antibody). The donor antibody can be any suitable non-human antibody, such as a mouse, rat, rabbit, chicken, or non-human primate antibody having a desired specificity, affinity, or biological effect. In some instances, selected framework region residues of the recipient antibody are replaced by the corresponding framework region residues from the donor antibody. Humanized antibodies may also comprise residues that are not found in either the recipient antibody or the donor antibody. Such modifications may be made to further refine antibody function. For further details, see Jones et al., Nature, 1986, 321:522-525; Riechmann et al., Nature, 1988, 332:323-329; and Presta, Curr. Op. Struct. Biol., 1992, 2:593-596, each of which is incorporated by reference in its entirety.

A “human antibody” is one which possesses an amino acid sequence corresponding to that of an antibody produced by a human or a human cell, or derived from a non-human source that utilizes a human antibody repertoire or human antibody-encoding sequences (e.g., obtained from human sources or designed de novo). Human antibodies specifically exclude humanized antibodies. In some embodiments, rodents are genetically engineered to replace their rodent antibody sequences with human antibodies.

An “isolated ABP” or “isolated nucleic acid” is an ABP or nucleic acid that has been separated and/or recovered from a component of its natural environment. Components of the natural environment may include enzymes, hormones, and other proteinaceous or nonproteinaceous materials. In some embodiments, an isolated ABP is purified to a degree sufficient to obtain at least 15 residues of N-terminal or internal amino acid sequence, for example by use of a spinning cup sequenator. In some embodiments, an isolated ABP is purified to homogeneity by gel electrophoresis (e.g., SDS-PAGE) under reducing or nonreducing conditions, with detection by Coomassie blue or silver stain. An isolated ABP includes an ABP in situ within recombinant cells, since at least one component of the ABP's natural environment is not present. In some embodiments, an isolated ABP or isolated nucleic acid is prepared by at least one purification step. In some embodiments, an isolated ABP or isolated nucleic acid is purified to at least 80%, 85%, 90%, 95%, or 99% by weight. In some embodiments, an isolated ABP or isolated nucleic acid is purified to at least 80%, 85%, 90%, 95%, or 99% by volume. In some embodiments, an isolated ABP or isolated nucleic acid is provided as a solution comprising at least 85%, 90%, 95%, 98%, 99% to 100% ABP or nucleic acid by weight. In some embodiments, an isolated ABP or isolated nucleic acid is provided as a solution comprising at least 85%, 90%, 95%, 98%, 99% to 100% ABP or nucleic acid by volume.

“Affinity” refers to the strength of the sum total of non-covalent interactions between a single binding site of a molecule (e.g., an ABP) and its binding partner (e.g., an antigen or epitope). Unless indicated otherwise, as used herein, “affinity” refers to intrinsic binding affinity, which reflects a 1:1 interaction between members of a binding pair (e.g., ABP and antigen or epitope). The affinity of a molecule X for its partner Y can be represented by the dissociation equilibrium constant (K_{D}). The kinetic components that contribute to the dissociation equilibrium constant are described in more detail below. Affinity can be measured by common methods known in the art, including those described herein. Affinity can be determined, for example, using surface plasmon resonance (SPR) technology (e.g., BIACORE©) or biolayer interferometry (e.g., FORTEBIO©).

With regard to the binding of an ABP to a target molecule, the terms “bind,” “specific binding,” “specifically binds to.” “specific for,” “selectively binds,” and “selective for” a particular antigen (e.g., a polypeptide target) or an epitope on a particular antigen mean binding that is measurably different from a non-specific or non-selective interaction (e.g., with a non-target molecule). Specific binding can be measured, for example, by measuring binding to a target molecule and comparing it to binding to a non-target molecule. Specific binding can also be determined by competition with a control molecule that mimics the epitope recognized on the target molecule. In that case, specific binding is indicated if the binding of the ABP to the target molecule is competitively inhibited by the control molecule. In some embodiments, the affinity of a CTLA-4 ABP for a non-target molecule is less than about 50% of the affinity for CTLA-4. In some embodiments, the affinity of a CTLA-4 ABP for a non-target molecule is less than about 40% of the affinity for CTLA-4. In some embodiments, the affinity of a CTLA-4 ABP for a non-target molecule is less than about 30% of the affinity for CTLA-4. In some embodiments, the affinity of a CTLA-4 ABP for a non-target molecule is less than about 20% of the affinity for CTLA-4. In some embodiments, the affinity of a CTLA-4 ABP for a non-target molecule is less than about 10% of the affinity for CTLA-4. In some embodiments, the affinity of a CTLA-4 ABP for a non-target molecule is less than about 1% of the affinity for CTLA-4. In some embodiments, the affinity of a CTLA-4 ABP for a non-target molecule is less than about 0.1% of the affinity for CTLA-4.

The term “k_{d}” (sec⁻¹), as used herein, refers to the dissociation rate constant of a particular ABP-antigen interaction. This value is also referred to as the koff value.

The term “kₐ” (M⁻¹×sec⁻¹), as used herein, refers to the association rate constant of a particular ABP-antigen interaction. This value is also referred to as the kₒₙ value.

The term “K_{D}” (M), as used herein, refers to the dissociation equilibrium constant of a particular ABP-antigen interaction. K_{D}=k_{d}/kₐ.

The term “K_{A}” (M-1), as used herein, refers to the association equilibrium constant of a particular ABP-antigen interaction. K_{A}=k_{d}/kₐ.

An “affinity matured” ABP is one with one or more alterations (e.g., in one or more CDRs or FRs) that result in an improvement in the affinity of the ABP for its antigen, compared to a parent ABP which does not possess the alteration(s). In one embodiment, an affinity matured ABP has nanomolar or picomolar affinity for the target antigen. Affinity matured ABPs may be produced using a variety of methods known in the art. For example, Marks et al. (Bio/Technology, 1992, 10:779-783, incorporated by reference in its entirety) describes affinity maturation by V_{H} and V_{L} domain shuffling. Random mutagenesis of CDR and/or framework residues is described by, for example, Barbas et al. (Proc. Nat. Acad. Sci. U.S.A., 1994, 91:3809-3813); Schier et al., Gene, 1995, 169:147-155; Yelton et al., J. Immunol., 1995, 155:1994-2004; Jackson et al., J. Immunol., 1995, 154:3310-33199; and Hawkins et al, J. Mol. Biol., 1992, 226:889-896; each of which is incorporated by reference in its entirety.

An “immunoconjugate” is an ABP conjugated to one or more heterologous molecule(s).

“Effector functions” refer to those biological activities mediated by the Fc region of an antibody, which activities may vary depending on the antibody isotype. Examples of antibody effector functions include C1q binding to activate complement dependent cytotoxicity (CDC), Fc receptor binding to activate antibody-dependent cellular cytotoxicity (ADCC), and antibody dependent cellular phagocytosis (ADCP).

When used herein in the context of two or more ABPs, the term “competes with” or “cross-competes with” indicates that the two or more ABPs compete for binding to an antigen (e.g., CTLA-4). In one exemplary assay, CTLA-4 is coated on a surface and contacted with a first CTLA-4 ABP, after which a second CTLA-4 ABP is added. In another exemplary assay, a first CTLA-4 ABP is coated on a surface and contacted with CTLA-4, and then a second CTLA-4 ABP is added. If the presence of the first CTLA-4 ABP reduces binding of the second CTLA-4 ABP, in either assay, then the ABPs compete. The term “competes with” also includes combinations of ABPs where one ABP reduces binding of another ABP, but where no competition is observed when the ABPs are added in the reverse order. However, in some embodiments, the first and second ABPs inhibit binding of each other, regardless of the order in which they are added. In some embodiments, one ABP reduces binding of another ABP to its antigen by at least 25%, at least 50%, at least 60%, at least 70%, at least 80%, at least 85%, at least 90%, or at least 95%. A skilled artisan can select the concentrations of the antibodies used in the competition assays based on the affinities of the ABPs for CTLA-4 and the valency of the ABPs. The assays described in this definition are illustrative, and a skilled artisan can utilize any suitable assay to determine if antibodies compete with each other. Suitable assays are described, for example, in Cox et al., “Immunoassay Methods,” in Assay Guidance Manual [Internet], Updated Dec. 24, 2014 (www(dot)ncbi(dot)nlm(dot)nih(dot)gov/books/NBK92434/; accessed Sep. 29, 2015); Silman et al., Cytometry, 2001, 44:30-37; and Finco et al., J. Pharm. Biomed. Anal., 2011, 54:351-358; each of which is incorporated by reference in its entirety.

The term “epitope” means a portion of an antigen the specifically binds to an ABP. Epitopes frequently consist of surface-accessible amino acid residues and/or sugar side chains and may have specific three-dimensional structural characteristics, as well as specific charge characteristics. Conformational and non-conformational epitopes are distinguished in that the binding to the former but not the latter may be lost in the presence of denaturing solvents. An epitope may comprise amino acid residues that are directly involved in the binding, and other amino acid residues, which are not directly involved in the binding. The epitope to which an ABP binds can be determined using known techniques for epitope determination such as, for example, testing for ABP binding to CTLA-4 variants with different point-mutations, or to chimeric CTLA-4 variants.

Percent “identity” between a polypeptide sequence and a reference sequence, is defined as the percentage of amino acid residues in the polypeptide sequence that are identical to the amino acid residues in the reference sequence, after aligning the sequences and introducing gaps, if necessary, to achieve the maximum percent sequence identity. Alignment for purposes of determining percent amino acid sequence identity can be achieved in various ways that are within the skill in the art, for instance, using publicly available computer software such as BLAST, BLAST-2, ALIGN, MEGALIGN (DNASTAR), CLUSTALW, CLUSTAL OMEGA, or MUSCLE software. Those skilled in the art can determine appropriate parameters for aligning sequences, including any algorithms needed to achieve maximal alignment over the full length of the sequences being compared.

A “conservative substitution” or a “conservative amino acid substitution,” refers to the substitution an amino acid with a chemically or functionally similar amino acid. Conservative substitution tables providing similar amino acids are well known in the art. By way of example, the groups of amino acids provided in TABLES 2-4 are, in some embodiments, considered conservative substitutions for one another.

TABLE 2
Selected groups of amino acids that are considered conservative
substitutions for one another, in certain embodiments.

| | |
|---|---|
| Acidic Residues | P and E |
| Basic Residues | K, R, and H |
| Hydrophilic Uncharged Residues | S, T, N, and Q |
| Aliphatic Uncharged Residues | G, A, V, L, and I |
| Non-polar Uncharged Residues | C, M, and P |
| Aromatic Residues | F, Y, and W |

TABLE 3
Additional selected groups of amino acids that
are considered conservative substitutions
for one another, in certain embodiments.

| | |
|---|---|
| Group 1 | A, S, and T |
| Group 2 | D and E |
| Group 3 | N and Q |
| Group 4 | R and K |
| Group 5 | I, L, and M |
| Group 6 | F, Y, and W |

TABLE 4
Further selected groups of amino acids that are considered conservative
substitutions for one another, in certain embodiments.

| | |
|---|---|
| Group A | A and G |
| Group B | D and E |
| Group C | N and Q |
| Group D | R, K, and H |
| Group E | I, L, M, V |
| Group F | F, Y, and W |
| Group G | S and T |
| Group H | C and M |

Additional conservative substitutions may be found, for example, in Creighton, Proteins: Structures and Molecular Properties 2nd ed. (1993) W. H. Freeman & Co., New York, N.Y. An ABP generated by making one or more conservative substitutions of amino acid residues in a parent ABP is referred to as a “conservatively modified variant.”

The term “treating” (and variations thereof such as “treat” or “treatment”) refers to clinical intervention in an attempt to alter the natural course of a disease or condition in a subject in need thereof. Treatment can be performed both for prophylaxis and during the course of clinical pathology. Desirable effects of treatment include preventing occurrence or recurrence of disease, alleviation of symptoms, diminish of any direct or indirect pathological consequences of the disease, preventing metastasis, decreasing the rate of disease progression, amelioration or palliation of the disease state, and remission or improved prognosis.

As used herein, the term “therapeutically effective amount” or “effective amount” refers to an amount of an ABP or pharmaceutical composition provided herein that, when administered to a subject, is effective to treat a disease or disorder.

As used herein, the term “subject” means a mammalian subject. Exemplary subjects include humans, monkeys, dogs, cats, mice, rats, cows, horses, camels, goats, rabbits, and sheep. In certain embodiments, the subject is a human. In some embodiments the subject has a disease or condition that can be treated with an ABP provided herein. In some embodiments, the disease or condition is a cancer. In some embodiments, the disease or condition is a viral infection.

The term “package insert” is used to refer to instructions customarily included in commercial packages of therapeutic or diagnostic products (e.g., kits) that contain information about the indications, usage, dosage, administration, combination therapy, contraindications and/or warnings concerning the use of such therapeutic or diagnostic products.

The term “cytotoxic agent,” as used herein, refers to a substance that inhibits or prevents a cellular function and/or causes cell death or destruction.

A “chemotherapeutic agent” refers to a chemical compound useful in the treatment of cancer. Chemotherapeutic agents include “anti-hormonal agents” or “endocrine therapeutics” which act to regulate, reduce, block, or inhibit the effects of hormones that can promote the growth of cancer.

The term “cytostatic agent” refers to a compound or composition which arrests growth of a cell either in vitro or in vivo. In some embodiments, a cytostatic agent is an agent that reduces the percentage of cells in S phase. In some embodiments, a cytostatic agent reduces the percentage of cells in S phase by at least about 20%, at least about 40%, at least about 60%, or at least about 80%.

The term “tumor” refers to all neoplastic cell growth and proliferation, whether malignant or benign, and all pre-cancerous and cancerous cells and tissues. The terms “cancer,” “cancerous,” “cell proliferative disorder,” “proliferative disorder” and “tumor” are not mutually exclusive as referred to herein. The terms “cell proliferative disorder” and “proliferative disorder” refer to disorders that are associated with some degree of abnormal cell proliferation. In some embodiments, the cell proliferative disorder is a cancer.

The term “pharmaceutical composition” refers to a preparation which is in such form as to permit the biological activity of an active ingredient contained therein to be effective in treating a subject, and which contains no additional components which are unacceptably toxic to the subject.

The terms “modulate” and “modulation” refer to reducing or inhibiting or, alternatively, activating or increasing, a recited variable.

The terms “increase” and “activate” refer to an increase of 10%, 20%, 30%, 40%, 50%, 60%, 70%, 75%, 80%, 85%, 90%, 95%, 100%, 2-fold, 3-fold, 4-fold, 5-fold, 10-fold, 20-fold, 50-fold, 100-fold, or greater in a recited variable.

The terms “reduce” and “inhibit” refer to a decrease of 10%, 20%, 30%, 40%, 50%, 60%, 70%, 75%, 80%, 85%, 90%, 95%, 2-fold, 3-fold, 4-fold, 5-fold, 10-fold, 20-fold, 50-fold, 100-fold, or greater in a recited variable.

The term “agonize” refers to the activation of receptor signaling to induce a biological response associated with activation of the receptor. An “agonist” is an entity that binds to and agonizes a receptor.

The term “antagonize” refers to the inhibition of receptor signaling to inhibit a biological response associated with activation of the receptor. An “antagonist” is an entity that binds to and antagonizes a receptor.

The term “effector T cell” includes T helper (i.e., CD4+) cells and cytotoxic (i.e., CD8+) T cells. CD4+ effector T cells contribute to the development of several immunologic processes, including maturation of B cells into plasma cells and memory B cells, and activation of cytotoxic T cells and macrophages. CD8+ effector T cells destroy virus-infected cells and tumor cells. See Seder and Ahmed, Nature Immunol., 2003, 4:835-842, incorporated by reference in its entirety, for additional information on effector T cells.

The term “regulatory T cell” includes cells that regulate immunological tolerance, for example, by suppressing effector T cells. In some embodiments, the regulatory T cell has a CD4+CD25+Foxp3+ phenotype. In some embodiments, the regulatory T cell has a CD8+CD25+ phenotype. See Nocentini et al., Br. J. Pharmacol., 2012, 165:2089-2099, incorporated by reference in its entirety, for additional information on regulatory T cells.

The term “dendritic cell” refers to a professional antigen-presenting cell capable of activating a naïve T cell and stimulating growth and differentiation of a B cell.

A “variant” of a polypeptide (e.g., an antibody) comprises an amino acid sequence wherein one or more amino acid residues are inserted into, deleted from and/or substituted into the amino acid sequence relative to the native polypeptide sequence, and retains essentially the same biological activity as the native polypeptide. The biological activity of the polypeptide can be measured using standard techniques in the art (for example, if the variant is an antibody, its activity may be tested by binding assays, as described herein). Variants of the present disclosure include fragments, analogs, recombinant polypeptides, synthetic polypeptides, and/or fusion proteins.

A “derivative” of a polypeptide is a polypeptide (e.g., an antibody) that has been chemically modified, e.g., via conjugation to another chemical moiety such as, for example, polyethylene glycol, albumin (e.g., human serum albumin), phosphorylation, and glycosylation. Unless otherwise indicated, the term “antibody” includes, in addition to antibodies comprising two full-length heavy chains and two full-length light chains, derivatives, variants, fragments, and muteins thereof, examples of which are described below.

A nucleotide sequence is “operably linked” to a regulatory sequence if the regulatory sequence affects the expression (e.g., the level, timing, or location of expression) of the nucleotide sequence. A “regulatory sequence” is a nucleic acid that affects the expression (e.g., the level, timing, or location of expression) of a nucleic acid to which it is operably linked. The regulatory sequence can, for example, exert its effects directly on the regulated nucleic acid, or through the action of one or more other molecules (e.g., polypeptides that bind to the regulatory sequence and/or the nucleic acid). Examples of regulatory sequences include promoters, enhancers and other expression control elements (e.g., polyadenylation signals). Further examples of regulatory sequences are described in, for example, Goeddel, 1990, Gene Expression Technology: Methods in Enzymology 185, Academic Press, San Diego, Calif. and Baron et al., 1995, Nucleic Acids Res. 23:3605-06. [0078]

A “host cell” is a cell that can be used to express a nucleic acid, e.g., a nucleic acid of the present disclosure. A host cell can be a prokaryote, for example, E. coli, or it can be a eukaryote, for example, a single-celled eukaryote (e.g., a yeast or other fungus), a plant cell (e.g., a tobacco or tomato plant cell), an animal cell (e.g., a human cell, a monkey cell, a hamster cell, a rat cell, a mouse cell, or an insect cell) or a hybridoma. Examples of host cells include CS-9 cells, the COS-7 line of monkey kidney cells (ATCC CRL 1651) (see Gluzman et al., 1981, Cell 23:175), L cells, C127 cells, 3T3 cells (ATCC CCL 163), Chinese hamster ovary (CHO) cells or their derivatives such as Veggie CHO and related cell lines which grow in serum-free media (see Rasmussen et al., 1998, Cytotechnology 28:31), HeLa cells, BHK (ATCC CRL 10) cell lines, the CV1/EBNA cell line derived from the African green monkey kidney cell line CV1 (ATCC CCL 70) (see McMahan et al., 1991, EMBO J. 10:2821), human embryonic kidney cells such as 293, 293 EBNA or MSR 293, human epidermal A431 cells, human Colo205 cells, other transformed primate cell lines, normal diploid cells, cell strains derived from in vitro culture of primary tissue, primary explants, HL-60, U937, HaK or Jurkat cells. Typically, a host cell is a cultured cell that can be transformed or transfected with a polypeptide-encoding nucleic acid, which can then be expressed in the host cell.

The phrase “recombinant host cell” can be used to denote a host cell that has been transformed or transfected with a nucleic acid to be expressed. A host cell also can be a cell that comprises the nucleic acid but does not express it at a desired level unless a regulatory sequence is introduced into the host cell such that it becomes operably linked with the nucleic acid. It is understood that the term host cell refers not only to the particular subject cell but to the progeny or potential progeny of such a cell. Because certain modifications may occur in succeeding generations due to, e.g., mutation or environmental influence, such progeny may not, in fact, be identical to the parent cell, but are still included within the scope of the term as used herein.

In some embodiments, the host cell is used in adoptive cell therapy for delivery of the ABP to the subject.

### 7.2. Other Interpretational Conventions

Ranges recited herein are understood to be shorthand for all of the values within the range, inclusive of the recited endpoints. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

Unless otherwise indicated, reference to a compound that has one or more stereocenters intends each stereoisomer, and all combinations of stereoisomers, thereof.

### 7.3. Antigen Binding Protein

In one aspect, the present disclosure provides antigen binding proteins (e.g., antibodies, antibody fragments, antibody derivatives, antibody muteins, and antibody variants). In some embodiments, the ABP bind to CTLA-4. In particular, the present disclosure provides ABPs that bind to an epitope of CTLA-4, which is different from Ipilimumab. In some embodiments, the epitope comprises K130, Y139, L141, and I143, but not R70. In some embodiments, the ABP contacts amino acids K130, Y139, L141, I143 but does not contact amino acid R70 of the CTLA-4. In some embodiments, the ABP can bind CTLA-4 even while the CTPA-4 interacts with CD80/CD86. In some embodiments, an interaction between the ABP and amino acid L74A and/or E68 of the CTLA-4 is greater than an interaction between Ipilimumab and amino acid L74A of CTLA-4.

In some embodiments, the present disclosure provides antigen binding proteins that comprise a light chain variable region selected from the group consisting of A1LC-A28LC or a heavy chain variable region selected from the group consisting of A1HC-A28HC, and fragments, derivatives, muteins, and variants thereof. Such an antigen binding protein can be denoted using the nomenclature “LxHy,” wherein “x” corresponds to the number of the light chain variable region and “y” corresponds to the number of the heavy chain variable region as they are labeled in the sequences below. That is to say, for example, that “A1HC” denotes the heavy chain variable region comprising the amino acid sequence of SEQ ID NO: 101; “A1LC” denotes the light chain variable region comprising the amino acid sequence of SEQ ID NO: 1, and so forth. More generally speaking, “L2H1” refers to an antigen binding protein with a light chain variable region comprising the amino acid sequence of L2 (SEQ ID NO:2) and a heavy chain variable region comprising the amino acid sequence of H1 (SEQ ID NO:101). For clarity, all ranges denoted by at least two members of a group include all members of the group between and including the end range members. Thus, the group range A1-A28, includes all members between A1 and A28, as well as members A1 and A28 themselves. The group range A4-A6 includes members A4, A5, and A6, etc. In a particular embodiment, the ABP is A14.

In some embodiments, antigen binding proteins comprise variable (V(D)J) regions of both heavy and light chain sequences identical to one of the clones in the library of CTLA-4 binding clones, deposited under ATCC Accession NO. PTA-125512. In some embodiments, antigen binding proteins comprise variable (V(D)J) regions of either heavy or light chain sequence identical to one of the clones in the library of CTLA-4 binding clones, deposited under ATCC Accession NO. PTA-125512. In some embodiments, antigen binding proteins are expressed from the expression vector in one of the clones in the library of CTLA-4 binding clones, deposited under ATCC Accession NO. PTA-125512.

Also shown below are the locations of the CDRs (underlined) that create part of the antigen-binding site, while the Framework Regions (FRs) are the intervening segments of these variable domain sequences. In both light chain variable regions and heavy chain variable regions there are three CDRs (CDR1-3) and four FRs (FR 1-4). The CDR regions of each light and heavy chain also are grouped by antibody type (A1, A2, A3, etc.). Antigen binding proteins of the present disclosure include, for example, antigen binding proteins having a combination of light chain and heavy chain variable domains selected from the group of combinations consisting of L1H1 (antibody A1; used interchangeably herein as “aCTLA-4.9”). L2H2 (antibody A2; used interchangeably herein as “aCTLA-4.4”), L3H3 (antibody A3; used interchangeably herein as “aCTLA-4.2”), L4114 (antibody A4; used interchangeably herein as “aCTLA-4.29”), L5H5 (antibody A5; used interchangeably herein as “aCTLA-4.28”), L6H6 (antibody A6; used interchangeably herein as “aCTLA-4.26”), L7117 (antibody A7; used interchangeably herein as “aCTLA-4.3”), L8118 (antibody A8; used interchangeably herein as “aCTLA-4.1”), L9119 (antibody A9; used interchangeably herein as “aCTLA-4.24”), L10H10 (antibody A10; used interchangeably herein as “aCTLA-4.22”), L11H11 (antibody A11; used interchangeably herein as “aCTLA-4.31). L121112 (antibody A12; used interchangeably herein as “aCTLA-4.12”), L13H13 (antibody A13; used interchangeably herein as “aCTLA-4.14”), L131113 (antibody A13; used interchangeably herein as “aCTLA-4.14”) . . . and L28H28 (antibody A28). Antigen binding proteins of the present disclosure include, for example, antigen binding proteins having a light chain and heavy chain variable domain selected from the group consisting of L18H18 (antibody A18; used interchangeably herein as “aCTLA-4.11”), L15H15 (antibody A15; used interchangeably herein as “aCTLA-4.18”), L16H16 (antibody A16; used interchangeably herein as “aCTLA-4.5”), and L17H17 (antibody A17; used interchangeably herein as “aCTLA-4.17”). In some embodiments, ABPs of the present disclosure comprise a: L14H14 (antibody A14; used interchangeably herein as “aCTLA-4.15” or GIGA-564).

Antigen binding proteins of the present disclosure include, for example, antigen binding proteins having a light chain and heavy chain variable domain selected from the group consisting of L19H19 (antibody A19; used interchangeably herein as “aCTLA-4.7”), L20H20 (antibody A20; used interchangeably herein as “aCTLA-4.25”), L21H21 (antibody A21; used interchangeably herein as “aCTLA-4.10”), L22H22 (antibody A22; used interchangeably herein as “aCTLA-4.21”), L23H23 (antibody A23; used interchangeably herein as “aCTLA-4.23”), L24H24 (antibody A24; used interchangeably herein as “aCTLA-4.27”), L25H25 (antibody A25; used interchangeably herein as “aCTLA-4.32”), L26H26 (antibody A26; used interchangeably herein as “aCTLA-4.20”), and L27H27 (antibody A27; used interchangeably herein as “aCTLA-4.8”). In some embodiments, ABPs of the present disclosure comprise a light chain and heavy chain variable domain of L14H14 (antibody A14; used interchangeably herein as “aCTLA-4.15” or GIGA-564).

In some embodiments, antigen binding proteins comprise all six CDR sequences (three CDRs of light chain and three CDRs of heavy chain) identical to one of the clones in the library of CTLA-4 binding clones, deposited under ATCC Accession NO. PTA-125512. In some embodiments, antigen binding proteins comprise three out of six CDR sequences (three CDRs of light chain or three CDRs of heavy chain) identical to one of the clones in the library of CTLA-4 binding clones, deposited under ATCC Accession NO. PTA-125512. In some embodiments, antigen binding proteins comprise one, two, three, four, or five out of six CDR sequences identical to one of the clones in the library of CTLA-4 binding clones, deposited under ATCC Accession NO. PTA-125512.

In one embodiment, the present disclosure provides an antigen binding protein comprising a light chain variable domain comprising a sequence of amino acids that differs from the sequence of a light chain variable domain selected from the group consisting of L1 through L28 only at 15, 14, 13, 12, 11, 10, 9, 8, 7, 6, 5, 4, 3, 2, or 1 residues, wherein each such sequence difference is independently either a deletion, insertion, or substitution of one amino acid residue. In another embodiment, the light-chain variable domain comprises a sequence of amino acids that is at least 70%, 75%, 80%, 85%, 90%, 95%, 96%, 97%, 98%, or 99% identical to the sequence of alight chain variable domain selected from the group consisting of L1-L28. In another embodiment, the light chain variable domain comprises a sequence of amino acids that is encoded by a nucleotide sequence that is at least 70%, 75%, 80%, 85%, 90%, 95%, 96%, 97%, 98%, or 99% identical to a nucleotide sequence that encodes a light chain variable domain selected from the group consisting of L1-L28 (which includes L1, L2, L3, L4, L5, L6, L7, L8, L9, L10, L11, L12, L13, L14, . . . and L28). In another embodiment, the light chain variable domain comprises a sequence of amino acids that is encoded by a polynucleotide that hybridizes under moderately stringent conditions to the complement of a polynucleotide that encodes a light chain variable domain selected from the group consisting of L1-L28. In another embodiment, the light chain variable domain comprises a sequence of amino acids that is encoded by a polynucleotide that hybridizes under moderately stringent conditions to the complement of a polynucleotide that encodes a light chain variable domain selected from the group consisting of L1-L28. In another embodiment, the light chain variable domain comprises a sequence of amino acids that is encoded by a polynucleotide that hybridizes under moderately stringent conditions to a complement of a light chain polynucleotide of L1-L28.

In one embodiment, the present disclosure provides an antigen binding protein comprising a light chain variable domain comprising a sequence of amino acids that differs from the sequence of a light chain variable domain encoded by one of the clones of the library of CTLA-4 binding clones, deposited under ATCC Accession NO. PTA-125512, only at 15, 14, 13, 12, 11, 10, 9, 8, 7, 6, 5, 4, 3, 2, or 1 residues, wherein each such sequence difference is independently either a deletion, insertion, or substitution of one amino acid residue. In another embodiment, the light-chain variable domain comprises a sequence of amino acids that is at least 70%, 75%, 80%, 85%, 90%, 95%, 96%, 97%, 98%, or 99% identical to the sequence of a light chain variable domain encoded by one of the clones of the library of CTLA-4 binding clones, deposited under ATCC Accession NO. PTA-125512. In another embodiment, the light chain variable domain comprises a sequence of amino acids that is encoded by a nucleotide sequence that is at least 70%, 75%, 80%, 85%, 90%, 95%, 96%, 97%, 98%, or 99% identical to a nucleotide sequence of one of the clones of the library of CTLA-4 binding clones, deposited under ATCC Accession NO. PTA-125512.

In another embodiment, the present disclosure provides an antigen binding protein comprising a heavy chain variable domain comprising a sequence of amino acids that differs from the sequence of a heavy chain variable domain selected from the group consisting of H1-H28 only at 15, 14, 13, 12, 11, 10, 9, 8, 7, 6, 5, 4, 3, 2, or 1 residue(s), wherein each such sequence difference is independently either a deletion, insertion, or substitution of one amino acid residue. In another embodiment, the heavy chain variable domain comprises a sequence of amino acids that is at least 70%, 75%, 80%, 85%, 90%, 95%, 96%, 97%, 98%, or 99% identical to the sequence of a heavy chain variable domain selected from the group consisting of H1-H28. In another embodiment, the heavy chain variable domain comprises a sequence of amino acids that is encoded by a nucleotide sequence that is at least 70%, 75%, 80%, 85%, 90%, 95%, 96%, 97%, 98%, or 99% identical to a nucleotide sequence that encodes a heavy chain variable domain selected from the group consisting of H1-H28. In another embodiment, the heavy chain variable domain comprises a sequence of amino acids that is encoded by a polynucleotide that hybridizes under moderately stringent conditions to the complement of a polynucleotide that encodes a heavy chain variable domain selected from the group consisting of H1-H28. In another embodiment, the heavy chain variable domain comprises a sequence of amino acids that is encoded by a polynucleotide that hybridizes under moderately stringent conditions to the complement of a polynucleotide that encodes a heavy chain variable domain selected from the group consisting of H1-H28. In another embodiment, the heavy chain variable domain comprises a sequence of amino acids that is encoded by a polynucleotide that hybridizes under moderately stringent conditions to a complement of a heavy chain polynucleotide disclosed herein.

In one embodiment, the present disclosure provides an antigen binding protein comprising a heavy chain variable domain comprising a sequence of amino acids that differs from the sequence of a heavy chain variable domain encoded by one of the clones of the library of CTLA-4 binding clones, deposited under ATCC Accession NO. PTA-125512, only at 15, 14, 13, 12, 11, 10, 9, 8, 7, 6, 5, 4, 3, 2, or 1 residues, wherein each such sequence difference is independently either a deletion, insertion, or substitution of one amino acid residue. In another embodiment, the heavy chain variable domain comprises a sequence of amino acids that is at least 70%, 75%, 80%, 85%, 90%, 95%, 96%, 97%, 98%, or 99% identical to the sequence of a heavy chain variable domain encoded by one of the clones of the library of CTLA-4 binding clones, deposited under ATCC Accession NO. PTA-125512. In another embodiment, the heavy chain variable domain comprises a sequence of amino acids that is encoded by a nucleotide sequence that is at least 70%, 75%, 80%, 85%, 90%, 95%, 96%, 97%, 98%, or 99% identical to a nucleotide sequence of one of the clones of the library of CTLA-4 binding clones, deposited under ATCC Accession NO. PTA-125512.

Particular embodiments of antigen binding proteins of the present disclosure comprise one or more amino acid sequences that are identical to the amino acid sequences of one or more of the CDRs and/or FRs referenced herein. In one embodiment, the antigen binding protein comprises a light chain CDR1 sequence illustrated above. In another embodiment, the antigen binding protein comprises a light chain CDR2 sequence illustrated above. In another embodiment, the antigen binding protein comprises a light chain CDR3 sequence illustrated above. In another embodiment, the antigen binding protein comprises a heavy chain CDR1 sequence illustrated above. In another embodiment, the antigen binding protein comprises a heavy chain CDR2 sequence illustrated above. In another embodiment, the antigen binding protein comprises a heavy chain CDR3 sequence illustrated above.

In one embodiment, the present disclosure provides an antigen binding protein that comprises one or more CDR sequences that differ from a CDR sequence shown above by no more than 5, 4, 3, 2, or 1 amino acid residues.

In some embodiments, at least one of the antigen binding protein's CDR1 sequences is a CDR1 sequence from A1-A28, CDR1-L1 to 28, or CDR1-H1 to 28 as shown in TABLE 5. In some embodiments, at least one of the antigen binding protein's CDR2 sequences is a CDR2 sequence from A1-A28, CDR2-L1 to 28, or CDR2-H1 to 28 as shown in TABLE 5. In some embodiments, at least one of the antigen binding protein's CDR3 sequences is a CDR3 sequence from A1-A28, CDR3-L1 to 28, or CDR3-H1 to 28 as shown in TABLE 5.

In another embodiment, the antigen binding protein's light chain CDR3 sequence is alight chain CDR3 sequence from A1-A28 or CDR3-L1 to 28, as shown in TABLE 5, and the antigen binding protein's heavy chain CDR3 sequence is a heavy chain sequence from A1-A28 or CDR-H1 to 28, as shown in TABLE 5.

In some embodiments, at least one of the antigen binding protein's CDR1 sequences is a light chain CDR1 sequence of QSVSSSYLA (SEQ ID NO: 12078 or 1014). In some embodiments, at least one of the antigen binding protein's CDR2 sequences is a light chain CDR2 sequence of GASSRAT (SEQ ID NO: 12079 or 2014). In some embodiments, at least one of the antigen binding protein's CDR3 sequences is a light chain CDR3 sequence of QQYGSSPWT (SEQ ID NO: 12080 or 3014).

In some embodiments, at least one of the antigen binding protein's CDR1 sequences is a heavy chain CDR1 sequence of GFTFSSY (SEQ ID NO: 12075 or 4014). In some embodiments, at least one of the antigen binding protein's CDR2 sequences is a heavy chain CDR2 sequence of WYEGRN (SEQ ID NO: 12076 or 5014). In some embodiments, at least one of the antigen binding protein's CDR3 sequences is a heavy chain CDR3 sequence of AGDLGAFDI (SEQ ID NO: 12077 or 6014).

In some embodiments, the ABP comprises a CDR1-L consisting of SEQ ID NO: 12004, a CDR2-L consisting of SEQ ID NO: 12014, a CDR3-L consisting of SEQ ID NO: 12024, a CDR1-H consisting of SEQ ID NO: 12039, a CDR2-H consisting of SEQ ID NO: 12049, and a CDR3-H consisting of SEQ ID NO: 12059. In some embodiments, the ABP comprises a CDR1-L consisting of SEQ ID NO: 12005, a CDR2-L consisting of SEQ ID NO: 12015, a CDR3-L consisting of SEQ ID NO: 12025, a CDR1-H consisting of SEQ ID NO: 12040, a CDR2-H consisting of SEQ ID NO: 12050, and a CDR3-H consisting of SEQ ID NO: 12060. In some embodiments, the ABP comprises a CDR1-L consisting of SEQ ID NO: 12006, a CDR2-L consisting of SEQ ID NO: 12016, a CDR3-L consisting of SEQ ID NO: 12026, a CDR1-H consisting of SEQ ID NO: 12041, a CDR2-H consisting of SEQ ID NO: 12051, and a CDR3-H consisting of SEQ ID NO: 12061, In some embodiments, the ABP comprises a CDR1-L consisting of SEQ ID NO: 12007, a CDR2-L consisting of SEQ ID NO: 12017, a CDR3-L consisting of SEQ ID NO: 12027, a CDR1-H consisting of SEQ ID NO: 12042, a CDR2-H consisting of SEQ ID NO: 12052, and a CDR3-H consisting of SEQ ID NO: 12062. In some embodiments, the ABP comprises a CDR1-L consisting of SEQ ID NO: 12008, a CDR2-L consisting of SEQ ID NO: 12018, a CDR3-L consisting of SEQ ID NO: 12028, a CDR1-H consisting of SEQ ID NO: 12043, a CDR2-H consisting of SEQ ID NO: 12053, and a CDR3-H consisting of SEQ ID NO: 12063.

In another embodiment, the antigen binding protein comprises 1, 2, 3, 4, or 5 CDR sequence(s) that each independently differs by 6, 5, 4, 3, 2, 1, or 0 single amino acid additions, substitutions, and/or deletions from a CDR sequence of A1-A23, and the antigen binding protein further comprises 1, 2, 3, 4, or 5 CDR sequence(s) that each independently differs by 6, 5, 4, 3, 2, 1, or 0 single amino acid additions, substitutions, and/or deletions from a CDR sequence. In some embodiments, the antigen binding protein comprises 1, 2, 3, 4, or 5 CDR sequence(s) that each has at least 50%, 55%, 60%, 65%, 70%, 75%, 80%, 85%, 90%, 95%, 96%, 97%, 98%, or 99% sequence identity to a CDR sequence of A1-A28.

The nucleotide sequences of A1-A28, or the amino acid sequences of A1-A28, can be altered, for example, by random mutagenesis or by site-directed mutagenesis (e.g., oligonucleotide-directed site-specific mutagenesis) to create an altered polynucleotide comprising one or more particular nucleotide substitutions, deletions, or insertions as compared to the non-mutated polynucleotide. Examples of techniques for making such alterations are described in Walder et al., 1986, Gene 42:133; Bauer et al. 1985, Gene 37:73; Craik, BioTechniques, January 1985, 12-19; Smith et al., 1981, Genetic Engineering: Principles and Methods, Plenum Press; and U.S. Pat. Nos. 4,518,584 and 4,737,462. These and other methods can be used to make, for example, derivatives of anti-CTLA-4 antibodies that have a desired property, for example, increased affinity, avidity, or specificity for CTLA-4, increased activity or stability in vivo or in vitro, or reduced in vivo side-effects as compared to the underivatized antibody.

Other derivatives of anti-CTLA-4 antibodies within the scope of this disclosure include covalent or aggregative conjugates of anti-CTLA-4 antibodies, or fragments thereof, with other proteins or polypeptides, such as by expression of recombinant fusion proteins comprising heterologous polypeptides fused to the N-terminus or C-terminus of an anti-CTLA-4 antibody polypeptide. For example, the conjugated peptide may be a heterologous signal (or leader) polypeptide, e.g., the yeast alpha-factor leader, or a peptide such as an epitope tag. Antigen binding protein-containing fusion proteins can comprise peptides added to facilitate purification or identification of antigen binding protein (e.g., poly-His). An antigen binding protein also can be linked to the FLAG peptide Asp-Tyr-Lys-Asp-Asp-Asp-Asp-Lys (DYKDDDDK) (SEQ ID NO: 7002) as described in Hopp et al., Bio/Technology 6:1204, 1988, and U.S. Pat. No. 5,011,912. The FLAG peptide is highly antigenic and provides an epitope reversibly bound by a specific monoclonal antibody (mAb), enabling rapid assay and facile purification of expressed recombinant protein. Reagents useful for preparing fusion proteins in which the FLAG peptide is fused to a given polypeptide are commercially available (Sigma, St. Louis, Mo.).

One suitable Fc polypeptide, described in PCT application WO 93/10151 (hereby incorporated by reference), is a single chain polypeptide extending from the N-terminal hinge region to the native C-terminus of the Fc region of a human IgG1 antibody. Another useful Fc polypeptide is the Fc mutein described in U.S. Pat. No. 5,457,035 and in Baum et al., 1994, EMBO J. 13:3992-4001. The amino acid sequence of this mutein is identical to that of the native Fc sequence presented in WO 93/10151, except that amino acid 19 has been changed from Leu to Ala, amino acid 20 has been changed from Leu to Glu, and amino acid 22 has been changed from Gly to Ala. The mutein exhibits reduced affinity for Fc receptors.

In other embodiments, the variable portion of the heavy and/or light chains of an anti-CTLA-4 antibody may be substituted for the variable portion of an antibody heavy and/or light chain.

Oligomers that contain one or more antigen binding proteins may be employed as CTLA-4 antagonists or agonists. Oligomers may be in the form of covalently-linked or non-covalently-linked dimers, trimers, or higher oligomers. Oligomers comprising two or more antigen binding protein are contemplated for use, with one example being a homodimer. Other oligomers include heterodimers, homotrimers, heterotrimers, homotetramers, heterotetramers, etc.

One embodiment is directed to oligomers comprising multiple antigen binding proteins joined via covalent or non-covalent interactions between peptide moieties fused to the antigen binding proteins. Such peptides may be peptide linkers (spacers), or peptides that have the property of promoting oligomerization. Leucine zippers and certain polypeptides derived from antibodies are among the peptides that can promote oligomerization of antigen binding proteins attached thereto, as described in more detail below.

In particular embodiments, the oligomers comprise from two to four antigen binding proteins. The antigen binding proteins of the oligomer may be in any form, such as any of the forms described above, e.g., variants or fragments. Preferably, the oligomers comprise antigen binding proteins that have CTLA-4 binding activity.

In one embodiment, an oligomer is prepared using polypeptides derived from immunoglobulins. Preparation of fusion proteins comprising certain heterologous polypeptides fused to various portions of antibody-derived polypeptides (including the Fe domain) has been described, e.g., by Ashkenazi et al., 1991, PNAS USA 88:10535; Byrn et al., 1990, Nature 344:677; and Hollenbaugh et al., 1992 Curr. Prots in Immunol., Suppl. 4, pages 10.19.1-10.19.11.

One embodiment of the present disclosure is directed to a dimer comprising two fusion proteins created by fusing a CTLA-4 binding fragment of an anti-CTLA-4 antibody to the Fc region of an antibody. The dimer can be made by, for example, inserting a gene fusion encoding the fusion protein into an appropriate expression vector, expressing the gene fusion in host cells transformed with the recombinant expression vector, and allowing the expressed fusion protein to assemble much like antibody molecules, whereupon interchain disulfide bonds form between the Fc moieties to yield the dimer.

Alternatively, the oligomer is a fusion protein comprising multiple antigen binding proteins, with or without peptide linkers (spacer peptides). Among the suitable peptide linkers are those described in U.S. Pat. Nos. 4,751,180 and 4,935,233.

Another method for preparing oligomeric antigen binding proteins involves use of a leucine zipper. Leucine zipper domains are peptides that promote oligomerization of the proteins in which they are found. Leucine zippers were originally identified in several DNA-binding proteins (Landschulz et al., 1988, Science 240:1759), and have since been found in a variety of different proteins. Among the known leucine zippers are naturally occurring peptides and derivatives thereof that dimerize or trimerize. Examples of leucine zipper domains suitable for producing soluble oligomeric proteins are described in PCT application WO 94/10308, and the leucine zipper derived from lung surfactant protein D (SPD) described in Hoppe et al., 1994, FEBS Letters 344:191, hereby incorporated by reference. The use of a modified leucine zipper that allows for stable trimerization of a heterologous protein fused thereto is described in Fanslow et al., 1994, Semin. Immunol. 6:267-78. In one approach, recombinant fusion proteins comprising an anti-CTLA-4 antibody fragment or derivative fused to a leucine zipper peptide are expressed in suitable host cells, and the soluble oligomeric anti-CTLA-4 antibody fragments or derivatives that form are recovered from the culture supernatant.

In one aspect, the present disclosure provides antigen binding proteins that interfere with the binding of CTLA-4 to its ligands. Such antigen binding proteins can be made against CTLA-4, or a fragment, variant or derivative thereof, and screened in conventional assays for the ability to interfere with binding of CTLA-4 to its ligands. Examples of suitable assays are assays that test the antigen binding proteins for the ability to inhibit binding of CTLA-4 ligands to cells expressing CTLA-4, or that test antigen binding proteins for the ability to reduce a biological or cellular response that results from the binding of CTLA-4 ligands to cell surface CTLA-4. For example, antibodies can be screened according to their ability to bind to immobilized antibody surfaces (CTLA-4). Antigen binding proteins that block the binding of CTLA-4 to a ligand can be employed in treating any CTLA-4-related condition, including but not limited to cancer. In an embodiment, a human anti-CTLA-4 monoclonal antibody generated by procedures involving immunization of transgenic mice is employed in treating such conditions.

Antigen-binding fragments of antigen binding proteins of the present disclosure can be produced by conventional techniques. Examples of such fragments include, but are not limited to, Fab and F(ab′)₂ fragments. Antibody fragments and derivatives produced by genetic engineering techniques also are contemplated.

Additional embodiments include chimeric antibodies, e.g., humanized versions of non-human (e.g., murine) monoclonal antibodies. Such humanized antibodies may be prepared by known techniques, and offer the advantage of reduced immunogenicity when the antibodies are administered to humans. In one embodiment, a humanized monoclonal antibody comprises the variable domain of a murine antibody (or all or part of the antigen binding site thereof) and a constant domain derived from a human antibody. Alternatively, a humanized antibody fragment may comprise the antigen binding site of a murine monoclonal antibody and a variable domain fragment (lacking the antigen-binding site) derived from a human antibody. Procedures for the production of chimeric and further engineered monoclonal antibodies include those described in Riechmann et al., 1988, Nature 332:323, Liu et al., 1987, Proc. Nat. Acad. Sci. USA 84:3439, Larrick et al., 1989, Bio/Technology 7:934, and Winter et al., 1993, TIPS 14:139. In one embodiment, the chimeric antibody is a CDR grafted antibody. Techniques for humanizing antibodies are discussed in, e.g., U.S. Pat. Nos. 5,869,619, 5,225,539, 5,821,337, 5,859,205, 6,881,557, Padlan et al., 1995, FASEB J. 9:133-39, and Tamura et al., 2000, J. Immunol. 164:1432-41.

Procedures have been developed for generating human or partially human antibodies in non-human animals. For example, mice in which one or more endogenous immunoglobulin genes have been inactivated by various means have been prepared. Human immunoglobulin genes have been introduced into the mice to replace the inactivated mouse genes. Antibodies produced in the animal incorporate human immunoglobulin polypeptide chains encoded by the human genetic material introduced into the animal. In one embodiment, a non-human animal, such as a transgenic mouse, is immunized with a CTLA-4 polypeptide, such that antibodies directed against the CTLA-4 polypeptide are generated in the animal.

One example of a suitable immunogen is a soluble human CTLA-4, such as a polypeptide comprising the extracellular domain of the protein having the following sequence: SEQ ID: 7001 or other immunogenic fragment of the protein. Examples of techniques for production and use of transgenic animals for the production of human or partially human antibodies are described in U.S. Pat. Nos. 5,814,318, 5,569,825, and 5,545,806, Davis et al., 2003, Production of human antibodies from transgenic mice in Lo, ed. Antibody Engineering: Methods and Protocols, Humana Press, NJ:191-200, Kellermann et al., 2002, Curr Opin Biotechnol. 13:593-97, Russel et al., 2000, Infect Immun. 68:1820-26, Gallo et al., 2000, Eur J. Immun. 30:534-40, Davis et al., 1999. Cancer Metastasis Rev. 18:421-25, Green, 1999, J. Immunol Methods. 231:11-23, Jakobovits, 1998, Advanced Drug Delivery Reviews 31:33-42, Green et al., 1998, J Exp Med. 188:483-95, Jakobovits A, 1998, Exp. Opin. Invest. Drugs. 7:607-14, Tsuda et al., 1997, Genomics. 42:413-21, Mendez et al., 1997, Nat Genet. 15:146-56, Jakobovits, 1994, Curr Biol. 4:761-63, Arbones et al., 1994, Immunity. 1:247-60, Green et al., 1994, Nat Genet. 7:13-21, Jakobovits et al., 1993, Nature. 362:255-58, Jakobovits et al., 1993, Proc Natl Acad Sci USA. 90:2551-55. Chen, J., M. Trounstine, F. W. Alt, F. Young, C. Kurahara, J. Loring, D. Huszar. Inter'l Immunol. 5 (1993): 647-656, Choi et al., 1993, Nature Genetics 4: 117-23, Fishwild et al., 1996, Nature Biotech. 14: 845-51, Harding et al., 1995, Annals of the New York Academy of Sciences, Lonberg et al., 1994, Nature 368: 856-59, Lonberg, 1994, Transgenic Approaches to Human Monoclonal Antibodies in Handbook of Experimental Pharmacology 113: 49-101, Lonberg et al., 1995, Internal Review of Immunology 13: 65-93, Neuberger, 1996, Nature Biotechnology 14: 826, Taylor et al., 1992, Nucleic Acids Res. 20: 6287-95, Taylor et al., 1994, Inter'l Immunol. 6: 579-91, Tomizuka et al., 1997, Nature Genetics 16: 133-43, Tomizuka et al., 2000, Pro. Nat'l Acad. Sci. USA 97: 722-27, Tuaillon et al., 1993, Pro. Nat'l Acad. Sci. USA 90: 3720-24, and Tuaillon et al., 1994, J. Immunol. 152: 2912-20.

Antigen binding proteins (e.g., antibodies, antibody fragments, and antibody derivatives) of the present disclosure can comprise any constant region known in the art. The light chain constant region can be, for example, a kappa- or lambda-type light chain constant region, e.g., a human kappa- or lambda-type light chain constant region. The heavy chain constant region can be, for example, an alpha-, delta-, epsilon-, gamma-, or mu-type heavy chain constant regions, e.g., a human alpha-, delta-, epsilon-, gamma-, or mu-type heavy chain constant region. In one embodiment, the light or heavy chain constant region is a fragment, derivative, variant, or mutein of a naturally occurring constant region.

Techniques are known for deriving an antibody of a different subclass or isotype from an antibody of interest, i.e., subclass switching. Thus, IgG antibodies may be derived from an IgM antibody, for example, and vice versa. Such techniques allow the preparation of new antibodies that possess the antigen-binding properties of a given antibody (the parent antibody), but also exhibit biological properties associated with an antibody isotype or subclass different from that of the parent antibody. Recombinant DNA techniques may be employed. Cloned DNA encoding particular antibody polypeptides may be employed in such procedures, e.g., DNA encoding the constant domain of an antibody of the desired isotype. See also Lantto et al., 2002, Methods Mol. Biol. 178:303-16.

In one embodiment, an antigen binding protein of the present disclosure comprises the IgG1 heavy chain domain of any of A1-A28 (H1-H28) or a fragment of the IgG1 heavy chain domain of any of A1-A28 (H1-H28). In another embodiment, an antigen binding protein of the present disclosure comprises the kappa light chain constant chain region of A1-A28 (L1-L28), or a fragment of the kappa light chain constant region of A1-A28 (L1-L28). In another embodiment, an antigen binding protein of the present disclosure comprises both the IgG1 heavy chain domain, or a fragment thereof, of A1-A28 (H1-H28) and the kappa light chain domain, or a fragment thereof, of A1-A28 (L1-L28).

In another embodiment, an antigen binding protein of the present disclosure comprises both the IgG1 heavy chain domain or a fragment thereof, of A1-A28 (H1-H28). In some embodiments, the IgG1 heavy chain domain comprises a lysine at amino acid position 97 (K97) according to IMGT exon numbering system. In another embodiment, the IgG1 heavy chain domain comprises a lysine at amino acid position 214 (K214) according to EU numbering system. In some embodiments, the IgG1 heavy chain domain comprises an Arginine at amino acid position 97 (R97) according to IMGT exon numbering system. In another embodiment, the IgG1 heavy chain domain comprises an Arginine at amino acid position 214 (R214) according to EU numbering system.

In some embodiments, the antigen binding protein of the present disclosure comprises the IgG1 heavy chain domain of A14 (H14) (SEQ ID NO: 114) or a fragment of the IgG1 heavy chain domain, and the IgG1 light chain domain of A14 (H14) (SEQ ID NO: 14) or fragment of the IgG1 light chain domain.

Accordingly, the antigen binding proteins of the present disclosure include those comprising, for example, the variable domain combinations L1H1, L2H2, L3H3, L4H4, L5H5, L6H6, L7H7, L8H8, L9H9, L10H10, L11H11, L12H12, L13H13, . . . and L28H28, having a desired isotype (for example, IgA, IgG1, IgG2, IgG3, IgG4, IgM, IgE, and IgD) as well as Fab or F(ab′)₂ fragments thereof. Moreover, if an IgG4 is desired, it may also be desired to introduce a point mutation (CPSCP (SEQ ID NO: 11969)->CPPCP (SEQ ID NO: 11970)) in the hinge region as described in Bloom et al., 1997, Protein Science 6:407, incorporated by reference herein) to alleviate a tendency to form intra-H chain disulfide bonds that can lead to heterogeneity in the IgG4 antibodies.

In one embodiment, the antigen binding protein has a K_{off} of 1×10⁻⁴ s⁻¹ or lower. In another embodiment, the K_{off} is 5×10⁻⁵ s⁻¹ or lower. In another embodiment, the K_{off} is substantially the same as an antibody having a combination of light chain and heavy chain variable domain sequences selected from the group of combinations consisting of L1H1, L2H2, L3H3, L4H4, L5H5, L6H6, . . . and L28H28. In another embodiment, the antigen binding protein binds to CTLA-4 with substantially the same K_{off} as an antibody that comprises one or more CDRs from an antibody having a combination of light chain and heavy chain variable domain sequences selected from the group of combinations consisting of L1H1, L2H2, L3H3, L4H4, L5H5, L6H6, . . . and L23H28. In another embodiment, the antigen binding protein binds to CTLA-4 with substantially the same K_{off} as an antibody that comprises one of the amino acid sequences illustrated above. In another embodiment, the antigen binding protein binds to CTLA-4 with substantially the same K_{off} as an antibody that comprises one or more CDRs from an antibody that comprises one of the amino acid sequences illustrated above.

In one aspect, the present disclosure provides antigen-binding fragments of an anti-CTLA-4 antibody of the present disclosure. Such fragments can consist entirely of antibody-derived sequences or can comprise additional sequences. Examples of antigen-binding fragments include Fab, F(ab′)2, single chain antibodies, diabodies, triabodies, tetrabodies, and domain antibodies. Other examples are provided in Lunde et al., 2002, Biochem. Soc. Trans. 30:500-06.

Single chain antibodies (scFv) may be formed by linking heavy and light chain variable domain (Fv region) fragments via an amino acid bridge (short peptide linker, e.g., a synthetic sequence of amino acid residues), resulting in a single polypeptide chain. Such single-chain Fvs (scFvs) have been prepared by fusing DNA encoding a peptide linker between DNAs encoding the two variable domain polypeptides (V_{L} and V_{H}). The resulting polypeptides can fold back on themselves to form antigen-binding monomers, or they can form multimers (e.g., dimers, trimers, or tetramers), depending on the length of a flexible linker between the two variable domains (Kortt et al., 1997, Prot. Eng. 10:423; Kortt et al., 2001, Biomol. Eng. 18:95-108, Bird et al., 1988, Science 242:423-26 and Huston et al., 1988, Proc. Natl. Acad. Sci. USA 85:5879-83). By combining different V_{L} and V_{H}-comprising polypeptides, one can form multimeric scFvs that bind to different epitopes (Kriangkum et al., 2001, Biomol. Eng. 18:31-40). Techniques developed for the production of single chain antibodies include those described in U.S. Pat. No. 4,946,778; Bird, 1988, Science 242:423; Huston et al., 1988, Proc. Natl. Acad. Sci. USA 85:5879; Ward et al., 1989, Nature 334:544, de Graaf et al., 2002, Methods Mol Biol. 178:379-87. ScFvs comprising the variable domain combinations L1H1, L2H2, L3H3, L4H4, L5H5, L6H6, . . . , and L28H28 are encompassed by the present disclosure.

ABPs provided herein can be anti-CTLA-4 antibodies purified from host cells that have been transfected by a gene encoding the antibodies by elution of filtered supernatant of host cell culture fluid using a Heparin HP column, using a salt gradient.

In some embodiments, the host cell is used in adoptive cell therapy for delivery of the ABP to the subject. In some embodiments, the methods described herein can administer the host cell that has been transfected by a gene encoding the anti-CTLA-4 antibodies.

An antigen binding protein can have, for example, the structure of a naturally occurring immunoglobulin. An “immunoglobulin” is a tetrameric molecule. In a naturally occurring immunoglobulin, each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa and lambda light chains. Heavy chains are classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)) (incorporated by reference in its entirety for all purposes). The variable regions of each light/heavy chain pair form the antibody binding site such that an intact immunoglobulin has two binding sites.

In one aspect, the present disclosure provides an ABP comprising a human heavy chain constant region gene segment of an IGHG1*01. In some embodiments, the IGHG1*01 Fc antibody comprises an scFv. In some embodiments, the ABP is specific to CTLA-4, PD-1, PD-L1, LAG-3, CD47, TIGIT, or other antigens. In some embodiments, the ABP is specific to CTLA-4. In some embodiments, the ABP comprises an antigen binding domain of an antibody therapeutic approved or in regulatory review. In some embodiments, the ABP comprises an antigen binding domain of Ipilimumab, Toripalimab. Amivantamab. Dostarlimab, Cemiplimab, Durvalumab, Atezolizumab, or Pembrolizumab.

In some embodiments, the ABP has an enhanced FcR signaling or Fc effector function by comprising a human heavy chain constant region gene segment an IGHG1*01. Accordingly, the present disclosure further provides a method of inducing FcR-mediated Treg depletion in the tumor microenvironment, comprising the step of administering the ABP comprising a human heavy chain constant region gene segment of an IGHG1*01. The present disclosure also provides a method of improving FcR signaling or Fc effector function of an ABP by introducing a human heavy chain constant region gene segment of an IGHG1*01 to the ABP.

In one aspect, antigen binding proteins in accordance with the present disclosure include antigen binding proteins that inhibit a biological activity of CTLA-4.

In some embodiments, the antigen binding proteins in accordance with the present disclosure include an IGHG1*01 Fe anti-CTLA-4 antibody or antigen-binding fragment thereof that enhances FcR signaling or Fc effector function. In some embodiments, the IGHG1*01 Fc anti-CTLA-4 antibody comprises an scFvs.

Different antigen binding proteins may bind to different domains of CTLA-4 or act by different mechanisms of action. As indicated herein inter alia, the domain region is designated such as to be inclusive of the group, unless otherwise indicated. For example, amino acids 4-12 refers to nine amino acids: amino acids at positions 4, and 12, as well as the seven intervening amino acids in the sequence. Other examples include antigen binding proteins that inhibit binding of CTLA-4 to its ligands. An antigen binding protein need not completely inhibit a CTLA-4-induced activity to find use in the present disclosure; rather, antigen binding proteins that reduce a particular activity of CTLA-4 are contemplated for use as well. (Discussions herein of particular mechanisms of action for CTLA-4-binding antigen binding proteins in treating particular diseases are illustrative only, and the methods presented herein are not bound thereby.)

A Fab fragment is a monovalent fragment having the V_{L}, V_{H}, C_{L} and C_{H1} domains; a F(ab′)₂ fragment is a bivalent fragment having two Fab fragments linked by a disulfide bridge at the hinge region; a Fd fragment has the V_{H} and C_{H1} domains; an Fv fragment has the V_{L} and V_{H} domains of a single arm of an antibody; and a dAb fragment has a V_{H} domain, a V_{L} domain, or an antigen-binding fragment of a V_{H} or V_{L} domain (U.S. Pat. Nos. 6,846,634, 6,696,245, US App. Pub. No. 05/0202512, 04/0202995, 04/0038291, 04/0009507, 03/0039958, Ward et al., Nature 341:544-546, 1989).

Polynucleotide and polypeptide sequences of particular light and heavy chain variable domains are described below. Antibodies comprising a light chain and heavy chain are designated by combining the name of the light chain and the name of the heavy chain variable domains. For example, “L4H7,” indicates an antibody comprising the light chain variable domain of L4 (comprising a sequence of SEQ ID NO:4) and the heavy chain variable domain of H7 (comprising a sequence of SEQ ID NO:107). Light chain variable sequences are provided in SEQ ID Nos: 1-28, and heavy chain variable sequences are provided in SEQ ID Nos:101-128.

In other embodiments, an antibody may comprise a specific heavy or light chain, while the complementary light or heavy chain variable domain remains unspecified. In particular, certain embodiments herein include antibodies that bind a specific antigen (such as CTLA-4) by way of a specific light or heavy chain, such that the complementary heavy or light chain may be promiscuous, or even irrelevant, but may be determined by, for example, screening combinatorial libraries. Portolano et al., J. Immunol. V. 150 (3), pp. 880-887 (1993); Clackson et al., Nature v. 352 pp. 624-628 (1991); Adler et al., A natively paired antibody library yields drug leads with higher sensitivity and specificity than a randomly paired antibody library, MAbs (2018)); Adler et al., Rare, high-affinity mouse anti-CTLA-4 antibodies that function in checkpoint blockade, discovered using microfluidics and molecular genomics, MAbs (2017).

Naturally occurring immunoglobulin chains exhibit the same general structure of relatively conserved framework regions (FR) joined by three hypervariable regions, also called complementarity determining regions or CDRs. From N-terminus to C-terminus, both light and heavy chains comprise the domains FR1, CDR1, FR2, CDR2, FR3, CDR3 and FR4. The assignment of amino acids to each domain is in accordance with the definitions of Kabat et al. in Sequences of Proteins of Immunological Interest, 5th Ed., US Dept. of Health and Human Services, PHS, NIH, NIH Publication no. 91-3242, 1991.

The term “human antibody,” also referred to as “fully human antibody,” includes all antibodies that have one or more variable and constant regions derived from human immunoglobulin sequences. In one embodiment, all of the variable and constant domains are derived from human immunoglobulin sequences (a fully human antibody). These antibodies may be prepared in a variety of ways, examples of which are described below, including through the immunization with an antigen of interest of a mouse that is genetically modified to express antibodies derived from human heavy and/or light chain-encoding genes.

A humanized antibody has a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject. In one embodiment, certain amino acids in the framework and constant domains of the heavy and/or light chains of the non-human species antibody are mutated to produce the humanized antibody. In another embodiment, the constant domain(s) from a human antibody are fused to the variable domain(s) of a non-human species. In another embodiment, one or more amino acid residues in one or more CDR sequences of a non-human antibody are changed to reduce the likely immunogenicity of the non-human antibody when it is administered to a human subject, wherein the changed amino acid residues either are not critical for immunospecific binding of the antibody to its antigen, or the changes to the amino acid sequence that are made are conservative changes, such that the binding of the humanized antibody to the antigen is not significantly worse than the binding of the non-human antibody to the antigen. Examples of how to make humanized antibodies may be found in U.S. Pat. Nos. 6,054,297, 5,886,152 and 5,877,293.

The term “chimeric antibody” refers to an antibody that contains one or more regions from one antibody and one or more regions from one or more other antibodies. In one embodiment, one or more of the CDRs are derived from a human anti-CTLA-4 antibody. In another embodiment, all of the CDRs are derived from a human anti-CTL A-4 antibody. In another embodiment, the CDRs from more than one human anti-CTLA-4 antibodies are mixed and matched in a chimeric antibody. For instance, a chimeric antibody may comprise a CDR1 from the light chain of a first human anti-CTLA-4 antibody, a CDR2 and a CDR3 from the light chain of a second human anti-CTLA-4 antibody, and the CDRs from the heavy chain from a third anti-CTLA-4 antibody. Further, the framework regions may be derived from one of the same anti-CTLA-4 antibodies, from one or more different antibodies, such as a human antibody, or from a humanized antibody. In one example of a chimeric antibody, a portion of the heavy and/or light chain is identical with, homologous to, or derived from an antibody from a particular species or belonging to a particular antibody class or subclass, while the remainder of the chain(s) is/are identical with, homologous to, or derived from an antibody (-ies) from another species or belonging to another antibody class or subclass. Also included are fragments of such antibodies that exhibit the desired biological activity (i.e., the ability to specifically bind CTLA-4).

Fragments or analogs of antibodies can be readily prepared by those of ordinary skill in the art following the teachings of this specification and using techniques well-known in the art. Preferred amino- and carboxy-termini of fragments or analogs occur near boundaries of functional domains. Structural and functional domains can be identified by comparison of the nucleotide and/or amino acid sequence data to public or proprietary sequence databases. Computerized comparison methods can be used to identify sequence motifs or predicted protein conformation domains that occur in other proteins of known structure and/or function. Methods to identify protein sequences that fold into a known three-dimensional structure are known. See, e.g., Bowie et al., 1991, Science 253:164.

Antigen binding fragments derived from an antibody can be obtained, for example, by proteolytic hydrolysis of the antibody, for example, pepsin or papain digestion of whole antibodies according to conventional methods. By way of example, antibody fragments can be produced by enzymatic cleavage of antibodies with pepsin to provide a 5S fragment termed F(ab′)2. This fragment can be further cleaved using a thiol reducing agent to produce 3.5S Fab′ monovalent fragments. Optionally, the cleavage reaction can be performed using a blocking group for the sulfhydryl groups that result from cleavage of disulfide linkages. As an alternative, an enzymatic cleavage using papain produces two monovalent Fab fragments and an Fe fragment directly. These methods are described, for example, by Goldenberg, U.S. Pat. No. 4,331,647, Nisonoff et al., Arch. Biochem. Biophys. 89:230, 1960; Porter, Biochem. J. 73:119, 1959; Edelman et al., in Methods in Enzymology 1:422 (Academic Press 1967); and by Andrews, S. M. and Titus, J. A. in Current Protocols in Immunology (Coligan J. E., et al., eds), John Wiley & Sons, New York (2003), pages 2.8.1 2.8.10 and 2.10A.1 2.10A.5. Other methods for cleaving antibodies, such as separating heavy chains to form monovalent light heavy chain fragments (Fd), further cleaving of fragments, or other enzymatic, chemical, or genetic techniques may also be used, so long as the fragments bind to the antigen that is recognized by the intact antibody.

An antibody fragment may also be any synthetic or genetically engineered protein. For example, antibody fragments include isolated fragments consisting of the light chain variable region, “Fv” fragments consisting of the variable regions of the heavy and light chains, recombinant single chain polypeptide molecules in which light and heavy variable regions are connected by a peptide linker (scFv proteins).

Another form of an antibody fragment is a peptide comprising one or more complementarity determining regions (CDRs) of an antibody. CDRs (also termed “minimal recognition units”, or “hypervariable region”) can be incorporated into a molecule either covalently or noncovalently to make it an antigen binding protein. CDRs can be obtained by constructing polynucleotides that encode the CDR of interest. Such polynucleotides are prepared, for example, by using the polymerase chain reaction to synthesize the variable region using mRNA of antibody producing cells as a template (see, for example, Larrick et al., Methods: A Companion to Methods in Enzymology 2:106, 1991; Courtenay Luck, “Genetic Manipulation of Monoclonal Antibodies,” in Monoclonal Antibodies: Production, Engineering and Clinical Application, Ritter et al. (eds.), page 166 (Cambridge University Press 1995); and Ward et al., “Genetic Manipulation and Expression of Antibodies,” in Monoclonal Antibodies: Principles and Applications, Birch et al., (eds.), page 137 (Wiley Liss, Inc. 1995).

Thus, in one embodiment, the binding agent comprises at least one CDR as described herein. The binding agent may comprise at least two, three, four, five or six CDR's as described herein. The binding agent may further comprise at least one variable region domain of an antibody described herein. The variable region domain may be of any size or amino acid composition and will generally comprise at least one CDR sequence responsible for binding to human CTLA-4, for example CDR1-H, CDR2-H, CDR3-H, CDR1-L, CDR2-L, and CDR3-L, specifically described herein and which is adjacent to or in frame with one or more framework sequences. In general terms, the variable (V) region domain may be any suitable arrangement of immunoglobulin heavy (Vii) and/or light (V_{L}) chain variable domains. Thus, for example, the V region domain may be monomeric and be a Vii or V_{L} domain, which is capable of independently binding human CTLA-4 with an affinity at least equal to 1×10⁷M or less as described below. Alternatively, the V region domain may be dimeric and contain V_{H} V_{H}, V_{H} V_{L}, or V_{L} V_{L}, dimers. The V region dimer comprises at least one V_{H} and at least one V_{L} chain that may be non-covalently associated (hereinafter referred to as Fv). If desired, the chains may be covalently coupled either directly, for example via a disulfide bond between the two variable domains, or through a linker, for example a peptide linker, to form a single chain Fv (scFV).

The variable region domain may be any naturally occurring variable domain or an engineered version thereof. By engineered version is meant a variable region domain that has been created using recombinant DNA engineering techniques. Such engineered versions include those created, for example, from a specific antibody variable region by insertions, deletions, or changes in or to the amino acid sequences of the specific antibody. Particular examples include engineered variable region domains containing at least one CDR and optionally one or more framework amino acids from a first antibody and the remainder of the variable region domain from a second antibody.

The variable region domain may be covalently attached at a C terminal amino acid to at least one other antibody domain or a fragment thereof. Thus, for example, a V_{H} domain that is present in the variable region domain may be linked to an immunoglobulin CH1 domain, or a fragment thereof. Similarly a V_{L} domain may be linked to a CK domain or a fragment thereof. In this way, for example, the antibody may be a Fab fragment wherein the antigen binding domain contains associated V_{H} and V_{L} domains covalently linked at their C termini to a CH1 and CK domain, respectively. The CH1 domain may be extended with further amino acids, for example to provide a hinge region or a portion of a hinge region domain as found in a Fab′ fragment, or to provide further domains, such as antibody CH2 and CH3 domains.

In some embodiments, the ABP comprises an Fc region lacking a fucose sugar unit on the N glycan.

In some embodiments, the ABP comprises Glycine at the amino acid position 201.

In some embodiments, the ABP is produced from a cell comprising a bacterial protein RMD (GDP-6-deoxy-D-lyxo-4-hexulose reductase) or a modification thereof. In certain embodiments, the cell is cultured in the absence of fucose. In some embodiments, the ABP is produced from a cell lacking or reduced expression of Fut8. In some embodiments, the ABP is produced from a cell cultured in the presence of a fucosylation inhibitor, 2-Fluorfucose (2FF).

In some embodiments, the ABP is produced from a cell overexpressing glycosyltransferase (GnTIII). In some embodiments, the ABP is isolated based on its fucosylation status.

In some embodiments, the ABP is an afucosylated monoclonal antibody.

As described herein, antibodies comprise at least one of these CDRs. For example, one or more CDR may be incorporated into known antibody framework regions (IgG1, IgG2, etc.), or conjugated to a suitable vehicle to enhance the half-life thereof. Suitable vehicles include, but are not limited to Fc, polyethylene glycol (PEG), albumin, transferrin, and the like. These and other suitable vehicles are known in the art. Such conjugated CDR peptides may be in monomeric, dimeric, tetrameric, or other form. In one embodiment, one or more water-soluble polymer is bonded at one or more specific position, for example at the amino terminus, of a binding agent.

In another example, individual V_{L} or V_{H} chains from an antibody (i.e. CTLA-4 antibody) can be used to search for other V_{H} or V_{L} chains that could form antigen-binding fragments (or Fab), with the same specificity. Thus, random combinations of V_{H} and V_{L} chain Ig genes can be expressed as antigen-binding fragments in a bacteriophage library (such as fd or lambda phage). For instance, a combinatorial library may be generated by utilizing the parent V_{L} or V_{H} chain library combined with antigen-binding specific V_{L} or V_{H} chain libraries, respectively. The combinatorial libraries may then be screened by conventional techniques, for example by using radioactively labeled probe (such as radioactively labeled CTL A-4). See, for example, Portolano et al., J. Immunol. V. 150 (3) pp. 880-887 (1993).

Diabodies are bivalent antibodies comprising two polypeptide chains, wherein each polypeptide chain comprises V_{H} and V_{L} domains joined by a linker that is too short to allow for pairing between two domains on the same chain, thus allowing each domain to pair with a complementary domain on another polypeptide chain (see, e.g., Holliger et al., 1993, Proc. Natl. Acad. Sci. USA 90:6444-48, and Poljak et al., 1994, Structure 2:1121-23). If the two polypeptide chains of a diabody are identical, then a diabody resulting from their pairing will have two identical antigen binding sites. Polypeptide chains having different sequences can be used to make a diabody with two different antigen binding sites. Similarly, tribodies and tetrabodies are antibodies comprising three and four polypeptide chains, respectively, and forming three and four antigen binding sites, respectively, which can be the same or different.

Antibody polypeptides are also disclosed in U.S. Pat. No. 6,703,199, including fibronectin polypeptide monobodies. Other antibody polypeptides are disclosed in U.S. Patent Publication 2005/0238646, which are single-chain polypeptides.

In certain embodiments, an antibody comprises one or more water soluble polymer attachments, including, but not limited to, polyethylene glycol, polyoxyethylene glycol, or polypropylene glycol. See, e.g., U.S. Pat. Nos. 4,640,835, 4,496,689, 4,301,144, 4,670,417, 4,791,192 and 4,179,337. In certain embodiments, a derivative binding agent comprises one or more of monomethoxy-polyethylene glycol, dextran, cellulose, or other carbohydrate based polymers, poly-(N-vinyl pyrrolidone)-polyethylene glycol, propylene glycol homopolymers, a polypropylene oxide/ethylene oxide co-polymer, polyoxyethylated polyols (e.g., glycerol) and polyvinyl alcohol, as well as mixtures of such polymers. In certain embodiments, one or more water-soluble polymer is randomly attached to one or more side chains. In certain embodiments, PEG can act to improve the therapeutic capacity for a binding agent, such as an antibody. Certain such methods are discussed, for example, in U.S. Pat. No. 6,133,426, which is hereby incorporated by reference for any purpose.

In some embodiments, the ABPs of the present disclosure are monoclonal antibodies that bind to CTLA-4. Monoclonal antibodies of the present disclosure may be generated using a variety of known techniques. In general, monoclonal antibodies that bind to specific antigens may be obtained by methods known to those skilled in the art (see, for example, Kohler et al., Nature 256:495, 1975; Coligan et al. (eds.), Current Protocols in Immunology, 1:2.5.12.6.7 (John Wiley & Sons 1991); U.S. Pat. Nos. RE 32,011, 4,902,614, 4,543,439, and 4,411,993; Monoclonal Antibodies, Hybridomas: A New Dimension in Biological Analyses, Plenum Press, Kennett, McKearn, and Bechtol (eds.) (1980); and Antibodies: A Laboratory Manual, Harlow and Lane (eds.), Cold Spring Harbor Laboratory Press (1988); Picksley et al., “Production of monoclonal antibodies against proteins expressed in E. coli,” in DNA Cloning 2: Expression Systems, 2nd Edition, Glover et al. (eds.), page 93 (Oxford University Press 1995)), Antibody fragments may be derived therefrom using any suitable standard technique such as proteolytic digestion, or optionally, by proteolytic digestion (for example, using papain or pepsin) followed by mild reduction of disulfide bonds and alkylation. Alternatively, such fragments may also be generated by recombinant genetic engineering techniques as described herein.

Monoclonal antibodies can be obtained by injecting an animal, for example, a rat, hamster, a rabbit, or preferably a mouse, including for example a transgenic or a knock-out, as known in the art, with an immunogen comprising human CTLA-4 [sequence SEQ ID 7001] or a fragment thereof, according to methods known in the art and described herein. The presence of specific antibody production may be monitored after the initial injection and/or after a booster injection by obtaining a serum sample and detecting the presence of an antibody that binds to human CTLA-4 or peptide using any one of several immunodetection methods known in the art and described herein. From animals producing the desired antibodies, lymphoid cells, most commonly cells from the spleen or lymph node, are removed to obtain B-lymphocytes. The B lymphocytes are then fused with a drug-sensitized myeloma cell fusion partner, preferably one that is syngeneic with the immunized animal and that optionally has other desirable properties (e.g., inability to express endogenous Ig gene products, e.g., P3×63-Ag 8.653 (ATCC No. CRL 1580); NSO, SP20) to produce hybridomas, which are immortal eukaryotic cell lines.

The lymphoid (e.g., spleen) cells and the myeloma cells may be combined for a few minutes with a membrane fusion-promoting agent, such as polyethylene glycol or a nonionic detergent, and then plated at low density on a selective medium that supports the growth of hybridoma cells but not unfused myeloma cells. A preferred selection media is HAT (hypoxanthine, aminopterin, thymidine). After a sufficient time, usually about one to two weeks, colonies of cells are observed. Single colonies are isolated, and antibodies produced by the cells may be tested for binding activity to human CTLA-4, using any one of a variety of immunoassays known in the art and described herein. The hybridomas are cloned (e.g., by limited dilution cloning or by soft agar plaque isolation) and positive clones that produce an antibody specific to CTLA-4 are selected and cultured. The monoclonal antibodies from the hybridoma cultures may be isolated from the supernatants of hybridoma cultures.

An alternative method for production of a murine monoclonal antibody is to inject the hybridoma cells into the peritoneal cavity of a syngeneic mouse, for example, a mouse that has been treated (e.g., pristane-primed) to promote formation of ascites fluid containing the monoclonal antibody. Monoclonal antibodies can be isolated and purified by a variety of well-established techniques. Such isolation techniques include affinity chromatography with Protein-A Sepharose, size-exclusion chromatography, and ion-exchange chromatography (see, for example, Coligan at pages 2.7.1-2.7.12 and pages 2.9.1-2.9.3; Baines et al., “Purification of Immunoglobulin G (IgG),” in Methods in Molecular Biology, Vol. 10, pages 79-104 (The Humana Press, Inc. 1992)). Monoclonal antibodies may be purified by affinity chromatography using an appropriate ligand selected based on particular properties of the antibody (e.g., heavy or light chain isotype, binding specificity, etc.). Examples of a suitable ligand, immobilized on a solid support, include Protein A, Protein G, an anticonstant region (light chain or heavy chain) antibody, an anti-idiotype antibody, and a TGF-beta binding protein, or fragment or variant thereof.

Monoclonal antibodies may be produced using any technique known in the art, e.g., by immortalizing spleen cells harvested from the transgenic animal after completion of the immunization schedule. The spleen cells can be immortalized using any technique known in the art, e.g., by fusing them with myeloma cells to produce hybridomas. Hybridoma cell lines are identified that produce an antibody that binds a CTLA-4 polypeptide. Such hybridoma cell lines, and anti-CTLA-4 monoclonal antibodies produced by them, are encompassed by the present disclosure. Myeloma cells for use in hybridoma-producing fusion procedures preferably are non-antibody-producing, have high fusion efficiency, and enzyme deficiencies that render them incapable of growing in certain selective media which support the growth of only the desired fused cells (hybridomas). Examples of suitable cell lines for use in mouse fusions include Sp-20, P3-X63/Ag8, P3-X63-Ag8.653, NS1/1. Ag 4 1, Sp210-Ag14, FO, NSO/U, MPC-11, MPC11-X45-GTG 1.7 and S194/5XX0 Bul; examples of cell lines used in rat fusions include R210.RCY3, Y3-Ag 1.2.3, IR983F and 4B210. Other cell lines useful for cell fusions are U-266, GM1500-GRG2, LICR-LON-HMy2 and UC729-6. Hybridomas or mAbs may be further screened to identify mAbs with particular properties, such as the ability to block a CTLA-4-induced activity.

An antibody of the present disclosure may also be a fully human monoclonal antibody. An isolated fully human antibody is provided that specifically binds to the CTLA-4, wherein the antigen binding protein possesses at least one in vivo biological activity of a human anti-CTLA-4 antibody.

### 7.4. Nucleic Acids

In one aspect, the present disclosure provides isolated nucleic acid molecules. The nucleic acids comprise, for example, polynucleotides that encode all or part of an antigen binding protein, for example, one or both chains of an antibody of the present disclosure, or a fragment, derivative, mutein, or variant thereof, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, and complementary sequences of the foregoing. The nucleic acids can be any length. They can be, for example, 5, 10, 15, 20, 25, 30, 35, 40, 45, 50, 75, 100, 125, 150, 175, 200, 250, 300, 350, 400, 450, 500, 750, 1,000, 1,500, 3,000, 5,000 or more nucleotides in length, and/or can comprise one or more additional sequences, for example, regulatory sequences, and/or be part of a larger nucleic acid, for example, a vector. The nucleic acids can be single-stranded or double-stranded and can comprise RNA and/or DNA nucleotides, and artificial variants thereof (e.g., peptide nucleic acids).

Nucleic acids encoding antibody polypeptides (e.g., heavy or light chain, variable domain only, or full length) can be isolated from B-cells of mice that have been immunized with CTLA-4. The nucleic acid can be isolated by conventional procedures such as polymerase chain reaction (PCR).

Nucleic acid sequences encoding the variable regions of the heavy and light chain variable regions are shown herein. The skilled artisan will appreciate that, due to the degeneracy of the genetic code, each of the polypeptide sequences disclosed herein is encoded by a large number of other nucleic acid sequences. The present disclosure provides each degenerate nucleotide sequence encoding each antigen binding protein of the present disclosure. In some embodiments, the nucleic acid sequences have been codon optimized. In some embodiments, the nucleic acid sequences have been codon optimized for expression in a mammalian cell.

The present disclosure further provides nucleic acids that hybridize to other nucleic acids (e.g., nucleic acids comprising a nucleotide sequence of any of CTLA-4 gene) under particular hybridization conditions. Methods for hybridizing nucleic acids are well-known in the art. See, e.g., Curr. Prot. in Mol. Biol., John Wiley & Sons, N.Y. (1989), 6.3.1-6.3.6. As defined herein, a moderately stringent hybridization condition uses a prewashing solution containing 5× sodium chloride/sodium citrate (SSC), 0.5% SDS, 1.0 mM EDTA (pH 8.0), hybridization buffer of about 50% formamide, 6×SSC, and a hybridization temperature of 55° C. (or other similar hybridization solutions, such as one containing about 50% formamide, with a hybridization temperature of 42° C.), and washing conditions of 60° C., in 0.5×SSC, 0.1% SDS. A stringent hybridization condition hybridizes in 6×SSC at 45° C., followed by one or more washes in 0.1×SSC, 0.2% SDS at 68° C. Furthermore, one of skill in the art can manipulate the hybridization and/or washing conditions to increase or decrease the stringency of hybridization such that nucleic acids comprising nucleotide sequences that are at least 65, 70, 75, 80, 85, 90, 95, 98, or 99% identical to each other typically remain hybridized to each other. The basic parameters affecting the choice of hybridization conditions and guidance for devising suitable conditions are set forth by, for example, Sambrook, Fritsch, and Maniatis (1989, Molecular Cloning: A Laboratory Manual, Cold Spring Harbor Laboratory Press, Cold Spring Harbor, N.Y., chapters 9 and 11; and Curr. Prot. in Mol. Biol. 1995, Ausubel et al., eds., John Wiley & Sons, Inc., sections 2.10 and 6.3-6.4), and can be readily determined by those having ordinary skill in the art based on, for example, the length and/or base composition of the DNA.

Changes can be introduced by mutation into a nucleic acid, thereby leading to changes in the amino acid sequence of a polypeptide (e.g., an antigen binding protein) that it encodes. Mutations can be introduced using any technique known in the art. In one embodiment, one or more particular amino acid residues are changed using, for example, a site-directed mutagenesis protocol. In another embodiment, one or more randomly selected residues are changed using, for example, a random mutagenesis protocol. However it is made, a mutant polypeptide can be expressed and screened for a desired property (e.g., binding to CTLA-4).

Mutations can be introduced into a nucleic acid without significantly altering the biological activity of a polypeptide that it encodes. For example, one can make nucleotide substitutions leading to amino acid substitutions at non-essential amino acid residues. In one embodiment, a nucleotide sequence provided herein for CTLA-4, or a desired fragment, variant, or derivative thereof, is mutated such that it encodes an amino acid sequence comprising one or more deletions or substitutions of amino acid residues that are shown herein for CTLA-4 to be residues where two or more sequences differ. Alternatively, one or more mutations can be introduced into a nucleic acid that selectively change the biological activity (e.g., binding of CTLA-4) of a polypeptide that it encodes. For example, the mutation can quantitatively or qualitatively change the biological activity. Examples of quantitative changes include increasing, reducing or eliminating the activity. Examples of qualitative changes include changing the antigen specificity of an antigen binding protein.

In another aspect, the present disclosure provides nucleic acid molecules that are suitable for use as primers or hybridization probes for the detection of nucleic acid sequences of the present disclosure. A nucleic acid molecule of the present disclosure can comprise only a portion of a nucleic acid sequence encoding a full-length polypeptide of the present disclosure, for example, a fragment that can be used as a probe or primer or a fragment encoding an active portion (e.g., a CTLA-4 binding portion) of a polypeptide of the present disclosure.

Probes based on the sequence of a nucleic acid of the present disclosure can be used to detect the nucleic acid or similar nucleic acids, for example, transcripts encoding a polypeptide of the present disclosure. The probe can comprise a label group, e.g., a radioisotope, a fluorescent compound, an enzyme, or an enzyme co-factor. Such probes can be used to identify a cell that expresses the polypeptide

### 7.5. Expression Vectors

The present disclosure provides vectors comprising a nucleic acid encoding a polypeptide of the present disclosure or a portion thereof. Examples of vectors include, but are not limited to, plasmids, viral vectors, non-episomal mammalian vectors and expression vectors, for example, recombinant expression vectors.

In another aspect of the present disclosure, expression vectors containing the nucleic acid molecules and polynucleotides of the present disclosure are also provided, and host cells transformed with such vectors, and methods of producing the polypeptides are also provided. The term “expression vector” refers to a plasmid, phage, virus or vector for expressing (e.g. or inducing expression of) a polypeptide from a polynucleotide sequence. Vectors for the expression of the polypeptides contain at a minimum sequences required for vector propagation and for expression of the cloned insert. An expression vector comprises a transcriptional unit comprising an assembly of (1) a genetic element or elements having a regulatory role in gene expression, for example, promoters or enhancers, (2) a sequence that encodes polypeptides and proteins to be transcribed into mRNA and translated into protein, and (3) appropriate transcription initiation and termination sequences. These sequences may further include a selection marker. Vectors suitable for expression in host cells are readily available and the nucleic acid molecules are inserted into the vectors using standard recombinant DNA techniques. Such vectors can include promoters which function in specific tissues, and viral vectors for the expression of polypeptides in targeted human or animal cells.

The recombinant expression vectors of the present disclosure can comprise a nucleic acid of the present disclosure in a form suitable for expression of the nucleic acid in a host cell. Accordingly, in one aspect, the present disclosure provides a host cell comprising the polynucleotide or the vector encoding the ABP of the present disclosure. The host cell can be used to produce the ABP ex vivo. In some embodiments, the host cell is administered to a subject to induce expression of the ABP in vivo. In some embodiments, the host cells are used as a therapeutic for treatment of a disease.

The recombinant expression vectors include one or more regulatory sequences, selected on the basis of the host cells to be used for expression, which is operably linked to the nucleic acid sequence to be expressed. Regulatory sequences include those that direct constitutive expression of a nucleotide sequence in many types of host cells (e.g., SV40 early gene enhancer, Rous sarcoma virus promoter and cytomegalovirus promoter), those that direct expression of the nucleotide sequence only in certain host cells (e.g., tissue-specific regulatory sequences, see Voss et al., 1986, Trends Biochem. Sci. 11:287, Maniatis et al., 1987, Science 236:1237, incorporated by reference herein in their entireties), and those that direct inducible expression of a nucleotide sequence in response to particular treatment or condition (e.g., the metallothionin promoter in mammalian cells and the tet-responsive and/or streptomycin responsive promoter in both prokaryotic and eukaryotic systems (see id.). It will be appreciated by those skilled in the art that the design of the expression vector can depend on such factors as the choice of the host cell to be transformed, the level of expression of protein desired, etc. The expression vectors of the present disclosure can be introduced into host cells to thereby produce proteins or peptides, including fusion proteins or peptides, encoded by nucleic acids as described herein.

In some embodiments, the expression vector is an expression vector purified from one of the clones of the library of CTLA-4 binding clones deposited under ATCC Accession No. PTA-125512. In some embodiments, the expression vector is generated by genetic modification of one of an expression vector in one of the clones purified from the library of CTLA-4 binding clones deposited under ATCC Accession No. PTA-125512. In some embodiments, the expression vector is generated by using variable region sequences of heavy and light chains of one of the clones of the library of CTLA-4 binding clones deposited under ATCC Accession No. PTA-125512.

The present disclosure further provides methods of making polypeptides. A variety of other expression/host systems may be utilized. Vector DNA can be introduced into prokaryotic or eukaryotic systems via conventional transformation or transfection techniques. These systems include but are not limited to microorganisms such as bacteria (for example, E. coli) transformed with recombinant bacteriophage, plasmid or cosmid DNA expression vectors; yeast transformed with yeast expression vectors; insect cell systems infected with virus expression vectors (e.g., baculovirus); plant cell systems transfected with virus expression vectors (e.g., cauliflower mosaic virus, CaMV; tobacco mosaic virus, TMV) or transformed with bacterial expression vectors (e.g., Ti or pBR322 plasmid); or animal cell systems. Mammalian cells useful in recombinant protein production include but are not limited to VERO cells, HeLa cells, Chinese hamster ovary (CHO) cell lines, or their derivatives such as Veggie CHO and related cell lines which grow in serum-free media (see Rasmussen et al., 1998, Cytotechnology 28:31) or CHO strain DX-B11, which is deficient in DHFR (see Urlaub et al., 1980, Proc. Natl. Acad. Sci. USA 77:4216-20) COS cells such as the COS-7 line of monkey kidney cells (ATCC CRL 1651) (see Gluzman et al., 1981, Cell 23:175), W138, BHK, HepG2, 3T3 (ATCC CCL 163), RIN, MDCK, A549, PC12, K562, L cells, C127 cells, BHK (ATCC CRL 10) cell lines, the CV1/EBNA cell line derived from the African green monkey kidney cell line CV1 (ATCC CCL 70) (see McMahan et al., 1991, EMBO J. 10:2821), human embryonic kidney cells such as 293, 293 EBNA or MSR 293, human epidermal A431 cells, human Colo205 cells, other transformed primate cell lines, normal diploid cells, cell strains derived from in vitro culture of primary tissue, primary explants, HL-60, U937, HaK or Jurkat cells. Mammalian expression allows for the production of secreted or soluble polypeptides which may be recovered from the growth medium.

In some embodiments, the mammalian cells used in recombinant protein production include engineered cells that produce a reduced amount of core fucosylation (e.g., relative to the amount of core fucosylation of a non-engineered cell). In some embodiments, the mammalian cells used in recombinant protein production include engineered cells that produce a reduced amount of fucose (e.g., relative to the amount of fucose of a non-engineered cell). In some embodiments, the mammalian cells used in recombinant protein production comprise CHO cells. In some embodiments, the mammalian cell is a GlymaxX® cell line. In certain embodiments, the cell line produces afucosylated recombinant proteins. In certain embodiments, the mammalian cell has less or reduced fucosylation, e.g., where the mammalian cell produces a reduced amount of fucose. In certain embodiments, during cell culture, the method includes adding a fucosylation inhibitor to the media that the cells are grown in. Non-limiting examples of fucosylation inhibitors include: fucosyltransferase (FUT) inhibitor, 2-fluoro peracetylated fucose (2FF), 2-fluorofucose (SGN-2FF), Fucotrim I (P-D-Rha6F2-TP), Fucotrim II (P-D-Rha6F3-TP), A2FF1P, and 132FF1. In some embodiments, the mammalian cells used in recombinant protein production are engineered to overexpress glycosyltransferase. In certain embodiments, the glycosyltransferase is Beta-1,4-mannosyl-glycoprotein 4-beta-N-acetylglucosaminyltransferase.

In some embodiments, the ABP comprises an afucosylated Fc region. In some embodiments, the antibody is afucosylated (e.g., the N-glycan of the Fc region of the antibody does not have core fucose sugar units).

In certain embodiments, the glycosyltransferase competes with FuT8. In some embodiments, the mammalian cell line used in recombinant protein production produces an ABP that has reduced fucosylation (e.g. less than 70% fucosylation, less than 65% fucosylation, less than 60% fucosylation, less than 55% fucosylation, less than 50% fucosylation, less than 45% fucosylation, less than 40% fucosylation, less than 35% fucosylation, less than 30% fucosylation, less than 25% fucosylation, less than 20% fucosylation, less than 15% fucosylation, less than 10% fucosylation, less than 5% fucosylation, or less than 2.5% fucosylation).

In some embodiments, the mammalian cell line used in recombinant protein production produces an ABP that has increased fucosylation (e.g. more than 99% fucosylation, more than 95% fucosylation, more than 90% fucosylation, more than 85% fucosylation, more than 80% fucosylation, more than 75% fucosylation, more than 70% fucosylation, more than 65% fucosylation, more than 60% fucosylation, more than 55% fucosylation, more than 50% fucosylation, more than 45% fucosylation, more than 40% fucosylation, more than 35% fucosylation, more than 30% fucosylation, more than 25% fucosylation, more than 20% fucosylation, more than 15% fucosylation, more than 10% fucosylation, more than 5% fucosylation, or more than 2.5% fucosylation).

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Once such cells are transformed with vectors that contain selectable markers as well as the desired expression cassette, the cells can be allowed to grow in an enriched media before they are switched to selective media, for example. The selectable marker is designed to allow growth and recovery of cells that successfully express the introduced sequences. Resistant clumps of stably transformed cells can be proliferated using tissue culture techniques appropriate to the cell line employed. An overview of expression of recombinant proteins is found in Methods of Enzymology, v. 185, Goeddell, D. V., ed., Academic Press (1990). Preferred selectable markers include those which confer resistance to drugs, such as G418, hygromycin and methotrexate. Cells stably transfected with the introduced nucleic acid can be identified by drug selection (e.g., cells that have incorporated the selectable marker gene will survive, while the other cells die), among other methods.

The transformed cells can be cultured under conditions that promote expression of the polypeptide, and the polypeptide recovered by conventional protein purification procedures (as defined above). One such purification procedure includes the use of affinity chromatography, e.g., over a matrix having all or a portion (e.g., the extracellular domain) of CTL A-4 bound thereto. Polypeptides contemplated for use herein include substantially homogeneous recombinant mammalian anti-CTLA-4 antibody polypeptides substantially free of contaminating endogenous materials.

In some cases, such as in expression using prokaryotic systems, the expressed polypeptides of this disclosure may need to be “refolded” and oxidized into a proper tertiary structure and disulfide linkages generated in order to be biologically active. Refolding can be accomplished using a number of procedures well known in the art. Such methods include, for example, exposing the solubilized polypeptide to a pH usually above 7 in the presence of a chaotropic agent. The selection of chaotrope is similar to the choices used for inclusion body solubilization; however a chaotrope is typically used at a lower concentration. Exemplary chaotropic agents are guanidine and urea. In most cases, the refolding/oxidation solution will also contain a reducing agent plus its oxidized form in a specific ratio to generate a particular redox potential which allows for disulfide shuffling to occur for the formation of cysteine bridges. Some commonly used redox couples include cysteine/cystamine, glutathione/dithiobisGSH, cupric chloride, dithiothreitol DTT/dithiane DTT, and 2-mercaptoethanol (bME)/dithio-bME. In many instances, a co-solvent may be used to increase the efficiency of the refolding. Commonly used cosolvents include glycerol, polyethylene glycol of various molecular weights, and arginine.

In addition, the polypeptides can be synthesized in solution or on a solid support in accordance with conventional techniques. Various automatic synthesizers are commercially available and can be used in accordance with known protocols. See, for example, Stewart and Young, Solid Phase Peptide Synthesis, 2d. Ed., Pierce Chemical Co. (1984); Tam et al., J Am Chem Soc, 105:6442, (1983); Merrifield, Science 232:341-347 (1986); Barany and Merrifield, The Peptides, Gross and Meienhofer, eds, Academic Press, New York, 1-284; Barany et al., Int J Pep Protein Res, 30:705-739 (1987).

The polypeptides and proteins of the present disclosure can be purified according to protein purification techniques well known to those of skill in the art. These techniques involve, at one level, the crude fractionation of the proteinaceous and non-proteinaceous fractions. Having separated the peptide polypeptides from other proteins, the peptide or polypeptide of interest can be further purified using chromatographic and electrophoretic techniques to achieve partial or complete purification (or purification to homogeneity). The term “purified polypeptide” as used herein, is intended to refer to a composition, isolatable from other components, wherein the polypeptide is purified to any degree relative to its naturally-obtainable state. A purified polypeptide therefore also refers to a polypeptide that is free from the environment in which it may naturally occur. Generally, “purified” will refer to a polypeptide composition that has been subjected to fractionation to remove various other components, and which composition substantially retains its expressed biological activity. Where the term “substantially purified” is used, this designation will refer to a peptide or polypeptide composition in which the polypeptide or peptide forms the major component of the composition, such as constituting about 50%, about 60%, about 70%, about 80%, about 85%, or about 90% or more of the proteins in the composition.

Various techniques suitable for use in purification will be well known to those of skill in the art. These include, for example, precipitation with ammonium sulphate, PEG, antibodies (immunoprecipitation) and the like or by heat denaturation, followed by centrifugation; chromatography such as affinity chromatography (Protein-A columns), ion exchange, gel filtration, reverse phase, hydroxylapatite, hydrophobic interaction chromatography, isoelectric focusing, gel electrophoresis, and combinations of these techniques. As is generally known in the art, it is believed that the order of conducting the various purification steps may be changed, or that certain steps may be omitted, and still result in a suitable method for the preparation of a substantially purified polypeptide. Exemplary purification steps are provided in the Examples below.

Various methods for quantifying the degree of purification of polypeptide will be known to those of skill in the art in light of the present disclosure. These include, for example, determining the specific binding activity of an active fraction, or assessing the amount of peptide or polypeptide within a fraction by SDS/PAGE analysis. A preferred method for assessing the purity of a polypeptide fraction is to calculate the binding activity of the fraction, to compare it to the binding activity of the initial extract, and to thus calculate the degree of purification, herein assessed by a “-fold purification number.” The actual units used to represent the amount of binding activity will, of course, be dependent upon the particular assay technique chosen to follow the purification and whether or not the polypeptide or peptide exhibits a detectable binding activity.

### 7.6. Method of Generating Antibodies

Fully human monoclonal antibodies may be generated by any number of techniques with which those having ordinary skill in the art will be familiar. Such methods include, but are not limited to, Epstein Barr Virus (EBV) transformation of human peripheral blood cells (e.g., containing B lymphocytes), in vitro immunization of human B-cells, fusion of spleen cells from immunized transgenic mice carrying inserted human immunoglobulin genes, isolation from human immunoglobulin V region phage libraries, or other procedures as known in the art and based on the disclosure herein. For example, fully human monoclonal antibodies may be obtained from transgenic mice that have been engineered to produce specific human antibodies in response to antigenic challenge. Methods for obtaining fully human antibodies from transgenic mice are described, for example, by Green et al., Nature Genet. 7:13, 1994; Lonberg et al., Nature 368:856, 1994; Taylor et al., Int. Immun. 6:579, 1994; U.S. Pat. No. 5,877,397; Bruggemann et al., 1997 Curr. Opin. Biotechnol. 8:455-58; Jakobovits et al., 1995 Ann. N. Y. Acad. Sci. 764:525-35. In this technique, elements of the human heavy and light chain locus are introduced into strains of mice derived from embryonic stem cell lines that contain targeted disruptions of the endogenous heavy chain and light chain loci (see also Bruggemann et al., Curr. Opin. Biotechnol. 8:455-58 (1997)). For example, human immunoglobulin transgenes may be mini-gene constructs, or transloci on yeast artificial chromosomes, which undergo B-cell-specific DNA rearrangement and hypermutation in the mouse lymphoid tissue. Fully human monoclonal antibodies may be obtained by immunizing the transgenic mice, which may then produce human antibodies specific for CTLA-4. Lymphoid cells of the immunized transgenic mice can be used to produce human antibody-secreting hybridomas according to the methods described herein. Polyclonal sera containing fully human antibodies may also be obtained from the blood of the immunized animals.

Another method for generating human antibodies of the present disclosure includes immortalizing human peripheral blood cells by EBV transformation. See, e.g., U.S. Pat. No. 4,464,456. Such an immortalized B-cell line (or lymphoblastoid cell line) producing a monoclonal antibody that specifically binds to CTLA-4 can be identified by immunodetection methods as provided herein, for example, an ELISA, and then isolated by standard cloning techniques. The stability of the lymphoblastoid cell line producing an anti-CTLA-4 antibody may be improved by fusing the transformed cell line with a murine myeloma to produce a mouse-human hybrid cell line according to methods known in the art (see, e.g., Glasky et al., Hybridoma 8:377-89 (1989)). Still another method to generate human monoclonal antibodies is in vitro immunization, which includes priming human splenic B-cells with human CTLA-4, followed by fusion of primed B-cells with a heterohybrid fusion partner. See, e.g., Boemer et al., 1991 J. Immunol. 147:86-95.

In certain embodiments, a B-cell that is producing an anti-human CTLA-4 antibody is selected and the light chain and heavy chain variable regions are cloned from the B-cell according to molecular biology techniques known in the art (WO 92/02551; U.S. Pat. No. 5,627,052; Babcook et al., Proc. Natl. Acad. Sci. USA 93:7843-48 (1996)) and described herein. B-cells from an immunized animal may be isolated from the spleen, lymph node, or peripheral blood sample by selecting a cell that is producing an antibody that specifically binds to CTLA-4. B-cells may also be isolated from humans, for example, from a peripheral blood sample.

Methods for detecting single B-cells that are producing an antibody with the desired specificity are well known in the art, for example, by plaque formation, fluorescence-activated cell sorting, in vitro stimulation followed by detection of specific antibody, and the like. Methods for selection of specific antibody-producing B-cells include, for example, preparing a single cell suspension of B-cells in soft agar that contains human CTLA-4. Binding of the specific antibody produced by the B-cell to the antigen results in the formation of a complex, which may be visible as an immunoprecipitate.

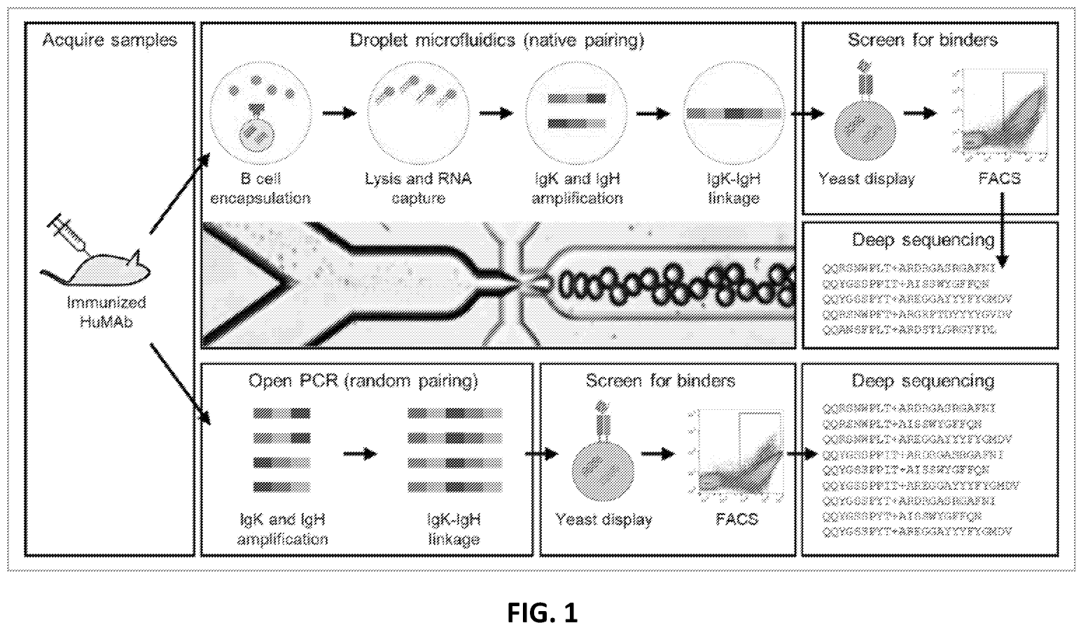

In some embodiments, specific antibody-producing B-cells are selected by using a method that allows identification natively paired antibodies. For example, a method described in Adler et al., A natively paired antibody library yields drug leads with higher sensitivity and specificity than a randomly paired antibody library, MAbs (2018), which is incorporated by reference in its entirety herein, can be employed. The method combines microfluidic technology, molecular genomics, yeast single-chain variable fragment (scFv) display, fluorescence-activated cell sorting (FACS) and deep sequencing as summarized in FIG. 1 adopted from Adler et al. In short, B cells can be isolated from immunized animals and then pooled. The B cells are encapsulated into droplets with oligo-dT beads and a lysis solution, and mRNA-bound beads are purified from the droplets, and then injected into a second emulsion with an OE-RT-PCR amplification mix that generates DNA amplicons that encode scFv with native pairing of heavy and light chain Ig. Libraries of natively paired amplicons are then electroporated into yeast for scFv display. FACS is used to identify high affinity scFv. Finally, deep antibody sequencing can be used to identify all clones in the pre- and post-sort scFv libraries.

After the B-cells producing the desired antibody are selected, the specific antibody genes may be cloned by isolating and amplifying DNA or mRNA according to methods known in the art and described herein.

The methods for obtaining antibodies of the present disclosure can also adopt various phage display technologies known in the art. See, e.g., Winter et al., 1994 Annu. Rev. Immunol. 12:433-55; Burton et al., 1994 Adv. Immunol. 57:191-280. Human or murine immunoglobulin variable region gene combinatorial libraries may be created in phage vectors that can be screened to select Ig fragments (Fab, Fv, sFv, or multimers thereof) that bind specifically to CTLA-4 binding protein or variant or fragment thereof. See, e.g., U.S. Pat. No. 5,223,409; Huse et al., 1989 Science 246:1275-81; Sastry et al., Proc. Natl. Acad. Sci. USA 86:5728-32 (1989); Alting-Mees et al., Strategies in Molecular Biology 3:1-9 (1990); Kang et al., 1991 Proc. Natl. Acad. Sci. USA 88:4363-66; Hoogenboom et al., 1992 J. Molec. Biol. 227:381-388; Schlebusch et al., 1997 Hybridoma 16:47-52 and references cited therein. For example, a library containing a plurality of polynucleotide sequences encoding Ig variable region fragments may be inserted into the genome of a filamentous bacteriophage, such as M13 or a variant thereof, in frame with the sequence encoding a phage coat protein. A fusion protein may be a fusion of the coat protein with the light chain variable region domain and/or with the heavy chain variable region domain. According to certain embodiments, immunoglobulin Fab fragments may also be displayed on a phage particle (see, e.g., U.S. Pat. No. 5,698,426).

Antibody fragments fused to another protein, such as a minor coat protein, can be also used to enrich phage with antigen. Then, using a random combinatorial library of rearranged heavy (V_{H}) and light (V_{L}) chains from mice immune to the antigen (e.g. CTLA-4), diverse libraries of antibody fragments are displayed on the surface of the phage. These libraries can be screened for complementary variable domains, and the domains purified by, for example, affinity column. See Clackson et al., Nature, V. 352 pp. 624-628 (1991).

Heavy and light chain immunoglobulin cDNA expression libraries may also be prepared in lambda phage, for example, using λImmunoZap™(H) and λImmunoZap™(L) vectors (Stratagene, La Jolla, Calif.). Briefly, mRNA is isolated from a B-cell population, and used to create heavy and light chain immunoglobulin cDNA expression libraries in the λImmunoZap(H) and λImmunoZap(L) vectors. These vectors may be screened individually or co-expressed to form Fab fragments or antibodies (see Huse et al., supra; see also Sastry et al., supra). Positive plaques may subsequently be converted to a non-lytic plasmid that allows high level expression of monoclonal antibody fragments from E. coli.

In one embodiment, in a hybridoma the variable regions of a gene expressing a monoclonal antibody of interest are amplified using nucleotide primers. These primers may be synthesized by one of ordinary skill in the art, or may be purchased from commercially available sources. (See, e.g., Stratagene (La Jolla, Calif.), which sells primers for mouse and human variable regions including, among others, primers for V_{Ha}, V_{Hb}, V_{Hc}, V_{Hd}, C_{H1}, V_{L} and C_{L} regions.) These primers may be used to amplify heavy or light chain variable regions, which may then be inserted into vectors such as ImmunoZAP™H or ImmunoZAP™L (Stratagene), respectively. These vectors may then be introduced into E. coli, yeast, or mammalian-based systems for expression. Large amounts of a single-chain protein containing a fusion of the V_{H} and V_{L} domains may be produced using these methods (see Bird et al., Science 242:423-426, 1988).

Once cells producing antibodies according to the disclosure have been obtained using any of the above-described immunization and other techniques, the specific antibody genes may be cloned by isolating and amplifying DNA or mRNA therefrom according to standard procedures as described herein. The antibodies produced therefrom may be sequenced and the CDRs identified and the DNA coding for the CDRs may be manipulated as described previously to generate other antibodies according to the disclosure.

CTLA-4 binding agents of the present disclosure preferably modulate CTLA-4 function in the cell-based assay described herein and/or the in vivo assay described herein and/or bind to one or more of the domains described herein and/or cross-block the binding of one of the antibodies described in this application and/or are cross-blocked from binding CTLA-4 by one of the antibodies described in this application. Accordingly such binding agents can be identified using the assays described herein.

In certain embodiments, antibodies are generated by first identifying antibodies that bind to one or more of the domains provided herein and/or neutralize in the cell-based and/or in vivo assays described herein and/or cross-block the antibodies described in this application and/or are cross-blocked from binding CTLA-4 by one of the antibodies described in this application. The CDR regions from these antibodies are then used to insert into appropriate biocompatible frameworks to generate CTLA-4 binding agents. The non-CDR portion of the binding agent may be composed of amino acids, or may be a non-protein molecule. The assays described herein allow the characterization of binding agents. Preferably the binding agents of the present disclosure are antibodies as defined herein.

Other antibodies according to the disclosure may be obtained by conventional immunization and cell fusion procedures as described herein and known in the art.

Molecular evolution of the complementarity determining regions (CDRs) in the center of the antibody binding site also has been used to isolate antibodies with increased affinity, for example, antibodies having increased affinity for c-erbB-2, as described by Schier et al., 1996, J. Mol. Biol. 263:551. Accordingly, such techniques are useful in preparing antibodies to CTLA-4. Antigen binding proteins directed against a CTLA-4 can be used, for example, in assays to detect the presence of CTLA-4 polypeptides, either in vitro or in vivo. The antigen binding proteins also may be employed in purifying CTLA-4 proteins by immunoaffinity chromatography.

Although human, partially human, or humanized antibodies will be suitable for many applications, particularly those involving administration of the antibody to a human subject, other types of antigen binding proteins will be suitable for certain applications. Non-human antibodies of the present disclosure can be, for example, derived from any antibody-producing animal, such as mouse, rat, rabbit, goat, donkey, or non-human primate (such as monkey (e.g., cynomolgus or rhesus monkey) or ape (e.g., chimpanzee)). An antibody from a particular species can be made by, for example, immunizing an animal of that species with the desired immunogen (e.g., a CTLA-4 polypeptide) or using an artificial system for generating antibodies of that species (e.g., a bacterial or phage display-based system for generating antibodies of a particular species), or by converting an antibody from one species into an antibody from another species by replacing, e.g., the constant region of the antibody with a constant region from the other species, or by replacing one or more amino acid residues of the antibody so that it more closely resembles the sequence of an antibody from the other species. In one embodiment, the antibody is a chimeric antibody comprising amino acid sequences derived from antibodies from two or more different species.

Antigen binding proteins may be prepared, and screened for desired properties, by any of a number of conventional techniques. Certain of the techniques involve isolating a nucleic acid encoding a polypeptide chain (or portion thereof) of an antigen binding protein of interest (e.g., an anti-CTLA-4 antibody), and manipulating the nucleic acid through recombinant DNA technology. The nucleic acid may be fused to another nucleic acid of interest, or altered (e.g., by mutagenesis or other conventional techniques) to add, delete, or substitute one or more amino acid residues, for example. Furthermore, the antigen binding proteins may be purified from cells that naturally express them (e.g., an antibody can be purified from a hybridoma that produces it), or produced in recombinant expression systems, using any technique known in the art. See, for example, Monoclonal Antibodies, Hybridomas: A New Dimension in Biological Analyses, Kennet et al. (eds.), Plenum Press, New York (1980); and Antibodies: A Laboratory Manual, Harlow and Land (eds.), Cold Spring Harbor Laboratory Press, Cold Spring Harbor, N.Y., (1988).

Any expression system known in the art can be used to make the recombinant polypeptides of the present disclosure. Expression systems are detailed comprehensively above. In general, host cells are transformed with a recombinant expression vector that comprises DNA encoding a desired polypeptide. Among the host cells that may be employed are prokaryotes, yeast or higher eukaryotic cells. Prokaryotes include gram negative or gram positive organisms, for example E. coli or Bacilli. Higher eukaryotic cells include insect cells and established cell lines of mammalian origin. Examples of suitable mammalian host cell lines include the COS-7 line of monkey kidney cells (ATCC CRL 1651) (Gluzman et al., 1981, Cell 23:175), L cells, 293 cells, C127 cells, 3T3 cells (ATCC CCL 163), Chinese hamster ovary (CHO) cells, HeLa cells, BHK (ATCC CRL 10) cell lines, and the CVI/EBNA cell line derived from the African green monkey kidney cell line CVI (ATCC CCL 70) as described by McMahan et al., 1991, EMBO J. 10: 2821. Appropriate cloning and expression vectors for use with bacterial, fungal, yeast, and mammalian cellular hosts are described by Pouwels et al. (Cloning Vectors: A Laboratory Manual, Elsevier, New York, 1985).

It will be appreciated that an antibody of the present disclosure may have at least one amino acid substitution, providing that the antibody retains binding specificity. Therefore, modifications to the antibody structures are encompassed within the scope of the present disclosure. These may include amino acid substitutions, which may be conservative or non-conservative that do not destroy the CTLA-4 binding capability of an antibody. Conservative amino acid substitutions may encompass non-naturally occurring amino acid residues, which are typically incorporated by chemical peptide synthesis rather than by synthesis in biological systems. These include peptidomimetics and other reversed or inverted forms of amino acid moieties. A conservative amino acid substitution may also involve a substitution of a native amino acid residue with a normative residue such that there is little or no effect on the polarity or charge of the amino acid residue at that position.

Non-conservative substitutions may involve the exchange of a member of one class of amino acids or amino acid mimetics for a member from another class with different physical properties (e.g. size, polarity, hydrophobicity, charge). Such substituted residues may be introduced into regions of the human antibody that are homologous with non-human antibodies, or into the non-homologous regions of the molecule.

Moreover, one skilled in the art may generate test variants containing a single amino acid substitution at each desired amino acid residue. The variants can then be screened using activity assays known to those skilled in the art. Such variants could be used to gather information about suitable variants. For example, if one discovered that a change to a particular amino acid residue resulted in destroyed, undesirably reduced, or unsuitable activity, variants with such a change may be avoided. In other words, based on information gathered from such routine experiments, one skilled in the art can readily determine the amino acids where further substitutions should be avoided either alone or in combination with other mutations.

A skilled artisan will be able to determine suitable variants of the polypeptide as set forth herein using well-known techniques. In certain embodiments, one skilled in the art may identify suitable areas of the molecule that may be changed without destroying activity by targeting regions not believed to be important for activity. In certain embodiments, one can identify residues and portions of the molecules that are conserved among similar polypeptides. In certain embodiments, even areas that may be important for biological activity or for structure may be subject to conservative amino acid substitutions without destroying the biological activity or without adversely affecting the polypeptide structure.

Additionally, one skilled in the art can review structure-function studies identifying residues in similar polypeptides that are important for activity or structure. In view of such a comparison, one can predict the importance of amino acid residues in a protein that correspond to amino acid residues which are important for activity or structure in similar proteins. One skilled in the art may opt for chemically similar amino acid substitutions for such predicted important amino acid residues.

One skilled in the art can also analyze the three-dimensional structure and amino acid sequence in relation to that structure in similar polypeptides. In view of such information, one skilled in the art may predict the alignment of amino acid residues of an antibody with respect to its three dimensional structure. In certain embodiments, one skilled in the art may choose not to make radical changes to amino acid residues predicted to be on the surface of the protein, since such residues may be involved in important interactions with other molecules.

A number of scientific publications have been devoted to the prediction of secondary structure. See Moult J., Curr. Op. in Biotech., 7(4):422-427 (1996), Chou et al., Biochem., 13(2):222-245 (1974); Chou et al., Biochem., 113(2):211-222 (1974); Chou et al., Adv. Enzymol. Relat. Areas Mol. Biol., 47:45-148 (1978); Chou et al., Ann. Rev. Biochem., 47:251-276 and Chou et al., Biophys. J., 26:367-384 (1979). Moreover, computer programs are currently available to assist with predicting secondary structure. One method of predicting secondary structure is based upon homology modeling. For example, two polypeptides or proteins which have a sequence identity of greater than 30%, or similarity greater than 40% often have similar structural topologies. The recent growth of the protein structural database (PDB) has provided enhanced predictability of secondary structure, including the potential number of folds within a polypeptide's or protein's structure. See Holm et al., Nucl. Acid. Res., 27(1):244-247 (1999). It has been suggested (Brenner et al., Curr. Op. Struct. Biol., 7(3):369-376 (1997)) that there are a limited number of folds in a given polypeptide or protein and that once a critical number of structures have been resolved, structural prediction will become dramatically more accurate.

Additional methods of predicting secondary structure include “threading” (Jones, D., Curr. Opin. Struct. Biol., 7(3):377-87 (1997); Sippl et al., Structure, 4(1):15-19 (1996)), “profile analysis” (Bowie et al., Science, 253:164-170 (1991); Gribskov et al., Meth. Enzym., 183:146-159 (1990); Gribskov et al., Proc. Nat. Acad. Sci., 84(13):4355-4358 (1987)), and “evolutionary linkage” (See Holm, supra (1999), and Brenner, supra (1997)).

In certain embodiments, variants of antibodies include glycosylation variants wherein the number and/or type of glycosylation site has been altered compared to the amino acid sequences of a parent polypeptide. In certain embodiments, variants comprise a greater or a lesser number of N-linked glycosylation sites than the native protein. An N-linked glycosylation site is characterized by the sequence: Asn-X-Ser or Asn-X-Thr, wherein the amino acid residue designated as X can be any amino acid residue except proline. The substitution of amino acid residues to create this sequence provides a potential new site for the addition of an N-linked carbohydrate chain. Alternatively, substitutions which eliminate this sequence will remove an existing N-linked carbohydrate chain. Also provided is a rearrangement of N-linked carbohydrate chains wherein one or more N-linked glycosylation sites (typically those that are naturally occurring) are eliminated and one or more new N-linked sites are created. Additional preferred antibody variants include cysteine variants wherein one or more cysteine residues are deleted from or substituted for another amino acid (e.g, serine) as compared to the parent amino acid sequence. Cysteine variants can be useful when antibodies must be refolded into a biologically active conformation such as after the isolation of insoluble inclusion bodies. Cysteine variants generally have fewer cysteine residues than the native protein, and typically have an even number to minimize interactions resulting from unpaired cysteines.

Desired amino acid substitutions (whether conservative or non-conservative) can be determined by those skilled in the art at the time such substitutions are desired. In certain embodiments, amino acid substitutions can be used to identify important residues of antibodies to CTLA-4, or to increase or decrease the affinity of the antibodies to CTLA-4 described herein.

According to certain embodiments, preferred amino acid substitutions are those which: (1) reduce susceptibility to proteolysis, (2) reduce susceptibility to oxidation, (3) alter binding affinity for forming protein complexes, (4) alter binding affinities, and/or (4) confer or modify other physiochemical or functional properties on such polypeptides. According to certain embodiments, single or multiple amino acid substitutions (in certain embodiments, conservative amino acid substitutions) may be made in the naturally-occurring sequence (in certain embodiments, in the portion of the polypeptide outside the domain(s) forming intermolecular contacts). In certain embodiments, a conservative amino acid substitution typically may not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et al. Nature 354:105 (1991), which are each incorporated herein by reference.

In certain embodiments, antibodies of the present disclosure may be chemically bonded with polymers, lipids, or other moieties.

The binding agents may comprise at least one of the CDRs described herein incorporated into a biocompatible framework structure. In one example, the biocompatible framework structure comprises a polypeptide or portion thereof that is sufficient to form a conformationally stable structural support, or framework, or scaffold, which is able to display one or more sequences of amino acids that bind to an antigen (e.g., CDRs, a variable region, etc.) in a localized surface region. Such structures can be a naturally occurring polypeptide or polypeptide “fold” (a structural motif), or can have one or more modifications, such as additions, deletions or substitutions of amino acids, relative to a naturally occurring polypeptide or fold. These scaffolds can be derived from a polypeptide of any species (or of more than one species), such as a human, other mammal, other vertebrate, invertebrate, plant, bacteria or virus.

Typically the biocompatible framework structures are based on protein scaffolds or skeletons other than immunoglobulin domains. For example, those based on fibronectin, ankyrin, lipocalin, neocarzinostain, cytochrome b, CP1 zinc finger, PST1, coiled coil, LACI-D1, Z domain and tendamistat domains may be used (See e.g., Nygren and Uhlen, 1997, Curr. Opin. in Struct. Biol., 7, 463-469).

Humanized antibodies can be produced using techniques known to those skilled in the art (Zhang, W., et al., Molecular Immunology. 42(12):1445-1451, 2005; Hwang W. et al., Methods. 36(1):35-42, 2005; Dall' Acqua W F, et al., Methods 36(1):43-60, 2005; and Clark, M., Immunology Today. 21(8):397-402, 2000).

Additionally, one skilled in the art will recognize that suitable binding agents include portions of these antibodies, such as one or more of CDR1-L1 to 28 with SEQ ID NOS 1001-1028; CDR2-L1 to 28 with SEQ ID NOS 2001-2028; CDR3-L1 to 28 with SEQ ID NOS 3001-3028; CDR1-H1 to 28 with SEQ ID NOS 4001-4028; CDR2-H1 to 28 with SEQ ID NOS 5001-5028; and CDR3-H1 to 28 with SEQ ID NOS 6001-6028, as specifically disclosed herein. At least one of the regions of CDR regions may have at least one amino acid substitution from the sequences provided here, provided that the antibody retains the binding specificity of the non-substituted CDR. The non-CDR portion of the antibody may be a non-protein molecule, wherein the binding agent cross-blocks the binding of an antibody disclosed herein to CTLA-4 and/or neutralizes CTLA-4. The non-CDR portion of the antibody may be a non-protein molecule in which the antibody exhibits a similar binding pattern to human CTLA-4 peptides in a competition binding assay as that exhibited by at least one of antibodies A1-A28, and/or neutralizes CTLA-4. The non-CDR portion of the antibody may be composed of amino acids, wherein the antibody is a recombinant binding protein or a synthetic peptide, and the recombinant binding protein cross-blocks the binding of an antibody disclosed herein to CTLA-4 and/or neutralizes CTLA-4. The non-CDR portion of the antibody may be composed of amino acids, wherein the antibody is a recombinant antibody, and the recombinant antibody exhibits a similar binding pattern to human CTLA-4 peptides in the human CTLA-4 peptide epitope competition binding assay (described hereinbelow) as that exhibited by at least one of the antibodies A1-A28, and/or neutralizes CTLA-4.

Where an antibody comprises one or more of CDR1-H, CDR2-H, CDR3-H, CDR1-L, CDR2-L and CDR3-L as described above, it may be obtained by expression from a host cell containing DNA coding for these sequences. A DNA coding for each CDR sequence may be determined on the basis of the amino acid sequence of the CDR and synthesized together with any desired antibody variable region framework and constant region DNA sequences using oligonucleotide synthesis techniques, site-directed mutagenesis and polymerase chain reaction (PCR) techniques as appropriate. DNA coding for variable region frameworks and constant regions is widely available to those skilled in the art from genetic sequences databases such as GenBank®.

Once synthesized, the DNA encoding an antibody of the present disclosure or fragment thereof may be propagated and expressed according to any of a variety of well-known procedures for nucleic acid excision, ligation, transformation, and transfection using any number of known expression vectors. Thus, in certain embodiments expression of an antibody fragment may be preferred in a prokaryotic host, such as Escherichia coli (see, e.g., Pluckthun et al., 1989 Methods Enzymol. 178:497-515). In certain other embodiments, expression of the antibody or a fragment thereof may be preferred in a eukaryotic host cell, including yeast (e.g., Saccharomyces cerevisiae, Schizosaccharomyces pombe, and Pichia pastoris), animal cells (including mammalian cells) or plant cells. Examples of suitable animal cells include, but are not limited to, myeloma (such as a mouse NSO line), COS, CHO, or hybridoma cells. Examples of plant cells include tobacco, corn, soybean, and rice cells.

One or more replicable expression vectors containing DNA encoding an antibody variable and/or constant region may be prepared and used to transform an appropriate cell line, for example, a non-producing myeloma cell line, such as a mouse NSO line or a bacteria, such as E. coli, in which production of the antibody will occur. In order to obtain efficient transcription and translation, the DNA sequence in each vector should include appropriate regulatory sequences, particularly a promoter and leader sequence operatively linked to the variable domain sequence. Particular methods for producing antibodies in this way are generally well-known and routinely used. For example, basic molecular biology procedures are described by Maniatis et al. (Molecular Cloning, A Laboratory Manual, 2nd ed., Cold Spring Harbor Laboratory, New York, 1989; see also Maniatis et al, 3rd ed., Cold Spring Harbor Laboratory, New York, (2001)). DNA sequencing can be performed as described in Sanger et al. (PNAS 74:5463, (1977)) and the Amersham International plc sequencing handbook, and site directed mutagenesis can be carried out according to methods known in the art (Kramer et al., Nucleic Acids Res. 12:9441, (1984); Kunkel Proc. Natl. Acad. Sci. USA 82:488-92 (1985); Kunkel et al., Methods in Enzymol. 154:367-82 (1987); the Anglian Biotechnology Ltd. handbook). Additionally, numerous publications describe techniques suitable for the preparation of antibodies by manipulation of DNA, creation of expression vectors, and transformation and culture of appropriate cells (Mountain A and Adair, J R in Biotechnology and Genetic Engineering Reviews (ed. Tombs, M P, 10, Chapter 1, 1992, Intercept, Andover, UK); “Current Protocols in Molecular Biology”, 1999, F. M. Ausubel (ed.), Wiley Interscience, New York).

Where it is desired to improve the affinity of antibodies according to the disclosure containing one or more of the above-mentioned CDRs can be obtained by a number of affinity maturation protocols including maintaining the CDRs (Yang et al., J. Mol. Biol., 254, 392-403, 1995), chain shuffling (Marks et al., Bio/Technology, 10, 779-783, 1992), use of mutation strains of E. coli. (Low et al., J. Mol. Biol., 250, 350-368, 1996), DNA shuffling (Patten et al., Curr. Opin. Biotechnol., 8, 724-733, 1997), phage display (Thompson et al., J. Mol. Biol., 256, 7-88, 1996) and sexual PCR (Crameri, et al., Nature, 391, 288-291, 1998). All of these methods of affinity maturation are discussed by Vaughan et al. (Nature Biotech., 16, 535-539, 1998).

It will be understood by one skilled in the art that some proteins, such as antibodies, may undergo a variety of posttranslational modifications. The type and extent of these modifications often depends on the host cell line used to express the protein as well as the culture conditions. Such modifications may include variations in glycosylation, methionine oxidation, diketopiperizine formation, aspartate isomerization and asparagine deamidation. A frequent modification is the loss of a carboxy-terminal basic residue (such as lysine or arginine) due to the action of carboxypeptidases (as described in Harris, R. J. Journal of Chromatography 705:129-134, 1995).

### 7.7. Sequences

Antibodies A1-A28 comprise heavy and light chain V(J)D polynucleotides (also referred to herein as L1-L28 and H1-H28, respectively). Antibodies A1-A28 comprise the sequences listed in TABLE 5. For example, antibody A1 comprises light chain L1 (SEQ ID NO:1) and heavy chain H1 (SEQ ID NO:101). CDR sequences in the light chain (L1-L28) and heavy chain (H1-H28) are also provided with a specific SEQ ID NOs. For example, three CDR sequences (CDR1, CDR 2 and CDR3) for L1 are CDR1-L1 (SEQ ID NO:1001), CDR2-L1 (SEQ ID NO:2001) and CDR3-L1 (SEQ ID NO:3001), respectively and three CDR sequences (CDR1, CDR 2 and CDR3) for H1 are CDR1-H1 (SEQ ID NO:4001), CDR2-H1 (SEQ ID NO:5001) and CDR3-H1 (SEQ ID NO:6001).

TABLE 5

| Antibodies | Light Chain | Heavy Chain |
|---|---|---|
| A1 | L1 (SEQ ID NO: 1) | H1 (SEQ ID NO: 101) |
| | L1 comprises CDR1-L1 (SEQ ID NO: 1001), | H1 comprises CDR1-H1 (SEQ ID NO: |
| | CDR2-L1 (SEQ ID NO: 2001) and CDR3-L1 | 4001), CDR2-H1 (SEQ ID NO: 5001) and |
| | (SEQ ID NO: 3001) | CDR3-H1 (SEQ ID NO: 6001) |
| A2 | L2 (SEQ ID NO: 2) | H2 (SEQ ID NO: 102) |
| | L2 comprises CDR1-L2 (SEQ ID NO: 1002), | He comprises CDR1-H2 (SEQ ID NO: |
| | CDR2-L2 (SEQ ID NO: 2002) and CDR3-L2 | 4002), CDR2-H2 (SEQ ID NO: 5002) and |
| | (SEQ ID NO: 3002) | CDR3-H2 (SEQ ID NO: 6002) |
| A3 | L3 (SEQ ID NO: 3) | H3 (SEQ ID NO: 103) |
| | L3 comprises CDR1-L3 (SEQ ID NO: 1003), | H3 comprises CDR1-H3 (SEQ ID NO: 4003), |
| | CDR2-L3 (SEQ ID NO: 2003) and CDR3-L3 | CDR2-H3 (SEQ ID NO: 5003) and CDR3- |
| | (SEQ ID NO: 3003) | H3 (SEQ ID NO: 6003) |
| A4 | L4 (SEQ ID NO: 4) | H4 (SEQ ID NO: 104) |
| | L4 comprises CDR1-L4 (SEQ ID NO: 1004), | H4 comprises CDR1-H4 (SEQ ID NO: 4004), |
| | CDR2-L4 (SEQ ID NO: 2004) and CDR3-L4 | CDR2-H4 (SEQ ID NO: 5004) and CDR3- |
| | (SEQ ID NO: 3004) | H4 (SEQ ID NO: 6004) |
| A5 | L5 (SEQ ID NO: 5) | H5 (SEQ ID NO: 105) |
| | L5 comprises CDR1-L5 (SEQ ID NO: 1005), | H4 comprises CDR1-H5 (SEQ ID NO: 4005), |
| | CDR2-L5 (SEQ ID NO: 2005) and CDR3-L5 | CDR2-H5 (SEQ ID NO: 5005) and CDR3- |
| | (SEQ ID NO: 3005) | H5 (SEQ ID NO: 6005) |
| A6 | L6 (SEQ ID NO: 6) | H6 (SEQ ID NO: 106) |
| | L6 comprises CDR1-L6 (SEQ ID NO: 1006), | H6 comprises CDR1-H6 (SEQ ID NO: 4006), |
| | CDR2-L6 (SEQ ID NO: 2006) and CDR3-L6 | CDR2-H6 (SEQ ID NO: 5006) and CDR3- |
| | (SEQ ID NO: 3006) | H6 (SEQ ID NO: 6006) |
| A7 | L7 (SEQ ID NO: 7) | H7 (SEQ ID NO: 107) |
| | L7 comprises CDR1-L7 (SEQ ID NO: 1007), | H7 comprises CDR1-H7 (SEQ ID NO: 4007), |
| | CDR2-L7 (SEQ ID NO: 2007) and CDR3-L7 | CDR2-H7 (SEQ ID NO: 5007) and CDR3- |
| | (SEQ ID NO: 3007) | H7 (SEQ ID NO: 6007) |
| A8 | L8 (SEQ ID NO: 8) | H8 (SEQ ID NO: 108) |
| | L8 comprises CDR1-L8 (SEQ ID NO: 1008), | H8 comprises CDR1-H8 (SEQ ID NO: 4008), |
| | CDR2-L8 (SEQ ID NO: 2008) and CDR3-L6 | CDR2-H8 (SEQ ID NO: 5008) and CDR3- |
| | (SEQ ID NO: 3008) | H8 (SEQ ID NO: 6008) |
| A9 | L9 (SEQ ID NO: 9) | H9 (SEQ ID NO: 109) |
| | L9 comprises CDR1-L9 (SEQ ID NO: 1009), | H9 comprises CDR1-H9 (SEQ ID NO: 4009), |
| | CDR2-L9 (SEQ ID NO: 2009) and CDR3-L9 | CDR2-H9 (SEQ ID NO: 5009) and CDR3- |
| | (SEQ ID NO: 3009) | H9 (SEQ ID NO: 6009) |
| A10 | L10 (SEQ ID NO: 10) | H10 (SEQ ID NO: 110) |
| | L10 comprises CDR1-L10 (SEQ ID | H10 comprises CDR1-H10 (SEQ ID |
| | NO: 1010), CDR2-L10 (SEQ ID NO: 2010) | NO: 4010), CDR2-H10 (SEQ ID NO: 5010) |
| | and CDR3-L10 (SEQ ID NO: 3010) | and CDR3-H10 (SEQ ID NQ:6010) |
| A11 | L11 (SEQ ID NO: 11) | H11 (SEQ ID NO: 111) |
| | L11 comprises CDR1-L11 (SEQ ID | H11 comprises CDR1-H11 (SEQ ID |
| | NO: 1011), CDR2-L11 (SEQ ID NO: 2011) | NO: 4011), CDR2-H11 (SEQ ID NO: 5011) |
| | and CDR3-L11 (SEQ ID NO: 3011) | and CDR3-H11 (SEQ ID NO: 6011) |
| A12 | L12 (SEQ ID NO: 12) | H12 (SEQ ID NO: 112) |
| | L12 comprises CDR1-L12 (SEQ ID | H12 comprises CDR1-H12 (SEQ ID |
| | NO: 1012), CDR2-L12 (SEQ ID NO: 2012) | NO: 4012), CDR2-H12 (SEQ ID NO: 5012) |
| | and CDR3-L12 (SEQ ID NO: 3012) | and CDR3-H12 (SEQ ID NO: 6012) |
| A13 | L13 (SEQ ID NO: 13) | H13 (SEQ ID NO: 113) |
| | L13 comprises CDR1-L13 (SEQ ID | H13 comprises CDR1-H13 (SEQ ID |
| | NO: 1013), CDR2-L13 (SEQ ID NO: 2013) | NO: 4013), CDR2-H13 (SEQ ID NO: 5013) |
| | and CDR3-L13 (SEQ ID NO: 3013) | and CDR3-H13 (SEQ ID NO: 6013) |
| A14 | L14 (SEQ ID NO: 14) | H14 (SEQ ID NO: 114) |
| | L14 comprises CDR1-L14 (SEQ ID | H14 comprises CDR1-H14 (SEQ ID |
| | NO: 1014), CDR2-L14 (SEQ ID NO: 2014) | NO: 4014), CDR2-H14 (SEQ ID NO: 5014) |
| | and CDR3-L14 (SEQ ID NO: 3014) | and CDR3-H14 (SEQ ID NO: 6014) |
| A15 | L15 (SEQ ID NO: 15) | H15 (SEQ ID NO: 115) |
| | L15 comprises CDR1-L15 (SEQ ID | H15 comprises CDR1-H15 (SEQ ID |
| | NO: 1015), CDR2-L15 (SEQ ID NO: 2015) | NO: 4015), CDR2-H15 (SEQ ID NO: 5015) |
| | and CDR3-L15 (SEQ ID NO: 3015) | and CDR3-H15 (SEQ ID NO: 6015) |
| A16 | L16 (SEQ ID NO: 16) | H16 (SEQ ID NO: 116) |
| | L16 comprises CDR1-L16 (SEQ ID | H16 comprises CDR1-H16 (SEQ ID |
| | NO: 1016), CDR2-L16 (SEQ ID NO: 2016) | NO: 4016), CDR2-H16 (SEQ ID NO: 5016) |
| | and CDR3-L16 (SEQ ID NO: 3016) | and CDR3-H16 (SEQ ID NO: 6016) |
| A17 | L17 (SEQ ID NO: 17) | H17 (SEQ ID NO: 117) |
| | L17 comprises CDR1-L17 (SEQ ID | H17 comprises CDR1-H17 (SEQ ID |
| | NO: 1017), CDR2-L17 (SEQ ID NO: 2017) | NO: 4017), CDR2-H17 (SEQ ID NO: 5017) |
| | and CDR3-L17 (SEQ ID NO: 3017) | and CDR3-H17 (SEQ ID NO: 6017) |
| A18 | L18 (SEQ ID NO: 18) | H18 (SEQ ID NO: 118) |
| | L18 comprises CDR1-L18 (SEQ ID | H18 comprises CDR1-H18 (SEQ ID |
| | NO: 1018), CDR2-L18 (SEQ ID NO: 2018) | NO: 4018), CDR2-H18 (SEQ ID NO: 5018) |
| | and CDR3-L18 (SEQ ID NO: 3018) | and CDR3-H18 (SEQ ID NO: 6018) |
| A19 | L19 (SEQ ID NO: 19) | H19 (SEQ ID NO: 119) |
| | L19 comprises CDR1-L19 (SEQ ID | H19 comprises CDR1-H19 (SEQ ID |
| | NO: 1019), CDR2-L19 (SEQ ID NO: 2019) | NO: 4019), CDR2-H19 (SEQ ID NO: 5019) |
| | and CDR3-L19 (SEQ ID NO: 3019) | and CDR3-H19 (SEQ ID NO: 6019) |
| A20 | L20 (SEQ ID NO: 20) | H20 (SEQ ID NO: 120) |
| | L20 comprises CDR1-L20 (SEQ ID | H20 comprises CDR1-H20 (SEQ ID |
| | NO: 1020), CDR2-L20 (SEQ ID NO: 2020) | NO: 4020), CDR2-H20 (SEQ ID NO: 5020) |
| | and CDR3-L20 (SEQ ID NO: 3020) | and CDR3-H20 (SEQ ID NO: 6020) |
| A21 | L21 (SEQ ID NO: 21) | H21 (SEQ ID NO: 121) |
| | L21 comprises CDR1-L21 (SEQ ID | H21 comprises CDR1-H21 (SEQ ID |
| | NO: 1021), CDR2-L21 (SEQ ID NO: 2021) | NO: 4021), CDR2-H21 (SEQ ID NO: 5021) |
| | and CDR3-L21 (SEQ ID NO: 3021) | and CDR3-H21 (SEQ ID NO: 6021) |
| A22 | L22 (SEQ ID NO: 22) | H22 (SEQ ID NO: 122) |
| | L22 comprises CDR1-L22 (SEQ ID | H22 comprises CDR1-H22 (SEQ ID |
| | NO: 1022), CDR2-L22 (SEQ ID NO: 2022) | NO: 4022), CDR2-H22 (SEQ ID NO: 5022) |
| | and CDR3-L22 (SEQ ID NO: 3022) | and CDR3-H22 (SEQ ID NO: 6022) |
| A23 | L23 (SEQ ID NO: 23) | H23 (SEQ ID NO: 123) |
| | L23 comprises CDR1-L23 (SEQ ID | H23 comprises CDR1-H23 (SEQ ID |
| | NO: 1023), CDR2-L23 (SEQ ID NO: 2023) | NO: 4023), CDR2-H23 (SEQ ID NO: 5023) |
| | and CDR3-L23 (SEQ ID NO: 3023) | and CDR3-H23 (SEQ ID NO: 6023) |
| A24 | L24 (SEQ ID NO: 24) | H24 (SEQ ID NO: 124) |
| | L24 comprises CDR1-L24 (SEQ ID | H24 comprises CDR1-H24 (SEQ ID |
| | NO: 1024), CDR2-L24 (SEQ ID NO: 2024) | NO: 4024), CDR2-H24 (SEQ ID NO: 5024) |
| | and CDR3-L24 (SEQ ID NO: 3024) | and CDR3-H24 (SEQ ID NO: 6024) |
| A25 | L25 (SEQ ID NO: 25) | H25 (SEQ ID NO: 125) |
| | L25 comprises CDR1-L25 (SEQ ID | H25 comprises CDR1-H25 (SEQ ID |
| | NO: 1025), CDR2-L25 (SEQ ID NO: 2025) | NO: 4025), CDR2-H25 (SEQ ID NO: 5025) |
| | and CDR3-L25 (SEQ ID NO: 3025) | and CDR3-H25 (SEQ ID NO: 6025) |
| A26 | L26 (SEQ ID NO: 26) | H26 (SEQ ID NO: 126) |
| | L26 comprises CDR1-L26 (SEQ ID | H26 comprises CDR1-H26 (SEQ ID |
| | NO: 1026), CDR2-L26 (SEQ ID NO: 2026) | NO: 4026), CDR2-H26 (SEQ ID NO: 5026) |
| | and CDR3-L26 (SEQ ID NO: 3026) | and CDR3-H26 (SEQ ID NO: 6026) |
| A27 | L27 (SEQ ID NO: 27) | H27 (SEQ ID NO: 127) |
| | L27 comprises CDR1-L27 (SEQ ID | H27 comprises CDR1-H27 (SEQ ID |
| | NO: 1027), CDR2-L27 (SEQ ID NO: 2027) | NO: 4027), CDR2-H27 (SEQ ID NO: 5027) |
| | and CDR3-L27 (SEQ ID NO: 3027) | and CDR3-H27 (SEQ ID NO: 6027) |
| A28 | L28 (SEQ ID NO: 28) | H28 (SEQ ID NO: 128) |
| | L28 comprises CDR1-L28 (SEQ ID | H28 comprises CDR1-H28 (SEQ ID |
| | NO: 1028), CDR2-L28 (SEQ ID NO: 2028) | NO: 4028), CDR2-H28 (SEQ ID NO: 5028) |
| | and CDR3-L28 (SEQ ID NO: 3028) | and CDR3-H28 (SEQ ID NO: 6028) |

### 7.8. Pharmaceutical Compositions

Pharmaceutical compositions containing the proteins and polypeptides of the present disclosure are also provided. Specifically, the present disclosure provides a pharmaceutical composition comprising anti-CTLA ABP. In some embodiments, the pharmaceutical composition comprises GIGA-564. In some embodiments, the pharmaceutical composition comprises GIGA-2328. Such compositions comprise a therapeutically or prophylactically effective amount of the polypeptide or protein in a mixture with pharmaceutically acceptable materials, and physiologically acceptable formulation materials.

The pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition.

Suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates, other organic acids); bulking agents (such as mannitol or glycine), chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides and other carbohydrates (such as glucose, mannose, or dextrins); proteins (such as serum albumin, gelatin or immunoglobulins); coloring; flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (sucrose or sorbitol); tonicity enhancing agents (such as alkali metal halides (preferably sodium or potassium chloride, mannitol sorbitol); delivery vehicles; diluents; excipients and/or pharmaceutical adjuvants. Neutral buffered saline or saline mixed with conspecific serum albumin are examples of appropriate diluents. In accordance with appropriate industry standards, preservatives such as benzyl alcohol may also be added. The composition may be formulated as a lyophilizate using appropriate excipient solutions (e.g., sucrose) as diluents. Suitable components are nontoxic to recipients at the dosages and concentrations employed. Further examples of components that may be employed in pharmaceutical formulations are presented in Remington's Pharmaceutical Sciences, 16^{th} Ed. (1980) and 20^{th} Ed. (2000), Mack Publishing Company, Easton, Pa.

In some embodiments, less than 50% of the ABP in the pharmaceutical composition is fucosylated. In some embodiments, less than 40% of the ABP in the pharmaceutical composition is fucosylated. In some embodiments, less than 30% of the ABP in the pharmaceutical composition is fucosylated. In some embodiments, less than 20% of the ABP in the pharmaceutical composition is fucosylated. In some embodiments, less than 10% of the ABP in the pharmaceutical composition is fucosylated.

In some embodiments, more than 99% of the ABP in the pharmaceutical composition is fucosylated. In some embodiments, more than 95% of the ABP in the pharmaceutical composition is fucosylated. In some embodiments, more than 90% of the ABP in the pharmaceutical composition is fucosylated. In some embodiments, more than 85% of the ABP in the pharmaceutical composition is fucosylated. In some embodiments, more than 80% of the ABP in the pharmaceutical composition is fucosylated. In some embodiments, more than 75% of the ABP in the pharmaceutical composition is fucosylated. In some embodiments, more than 70% of the ABP in the pharmaceutical composition is fucosylated. In some embodiments, more than 65% of the ABP in the pharmaceutical composition is fucosylated. In some embodiments, more than 60% of the ABP in the pharmaceutical composition is fucosylated. In some embodiments, more than 50% of the ABP in the pharmaceutical composition is fucosylated. In some embodiments, more than 40% of the ABP in the pharmaceutical composition is fucosylated. In some embodiments, more than 30% of the ABP in the pharmaceutical composition is fucosylated. In some embodiments, more than 20% of the ABP in the pharmaceutical composition is fucosylated. In some embodiments, more than 10% of the ABP in the pharmaceutical composition is fucosylated.

In some embodiments, 30-70% of the ABP in the pharmaceutical composition is fucosylated. In some embodiments, 20-50% of the ABP in the pharmaceutical composition is fucosylated. In some embodiments, 10-40% of the ABP in the pharmaceutical composition is fucosylated. In some embodiments, 10-30% of the ABP in the pharmaceutical composition is fucosylated. In some embodiments, 5-20% of the ABP in the pharmaceutical composition is fucosylated. In some embodiments, 1-10% of the ABP in the pharmaceutical composition is fucosylated.

In some embodiments, the pharmaceutical formulation materials include one or more of histidine buffer, citrate buffer, sucrose, sodium chloride, succinate, polysorbate 20, and polysorbate-80. In certain embodiments, the pharmaceutical formulation comprises 1-20 mM of histidine or citrate buffer. In certain embodiments, the pharmaceutical formulation comprises 100-350 mM of sucrose. In certain embodiments, the pharmaceutical formulation comprises 0-75 mM of sucrose. In certain embodiments, the pharmaceutical formulation comprises 0.002 to 0.1% by weight of polysorbate-20. In certain embodiments, the pharmaceutical formulation comprises 0.002 to 0.1% by weight of polysorbate-80. In certain embodiments, the pharmaceutical formulation material include 20 mM of citrate or histidine, 170 to 270 mM of sucrose, 0 to 50 mM of sodium chloride, and 0.02% by weight of polysorbate-20. In certain embodiments, the pharmaceutical formulation material include 20 mM of citrate or histidine, 170 to 270 mM of sucrose, 0 to 50 mM of sodium chloride, and 0.02% by weight of polysorbate-80.

In some embodiments, the pharmaceutical composition has a pH from 5.0 to 6.5. In some embodiments, the pharmaceutical composition has a pH from 5.5 to 6.5.

In some embodiments, the pharmaceutical composition comprises 20 mM of histidine or citrate buffer.

In some embodiments, the pharmaceutical composition comprises 50 mM of NaCl.

In some embodiments, the pharmaceutical composition comprises sucrose at a concentration from 170 mM to 270 mM.

In some embodiments, the pharmaceutical composition comprises 170 mM or 270 mM of sucrose.

In some embodiments, the pharmaceutical composition comprises 20 mg/mL of the ABP. In some embodiments, the pharmaceutical composition comprises 5 mg/mL of the ABP. In some embodiments, the pharmaceutical composition comprises 5 mg/mL to 20 mg/mL of the ABP.

In some embodiments, the pharmaceutical composition comprises trehalose.

In some embodiments, the pharmaceutical composition comprises about 0.04 to 30 mg/ml of the ABP, 0.9% Sodium Chloride Injection, USP or 5% Dextrose Injection, USP.

In some embodiments, the ABP is formulated at a concentration of 1 mg/ml to 80 mg/ml. In certain embodiments, the ABP is formulated at a concentration of 5 mg/ml to 20 mg/ml.

In some embodiments, the pharmaceutical composition comprises a pH of about 5 to 7. In some embodiments, the pharmaceutical formulation comprises a pH of about 5.0 to 6.5. In some embodiments, the pharmaceutical formulation comprises a pH of about 5.0, 5.5, 5.6, 5.7, 5.8, 5.9, 6.0, 6.1, 6.2, 6.3, 6.4, 6.5, 6.6, 6.7, 6.8, 6.9, or 7.

Optionally, the composition additionally comprises one or more physiologically active agents, for example, an anti-angiogenic substance, a chemotherapeutic substance (such as capecitabine, 5-fluorouracil, or doxorubicin), an analgesic substance, etc., non-exclusive examples of which are provided herein. In various particular embodiments, the composition comprises one, two, three, four, five, or six physiologically active agents in addition to a CTLA-4-binding protein.

In another embodiment of the present disclosure, the compositions disclosed herein may be formulated in a neutral or salt form. Illustrative pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective.

The carriers can further comprise any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions. The phrase “pharmaceutically-acceptable” refers to molecular entities and compositions that do not produce an allergic or similar untoward reaction when administered to a human.

The optimal pharmaceutical composition will be determined by one skilled in the art depending upon, for example, the intended route of administration, delivery format, and desired dosage. See for example, Remington's Pharmaceutical Sciences, supra. Such compositions may influence the physical state, stability, rate of in vivo release, and rate of in vivo clearance of the polypeptide. For example, suitable compositions may be water for injection, physiological saline solution for parenteral administration.

7.8.1. Content of Pharmaceutically Active Ingredient

In typical embodiments, the active ingredient (i.e., the proteins and polypeptides, ABP, of the present disclosure) is present in the pharmaceutical composition at a concentration of at least 0.01 mg/ml, at least 0.005 mg/ml, at least 0.004 mg/ml, at least 0.05 mg/ml, 0.04 mg/ml, 0.1 mg/ml, at least 0.5 mg/ml, or at least 1 mg/ml. In certain embodiments, the active ingredient is present in the pharmaceutical composition at a concentration of at least 1 mg/ml, 2 mg/ml, 3 mg/ml, 4 mg/ml, 5 mg/ml, 10 mg/ml, 15 mg/ml, 20 mg/ml, 25 mg/ml, or 30 mg/ml. In certain embodiments, the active ingredient is present in the pharmaceutical composition at a concentration of at least 20 mg/ml, 25 mg/ml, 30 mg/ml, 35 mg/ml, 40 mg/ml, 45 mg/ml, 50 mg/ml, 55 mg/ml, 60 mg/ml, 65 mg/ml, 70 mg/ml, 75 mg/ml, 80 mg/ml, 85 mg/ml, 90 mg/ml, 95 mg/ml, or 100 mg/ml. In certain embodiments, the active ingredient is present in the pharmaceutical composition at a concentration ranging from 1 mg/ml to 80 mg/ml. In certain embodiments, the active ingredient is present in the pharmaceutical composition at a concentration ranging from 5 mg/ml to 20 mg/ml. In certain embodiments, the active ingredient is present in the pharmaceutical composition at a concentration ranging from 0.04 mg/ml to 30 mg/ml.

In some embodiments, the pharmaceutical composition comprises one or more additional active ingredients in addition to the proteins or polypeptides of the present disclosure. The one or more additional active ingredients can be a drug targeting a different check-point receptor, such as PD-1 inhibitor (e.g., anti-PD-1 antibody), PD-L1 inhibitor, LAG-3 inhibitor, CD47 inhibitor, or TIGIT inhibitor (e.g., anti-TIGIT antibody).

7.8.2. Formulation Generally

The pharmaceutical composition can be in any form appropriate for human or veterinary medicine, including a liquid, an oil, an emulsion, a gel, a colloid, an aerosol or a solid.

The pharmaceutical composition can be formulated for administration by any route of administration appropriate for human or veterinary medicine, including enteral and parenteral routes of administration.

In various embodiments, the pharmaceutical composition is formulated for administration by inhalation. In certain of these embodiments, the pharmaceutical composition is formulated for administration by a vaporizer. In certain of these embodiments, the pharmaceutical composition is formulated for administration by a nebulizer. In certain of these embodiments, the pharmaceutical composition is formulated for administration by an aerosolizer.

In various embodiments, the pharmaceutical composition is formulated for oral administration, for buccal administration, or for sublingual administration.

In some embodiments, the pharmaceutical composition is formulated for intravenous, intramuscular, or subcutaneous administration.

In some embodiments, the pharmaceutical composition is formulated for intrathecal or intracerebroventricular administration.

In some embodiments, the pharmaceutical composition is formulated for an infusion.

7.8.3. Pharmacological Compositions Adapted for Injection

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives can be included, as required.

In various embodiments, the unit dosage form is a vial, ampule, bottle, or pre-filled syringe. In some embodiments, the unit dosage form contains 0.005 mg, 0.05 mg, 0.01 mg, 0.1 mg, 0.5 mg, 1 mg, 2.5 mg, 5 mg, 10 mg, 12.5 mg, 25 mg, 50 mg, 75 mg, or 100 mg of the pharmaceutical composition. In some embodiments, the unit dosage form contains 125 mg, 150 mg, 175 mg, or 200 mg of the pharmaceutical composition. In some embodiments, the unit dosage form contains 250 mg of the pharmaceutical composition.

In typical embodiments, the pharmaceutical composition in the unit dosage form is in liquid form. In various embodiments, the unit dosage form contains between 0.1 mL and 50 ml of the pharmaceutical composition. In some embodiments, the unit dosage form contains 1 ml, 2.5 ml, 5 ml, 7.5 ml, 10 ml, 25 ml, or 50 ml of pharmaceutical composition.

In particular embodiments, the unit dosage form is a vial containing 1 ml of the pharmaceutical composition at a concentration of 0.01 mg/ml, 0.1 mg/ml, 0.5 mg/ml, or 1 mg/ml. In some embodiments, the unit dosage form is a vial containing 2 ml of the pharmaceutical composition at a concentration of 0.01 mg/ml, 0.1 mg/ml, 0.5 mg/ml, or 1 mg/ml. In some embodiments, the unit dosage form is a vial containing a 1 to 150 ml of the pharmaceutical composition at a concentration of 0.01 mg/ml, 0.1 mg/ml, 0.5 mg/ml, 1 mg/ml, 2 mg/ml, 3 mg/ml, 4 mg/ml, 5 mg/ml, 10 mg/ml, 15 mg/ml, 20 mg/ml, 25 mg/ml, 30 mg/ml, 35 mg/ml, 40 mg/ml, 45 mg/ml, 50 mg/ml, 55 mg/ml, 60 mg/ml, 65 mg/ml, 70 mg/ml, 75 mg/ml, 80 mg/ml, 85 mg/ml, 90 mg/ml, 95 mg/ml, or 100 mg/ml. In some embodiments, the unit dosage form is a vial containing a 1 to 150 ml of the pharmaceutical composition at a concentration of 0.01-0.1 mg/ml, 0.1-0.5 mg/ml, 0.5-1 mg/ml, 1-10 mg/ml, 1-50 mg/ml, 10-50 mg/ml, 10-100 mg/ml, 50-100 mg/ml, or 50-200 mg/ml.

In some embodiments, the pharmaceutical composition in the unit dosage form is in solid form, such as a lyophilate, suitable for solubilization.

Unit dosage form embodiments suitable for subcutaneous, intradermal, or intramuscular administration include preloaded syringes, auto-injectors, and autoinject pens, each containing a predetermined amount of the pharmaceutical composition described hereinabove.

In various embodiments, the unit dosage form is a preloaded syringe, comprising a syringe and a predetermined amount of the pharmaceutical composition. In certain preloaded syringe embodiments, the syringe is adapted for subcutaneous administration. In certain embodiments, the syringe is suitable for self-administration. In particular embodiments, the preloaded syringe is a single use syringe.

In various embodiments, the preloaded syringe contains about 0.1 mL to about 0.5 mL of the pharmaceutical composition. In certain embodiments, the syringe contains about 0.5 mL of the pharmaceutical composition. In specific embodiments, the syringe contains about 1.0 mL of the pharmaceutical composition. In particular embodiments, the syringe contains about 2.0 mL of the pharmaceutical composition.

In certain embodiments, the unit dosage form is an autoinject pen. The autoinject pen comprises an autoinject pen containing a pharmaceutical composition as described herein. In some embodiments, the autoinject pen delivers a predetermined volume of pharmaceutical composition. In other embodiments, the autoinject pen is configured to deliver a volume of pharmaceutical composition set by the user.

In various embodiments, the autoinject pen contains about 0.1 mL to about 5.0 mL of the pharmaceutical composition. In specific embodiments, the autoinject pen contains about 0.5 mL of the pharmaceutical composition. In particular embodiments, the autoinject pen contains about 1.0 mL of the pharmaceutical composition. In other embodiments, the autoinject pen contains about 5.0 mL of the pharmaceutical composition.

### 7.9. Dosage Regimens

In some embodiments, the ABP is administered at a dose sufficient to produce a therapeutic effect.

In various embodiments, the ABP is administered using a weight based dose. In some embodiments, the ABP is administered in an amount of at least 0.05 mg/kg. In some embodiments, the ABP is administered in an amount of at least 0.01 mg/kg. In some embodiments, the ABP is administered in an amount of at least 0.1 mg/kg. In some embodiments, the ABP is administered in an amount of at least 0.5 mg/kg. In certain embodiments, the ABP is administered orally in an amount of at least 1 mg/kg. In certain embodiments, the dose is at least 2 mg/kg, at least 3 mg/kg, at least 4 mg/kg, at least 5 mg/kg, at least 6 mg/kg, at least 7 mg/kg, at least 8 mg/kg, at least 9 mg/kg, or at least 10 mg/kg.

In various embodiments, the dose of the ABP is at least 10 mg/kg. In certain embodiments, the dose is at least 15 mg/kg, at least 20 mg/kg, at least 25 mg/kg, 30 mg/kg, at least 35 mg/kg, at least 40 mg/kg, at least 45 mg/kg, at least 50 mg/kg, at least 55 mg/kg, at least 60 mg/kg, at least 65 mg/kg, at least 70 mg/kg, at least 75 mg/kg, at least 80 mg/kg, at least 85 mg/kg, at least 90 mg/kg, at least 95 mg/kg, at least 100 mg/kg, at least 150 mg/kg, at least 175 mg/kg, or at least 200 mg/kg. In certain embodiments, the dose is 250 mg/kg, 300 mg/kg, 350 mg/kg, 400 mg/kg, 450 mg/kg, 500 mg/kg, 600 mg/kg, 650 mg/kg, 700 mg/kg, 750 mg/kg, 800 mg/kg, 850 mg/kg, 900 mg/kg, 950 mg/kg, or 1000 mg/kg. In certain embodiments, the dose is 0.5 mg/kg to 100 mg/kg per day. In certain embodiments, the dose is 2 mg/kg to 100 mg/kg per day. In certain embodiments, the dose is 25 mg/kg to 1000 mg/kg per day.

### 7.10. Unit Dosage Forms

The pharmaceutical compositions may conveniently be presented in unit dosage form.

The unit dosage form will typically be adapted to one or more specific routes of administration of the pharmaceutical composition.

In various embodiments, the unit dosage form is adapted for administration by inhalation. In certain of these embodiments, the unit dosage form is adapted for administration by a vaporizer. In certain of these embodiments, the unit dosage form is adapted for administration by a nebulizer. In certain of these embodiments, the unit dosage form is adapted for administration by an aerosolizer.

In various embodiments, the unit dosage form is adapted for oral administration, for buccal administration, or for sublingual administration.

In some embodiments, the unit dosage form is adapted for intravenous, intramuscular, or subcutaneous administration.

In some embodiments, the unit dosage form is adapted for intrathecal or intracerebroventricular administration.

In some embodiments, the pharmaceutical composition is formulated for topical administration.

The amount of active ingredient which can be combined with a carrier material to produce a single dosage form will generally be that amount of the compound which produces a therapeutic effect.

### 7.11. Methods of Use

In one aspect, therapeutic antibodies may be used that specifically bind to intact CTLA-4.

In vivo and/or in vitro assays may optionally be employed to help identify optimal dosage ranges. The precise dose to be employed in the formulation will also depend on the route of administration, and the seriousness of the condition, and should be decided according to the judgment of the practitioner and each subject's circumstances. Effective doses may be extrapolated from dose-response curves derived from in vitro or animal model test systems.

An oligopeptide or polypeptide is within the scope of the present disclosure if it has an amino acid sequence that is at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identical to least one of the CDRs provided herein; and/or to a CDR of a CTLA-4 binding agent that cross-blocks the binding of at least one of antibodies A1-A28 to CTLA-4, and/or is cross-blocked from binding to CTLA-4 by at least one of antibodies A1-A28; and/or to a CDR of a CTLA-4 binding agent wherein the binding agent can block the binding of CTLA-4 to its ligands.

CTLA-4 binding agent polypeptides and antibodies are within the scope of the present disclosure if they have amino acid sequences that are at least 85%, 86%, 87%, 88%, 89%, 90%, 91%, 92%, 93%, 94%, 95%, 96%, 97%, 98% or 99% identical to a variable region of at least one of antibodies A1-A28, and cross-block the binding of at least one of antibodies A1-A28 to CTLA-4, and/or are cross-blocked from binding to CTLA-4 by at least one of antibodies A1-A28; and/or can block the inhibitory effect of CTLA-4 on its ligands.

Antibodies according to the disclosure may have a binding affinity for human CTLA-4 of less than or equal to 5×10⁻⁷M, less than or equal to 1×10⁻⁷M, less than or equal to 0.5×10⁻⁷M, less than or equal to 1×10⁻⁸M, less than or equal to 1×10⁻⁹M, less than or equal to 1×10⁻¹⁰M, less than or equal to 1×10⁻¹¹M, or less than or equal to 1×10⁻¹² M.

The affinity of an antibody or binding partner, as well as the extent to which an antibody inhibits binding, can be determined by one of ordinary skill in the art using conventional techniques, for example those described by Scatchard et al. (Ann. N.Y. Acad. Sci. 51:660-672 (1949)) or by surface plasmon resonance (SPR; BIAcore, Biosensor, Piscataway, N.J.). For surface plasmon resonance, target molecules are immobilized on a solid phase and exposed to ligands in a mobile phase running along a flow cell. If ligand binding to the immobilized target occurs, the local refractive index changes, leading to a change in SPR angle, which can be monitored in real time by detecting changes in the intensity of the reflected light. The rates of change of the SPR signal can be analyzed to yield apparent rate constants for the association and dissociation phases of the binding reaction. The ratio of these values gives the apparent equilibrium constant (affinity) (see, e.g., Wolff et al., Cancer Res. 53:2560-65 (1993)).

An antibody according to the present disclosure may belong to any immunoglobin class, for example IgG, IgE, IgM, IgD, or IgA. It may be obtained from or derived from an animal, for example, fowl (e.g., chicken) and mammals, which includes but is not limited to a mouse, rat, hamster, rabbit, or other rodent, cow, horse, sheep, goat, camel, human, or other primate. The antibody may be an internalizing antibody. Production of antibodies is disclosed generally in U.S. Patent Publication No. 2004/0146888 A1.

In the methods described above to generate antibodies according to the disclosure, including the manipulation of the specific A1-A28 CDRs into new frameworks and/or constant regions, appropriate assays are available to select the desired antibodies (i.e. assays for determining binding affinity to CTLA-4; cross-blocking assays; Biacore-based competition binding assay;” in vivo assays).

7.11.1. Methods of Treating a Disease Responsive to a CTLA-4 Inhibitor or Activator

In another aspect, methods are presented for treating a subject having a disease responsive to a CTLA-4 inhibitor or activator. The disease can be cancer, autoimmune disease, or viral or bacterial infection.

The terms “treatment,” “treating,” and the like are used herein to generally mean obtaining a desired pharmacologic and/or physiologic effect. The effect may be prophylactic, in terms of completely or partially preventing a disease, condition, or symptoms thereof, and/or may be therapeutic in terms of a partial or complete cure for a disease or condition and/or adverse effect, such as a symptom, attributable to the disease or condition. “Treatment” as used herein covers any treatment of a disease or condition of a mammal, particularly a human, and includes: (a) preventing the disease or condition from occurring in a subject which may be predisposed to the disease or condition but has not yet been diagnosed as having it; (b) inhibiting the disease or condition (e.g., arresting its development); or (c) relieving the disease or condition (e.g., causing regression of the disease or condition, providing improvement in one or more symptoms). Improvements in any conditions can be readily assessed according to standard methods and techniques known in the art. The population of subjects treated by the method of the disease includes subjects suffering from the undesirable condition or disease, as well as subjects at risk for development of the condition or disease.

By the term “therapeutically effective dose” or “effective amount” is meant a dose or amount that produces the desired effect for which it is administered. The exact dose or amount will depend on the purpose of the treatment, and will be ascertainable by one skilled in the art using known techniques (see, e.g., Lloyd (1999) The Art, Science and Technology of Pharmaceutical Compounding).

The term “sufficient amount” means an amount sufficient to produce a desired effect.

The term “therapeutically effective amount” is an amount that is effective to ameliorate a symptom of a disease. A therapeutically effective amount can be a “prophylactically effective amount” as prophylaxis can be considered therapy.

The term “ameliorating” refers to any therapeutically beneficial result in the treatment of a disease state, e.g., a neurodegenerative disease state, including prophylaxis, lessening in the severity or progression, remission, or cure thereof.

The actual amount administered, and rate and time-course of administration, will depend on the nature and severity of protein aggregation disease being treated. Prescription of treatment, e.g. decisions on dosage etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980.

In some embodiments, the pharmaceutical composition is administered by inhalation, orally, by buccal administration, by sublingual administration, by injection or by topical application.

In some embodiments, the pharmaceutical composition is administered in an amount sufficient to modulate survival of neurons or dopamine release. In some embodiments, the major cannabinoid is administered in an amount less than 1 g, less than 500 mg, less than 100 mg, less than 10 mg per dose.

In some embodiments, the pharmaceutical composition is administered once a day, 2-4 times a day, 2-4 times a week, once a week, or once every two weeks.

A composition can be administered alone or in combination with other treatments, either simultaneously or sequentially dependent upon the condition to be treated. For example, the pharmaceutical composition can be administered in combination with one or more drugs targeting a different check-point receptor, such as PD-1 inhibitor (e.g., anti-PD-1 antibody), PD-L1 inhibitor, LAG-3 inhibitor, CD47 inhibitor, or TIGIT inhibitor (e.g., anti-TIGIT antibody).

In some embodiments, the disease is selected from the group consisting of cancer, AIDS, Alzheimer's disease and viral or bacterial infection. In certain embodiments, the disease is cancer.

In certain embodiments, the disease is cancer. In certain embodiments, the subject has a tumor. Cancers that may be treated include tumors that are not vascularized, or not yet substantially vascularized, as well as vascularized tumors. Types of cancers to be treated with the pharmaceutical composition described herein include, but are not limited to, carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers are also included. In some embodiments, the cancer is RCC (renal cell cancer), NSCLC (non-small cell lung cancer), Merkel cell carcinoma, cSCC, mesothelioma, MSI colorectal cancer, ovarian cancer, or cervical cancer. In some embodiments, the subject has a tumor with high levels of Tregs, high levels of CTLA-4, high levels of NK cells, or high levels of activating FcRs.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases). In some embodiments, the subject has a tumor with high levels of Tregs, high levels of CTLA-4, high levels of NK cells, or high levels of activating FcRs.

In some embodiments, the subject is suffering from a cancer selected from the group consisting of colon carcinoma, breast cancer, pancreatic cancer, ovarian cancer, prostate cancer, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, merkel cell carcinoma, craniopharyngioma, ependymoma, pinealoma, hemangioblastoma, acoustic neuroma, oligodendroglioma, meningioma, melanoma, neuroblastoma, retinoblastoma, acute lymphocytic leukemia, acute myelocytic leukemia, chronic leukemia, polycythemia vera, lymphoma, multiple myeloma, Waldenstrom's macroglobulinemia, heavy chain disease, and combinations thereof.

In some embodiments, the subject has a tumor with high levels of Tregs, high levels of CTLA-4, high levels of NK cells, or high levels of activating FcRs.

In additional embodiments, the cancer is a solid tumor selected from the group consisting of fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases.

In certain embodiments, the cancer is a solid tumor selected from the group consisting of fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma/colorectal cancer, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases.

In another aspect, the present disclosure provides a method of reducing CTLA-4^{HI} Tregs in a subject with limited proliferation of remaining Tregs comprising administering to the subject an effective dose of an antigen binding protein.

In some embodiments, the subject is a human subject, optionally, a human subject with cancer.

In some embodiments, the subject is a human subject, optionally, a human subject with melanoma, RCC (renal cell cancer), NSCLC (non-small cell lung cancer), Merkel cell carcinoma, cSCC, mesothelioma, MSI colorectal cancer, ovarian cancer, or cervical cancer.

In some embodiments, the method further comprising the step of administering one or more additional therapeutic agents to the subject.

In some embodiments, the subject has a tumor with high levels of Tregs, high levels of CTLA-4, high levels of NK cells, or high levels of activating FcRs.

In some embodiments, the ABP binds to an antigen selected from CTLA-4, PD-L1, PD1 TIGIT, LAG-3 a CD47, BRAF, MEK, PI3K, and other antigens. In some embodiments, the ABP is specific to CTLA-4 or other antigens.

In some embodiments, the ABP is selected from an anti-CTLA-4 antibody or antigen-binding fragment thereof, anti-PD-L1 antibody or antigen-binding fragment thereof, an anti-PD1 antibody or antigen-binding fragment thereof, a TIGIT antibody or antigen-binding fragment thereof, a LAG-3 antibody or antigen-binding fragment thereof, a CD47 antibody or antigen-binding fragment thereof, a BRAF antibody or antigen-binding fragment thereof, a MEK antibody or antigen-binding fragment thereof, and a PI3K antibody or antigen-binding fragment thereof.

### 8. EXAMPLES

Below are examples of specific embodiments for carrying out the present disclosure. The examples are offered for illustrative purposes only, and are not intended to limit the scope of the present disclosure in any way. Efforts have been made to ensure accuracy with respect to numbers used (e.g., amounts, temperatures, etc.), but some experimental error and deviation should, of course, be allowed for.

The practice of the present disclosure will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3^{rd} Ed. (Plenum Press) Vols A and B (1992). Furthermore, methods of generating and selecting antibodies explained in Adler et al., A natively paired antibody library yields drug leads with higher sensitivity and specificity than a randomly paired antibody library, MAbs (2018), and Adler et al., Rare, high-affinity mouse anti-CTLA-4 antibodies that function in checkpoint blockade, discovered using microfluidics and molecular genomics, MAbs (2017), which are incorporated by reference in its entirety herein, can be employed.

### 8.1. Example 1: Generation of Antigen Binding Protein

### Mouse Immunization and Sample Preparation:

First, transgenic mice carrying inserted human immunoglobulin genes were immunized with soluble CTLA-4 immunogen of SEQ ID NO: 7001 (i.e., His-tagged CTLA-4 protein (R&D Systems)) using TiterMax as an adjuvant. One μg of immunogen was injected into each hock and 3 pg of immunogen was administered intraperitoneally, every third day for 15 days. Titer was assessed by enzyme-linked immunosorbent assay (ELISA) on a 1:2 dilution series of each animal's serum, starting at a 1:200 dilution. A final intravenous boost of 2.5 μg/hock without adjuvant was given to each animal before harvest. Lymph nodes (popliteal, inguinal, axillary, and mesenteric) were surgically removed after sacrifice. Single cell suspensions for each animal were made by manual disruption followed by passage through a 70 μm filter. Next, the EasySep™ Mouse Pan-B Cell Isolation Kit (Stemcell Technologies) negative selection kit was used to isolate B cells from each sample. The lymph node B cell populations were quantified by counting on a C-Chip hemocytometer (Incyto) and assessed for viability using Trypan blue. The cells were then diluted to 5,000-6,000 cells/mL in phosphate-buffered saline (PBS) with 12% OptiPrep™ Density Gradient Medium (Sigma). This cell mixture was used for microfluidic encapsulation. Approximately one million B cells were run from each of the six animals through an emulsion droplet microfluidics platform.

### Generating Paired Heavy and Light Chain Libraries:

A DNA library encoding scFv from RNA of single cells, with native heavy-light Ig pairing intact, was generated using the emulsion droplet microfluidics platform or vortex emulsions. The method for generating the DNA library was divided into 1) poly(A)+mRNA capture, 2) multiplexed overlap extension reverse transcriptase polymerase chain reaction (OE-RT-PCR), and 3) nested PCR to remove artifacts and add adapters for deep sequencing or yeast display libraries. The scFv libraries were generated from approximately one million B cells from each animal that achieved a positive ELISA titer.

For poly(A)+mRNA capture, a custom designed co-flow emulsion droplet microfluidic chip fabricated from glass (Dolomite) was used. The microfluidic chip has two input channels for fluorocarbon oil (Dolomite), one input channel for the cell suspension mix described above, and one input channel for oligo-dT beads (NEB) at 1.25 mg/ml in cell lysis buffer (20 mM Tris pH 7.5, 0.5 M NaCl, 1 mM ethylenediaminetetraacetic acid (EDTA), 0.5% Tween-20, and 20 mM dithiothreitol). The input channels were etched to 50 μm by 150 μm for most of the chip's length, narrow to 55 μm at the droplet junction, and were coated with hydrophobic Pico-Glide (Dolomite). Three Mitos P-Pump pressure pumps (Dolomite) were used to pump the liquids through the chip. Droplet size depends on pressure, but typically droplets of ˜45 mm diameter are optimally stable. Emulsions were collected into chilled 2 ml microcentrifuge tubes and incubated at 40° C. for 15 minutes for mRNA capture. The beads were extracted from the droplets using Pico-Break (Dolomite). In some embodiments, similar single cell partitioning emulsions were made using a vortex.

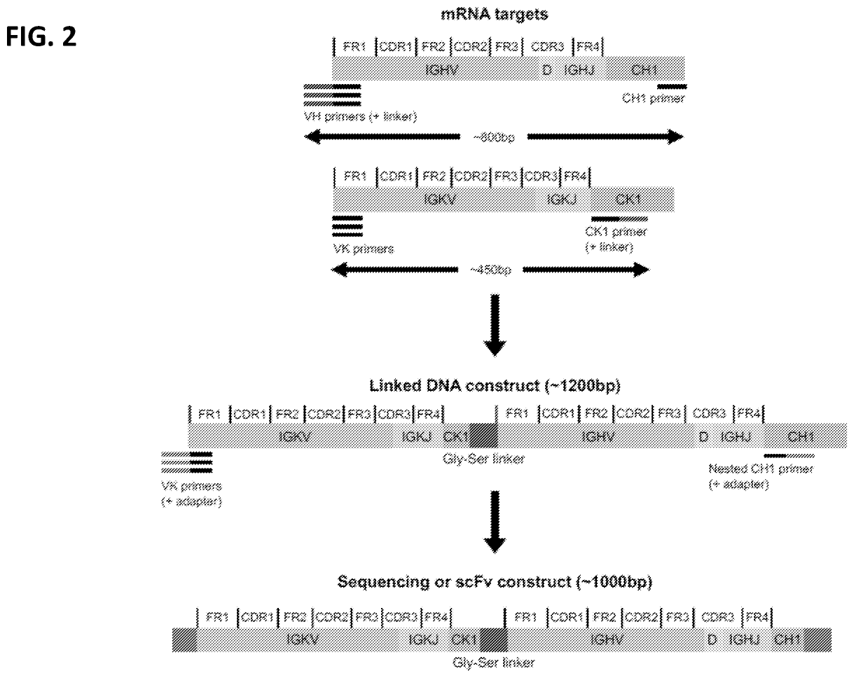

For multiplex OE-RT-PCR, glass Telos droplet emulsion microfluidic chips were used (Dolomite). mRNA-bound beads were re-suspended into OE-RT-PCR mix and injected into the microfluidic chips with a mineral oil-based surfactant mix (available commercially from GigaGen) at pressures that generate 27 μm droplets. The OE-RT-PCR mix contains 2× one-step RT-PCR buffer, 2.0 mM MgSO₄, SuperScript III reverse transcriptase, and Platinum Taq (Thermo Fisher Scientific), plus a mixture of primers directed against the IgK C region, the IgG C region, and all V regions (FIG. 2). The overlap region was a DNA sequence that encodes a Gly-Ser rich scFv linker sequence. The DNA fragments were recovered from the droplets using a droplet breaking solution (available commercially from GigaGen) and then purified using QIAquick PCR Purification Kit (Qiagen). In some embodiments, similar OE-RT-PCR emulsions were made using a vortex.

For nested PCR (FIG. 2), the purified OE-RT-PCR product was first run on a 1.7% agarose gel for 80 minutes at 150 V. A band at 1200-1500 base pair (bp) corresponding to the linked product was excised and purified using NucleoSpin Gel and PCR Clean-up Kit (Macherey Nagel). PCR was then performed to add adapters for Illumina sequencing or yeast display; for sequencing, a randomer of seven nucleotides is added to increase base calling accuracy in subsequent next generation sequencing steps. Nested PCR was performed with 2× NEBNext High-Fidelity amplification mix (NEB) with either Illumina adapter containing primers or primers for cloning into the yeast expression vector. The nested PCR product was run on a 1.2% agarose gel for 50 minutes at 150V. A band at 800-1100 bp was excised and purified using NucleoSpin Gel and PCR Clean-up Kit (Macherey Nagel).

In some embodiments, scFv libraries were not natively paired, for example, randomly paired by amplifying scFv directly from RNA isolated from B cells.

### 8.2. Example 2: Isolation of CTLA-4 Binders by Yeast Display

### Library Screening:

Human IgG1-Fc (Thermo Fisher Scientific) and CTLA-4 (R&D Systems) proteins were biotinylated using the EZ-Link Micro Sulfo-NHS-LC-Biotinylation kit (Thermo Fisher Scientific). The biotinylation reagent was resuspended to 9 mM and added to the protein at a 50-fold molar excess. The reaction was incubated on ice for 2 hours and then the biotinylation reagent was removed using Zeba desalting columns (Thermo Fisher Scientific). The final protein concentration was calculated with a Bradford assay.

Next, the six DNA libraries were expressed as surface scFv in yeast. A yeast surface display vector (pYD) that contains a GAL1/10 promoter, an Aga2 cell wall tether, and a C-terminal c-Myc tag was built. The GAL1/10 promoter induces expression of the scFv protein in medium that contains galactose. The Aga2 cell wall tether was required to shuttle the scFv to the yeast cell surface and tether the scFv to the extracellular space. The c-Myc tag was used during the flow sort to stain for yeast cells that express in-frame scFv protein. Saccharomyces cerevisiae cells (ATCC) were electroporated (Bio-Rad Gene Pulser II; 0.54 kV, 25 uF, resistance set to infinity) with gel-purified nested PCR product and linearized pYD vector for homologous recombination in vivo. Transformed cells were expanded and induced with galactose to generate yeast scFv display libraries.

Two million yeast cells from the expanded scFv libraries were stained with anti-c-Myc (Thermo Fisher Scientific A21281) and an AF488-conjugated secondary antibody (Thermo Fisher Scientific A11039). To select scFv-expressing cells that bind to CTLA-4, biotinylated CTLA-4 antigen was added to the yeast culture (7 nM final) during primary antibody incubation and then stained with PE-streptavidin (Thermo Fisher Scientific). Yeast cells were flow sorted on a BD Influx (Stanford Shared FACS Facility) for double-positive cells (AF488C/PEC), and recovered clones were then plated on SD-CAA plates with kanamycin, streptomycin, and penicillin (Teknova) for expansion. The expanded first round FACS clones were then subjected to a second round of FACS with the same antigen at the same molarity (7 nM final). Plasmid minipreps (Zymo Research) were prepared from yeast recovered from the final FACS sort. Tailed-end PCR was used to add Illumina adapters to the plasmid libraries for deep sequencing.

In a typical FACS dot plot, the upper right quadrant contains yeast that stain for both antigen binding and scFv expression (identified by a C-terminal c-Myc tag). The lower left quadrant contains yeast that do not stain for either the antigen or scFv expression. The lower right quadrant contains yeast that express the scFv but do not bind the antigen. The frequency of binders in each repertoire was estimated by dividing the count of yeast that double stain for antigen and scFv expression by the count of yeast that express an scFv. Libraries generated from immunized mice yielded low percentages of scFv binders (ranging from 0.08%-1.28%) when sorted at 7 nM final antigen concentration. There was no clear association between serum titer and the frequency of binders in a repertoire. Following expansion of these sorted cells, a second round of FACS at 7 nM final antigen concentration was used to increase the specificity of the screen. The frequency of binders in the second FACS was always substantially higher than the first FACS, ranging from 8.39%-84.4%. Generally, lower frequency of binders in the first sort yielded lower frequency of binders in the second sort. Presumably, this is due to lower gating specificity for samples that have fewer bona fide binders in the original repertoire.

### Deep Repertoire Sequencing:

CTLA-4-binding clones were recovered as a library (“a library of CTLA-4 binding clones”), and subjected to deep repertoire sequencing. Deep repertoire sequencing determines the sequences of all paired variable (V(D)J) regions of both heavy and light chain sequences. The library of CTLA-4 binding clones was deposited under ATCC Accession No. PTA-125512 under the Budapest Treaty on Nov. 20, 2018, under ATCC Account No. 197361 (American Type Culture Collection (ATCC), 10801 University Boulevard, Manassas, Va. 20110 USA). Each clone in the library contains an scFv comprising a paired variable (V(D)J) regions of both heavy and light chain sequences originating from a single cell. Deep repertoire sequencing determines the sequences of all paired variable (V(D)J) regions of both heavy and light chain sequences. Some of the heavy and light chain sequences obtained from sequencing the yeast scFv library are provided in SEQ ID NOS: 1-28 and SEQ ID NOS: 101-128. Additional sequences obtained from sequencing the yeast scFv library are provided in SEQ ID NOS 8000-8991. Specifically, their variable light chain (V_{L}) sequences include SEQ ID NOS: 8000-8495. Their heavy chain (V_{H}) sequences include SEQ ID NOS: 8496-8991.

Deep antibody sequencing libraries were quantified using a quantitative PCR Illumina Library Quantification Kit (KAPA) and diluted to 17.5 pM. Libraries were sequenced on a MiSeq (Illumina) using a 500 cycle MiSeq Reagent Kit v2, according to the manufacturer's instructions. To obtain high quality sequence reads with maintained heavy and light chain linkage, sequencing was performed in two separate runs. In the first run (“linked run”), the scFv libraries were directly sequenced to obtain forward read of 340 cycles for the light chain V-gene and CDR3, and reverse read of 162 cycles that cover the heavy chain CDR3 and part of the heavy chain V-gene. In the second run (“unlinked run”), the scFv library was first used as a template for PCR to separately amplify heavy and light chain V-genes. Then, forward reads of 340 cycles and reverse reads of 162 cycles for the heavy and light chain Ig were obtained separately. This produces forward and reverse reads that overlap at the CDR3 and part of the V-gene, which increases confidence in nucleotide calls.

To remove base call errors, the expected number of errors (E) for a read were calculated from its Phred scores. By default, reads with E>1 were discarded, leaving reads for which the most probable number of base call errors is zero. As an additional quality filter, singleton nucleotide reads were discarded because sequences found two or more times have a high probability of being correct. Finally, high-quality, linked antibody sequences by merging filtered sequences were generated from the linked and unlinked runs. Briefly, a series of scripts that first merged forward and reverse reads from the unlinked run were written in Python. Any pairs of forward and reverse sequences that contained mismatches were discarded. Next, the nucleotide sequences from the linked run were used to query merged sequences in the unlinked run. The final output from the scripts is a series of full-length, high-quality variable (V(D)J) sequences, with native heavy and light chain Ig pairing.

To identify reading frame and FR/CDR junctions, a database of well-curated immunoglobulin sequences were first processed to generate position-specific sequence matrices (PSSMs) for each FR/CDR junction. These PSSMs were used to identify FR/CDR junctions for each of the merged nucleotide sequences generated using the processes described above. This identified the protein reading frame for each of the nucleotide sequences. CDR sequences that have a low identify score to the PSSMs are indicated by an exclamation point. Python scripts were then used to translate the sequences. Reads were required to have a valid predicted CDR3 sequence, so, for example, reads with a frame-shift between the V and J segments were discarded. Next, UBLAST was run using the scFv nucleotide sequences as queries and V and J gene sequences from the IMGT database as the reference sequences. The UBLAST alignment with the lowest E-value was used to assign V and J gene families and compute % ID to germline.

Each animal yielded 38-50 unique scFv sequences present at 0.1% frequency or greater after the second FACS selection, including a total of 28 unique scFv candidate binders (SEQ ID Nos: 1-28 for light chains; SEQ ID Nos: 101-128 for heavy chains). The light chain having a sequence of SEQ ID NO: [n] and the heavy chain having a sequence of SEQ ID NO: [100+n] are a cognate pair from a single cell, and forming a single scFv. For example, the light chain of SEQ ID NO:1 and the heavy chain of SEQ ID NO:101 are a cognate pair, the light chain of SEQ ID NO:28 and the heavy chain of SEQ ID NO:128 are a cognate pair, etc.

In this method, the two rounds of FACS resulted in enrichment of the CTLA-4-binding scFvs. In addition, many scFv were not detected in the sequencing data from the initial population of B cells from the immunized mice and most of the scFv present in the pre-sort mouse repertoires were eliminated following FACS. Therefore, this work suggests that most of the antibodies present in the repertoires of immunized mice are not strong binders to the immunogen and that this method can enrich for rare nM-affinity binders from the initial population of B cells from immunized mice.

### 8.3. Example 3: Biological Characteristics of Antigen Binding Protein

scFv sequences that were present at low frequency in pre-sort libraries and became high frequency in post-sort libraries were then synthesized as full-length mAbs in Chinese hamster ovary (CHO) cells. These mAbs comprise the 2-3 most abundant sequences in the second round of FACS for each animal.

### CTLA-4 Target Binding Profiles

The binding specificity and affinity of each full-length antibody towards CTLA-4 were determined using biolayer interferometry (BLI) and/or surface plasmon resonance (SPR). Anti-cyno CTLA-4 and anti-mouse CTLA-4 affinities were tested using ForteBio (BLI). Anti-human CTLA-4 affinities were measured using Carterra (SPR).

For BLI, antibodies were loaded onto an Anti-Human IgG Fc (AHC) biosensor using the Octet Red96 system (ForteBio). Loaded biosensors were dipped into antigen dilutions beginning at 300 nM, with 6 serial dilutions at 1:3. Kinetic analysis was performed using a 1:1 binding model and global fitting.

For SPR, a moderate density (»1,000 Response Units) of an antihuman IgG-Fc reagent (Southern Biotech 2047-01) was amine-coupled to a Xantec CMD-50M chip (50 nm carboxymethyldextran medium density of functional groups) activated with 133 mM EDC (Sigma) and 33.3 mM S-NHS (ThermoFisher) in 100 mM MES pH 5.5. Then, goat anti-Human IgG Fc (Southern Biotech 2047-01) was coupled for 10 minutes at 25 mg/m L in 10 mM Sodim Acetate pH 4.5 (Carterra Inc.). The surface was then deactivated with 1 M ethanolamine pH 8.5 (Carterra Inc.). Running buffer used for the lawn immobilization was HBS-EPC (10 mM HEPES, 150 mM NaCl, 3 mM EDTA, 0.05% Tween 20, pH 7.4; Teknova).

The sensor chip was then transferred to a continuous flow microspotter (CFM; Carterra Inc.) for array capturing. The mAb supernatants were diluted 50-fold (3-10 mg/mL final concentration) into HBS-EPC with 1 mg/mL BSA. The samples were each captured twice with 15-minute and 4-minute capture steps on the first and second prints, respectively, to create multiple densities, using a 65 mL/min flow rate. The running buffer in the CFM was also HBS-EPC.

Next, the sensor chip was loaded onto an SPR reader (MX-96 system; Ibis Technologies) for the kinetic analysis. CTLA-4 was injected at five increasing concentrations in a four-fold dilution series with concentrations of 1.95, 7.8, 31.25, 125, and 500 nM in running buffer (HBS-EPC with 1.0 mg/mL BSA). CTLA-4 injections were 5 minutes with a 15-minute dissociation at 8 mL/second in a non-regenerative kinetic series. An injection of the goat anti-Human IgG Fc capture antibody at 75 mg/mL was injected at the end of the series to verify the capture level of each mAb. Binding data was double referenced by subtracting an interspot surface and a blank injection and analyzed for ka (on-rate), kd (off-rate), and KD (affinity) using the Kinetic Interaction Tool software (Carterra Inc.).

For cell surface binding studies, stable CTLA-4 expressing Flp-In CHO (Thermo Fisher Scientific) cells were generated and mixed at a 50:50 ratio. One million cells were stained with 1 μg of the disclosed anti-CTLA-4 recombinant antibodies in 200 μl of MACS Buffer (DPBS with 0.5% bovine serum albumin and 2 mM EDTA) for 30 minutes at 4° C. Cells were then co-stained with anti-human irrelevant target APC and anti-human IgG Fc-PE [M1310G05](BioLegend 41070) antibodies for 30 minutes at 4° C. An anti-human CTLA-4-FITC antibody was used as a control for these mixing experiments and cell viability was assessed with DAPI. Flow cytometry analysis was conducted on a BD Influx at the Stanford Shared FACS Facility and data was analyzed using FlowJo.

Antibodies that specifically bind to CTLA-4 were identified. Affinity to CTLA-4 (K_{D}) of each antibody is provided in TABLE 6. The Promega assay % inhibition was calculated relative to the strongest inhibitor, which is antibody A5. The affinity of each antibody against human CTLA-4, the on rate, off rate, and KD are shown in TABLE 7.

TABLE 6

| | | Promega | | Affinity | Affinity | Affinity |
|---|---|---|---|---|---|---|
| | | assay | | to | to | to |
| | Binds | antagonist | Promega | Human | Cyno | Mouse |
| | by | (EC50, | assay % | CTLA- | CTLA4 | CTLA-4 |
| Ab# | FACS? | ug/mL) | inhibition | 4(nM) | (nM) | (nM) |
| Ipilimumab | Yes | 0.51 | 50.2% | 4.9 | 1.3 | no binding |
| A1 | Yes | 0.13 | 43.2% | 0.77 | 0.96 | 51 |
| A2 | Yes | 0.12 | 75.4% | 5.1 | 3.1 | no binding |
| A3 | Yes | 0.18 | 49.8% | 1.5 | 1.2 | no binding |
| A4 | Yes | 0.15 | 81% | 3 | 1.2 | no binding |
| A5 | Yes | 0.18 | 100% | 4.6 | 2.2 | 39.4 |
| A6 | Yes | 0.18 | 61.7% | 5.7 | 1.6 | 87.8 |
| A7 | Yes | 0.17 | 11.5% | 5.7 | 0.75 | no binding |
| A8 | Yes | 0.15 | 54.7% | 4.6 | 1 | no binding |
| A9 | Yes | 0.19 | 60.8% | 6.8 | 0.92 | no binding |
| A10 | Yes | 0.26 | 24.5% | 22 | 3.1 | no binding |
| A11 | Yes | 0.42 | 26.6% | 7.5 | 1.6 | no binding |
| A12 | Yes | no blocking | 0% | 5.3 | 4.5 | not tested |
| A13 | Yes | 0.09 | 13.3% | 3.2 | 9.6 | not tested |
| A14 | Yes | 0.09 | 6% | 9.8 | 23.6 | not tested |
| A15 | Yes | no blocking | 0% | 10 | 2.6 | not tested |
| A16 | Yes | no blocking | 0% | 23 | 2.8 | not tested |
| A17 | Yes | no blocking | 0% | 51 | 6.3 | not tested |
| A18 | Yes | 0.89 | 8.6% | 48 | no binding | not tested |
| A19 | No | not tested | not tested | 3.3 | not tested | not tested |
| A20 | No | not tested | not tested | 20 | not tested | not tested |
| A21 | No | not tested | not tested | 31 | not tested | not tested |
| A22 | No | not tested | not tested | 32 | not tested | not tested |
| A23 | No | no blocking | 0% | 35 | not tested | not tested |
| A24 | No | not tested | not tested | 55 | not tested | not tested |
| A25 | No | not tested | not tested | 58 | not tested | not tested |
| A26 | No | not tested | not tested | 74 | not tested | not tested |
| A27 | No | not tested | not tested | 120 | not tested | not tested |
| A28 | No | no blocking | 0% | 46 | not tested | not tested |

TABLE 7

| | kon (M−1 s−1) | koff (s−1) | KD (M) |
|---|---|---|---|
| Ipilimumab | 6.50E+04 | 3.20E−04 | 4.90E−09 |
| A1 | 2.10E+05 | 1.60E−04 | 7.70E−10 |
| A2 | 1.00E+05 | 5.20E−04 | 5.10E−09 |
| A3 | 1.90E+05 | 2.80E−04 | 1.50E−09 |
| A4 | 7.60E+04 | 2.30E−04 | 3.00E−09 |
| A5 | 9.00E+04 | 4.20E−04 | 4.60E−09 |
| A6 | 6.10E+04 | 3.50E−04 | 5.70E−09 |
| A7 | 6.40E+04 | 3.60E−04 | 5.70E−09 |
| A8 | 8.50E+04 | 3.90E−04 | 4.60E−09 |
| A9 | 6.30E+04 | 4.30E−04 | 6.80E−09 |
| A10 | 2.30E+04 | 5.10E−04 | 2.20E−08 |
| A11 | 4.70E+04 | 3.50E−04 | 7.50E−09 |
| A12 | 5.10E+04 | 2.70E−04 | 5.30E−09 |
| A13 | 1.30E+05 | 4.20E−04 | 3.20E−09 |
| A14 | 5.40E+04 | 5.20E−04 | 9.80E−09 |
| A15 | 7.00E+04 | 7.30E−04 | 1.00E−08 |
| A16 | 3.10E+04 | 7.20E−04 | 2.30E−08 |
| A17 | 6.60E+04 | 3.40E−03 | 5.10E−08 |
| A18 | 4.20E+03 | 2.00E−04 | 4.80E−08 |
| A19 | 1.20E+05 | 3.80E−04 | 3.30E−09 |
| A20 | 3.20E+04 | 6.40E−04 | 2.00E−08 |
| A21 | 1.80E+04 | 5.50E−04 | 3.10E−08 |
| A22 | 1.10E+04 | 3.60E−04 | 3.20E−08 |
| A23 | 3.40E+04 | 1.20E−03 | 3.50E−08 |
| A24 | 2.50E+04 | 1.40E−03 | 5.50E−08 |
| A25 | 3.00E+04 | 1.80E−03 | 5.80E−08 |
| A26 | 3.50E+03 | 2.60E−04 | 7.40E−08 |
| A27 | 3.10E+04 | 3.60E−03 | 1.20E−07 |
| A28 | 5.20E+04 | 2.40E−03 | 4.60E−08 |

### CTLA-4 Ligand Blocking Assay:

For analysis of the antibodies' ability to block the CTLA-4/ligand interaction, the CTLA-4 Blockade Bioassay (Promega) was used according to the manufacturer's instructions. On the day prior to the assay, aAPC/Raji cells that express CTLA-4 ligands CD80 and CD86 were thawed into 90% Ham's F-12/10% fetal bovine serum (FBS) and plated into the inner 60 wells of two 96-well plates. The cells were incubated overnight at 37° C., 5% CO₂. On the day of assay, antibodies were diluted in 99% RPMI/1% FBS. The antibody dilutions were added to the wells containing the CTLA-4 ligand expressing aAPC/Raji cells, followed by addition of CTLA-4 effector cells (thawed into 99% RPMI/1% FBS). The cell/antibody mixtures were incubated at 37° C., 5% CO₂ for 6 hours, after which Bio-Glo Reagent was added and luminescence was read using a Spectramax i3x plate reader (Molecular Devices). Fold-induction was plotted by calculating the ratio of [signal with antibody]/[signal with no antibody], and the plots were used to calculate the EC50 using SoftMax Pro (Molecular Devices). In-house produced ipilimumab was used as a positive control, and an antibody binding to an irrelevant antigen was used as a negative control.

Binding of CTLA-4 to its ligand leads to inhibition of T cell signaling. Antibodies that bind CTLA-4 and antagonize CTLA-4/ligand interactions can therefore remove this inhibition, allowing T cells to be activated. CTLA-4/ligand checkpoint blockade was tested through an in vitro cellular Nuclear Factor of Activated T cells (NFAT) luciferase reporter assay. In this assay, antibodies whose anti-CTLA-4 epitopes fall inside the ligand binding domain antagonize CTLA-4/ligand interactions, resulting in an increase of the NFAT-luciferase reporter. The full-length mAb candidates that can bind CTLA-4 expressed in CHO cells were assayed. To generate an EC50 value for each mAb, measurements were made across several concentrations. It was found that some full-length mAbs are functional in checkpoint blockade in a dose dependent manner as summarized in TABLE 6.

The ability of the CTLA4 antibodies (indicated in TABLE 8) to prevent the binding of CD80 or CD86 to plate-bound CTLA4 was evaluated using ELISA. The EC50 and the percent inhibition of each interaction is shown in the TABLE 8. Plates were coated with rhCTLA4-Fc and then blocked with 1×PBST with 5% w/v nonfat dry milk. After blocking a dilution series of the indicated antibody was added to the plate. Then, to determine how much CD80 or CD86 was still able to bind plate bound CTLA4, after the plates were washed, rhCD80-His or rhCD86-His, respectively, was added to the plate. Unbound CD80-His/CD86-His was washed away and mouse anti-His-HRP was added. TMB was used to determine how much CD80-His/CD86-His bound to the plate bound CTLA4 in the presence of each antibody.

In some embodiments of the present disclosure, the anti-CTLA-4 antibodies function pharmacologically by antibody-dependent cell-mediated cytotoxicity (ADCC). In some embodiments of the present disclosure, immune-related toxicities related to anti-CTLA-4 antibody therapy are abrogated with an antibody that functions in ADCC but which does not function in checkpoint blockade.

TABLE 8

| | CD80 EC50 | CD80 % | CD86 EC50 | CD86 % |
|---|---|---|---|---|
| Antibody | (ug/mL) | inhibition | (ug/ml) | inhibition |
| Ipilimumab | 0.08211 | 96.5% | 0.1136 | 90.6% |
| CTLA4.A7 | 0.9955 | 92.1% | 1.98 | 78.8% |
| CTLA4.A2 | 0.1006 | 95.4% | 0.1452 | 91.5% |
| CTLA4.A12 | 0.2427 | 89.2% | 0.3704 | 77.4% |
| CTLA4.A14 | 0.2262 | 83.7% | 0.2442 | 54.5% |
| CTLA4.A5 | 0.07049 | 96.3% | 0.1218 | 90.6% |

### Epitope Binning:

Epitope binning was performed using high-throughput Array SPR in a modified classical sandwich approach. A sensor chip was functionalized using the Carterra CFM and methods similar to the SPR affinity studies, except a CMD-200M chip type was used (200 nm carboxymethyl dextran, Xantec) and mAbs were coupled at 50 mg/mL to create a surface with higher binding capacity (˜3,000 reactive units immobilized). The mAb supernatants were diluted at 1:1 or 1:10 in running buffer, depending on the concentration of the mAb in the supernatant.

The sensor chip was placed in the MX-96 instrument, and the captured mAbs (“ligands”) were crosslinked to the surface using the bivalent amine reactive linker bis(sulfosuccinimidyl) suberate (BS3, ThermoFisher), which was injected for 10 minutes at 0.87 mM in water. Excess activated BS3 was neutralized with 1 M ethanolamine pH 8.5. For each binning cycle, a 7-minute injection of 250 mg/mL human IgG (Jackson ImmunoResearch 009-000-003) was used to block reference surfaces and any remaining capacity of the target spots.

Next, 250 nM CTLA-4 protein was injected onto the sensor chip, followed by injections of the diluted mAb supernatants (“analytes”) or buffer blanks as negative controls. Thus, the analyte mAb only bound to the antigen if it was not competitive with the ligand mAb. At the end of each cycle, a one minute regeneration injection was performed using a solution of 4 parts Pierce IgG Elution Buffer (ThermoFisher #21004), one part 5 M NaCl (0.83 M final), and 1.25 parts 0.85% H3PO4 (0.17% final).

A network community plot algorithm was then used in an SPR epitope data analysis software package (Carterra Inc.) to determine epitope bins. Note that the clustering algorithm groups mAbs for which only analyte data are available separately from the mAbs for which both ligand and analyte data are available. This phenomenon is an artifact of the incomplete competitive matrix. mAbs with both ligand and analyte data had more mAb-mAb measurements, resulting in more mAb-mAb connections, which led to a closer relationship in the community plot.

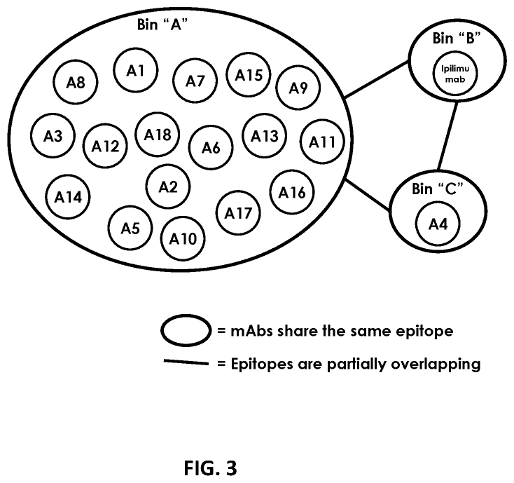

The epitope binning showed that all the mAbs were in distinct bins from ipilimumab (FIG. 3).

### 8.4. Example 4: Influence of CTLA-4 ABPs on Tumor Growth

Transgenic mice expressing human CTLA-4 (hCTLA-4 KI mice) were implanted subcutaneously with MC38 tumor cells on the right flank. The hCTLA-4 KI mice were treated with one mg/kg of the indicated CTLA-4 antibody on Days 8, 11, and 14 post-implantation. Specifically, the mice were treated with a control antibody (n=8), ipilumumab (n=8), CTLA4. A2 antibody (n=8), CTLA4. A14 antibody (n=9), CTLA4. A14.2a antibody (n=8), CTLA4. A7 antibody (n=9), CTLA4. A7 antibody (n=9), and CTLA4. A12 antibody (n=8). CTLA-4. A14.2a antibody is the A14 antibody cloned onto a mouse IgG2a background, which enhances antibody-dependent cellular cytotoxicity (ADCC) activity. Tumor volume was measured and tumor growth inhibition was calculated using the formula below:

Mean % Inhibition=(mean(C)−mean(T))/mean(C)*100%
T—current group value
C—control group value

Tumors were implanted subcutaneously in the right flank region with MC38 tumor cells (1×10⁶) in 0.1 ml of PBS for tumor development. The cells in exponential growth phase were harvested and quantitated by cell counter before tumor implantation. Tumor volumes were measured twice per week in two dimensions using a caliper, and the volume will be expressed in mm³ using the formula: “V=(L×W×W)/2, where V is tumor volume, L is tumor length (the longest tumor dimension) and W is tumor width (the longest tumor dimension perpendicular to L). Dosing as well as tumor and body weight measurements were conducted in a Laminar Flow Cabinet. The body weights and tumor volumes were measured by using StudyDirector™ software (version 3.1.399.19). Animals were dosed i.p. (intraperitoneally) with the indicated protein in a sterile saline solution including 0.1 mg/ml of the indicated protein. Each mouse received 10 microliters of the indicated solution per a gram of body weight, which leads to a dosing of 1 mg/kg. Animals were dosed days 0, 3, and 6 post randomization.

TABLE 9 shows the percentage of mice in which the tumor had a complete response (CR) to the treatment. At least 2 consecutive tumor measurements of 0 mm³ following treatment initiation qualifies as a CR.

TABLE 9

| Antibody | % of tumors with CR | |
|---|---|---|
| Control | 12.5% | (1 of 8) |
| Ipilimumab | 75% | (6 of 8) |
| CTLA4.A2 | 100% | (8 of 8) |
| CTLA4.A14 | 66.67% | (6 of 9) |
| CTLA4.A14.2a | 75% | (6 of 8) |
| CTLA4.A7 | 55.6% | (5 of 9) |
| CTLA4.A12 | 50% | (4 of 8) |

TABLE 10 shows the percentage of mice with tumors that had a CR but then later relapsed by day 56. The group treated with CTLA4. A14.2a that had previously shown a CR had 0% relapse by day 56, indicating that enhancing ADCC can prolong the anti-tumor immunity.

TABLE 10

| | % of relapse by Day 56 in tumors | |
|---|---|---|
| Antibody | that had previously shown CR | |
| Ipilimumab | 16.67% | (1 of 6) |
| CTLA4.A2 | 25% | (2 of 8) |
| CTLA4.A14 | 33.3% | (2 of 6) |
| CTLA4.A14.2a | 0% | (0 of 6) |

TABLE 11 shows the mean inhibition of tumor volume over time when the hCTLA-4 KI mice implanted with MC38 tumor cells were treated with one mg/kg of the control or one mg/kg of the indicated CTLA-4 antibodies.

TABLE 11
Mean Inhibition

| Study day | 8 | 11 | 14 | 17 | 21 | 24 | 27 | 29 | 31 | 35 | 38 |
|---|---|---|---|---|---|---|---|---|---|---|---|
| Control | n/a | n/a | n/a | n/a | n/a | n/a | n/a | n/a | n/a | n/a | n/a |
| Ipilimumab | −3.81% | 0.44% | 31.41% | 69.65% | 84.04% | 90.73% | 96.00% | 96.92% | 96.61% | 87.45% | 88.27% |
| CTLA4.A7 | −7.49% | 10.91% | 34 54% | 60.11% | 70.73% | 81.04% | 78.25% | 78.75% | 70.60% | 50.50% | 43.41% |
| CTLA4.A2 | −6.79% | −6.10% | 26.70% | 61.14% | 85.22% | 97.98% | 99.58% | 100.00% | 100.00% | 99.55% | 99.36% |
| CTLA4.A12 | −5.89% | 12.35% | 34.21% | 49.59% | 70.56% | 86.82% | 90.22% | 91.82% | 91.13% | 68.63% | 70.44% |
| CTLA4.A14 | −7.37% | −6.91% | 26.70% | 58.54% | 80.64% | 93.71% | 95.73% | 97.23% | 97.26% | 92.22% | 90.17% |
| CTLA4.A14.2a | −4.74% | −20.04% | 9.74% | 40.80% | 65.98% | 87.85% | 93.48% | 95.01% | 93.66% | 81.40% | 82.75% |

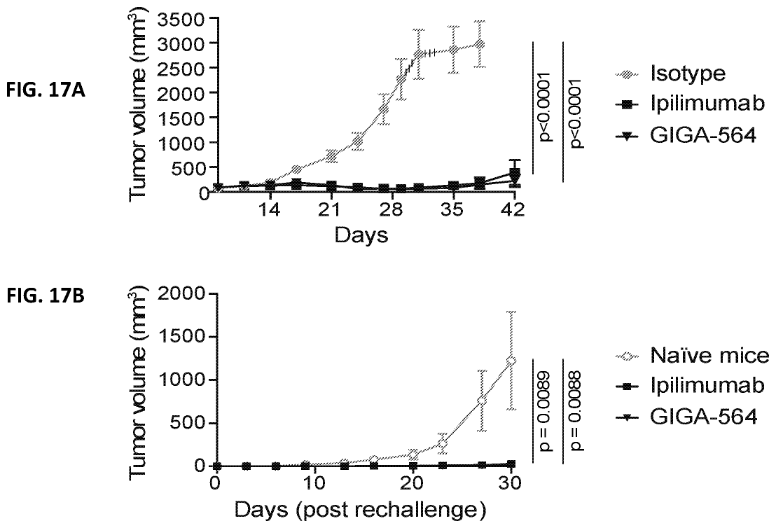

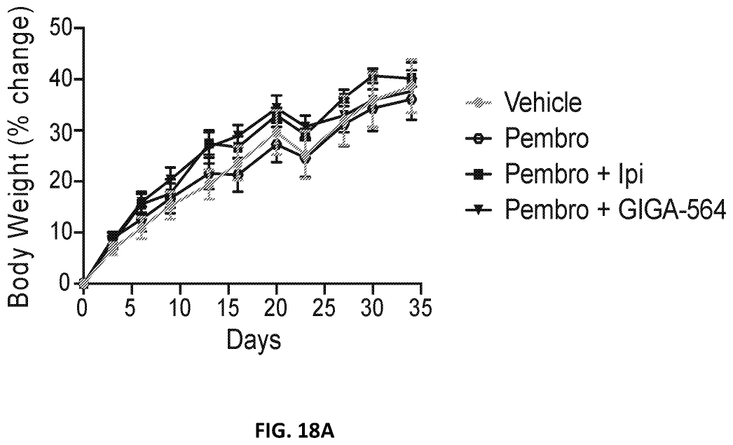

This data is also shown in FIG. 17A.

### 8.5. Example 5: Influence of CTLA4 ABPs on Systemic Anti-Tumor Immunity

The hCTLA4 KI mice bearing MC38 tumors were treated with the indicated anti-CTLA4 on days 8, 11, and 14 post tumor cell implantation, as explained supra. The mice in which tumors displayed a CR were re-challenged with implantation of MC38 cells on the opposite flank. TABLE 12 shows the individual mouse tumor volumes (mm³) of the original or re-challenge tumors on the final day of the study (73 days after the original tumor cell implantation and 30 days after the re-challenge implantation). There was no growth of re-challenge tumors in mice in which the original tumor remained a CR. The 3 instances of growth seen in the re-challenge tumors were in mice in which the original tumor had started to re-grow (see TABLE 12). The results also indicated that CTLA4. A2 may induce protective systemic anti-tumor immunity even when the primary tumor (original tumor) relapses (see TABLE 13).

TABLE 12

| | | Ipilimumab | | CTLA4.A2 | | CTLA4.A1 |
|---|---|---|---|---|---|---|
| | Ipilimumab | (re- | CTLA4.A2 | (re- | CTLA4.A1 | 4 (re- |
| | (original | challenge | (original | challenge | 4 (original | challenge |
| | tumor) | tumor) | tumor) | tumor) | tumor) | tumor) |
| Tumor | 0 | 0 | 0 | 0 | 105.7 | 85.3 |
| Volume | 1822.3 | 149.6 | 0 | 0 | 0 | 0 |
| (mm³) | 0 | 0 | 0 | 0 | 913.3 | 98.1 |
| | 0 | 0 | 0 | 0 | 0 | 0 |
| | 0 | 0 | 1415 | 0 | 0 | 0 |
| | 0 | 0 | 0 | 0 | 0 | 0 |
| | | | 678.9 | 0 | | |
| | | | 0 | 0 | | |

TABLE 13

| | % of secondary tumors that |
|---|---|
| Antibody | grew if primary tumor relapsed |
| Ipilimumab | 100% (1 of 1) |
| CTLA4.A2 | 0% (0 of 2) |
| CTLA4.A14 | 100% (2 of 2) |

Data from this experiment is also shown in FIG. 17B.

### 8.6. Example 6: Influence of Increased Dosage of CLTA-4 ABPs

MC38 Tumors Treated with Anti-CTLA-4

Transgenic mice expressing human CTLA-4 (hCTLA-4 KI mice) were implanted with MC38 tumor cells on the right flank. Randomization started when the mean tumor size reached 98.5 mm². The hCTLA-4 KI mice were treated with 5 mg/kg of the indicated anti-CTLA4 bi-weekly for 5 doses starting on day 0 post-randomization. The administered antibodies are shown in TABLE 14. CTLA4. A14.2a is antibody A14 cloned onto a mouse IgG2a backbone, which enhances ADCC activity. The 297 suffix denotes that the hIgG1 Fc was mutated at the N297 amino acid to eliminate glycosylation and thus Fc effector function including ADCC.

### A) Tumor Growth Inhibition

Over the course of the study, tumor growth inhibition was determined using the formula below:

Mean % Inhibition=(mean(C)−mean(T))/mean(C)*100%
T—current group value
C—control group value

The results showed that antibodies lacking Fc activity had reduced efficacy overall. These antibodies were still able to induce tumor regression in some animals, indicating that anti-CTLA4 works by both Fc-dependent and Fc-independent mechanisms of action, and indicating that anti-CTLA4s lacking Fc activity, including ADCC and ADCP, can induce anti-tumor responses (TABLEs 14 and 15).

TABLE 14

| | Mean Inhibition | | | | | | |
|---|---|---|---|---|---|---|---|
| Study Day | 0 | 3 | 6 | 10 | 13 | 17 | 20 |
| 1x PBS (negative control) | n/a | n/a | n/a | n/a | n/a | n/a | n/a |
| Ipilimumab | 0.31% | −21.76% | 12.56% | 75.43% | 91.55% | 95.54% | 98.71% |
| CTLA4.A5 | 0.74% | −28.09% | −6.22% | 64.17% | 87.32% | 98.32% | 100.00% |
| CTLA4.A14 | −0.56% | −13.30% | 5.33% | 68.17% | 86.21% | 92.79% | 97.52% |
| CTLA4.A12 | 0.40% | −32.59% | −14.13% | 57.48% | 83.00% | 94.99% | 98.04% |
| CTLA4.A8 | 0.26% | −18.48% | −14.18% | 52.53% | 78.37% | 96.15% | 97.96% |
| CTLA4.A7 | −0.61% | −7.67% | 10.04% | 76.74% | 94.36% | 98.88% | 100.00% |
| CTLA4.A2 | 0.27% | −23.97% | −9.95% | 60.95% | 89.33% | 98.35% | 100.00% |
| CTLA4.A13 | −1.15% | −7.61% | 6.23% | 49.18% | 64.82% | 93.82% | 95.23% |
| CTLA4.A5.2a | 0.88% | 11.78% | 27.51% | 66.64% | 89.02% | 96.85% | 100.00% |
| CTLA4.A14.2a | 1.04% | −7.89% | 21.12% | 45.60% | 84.83% | 97.30% | 100.00% |
| Ipilimumab.297 | 0.68% | 8.27% | 21.37% | 22.75% | 16.02% | 25.70% | 26.39% |
| CTLA4.A5.297 | −0.33% | 17.56% | 18.58% | 5.70% | −15.40% | 4.65% | 13.61% |

TABLE 15
% of each group in which tumor had started to
regress by day 13 post treatment initiation

| | |
|---|---|
| 1× PBS (negative control) | 0% |
| Ipilimumab | 100% |
| CTLA4.A5 | 100% |
| CTLA4.A14 | 100% |
| CTLA4.A12 | 88% |
| CTLA4.A8 | 100% |
| CTLA4.A7 | 100% |
| CTLA4.A2 | 100% |
| CTLA4.A13 | 86% |
| CTLA4.A5.2a | 100% |
| CTLA4.A14.2a | 100% |
| Ipilimumab.297 | 25% |
| CTLA4.A5.297 | 14% |

Data from this experiment is also shown in_FIGS. 12B, 14A and 27.

### B) Histopathological Analysis:

The hCTLA-4 mice were euthanized and their right kidneys were harvested for histopathological analysis. Tissue was formalin-fixed and paraffin-embedded, and cut in 5 μm sections that were placed on glass slides for standard hematoxylin and eosin (H&E) staining as well as anti-IgG and anti-C3 immunohistochemistry (IHC) staining. Stained slides were prepared as digital images. A board-certified veterinary pathologist with experience in laboratory animals and toxicologic pathology evaluated the H&E images for any findings and evaluated the anti-IgG and C3 slides for location, intensity, and percent of positive staining. Findings in H&E images were scored on a scale from 0 to 5 (0=within normal limits, 1=minimal findings or the least change discernible, 2=mild findings, 3=moderate, 4=marked, and 5=severe or to the greatest extent possible). Findings in IHC images were scored on a scale of 1 to 4 for intensity (0=negative, 1=minimal or slightly positive and 4=very dark), and as a percent of the positive cells in the glomeruli (after reviewing at least 5 glomeruli).

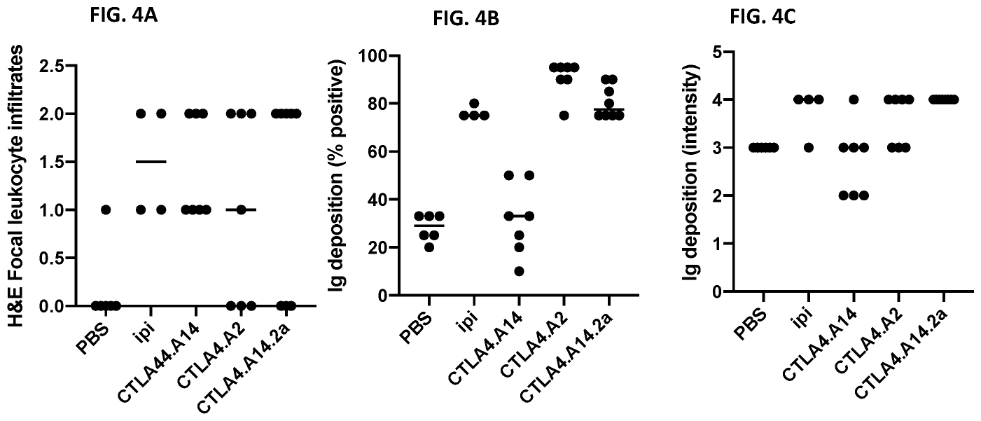

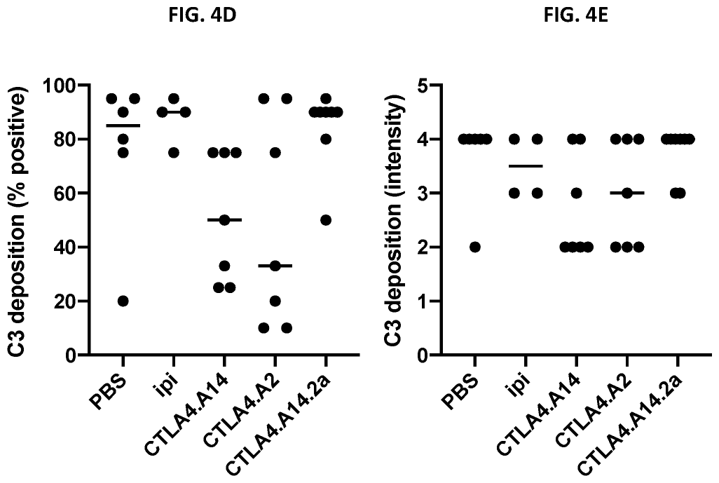

The H&E, immunoglobulin, or C3 stain images were scored by a blinded pathologist and the results are shown in FIG. 4A-4C. The main H&E finding was that leukocytes in the renal interstitium are not usually involved in glomeruli. Ig and C3 deposition in the glomeruli scoring are also shown in FIG. 4A-4C.

### C) Alkaline Phosphatase:

The hCTLA-4 mice were also analyzed for changes in alkaline phosphatase level. The level of alkaline phosphatase in the serum was determined using comprehensive diagnostic rotors on ABAXIS VetScan VS2.

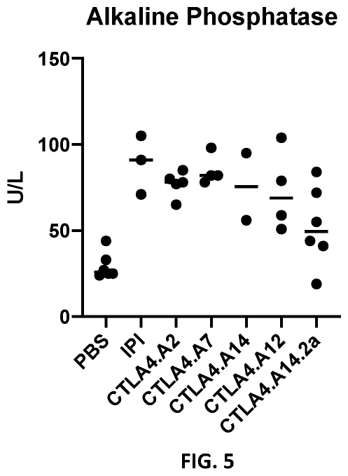

The study found that ipilimumab (IPI) elevates alkaline phosphatase levels, which may be an indication of immune-mediated hepatitis. The CTLA4 antibodies (e.g., CTLA4. A14.2A) showed a decrease in elevation of alkaline phosphatase levels (FIG. 5). This decreased elevation in alkaline phosphatase induced by the presently disclosed CTLA4 antibodies may indicate they are less likely to induce immune-mediated hepatitis than treatments such as ipilimumab.

### 8.7. Example 7: Influence of CTLA-4 ABPs on a Second Tumor Model

RM1 Tumors Treated with Anti-CTLA-4

Transgenic mice expressing human CTLA-4 (hCTLA-4 KI mice) were implanted with RM1 tumor cells on the right flank. (Human IgG1 isotype negative control n=7, atezolimumab n=8, n=11 for all other groups). The hCTLA4 KI mice were treated with the antibodies indicated in TABLE 16. The CTLA4 antibodies were dosed at 5 mg/kg on days 0, 3, and 6 post randomization and atezolizumab was dosed at 5 mg/kg biweekly for 3 weeks starting at day 0 post randomization. Human IgG1 isotype negative control was dosed at 5 mg/kg on Days 0, 3, and 6 post randomization. The mean inhibition of tumor growth was determined at Days 0, 4, 7, 11, 14, and 18 using the following formula:

Mean % Inhibition=(mean(C)−mean(T))/mean(C)*100%
T—current group value
C—control group value

TABLE 16 shows that mean inhibition values for the control, the CTLA4 antibodies, and the atezolizumab treatments over the course of the study.

TABLE 16

| | Mean Inhibition | | | | | |
|---|---|---|---|---|---|---|
| Study day | 0 | 4 | 7 | 11 | 14 | 18 |
| Human IgG1 isotype | n/a | n/a | n/a | n/a | n/a | n/a |
| (negative control) | | | | | | |
| Ipilimumab | 2.14% | 11.33% | 30.07% | 41.96% | 52.16% | 57.56% |
| CTLA4.A2 | 0.09% | −12.28% | 25.30% | 43.83% | 50.20% | 59.41% |
| CTLA4.A14 | −0.17% | −7.72% | 22.10% | 36.93% | 48.84% | 55.18% |
| Atezolizumab | 1.83% | 17.66% | 0.41% | −3.02% | −7.89% | −2.89% |
| Atezolizumab + | −0.47% | −3.31% | 18.00% | 29.81% | 30.39% | 35.21% |
| Ipilimumab | | | | | | |
| Atezolizumab + | 1.50% | −6.72% | 23.00% | 41.64% | 40.93% | 44.30% |
| CTLA4.A2 | | | | | | |
| Atezolizumab + | 0.87% | 2.55% | 31.58% | 45.65% | 47.43% | 55.18% |
| CTLA4.A14 | | | | | | |

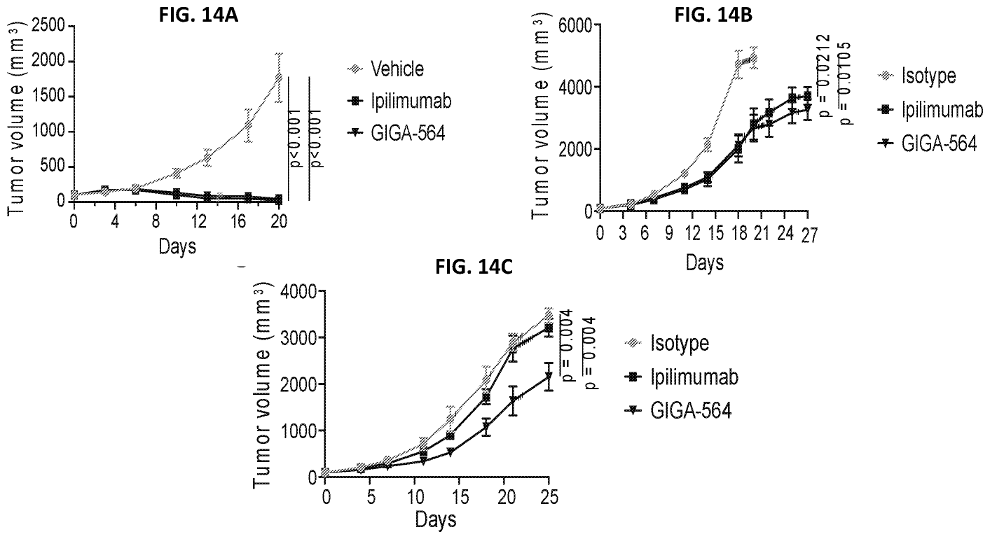

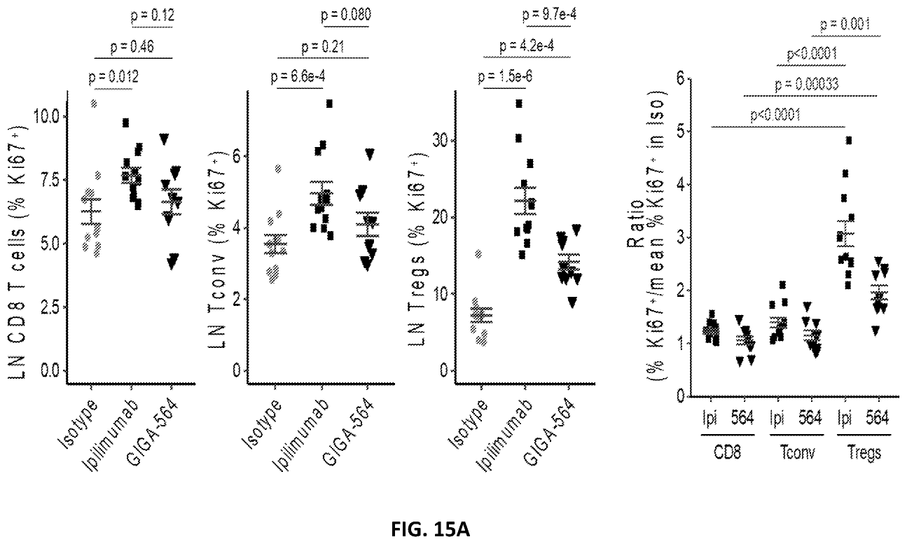

Data from this experiment is also shown in FIG. 14B.

### 8.8. Example 8: Combination Treatment (Pembrolizumab and Anti-CTLA4s)

Transgenic mice expressing human CTLA-4 and PD-1 (hCTLA4-hPD1 knock in (KI) mice, n=8 per treatment group) were implanted subcutaneously with 1×10⁶ MC38 tumor cells in the right flank. The hCTLA4-hPD1 KI mice were treated with a control (1× phosphate buffered saline, or PBS); 2 mg/kg pembrolizumab (pembro) or 2 mg/kg pembro+5 mg/kg anti-CTLA4, administered i.p. with a dose volume of 10 ml/kg per animal as indicated in TABLE 17 twice weekly for three weeks starting on day 1 post-randomization. The mean (%) delta inhibition of tumor growth induced by each treatment in comparison to control treatment was calculated using the formula below and the results are shown in TABLE 17.

Mean % ΔInhibition=((mean(C)−mean(C₀))−(mean(T)−mean(T₀)))/(mean(C)−Mean(C₀))*100%
T—current group value
T₀—current group initial value
C—control group value
C₀—control group initial value

TABLE 17

| | Mean Delta Inhibition | | | | | | |
|---|---|---|---|---|---|---|---|
| Study day | 4 | 7 | 10 | 14 | 17 | 21 | 24 |
| Control | n/a | n/a | n/a | n/a | n/a | n/a | n/a |
| Pembrolizumab | 44.33% | 44.20% | 30.78% | 20.92% | 10.08% | 8.62% | −11.94% |
| Pembrolizumab + | 29.57% | 79.32% | 85.83% | 87.28% | 86.27% | 78.55% | 97.36% |
| CTLA4.A5 | | | | | | | |
| Pembrolizumab + | −82.05% | −5.30% | 41.81% | 50.97% | 55.73% | 52.23% | 66.74% |
| CTLA4.A1 | | | | | | | |

The study showed that mice treated with pembro alone did not exhibit tumor growth inhibition at day 24, however the addition of the indicated CTLA4 antibodies increased the tumor growth inhibition over the course of the study.

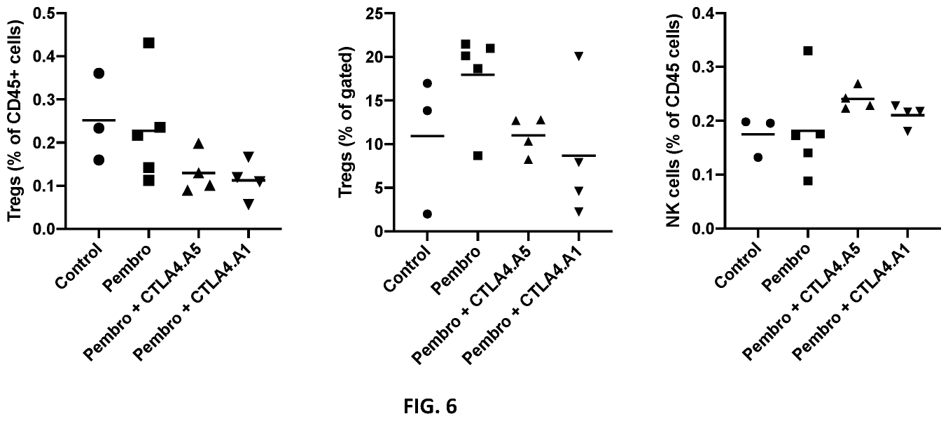

At the end of the experiment, select tumors were harvested and flow cytometry was conducted to investigate intratumoral immune cell populations. The data indicate that anti-CTLA4s decrease intratumoral Treg populations while increasing intratumoral NK cell populations (FIG. 6).

### 8.9. Example 9: Immune Related Adverse Events

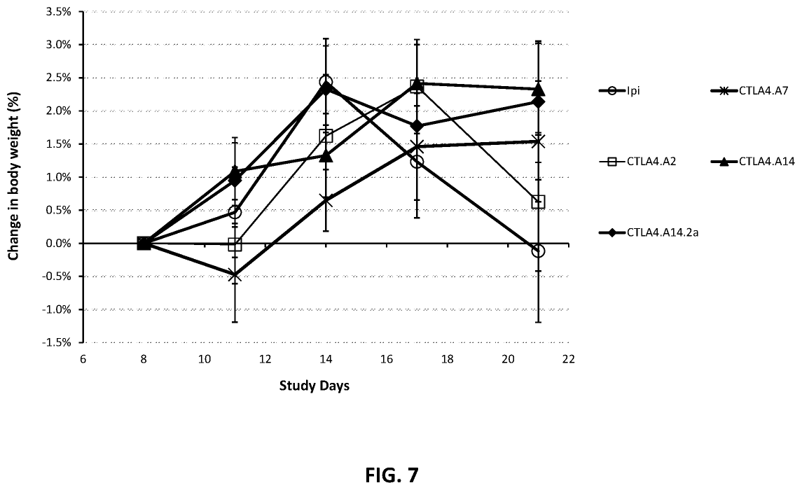

Transgenic mice expressing human CTLA-4 (hCTLA-4 KI mice) were implanted with MC38 tumor cells on the right flank. The hCTLA-4 KI mice were treated with one mg/kg of the indicated CTLA-4 antibody on days 8, 11, and 14 post-implantation. The mice were weighed on days 8, 11, 14, and 17 post-implantation. Animal counts were: n=8 for ipilimumab, n=9 for A7, n=8 for A2, n=9 for A14, and n=8 for A14.2. The percent changes in body weight of the mice receiving the indicated anti-CTLA4 treatments are shown in FIG. 7.

The mice treated with CTLA4. A7, CTLA4. A14, and CTLA4. A14.2a did not appear to exhibit weight loss after the final dose of anti-CTLA4 (FIG. 7). This finding was unexpected because immune-related Adverse Events (irAE) have been reported to be greater when anti-CTLA4s with enhanced ADCC (e.g., CTLA.A14.2a) are administered. This data suggest that anti-CTLA4s with reduced blocking activity may limit induction of irAEs even when ADCC is enhanced.

### 8.10. Example 10: Peripheral Flow Cytometry

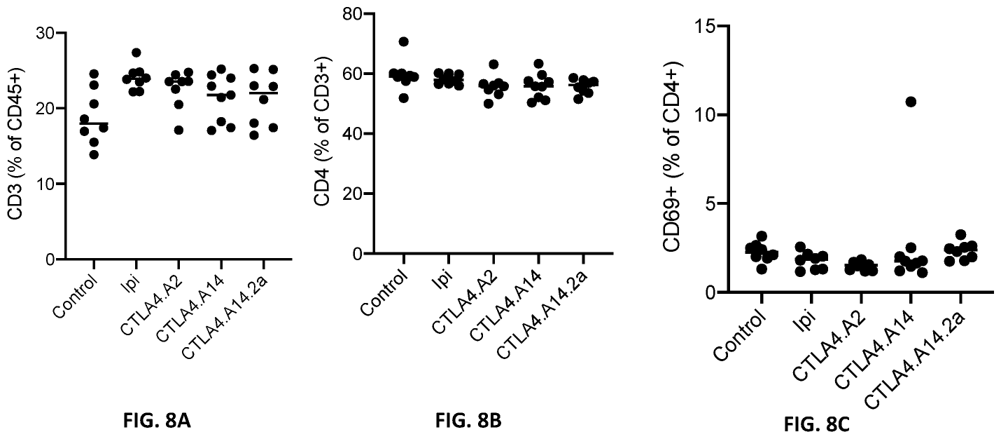

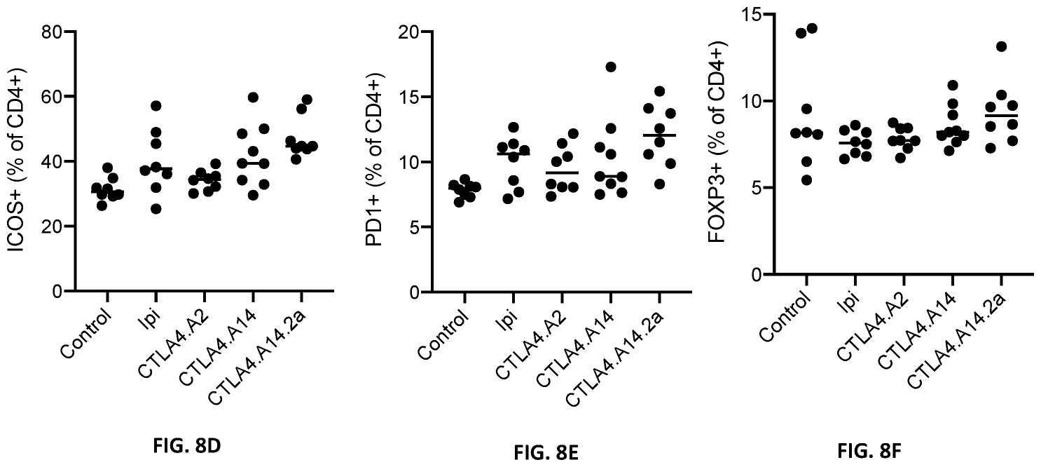

Transgenic mice expressing human CTLA-4 (hCTLA-4 KI mice) were implanted with MC38 tumor cells in the right flank. The hCTLA-4 KI mice were treated with one mg/kg of the indicated CTLA-4 antibody on days 8, 11, and 14 post-implantation. Peripheral flow cytometry was performed on day 27. 100 μL of blood was used for staining. The findings from the peripheral blood flow cytometry are shown in FIGS. 8A-8F, 9A-9D and 10.

The results provided in FIG. 8A indicated that CTLA4. A2 and CTLA4. A14 decrease the elevation of peripheral T cells (CD3+). Enhancing ADCC with CTLA4. A14.2a increased newly activated T cells (CD69+). CTLA4. A2 and CTLA4. A14 resulted in fewer non-conventional regulatory cells (CD4+PD1+, CD4+ICOS+). (See FIGS. 8D and 8E).

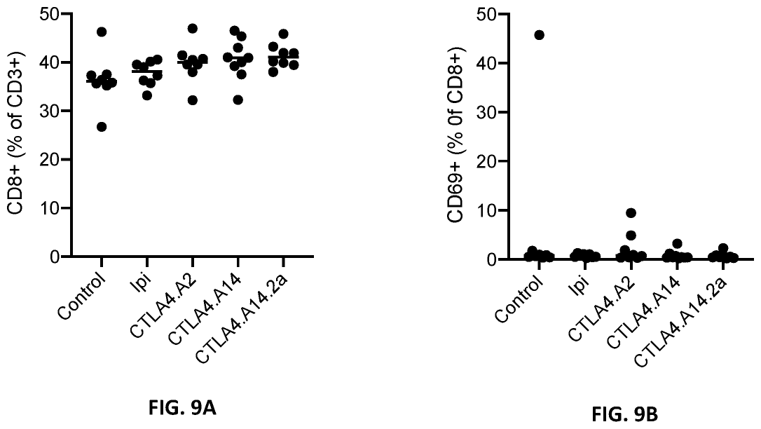

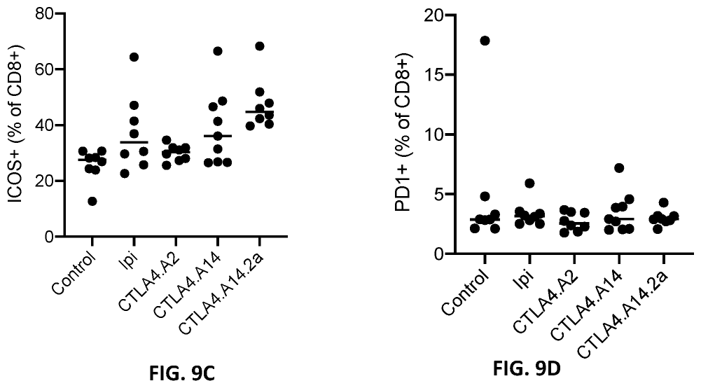

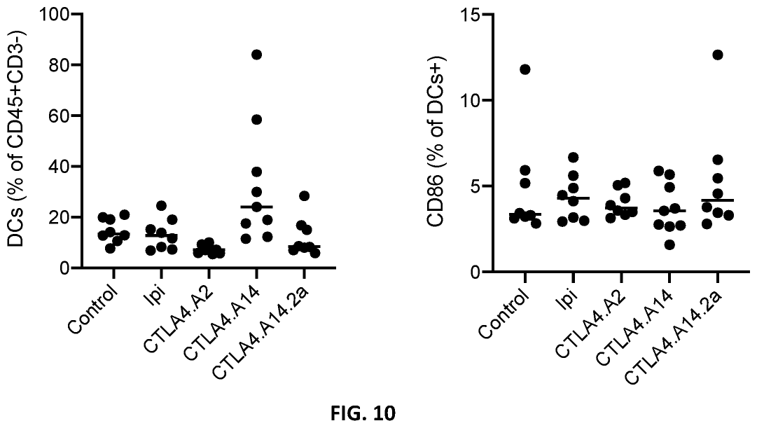

The results also indicated that CTLA4. A2 and CTLA4. A14 better enhance CD8+ T cells (FIG. 9A). CTLA4. A2 better enhanced newly activated T cells (CD8+CD69+) and led to decreased T cell exhaustion (CD8+PD1+) relative to Ipilimumab. ICOS has been described as a pharmacodynamic marker for anti-CTLA4. Enhancing ADCC with CTLA4. A14.2a appeared to further elevate CD8+ICOS+ cells (FIG. 9A-9D). The results also indicated that CTLA4. A2 and CTLA4. A14.2a lead to decreased peripheral immune activation relative to Ipilimumab, as judged by the frequency of dendritic cells (DCs) and activated DCs (CD86+). (see FIG. 10).

### 8.11. Example 11: Treatment with Low Dose CTLA-4 Study

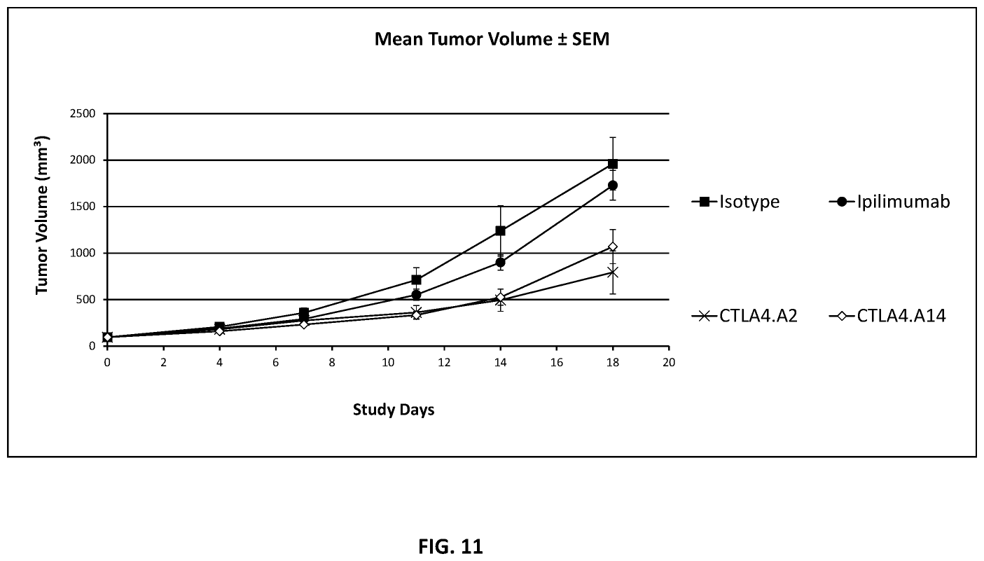

Transgenic mice expressing human CTLA-4 (hCTLA-4 KI mice) were implanted subcutaneously in the right flank region with MC38 tumor cells (1E6) in 0.1 ml of PBS for tumor development. The cells in exponential growth phase were harvested and quantitated by cell counter before tumor implantation. The hCTLA-4 KI mice were randomized when the mean tumor volume was 96.15 mm³ and treated with 0.3 mg/kg of the indicated anti-CTLA4, ipilimumab, or human IgG1 isotype control (Isotype) on days 0, 3 and 6 post-randomization. Tumor volume and mean % of inhibition was determined as described in Example 4. CTLA4. A2 and CTLA4. A14 resulted in significantly higher tumor inhibition over the 18 days of the study. The results of the study are shown in TABLE 18 and FIG. 11.

TABLE 18

| | Dates/Study Days | | | | | |
|---|---|---|---|---|---|---|
| | Dec. 6, 2019 | Dec. 10, 2019 | Dec. 13, 2019 | Dec. 17, 2019 | Dec. 20, 2019 | Dec. 24, 2019 |
| | Group | | | | | |
| Isotype | 0 | 4 | 7 | 11 | 14 | 18 |
| Ipilimumab | −0.17% | 9.33% | 18.68% | 22.37% | 27.23% | 11.84% |
| CTLA4.A2 | 0.22% | 13.00% | 23.03% | 49.09% | 60.03% | 59.54% |
| CTLA4.A14 | 0.01% | 23.39% | 35.06% | 53.05% | 57.50% | 45.41% |

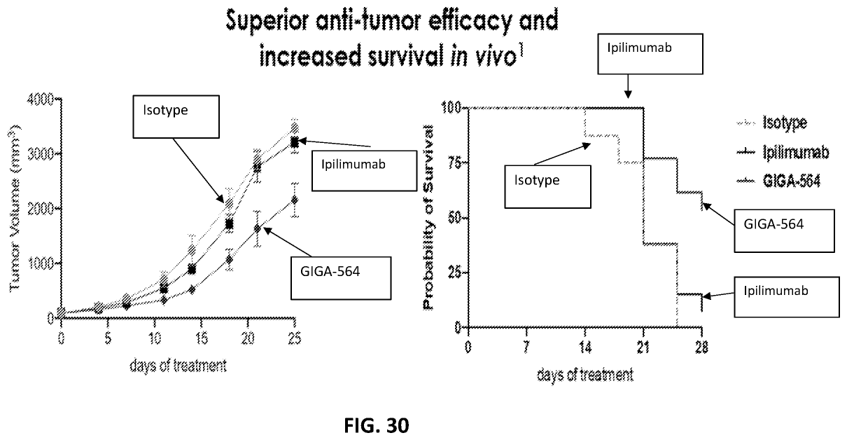

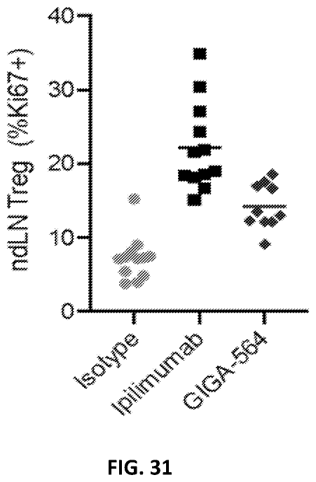

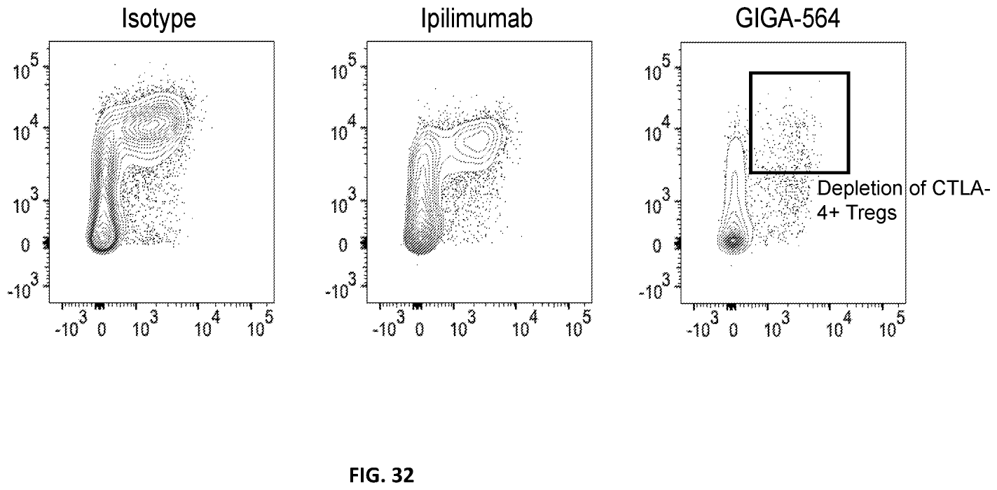

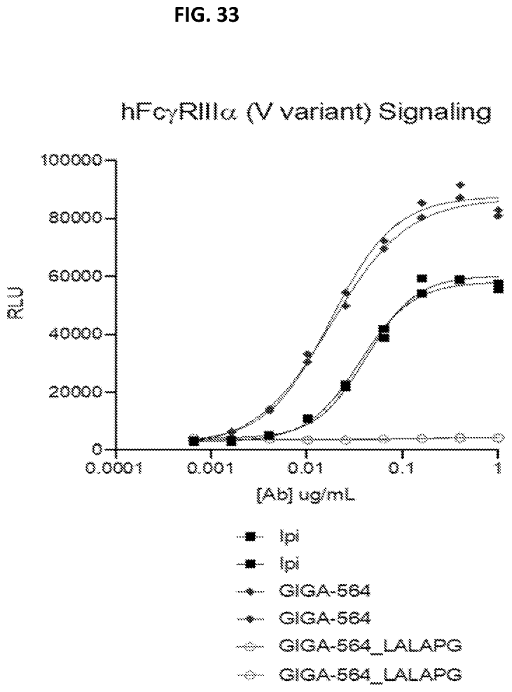

Data from this experiment is also shown in FIGs. 11, 14C and 30.

### 8.12

### 8.13. Example 12: Epitope Mapping of CTLA-4

Epitope mapping of commercially available anti CTLA-4 monoclonal antibody ipilimumab and anti CTLA-4 monoclonal antibody GIGA-564 (also described as clone A14 and CTLA4. A14 herein) was performed. The CDR sequences are described below in Table 21:

TABLE 21
CDR sequences of Ipilimumab and GIGA-564

| | Ipilimumab | | GIGA-564 | |
|---|---|---|---|---|
| | Heavy | Light | Heavy | Light |
| CDR1 | GFTFSSY (SEQ | QSVGSSYLA | GFTFSSY (SEQ | QSVSSSYLA |
| | ID NO: 12069) | (SEQ ID NO: | ID NO: 12075) | (SEQ ID NO: |
| | | 12072) | | 12078) |
| CDR2 | SYDGNN (SEQ | GAFSRAT (SEQ | WYEGRN (SEQ | GASSRAT (SEQ |
| | ID NO: 12070) | ID NO: 12073) | ID NO: 12076) | ID NO: 12079) |
| CDR3 | TGWLGPFDY | QQYGSSPWT | AGDLGAFDI | QQYGSSPWT |
| | (SEQ ID NO: | (SEQ ID NO: | (SEQ ID NO: | (SEQ ID NO: |
| | 12071) | 12074) | 12077) | 12080) |

Briefly, comprehensive alanine scanning mutagenesis was performed across the CTLA-4 protein. The mutant proteins were expressed in human cells, and binding by the two antibodies was determined, Protein expression and folding of the mutant proteins was validated and binding to wild-type CTLA-4 was used to normalize the data. Stringency was increased using, e.g., pH or salt modifications, to determine only the most important residues. The results are presented in Table 22.

TABLE 22
Critical residues for binding of
Ipilimumab and GIGA-564 to CTLA-4

| | Ipilimumab | GIGA-564 |
|---|---|---|
| CTLA-4 residues | R70, K130, Y139, | K130, Y139, L141, |
| | L041, I143 | I143 |

This data is also described in FIGS. 13C-D.

The results show that the antibodies share a common epitope. However, R70 on CTLA-4 is a critical residue for Ipilimumab but not for GIGA-564 binding. It is known that R70 is also involved in CD80 and CD86 binding to CTLA-4 (see, e.g., Li, Dong et al. “A functional antibody cross-reactive to both human and murine cytotoxic T-lymphocyte-associated protein 4 via binding to an N-glycosylation epitope.” mAbs vol. 12, 1 (2020): 1725365 and Udupi A. et al. “Structural basis for cancer immunotherapy by the first-in-class checkpoint inhibitor ipilimumab.” PNAS May 2017, 114 (21) E4223-E4232). It is also known that R70 is contacted by the heavy chain CDR2 in the crystal structure of Ipilimumab bound to CTLA-4; the heavy chain CDR2 sequences of the two antibodies are dissimilar.

In addition, the data show that L74A is a secondary residue for both, and impacts GIGA 564 binding more than Ipilimumab.

It is known that the shared epitope residues are contacted by the heavy chain CDR3 and light chain CDR3 in the crystal structure. The light chain CDR3 sequences of the two antibodies are identical.

The experiments were also performed with a Fab version of each antibody. The data show that E68 is also an important residue for GIGA-564 binding.

In conclusion, Ipilimumab and GIGA-564 have overlapping but distinct epitopes. R70 in critical for the binding of Ipilimumab but not GIGA-564. It is likely that R70 is available to associate with CD80/CD86 when GIGA-564 is bound. E68 and L74 are possible differentiators between monoclonal antibody Ipilimumab and monoclonal antibody GIGA-564.

### 8.14. Example 13: Mechanism of Action of GIGA-564 Compared to Ipilimumab in a Murine Model

The inventors of the present disclosure developed a novel CTLA-4 monoclonal antibody with minimal ability to block CTLA-4 binding to its CD80/CD86 ligands that has superior anti-tumor activity, and reduced toxicity compared to ipilimumab in murine models expressing human CTLA-4.

The study described herein assesses and characterizes the mechanism of action of GIGA-564 CTLA-4 antibody compared to ipilimumab in a murine model. Additionally, the anti-tumor effect of conventional anti-CTLA-4 mAbs, including ipilimumab, was investigated in the presence and absence of Fc effector functions.

### Materials and Methods

Some of the methods described previously are repeated below.

### Antibody Sequences

Amino acid sequences for the IgK and IgG variable regions for the 14 antibodies described here that were expressed as full-length antibodies and tested for in vitro blocking activity are listed in Table 26.

TABLE 26
CTLA-4 antibody sequences

| Antibody | IgK variable sequence | IgG variable region sequence |
|---|---|---|
| aCTLA-4.1 | EIVLTQSPGTLSLSPGERATLSCRASQSVSYLAW | QVQLVESGGGVVQPGRSLRLSCAASGFTFSRYG |
| | YQQKPGQAPRLLIYGASSRATGIPDRFSGSGSGT | MHWVRQAPGKGLEWVAVIWYDGRNKYYADS |
| | DFTLTISRLEPEDFAVYYCQQYGSSPWTFGQGT | VKGRFTISRDNSKNTLYLQMNSLRAEDTALYSC |
| | KVEIK | ARAGELGPFDYWGQGTLVTVSSA |
| aCTLA-4.2 | EIVLTQSPGTLSLSPGERATLSCRASQSVSSSYLA | QVQLVESGGGVVQPGRSLRLSCIASGFTFSSYG |
| | WYQQKPGQAPRLLIYGASSRATGIPDRFSGSGS | MHWVRQAPGKGLEWVAVNWYDGSNKHYADS |
| | GTDFTLTISRLEPEDFAVYYCQQYGSSPFTFGPG | VKGRFTISRDNSKNTLYLQMNSLRAEDTAVYY |
| | TKVDIK | CARGGVWGPYFDYWGQGTLVTVSSA |
| aCTLA-4.3 | EIVLTQSPGTLSLSPGERATLSCRASQSVSSSYLA | QVQLVESGGGVVQPGRSLRLSCAASGFTLSSFG |
| | WYQQKPGQAPRLLIYGASSRATGIPDRFSGSGS | MHWVRQAPGKGLEWVAVIWYDGSNKYYADS |
| | GTDFTLTISRLEPEDFAVYYCQQYGSSPFTFGPG | VKGRFIISRDNSKTALYLQMNSLRAEDTAVYYC |
| | TKVDIK | ARAHYFGAFDIWGQGTMVTVSSA |
| aCTLA-4.4 | EIVLTQSPGTLSLSPGERATLSCRASQSVSSSYLA | QVQLVESGGGVVQPGRSLRLSCAASGFTFSNYG |
| | WYQQKPGQAPRLLIYGASSRATGIPDRFSGSGS | MNWVRQAPGKGLEWVAVIWYDGRNKHYADS |
| | GTDFTLTISRLEPEDFAVYYCQQYGRSPFTFGPG | VKGRFTISRDNSKNTLYLQMNSLRAEDTAVYY |
| | TKVDIK | CARGGDWGPYFDYWGQGTLVTVSSA |
| aCTLA-4.9 | EIVLTQSPGTLSLSPGEGATLSCRASQSFSSNYLA | QVQLVESGGGVVQPGRSLRLSCAASGFTFSSFG |
| | WYQQKPGQAPRLLIYGASSRATGIPDRFSGSGS | MHWVRQAPGKGLEWVAVIWYDGRNKYYVDS |
| | GTDFTLTISRLEPEDFAVYYCQQYGTSPFTFGPG | VKGRFTISRDNSNNTLYLQMNSLRAEDTAVYY |
| | TKVDIK | CARGEFFGEFFDYWGQGTLVTVSSA |
| aCTLA-4.11 | EIVLTQSPGTLSLSPGERATLSCRASQSVSYLAW | QVQLVESGGGVVQPGRSLRLSCAASGFTFSSYG |
| | YQQKPGQAPRLLIYGASSRATGIPDRFSGSGSGT | MHWVRQAPGKGLEWVAVIWYDGSNKFYADSV |
| | DFTLTISRLEPEDFAVYYCQQYGSSPWTFGQGT | KGRFTISSDNSKNTLYLQMNSLRAEDTAVYYCA |
| | KVEIK | RGGHLGSFDYWGQGTLVTVSSA |
| aCTLA-4.12 | EIVLTQSPGTLSLSPGERATLSCRASQSVSSSYLA | QVQLVESGGGVVQPGRSLRLSCAASGFTFSSYG |
| | WYQQKPGQAPRLLIYGASSRATGIPDRFSGSGS | MHWVRQAPGKGLEWVAVIWYDGSNKYYADS |
| | GTDFTLTISRLEPEDFAVYYCQQYGTSPWTFGQ | VKGRFTFSRDNSKNTLYLQMNSLRAEDTAVYY |
| | GTKVEIK | CARGGLMGAFDYWGQGTLVTVSSA |
| aCTLA-4.14 | EIVLTQSPGTLSLSPGERATLSCRASQSVSSSYLA | QVQLVESGGGVVQPGRSLRLSCAASGFTFSSYG |
| | WYQQKPGQAPRLLIYGASSRATGIPDRFSGSGS | MHWVRQAPGKGLEWVAVIWYDGRNKDYADS |
| | GTDFTLTISRLEPEDFAVYYCQQYGSSPFTFGPG | VKGRITISRDNSKNTLYLQMNSLRAEDTAVYYC |
| | TKVDIK | ARGGLLGPYFDYWGQGTLVTVSSA |
| aCTLA-4.15 | EIVLTQSPGTLSLSPGERATLSCRASQSVSSSYLA | QVQLVESGGGVVQPGRSLRLSCAASGFTFSSYG |
| (GIGA-564) | WYQQKPGQAPRLLIYGASSRATGIPDRFSGSGS | MHWVRQAPGTGLEWVAVIWYEGRNKYYADP |
| | GTDFTLTISRLEPEDFAVYYCQQYGSSPWTSGQ | VKGRFTISRDNSKNTLYLQMNSLRDDDTAVYY |
| | GTKVEIK | CARAGDLGAFDIWGQGTMVTVSSA |
| aCTLA-4.22 | EIVLTQSPGTLSLSPGERATLSCRASQSVSSSYLA | QVQLVESGGGVVQPGRSLRLSCVASGFTLSSYG |
| | WYQQKPGQAPRLLIYGASSRATGIPDRFSGSGS | MHWVRQAPGKGLEWVAVIWYDGSNKHYADS |
| | GTDFTLTISRLEPEDFAVYYCQQYGSSPFTFGPG | VKGRFTISRDNSKNTLSLQMNSLRAEDTAVYYC |
| | TKVDIK | ARGGQLGPFDYWGQGTLVTVSSA |
| aCTLA-4.24 | DIQMTQSPSSLSASVGDRVTITCRASQSISSYLN | QVQLVESGGGVVQPGRSLRLSCAASGFTFSSHG |
| | WYQQKPGKAPKLLIYAASSLQSGVPSRFSGSGS | MHWVRQAPGKGLEWVAVIWYDGSNKHYADS |
| | GTDFTLTISSLQPEDFATYYCQQSYSTPFTFGPGT | VKGRFTISRDNSKNTLYLQMNSLRAEDTAVYY |
| | KVDIK | CARGDILTGYYGYWGQGTLVTVSSA |
| aCTLA-4.26 | DIQMTQSPSSLSASVGDRVTITCRASQSISSYLN | QVQLVESGGGVVQPGRSLRLSCAASGFTFSSYG |
| | WYQQKPGKAPKLLIYAASSLQSGVPSRFSGSGS | MHWVRQAPGKGLEWVAVIWYDGSYKYYADS |
| | GTDFTLTISSLQPEDFATYYCQQSYSTPFTFGPGT | VKGRFTISRDDSKNTLYLQMSSLRAEDTAVYYC |
| | KVDIK | ARAPHYAILTGYYEDYWGQGTLVTVSSA |
| aCTLA-4.28 | EIVLTQSPGTLSLSPGDRATLSCRASQSGSSSYLA | QVQLVESGGGVVQPGRSLRLSCAASGFTFSSYG |
| | WYQQKPGQAPRLLIYGASSRATGIPDRFSGSGS | MHWVRQAPGKGLEWVAVIWYDGNNKYYADS |
| | GTDFTLTISRLEPEDFAVYYCQQYGTSPFTSGPG | VKGRFTISRDNSKNTLYLQMYSLRAEDTAVYY |
| | TKVDIK | CARGGILAAGIFDYWGQGTLVTVSSA |
| aCTLA-4.29 | EIVLTQSPGTLSLSPGDRATLSCRASQSGSSSYLA | QVQLVESGGGVVQPGRSLRISCAASGFTFSSYGI |
| | WYQQKPGQAPRLLIYGASSRATGIPDRFSGSGS | HWVRQAPGKGLQWVAVIWYDGRNKYYADSV |
| | GTDFTLTISRLEPEDFAVYYCQQYGTSPFTSGPG | KGRFTISRDNSKNTLYLQMNSLRAEDTAVYYCS |
| | TKVDIK | RSGSFGAFDIWGQGTMVTVSSA |

### Generation of Full-Length Antibodies and Expression in CHO Cells

Five Trianni Mouse® mice expressing antibodies with fully-human variable regions were immunized at Antibody Solutions (Sunnyvale, Calif., USA), as described elsewhere (41). Briefly, mice were immunized with soluble His-tagged CTLA-4 (CT4-H5229; Acro Biosystems, Newark, Del., USA) and alendronate (ALD)/muramyl dipeptide (MDP) adjuvant twice per week for four weeks. Mice were euthanized and inguinal and popliteal lymph nodes and spleen were harvested and processed into a single cell suspension. Cells from all mice were pooled by tissue type and B cells were selected from lymph node and spleen using a mouse Pan-B negative selection kit (Stemcell Technologies, Vancouver, BC, Canada). Generation of natively paired heavy and light chain libraries, yeast surface display of scFvs and FACS sorting, and antibody repertoire analysis was described elsewhere.

From this analysis, scFv sequences were selected for full-length antibody expression based on enrichment after sorting. Expression constructs were Gibson assembled using GeneBlocks (Integrated DNA Technologies, Coralvile, Iowa, USA) and NEBuilder HiFi DNA Assembly Master Mix (NEB, Ipswich, Mass., USA) to integrate sequences into a vector appropriate for transient expression in CHO cells. The vector used was a variant of the pCDNA5/FRT mammalian expression vector (Thermo Fisher Scientific, Waltham, Mass., USA). The vector has an elongation factor 1 alpha (EF1α) promoter to express light chain followed by a bovine growth hormone (BGH) polyA sequence and a cytomegalovirus (CMV) promoter to express heavy chain followed by a second BGH polyA sequence. All constructs were synthesized as human IgG1 isotype, regardless of a given antibody's IgG isotype in the original repertoire. Constructs were transformed into NEB 10-beta E. coli for amplification and purified with the ZymoPURE Plasmid Maxiprep Kit (Zymo Research, Irvine, Calif., USA). The purified plasmid was then used for transient transfection in the ExpiCHO system (Thermo Fisher Scientific, Waltham, Mass., USA). Transfected cells were cultured for 7-9 days in ExpiCHO medium, and then antibodies were purified from filtered supernatant using Protein A columns (MilliporeSigma, St. Louis, Mo., USA). Antibody purity and proper size was verified by Coomassie stained sodium dodecyl sulphate-polyacrylamide gel electrophoresis (SDS-PAGE) (Thermo Fisher Scientific, Waltham, Mass., USA).

### Transient Transfection and mAb Protein Production

For larger-scale expression, the full-length kappa chain of GIGA-564 (A14; “aCTLA-4.15”) was cloned into a separate pSF expression vector (MilliporeSigma, St. Louis, Mo., USA) with a CMV promoter. The immunoglobulin kappa-only and dual-gene (immunoglobulin kappa and immunoglobulin gamma) plasmids were transfected at a 2:1 molar ratio into ExpiCHO cells using ExpiFectamine (Thermo Fisher Scientific, Waltham, Mass., USA). Briefly, for every 100 mL of culture 100 μg of total plasmid was mixed with 320 μl of ExpiFectamine in 4 mL of OptiPro serum free media (Thermo Fisher Scientific, Waltham, Mass., USA), incubated at room temperature for 5 minutes, then added to cells freshly passaged to 6×10⁶ cells/mL. Cells were fed on day 1 and day 5 after transfection with 16 mL of ExpiCHO feed, then harvested on Day 8 or when the viability dropped below 75%.

Harvested cell-culture fluid (HCCF) was loaded onto a HiTrap MabSelect Prism A column (Cytiva, Marlborough, Mass., USA), equilibrated with PBS pH 7.0-7.4 using a fast protein liquid chromatography (FPLC) instrument (AKTA pure 25, Cytiva, Marlborough, Mass., USA), and eluted with 100 mM citrate pH 3. Fractions were pooled to collect >90% of eluted material and neutralized to pH 6.2 using 1 M tris pH 9. Neutralized eluate was dialyzed into 40 mM histidine+240 mM sucrose pH 6.2 and formulated with 0.2% tween-20. Concentration was determined by absorbance (NanoDrop 8000, Thermo Fisher Scientific, Waltham, Mass., USA) and endotoxin quantified by limulus amebocyte lysate assay (NexGen PTS, Charles River, Wilmington, Mass., USA). Routine biophysical characterization included size exclusion chromatography high performance liquid chromatography (SEC-HPLC; 7.8×300 mm, 2.7 μM, 300A column, Agilent, Santa Clara, Calif., USA), SDS-PAGE (12% tris-glycine, Thermo Fisher Scientific, Waltham, Mass., USA), and capillary electrophoresis sodium dodecyl sulphate (CE-SDS; protein 230, BioAnalyzer 2100, Agilent, Santa Clara, Calif., USA).

### Clonal Cluster Analysis and Visualization

USEARCH was used to compute the total amino acid differences between each pairwise alignment of FACS-sorted scFv sequences. The R package igraph (version 1.2.6) was then used to generate a clustering plot for the pairwise alignments. The sequences were represented as “nodes”, while “edges” were the links between nodes. Edges indicate pairwise alignments with ≤9 amino acid differences. The layout_with_graphopt (charge=0.03, niter=1000) option was used to format the output.

### Affinity Measurements

Kinetic analysis of CTLA-4 mAb HCCF from CHO expression was performed on an MX-96 instrument (IBIS Technologies, Enschede, Netherlands) by Carterra (Dublin, Calif., USA). A medium density capture chip was created with anti-Human IgG Fc (SouthernBiotech, Birmingham, Ala., USA) with a surface density of 925-1200 RU. A CFM printer (IBIS Technologies, Enschede, Netherlands) printed one 10 minute print of mAb HCCF for each sample. His-tagged human CTLA-4 antigen (CT4-H5229; Acro Biosystems, Newark, Del., USA) injections for kinetic analysis were 5 minutes and dissociations were 10 minutes. Kinetic analysis was performed with five 5-fold serial titrations starting at 500 nM antigen and fitted to a 1:1 monovalent model.

Binding/dissociation experiments to determine the affinity of mAb HCCF from CHO expression to His-tagged cynomolgus CTLA-4 (CT4-C5227; Acro Biosystems, Newark, Del., USA) were performed at 30° C. on an Octet Red96 instrument (ForteBio, Fremont, Calif.) by a CRO (Bionova Scientific, Fremont, Calif., USA). Antibodies were loaded at 5 μg/mL onto Protein A biosensors that were dipped into 80 nM antigen, and kinetic constants were calculated using a monovalent model.

Flow Cytometry for In Vitro Characterization of mAbs

To determine if the CTLA-4 mAbs bound the appropriate antigen expressed on the surface of mammalian cells, CHO cell lines stably expressing human CTLA-4 or an irrelevant antigen (CD27) were generated. Cells (0.5×10⁶ each cell line) were combined and washed with MACS buffer (Dulbecco's Phosphate Buffered Saline [DPBS], with 0.5% bovine serum albumin [BSA], and 2 mM ethylenediaminetetraacetic acid [EDTA]). The cells were incubated with 10 μg/mL anti-CTLA-4 for 30 min at 4° C., then excess, unbound mAbs were removed by washing twice with MACS buffer. The cells were then stained with PE-conjugated anti-Human IgG Fc antibody (clone M1310G05, BioLegend 410720, San Diego, Calif., USA) to detect bound anti-CTLA-4 and with fluorescein isothiocyanate (FITC)-conjugated anti-CD27 (clone 0323, BioLegend 302806, San Diego, Calif., USA) to distinguish the cells expressing the irrelevant antigen. Cells were washed twice with MACS buffer, fixed with 4% paraformaldehyde fixation buffer (BioLegend 420801, San Diego, Calif., USA) for 20 minutes at room temperature and washed twice more with MACS buffer. For validation of the N297Q variants of the ipilimumab analog and aCTLA-4.28, a similar procedure was followed except CHO cells lacking expression of human CTLA-4 were used in place of the CD27-expressing cells, the two cell lines were stained separately, FITC-conjugated anti-Human IgG Fc antibody (clone M1310G05, BioLegend 410719, San Diego, Calif., USA) was used to detect bound anti-CTLA-4, cells were not treated with fixation buffer, and cells were instead stained with 4′,6-diamidino-2-phenylindole (DAPI; BioLegend, San Diego, Calif., USA) and gated for live (DAPI⁻) cells on the cytometer. Binding of mAbs to CTLA-4 on the cell surface was determined using LSR II (BD Biosciences, San Jose, Calif., USA) or CytoFLEX LX (Beckman Coulter, Brea. Calif., USA) flow cytometers and analyzed using FlowJo (v10.6.1, BD Biosciences, San Jose, Calif., USA).

To determine accumulation of anti-CTLA-4s on the cell surface, suspension CHO cells expressing wildtype human CTLA-4 were plated at 1×10⁶ cells/well in DPBS (Lonza, Basel, Switzerland) containing 0.5% BSA (MilliporeSigma, St. Louis, Mo., USA) and 0.5 M EDTA (MilliporeSigma, St. Louis, Mo., USA). Titrations of ipilimumab or GIGA-564 from 0-50 μg/mL were added to the cells and the plate was incubated at 37° C. for 30 minutes to allow for internalization to occur. Anti-human IgG Fc allophycocyanin (APC) (BioLegend, San Diego, Calif., USA) was added as a secondary antibody at 10 μg/mL and the plate was incubated at 4° C. for 30 minutes. Following viability staining with DAPI (BioLegend, San Diego. Calif., USA), data was acquired on the CytoFLEX LX (Beckman Coulter, Brea, Calif., USA) and analyzed using FlowJo (v10.6.1, BD Biosciences, San Jose, Calif., USA).

### Cell-Based CTLA-4 Blocking Assay

CTLA-4 Blockade Bioassay was purchased from Promega (JA3001; Madison, Wis., USA) and performed according to the manufacturer's recommendations. Briefly, a serial dilution of each antibody was generated in assay buffer (90% RPMI 1640/10% FBS, supplied in the kit) in a sterile 96-well plate. CTLA-4 effector cells (provided with kit) were thawed and diluted into 3.2 mL of assay buffer and 25 μL of the cell suspension was added to each of the inner 60 wells of a 96-well, white flat-bottom plates. 25 μL of the appropriate antibody dilutions were added to the wells containing CTLA-4 effector cells. Artificial antigen presenting (aAPC)/Raji cells (provided with kit) were thawed and diluted into 7.2 mL of assay buffer, and 25 μL of the cell suspension was added to wells containing the diluted antibodies and CTLA-4 effector cells. Plates were incubated for 6 hours at 37° C. in a tissue culture incubator with 5% CO₂ before 75 μL of Bio-Glo Reagent was added to wells containing cell and antibody mixtures. Plates were incubated for 5-15 minutes at room temperature and luminescence was measured on a Spectramax i3x plate reader (Molecular Devices, San Jose. Calif., USA). Data analysis was performed using the Softmax Pro (Molecular Devices, San Jose, Calif., USA) or Prism (GraphPad, San Diego, Calif., USA) software packages.

### CD80/CD86 Blocking ELISAs

ELISA plates (Nunc, MaxiSorp ELISA plates, flat bottom, uncoated, BioLegend, San Diego, Calif., USA) were coated with recombinant human CTLA-4-Fc (7268-CT, R&D Systems, Minneapolis, Minn., USA) at 1 μg/mL overnight at 4° C. Plates were then blocked with 5% milk in phosphate buffered saline with tween 20 (PBST) for 1 hour at room temperature. A titration series of the indicated mAbs was then added to the plates and then the plates were incubated for 1 hour at room temperature to allow mAb binding. Excess, unbound mAb was removed by washing with PBST. Recombinant human His-tagged CD80 (R&D Systems 9050-B1, Minneapolis, Minn., USA) or CD86 (R&D Systems 9090-B2, Minneapolis, Minn., USA) was then added to the plates at 1 μg/mL. After incubation for 1 hour at room temperature, the plates were washed to remove unbound ligand. Bound ligand was detected with a horseradish peroxidase (HRP)-conjugated anti-His antibody (652504, BioLegend, San Diego, Calif., USA). After incubation for 1 hour at room temperature and washing, the plates were developed with 3,3′,5,5′-Tetramethylbenzidine (TMB) substrate (34028, Pierce, Waltham, Mass., USA). After sufficient signal was achieved, development was stopped by the addition of 1 N hydrochloric acid. Absorbance at 450 nm was read using a Spectramax i3x plate reader (Molecular Devices. San Jose, Calif., USA). Half maximal inhibitory concentration (IC50) values were calculated by plotting absorbance versus the log of concentration using Prism (GraphPad, San Diego, Calif., USA).

### Murine Models

Murine experiments were done in compliance with all relevant ethical regulations and approved by the Institutional Animal Care and Use Committee of Crown Bioscience. Experiments using full-length hCTLA-4 knock-in HuGEMM™ mice (Shanghai Model Organisms Center, Inc.) were performed at Crown Bioscience (Taicang Jiangsu Province, China). Full-length hCTLA-4 knock-in HuGEMM™ mice were generated by knocking in human CTLA4 cDNA with a polyA sequence at exon 1 of the murine Ctla4 locus, replacing murine Ctla4 expression with human CTLA4 expression. Crown Bioscience acquired MC38 cells from FDCC (The Institutes of Biomedical Sciences (IBS), Fudan University, China) and RM-1 cells from SIBS (Shanghai Institutes for Biological Sciences, China) and authenticated cell line identity of research cell banks by single nucleotide polymorphism (SNP) analysis. Cell lines were mycoplasma negative. 8-12-week-old, female, full-length hCTLA-4 knock-in HuGEMM™ mice were injected subcutaneously at the right lower flank with MC38 or RM-1 cells (10⁶ cells suspended in 100 μL of PBS) as indicated for tumor development. Tumors were allowed to establish until tumor volume reached the indicated size. Mice were then randomized and dosing, as described for each experiment, was initiated on the same day as randomization. Tumor volumes and body weight were measured in a blinded fashion at least twice a week. Tumor volumes were calculated using the formula: V=(L×W×W)/2, where V is tumor volume, L is tumor length (the longest tumor dimension), and W is tumor width (the longest tumor dimension perpendicular to L). Individual animals were removed from the study as their tumor volumes measured greater than 3000 mm³. Where indicated mice with a complete response on day 35 post randomization were re-challenged on day 43. For the re-challenge experiment, MC38 tumor cells (10⁶ in 100 μL PBS) were implanted subcutaneously in the lower left (opposite) flank region. Tumor cells were also implanted in 6-8 week old naïve, WT C57BL/6 mice (Shanghai Lingchang Biotechnology Co., Ltd. (Shanghai, China)) as a positive control for tumor growth at the time of the re-challenge. Mice and any tumor progression were observed for another 30 days. Several of these experiments are previously described in Examples 4, 5, 6, 7, and 11.

Single cell suspensions from tumors were prepared using the murine tumor dissociation kit (Miltenyi, Bergisch Gladbach, Germany) and GentleMACS™ Octo Dissociator with Heaters set to the dissociation program (37_c_m_TDK_1). Single cell suspensions from lymph nodes were pushed through a 70 μm cell strainer using the plunger from a 5 mL syringe. Single cell suspensions were then incubated at room temperature with 1×RBC lysis buffer for 90 seconds. RBC lysis was then quenched, washed, strained through a 70 μm cell strainer, and counted. For cell staining cells were first blocked with 1 μg/ml Fc-Block (mouse Fc Block, BD Biosciences, San Jose, Calif., USA) for 15 minutes at 4° C. Cells were then stained with the indicated surface antibodies for 30 minutes on ice in the dark (Table 27). Cells were then washed twice and fixed with Fixation/Permeabilization working solution (eBioscience, San Diego, Calif., USA) and then washed twice with 1× Permeabilization buffer (eBioscience, San Diego, Calif., USA), after which intracellular staining was performed in 1× Permeabilization buffer. Following intracellular staining, cells were washed twice with 1× Permeabilization buffer and data was collected using a flow cytometer (LSRFortessa X-20, BD Biosciences, San Jose, Calif., USA). Data were analyzed using Kaluza or FlowJo 10.

TABLE 27
Reagent antibodies for immunophenotyping.

| Marker | Fluorochrome | Clone | Catalog | Vender |
|---|---|---|---|---|
| CD107a | FITC | 1D4B | 121606 | BioLegend |
| CD11c | FITC | N418 | 117306 | BioLegend |
| CD335 | BV605 | 29A1.4 | 137619 | BioLegend |
| (NKp46) | | | | |
| CD4 | BUV395 | GK1.5 | 563790 | BD |
| CD4 | BV421 | GK1.5 | 100438 | BioLegend |
| CD44 | BV421 | IM7 | 563970 | BD |
| CD45 | BV785 | 30-F11 | 103149 | BioLegend |
| CD62L | BV785 | MEL-14 | 104440 | BioLegend |
| CD69 | PE-Cy7 | H1.2F3 | 104512 | BioLegend |
| CD8 | PE-eFluor610 | 53-6.7 | 61-0081-82 | eBioscience |
| CD80 | APC | 16-10A1 | 560016 | BD |
| CD86 | BV605 | GL-1 | 105037 | BioLegend |
| FoxP3* | PE | FJK-16s | 12-5773-82 | eBioscience |
| hCD152 | Percp-cy5.5 | L3D10 | 349928 | BioLegend |
| (CTLA-4) | | | | |
| hCD152 | Alexa Fluor ® | L3D10 | 349920 | BioLegend |
| (CTLA-4)* | 647 | | | |
| Ki67* | Alexa Fluor ® | SolA15 | 56-5698-82 | eBioscience |
| | 700 | | | |
| Live/Dead | Ef780 | n/a | 65-0865-18 | eBioscience |
| Live/Dead | APC-eFluor | n/a | 65-0865-14 | eBioscience |
| | 780 | | | |
| TCR-beta | BUV395 | H57-597 | 742485 | BD |
| Counting beads | — | — | 01-1234-42 | Invitrogen |

*Used for internal staining

Experiments using hCTLA-4/hPD-1 double knock-in HuGEMM™ mice on the BALB/c background (GemPharmatech Co., Ltd) were generated by crossing mice in which exon 2 of murine Ctla4 was replaced with exon 2 from human CTLA4 with mice in which exon 2 and 3 of murine Pdcd1 were replaced with exon 2 and 3 of human PDCD1. The murine toxicity study using these mice was performed at Crown Bioscience (Taicang Jiangsu Province, China). Female mice were 4-5 weeks old at start of dosing and were treated with PBS, pembrolizumab (15 mg/kg), ipilimumab (10 mg/kg) plus pembrolizumab, or GIGA-564 (10 mg/kg) plus pembrolizumab every 3 days for 9 doses, and mice were euthanized 10 days after completion of dosing and tissues collected for histopathology analysis.

### Epitope Mapping

Identification of the key energetic residues involved in binding was performed by Integral Molecular (Philadelphia, Pa., USA). Full-length human CTLA-4 with a mutation to reduce internalization (Y201G) was cloned into a transient expression vector, with each extracellular position (36-161) individually mutated to alanine (or alanine mutated to serine). The mutant library was arrayed in 384-well microplates and transiently transfected into HEK-293T cells. The optimal staining concentrations of ipilimumab, GIGA-564, and L3D10 control (BioLegend, San Diego, Calif., USA) were determined using wild-type CTLA-4, then applied to the CTLA-4 mutant library. Antibody binding was detected using goat-anti-human IgG-Alexa fluor-488 or anti-human IgG F(ab′)₂-Alexa fluor-488 (Jackson ImmunoResearch, West Grove, Pa., USA), and mean cellular fluorescence was determined using flow cytometry (Intellicyt iQue, Ann Arbor, Mich., USA). Mutated residues were considered critical if mutation resulted in significant loss of binding to the test antibody compared to the control antibody. This epitope mapping experiment is also described in Example 12.

### In Vitro Treg Activation Assay

Anti-CD3 (OKT3, Ultra-LEAF purified, BioLegend, San Diego, Calif., USA) with and without CD80 (R&D Systems rhCD80-Fc, 10107-B1-100, Minneapolis, Minn., USA) were covalently linked to the surface of M-450 Tosyl beads (Dynabead M-450, 140130, Thermo Fisher Scientific, Waltham, Mass., USA) according to the manufacturer's guidelines. To confirm that the surfaces of each bead type were properly coated, fluorescent antibodies were used to detect either the mouse Fc (anti-CD3) or the CD80 on the bead surface via flow cytometry.

Donor-matched human Treg and Tconv cells (Stemcell Technologies, 70096, Vancouver, BC, Canada) were thawed and stained with CellTrace Violet (C34571, Thermo Fisher Scientific, Waltham, Mass., USA) according to the manufacturer's protocol. 10⁵ cells were then cultured with 10⁵ beads as indicated in Complete Iscove's Modified Dulbecco's Medium (IMDM, Thermo Fisher Scientific, Waltham, Mass., USA) with non-essential amino acids, sodium pyruvate, Glutamax, and 5% human antibody serum at 37° C., 5% CO₂ in a 96-well U-bottom plate (Falcon-Corning U-bottom tissue culture plates, VWR, Radnor, Pa., USA). In conditions where rhCD80-Fc (Abatacept, BMS, Princeton, N.J., USA) or aCTLA-4.28 were used, these were mixed with beads first prior to being added to the cells. Test samples with both rhCD80-Fc and aCTLA-4.28 were mixed and incubated together prior to mixing with beads and then cells. After a 4 day incubation, cells were washed, stained, and analyzed using the CytoFLEX LX (BD Biosciences, San Jose, Calif., USA). Flow cytometry data were analyzed in FlowJo v10.7.1 (BD Biosciences, San Jose, Calif., USA).

### FcR Effector Activity Bioassays

Fc effector activity bioassays for mFcγRIIIa (CS1779B08), mFcγRIV (M1151), hFcγRIIb (CS1781E02), hFcγRIIa-H variant (G9981), hFcγRIIa-R variant (CS1781B08), hFcγRIIIa-V variant (G7011), and hFcγRIIIa-F variant (G9791) were purchased from Promega Corporation (Madison, Wis., USA). The assays were carried out following the manufacturer's instructions. Briefly, CHO target cells stably expressing human CTLA-4 with the Y201G mutation to enhance cell surface expression were suspended in RPMI 1640+4% FBS media with either GIGA-564, ipilimumab, or GIGA-564 with the LALA-PG mutation to eliminate Fc function, and incubated at 37° C. for 30 minutes. The following starting concentrations and dilution factors were used for each assay: 1 μg/mL, 2.5-fold dilution series (mFcγRIIIa, hFcγRIIIa-V variant), 500 μg/mL, 2.5-fold (mFcγRIV), 3 gg/mL, 3-fold (hFcγRIIb, hFcγRIIa-H and R variant), or 10 μg/mL, 2.5-fold (hFcγRIIIa-F variant). Jurkat/NFAT-Luc effector cells solely expressing one type of FcγR were added to each well (effector:target ratio was 5:1) and incubated at 37° C. for 6 hours. Luciferase activity was measured by using the included Bio-Glo Luciferase Assay Reagent with the SpectraMax i3x or iD3 plate readers (Molecular Devices, San Jose, Calif., USA). Luciferase activity measured in relative luminescence units (RLU) were plotted against the concentration of CTLA-4 mAbs. The IC50 value of each mAb was calculated by logistic regression using Prism (GraphPad, San Diego, Calif., USA).

Determination of pH-Sensitivity of Antibodies

96-well plates (Nunc, MaxiSorp, flat bottom, uncoated. BioLegend, San Diego, Calif., USA. 423501) were coated with 0.5 μg/mL rhCTLA-4-Fc chimera (7268-CT, R&D Systems, Minneapolis, Minn., USA) diluted in 1×PBS pH 7.0 and incubated at 2-8° C. overnight. Plates were washed with 1×PBST and blocked with PBST+1% BSA (PBSTB). Purified CTLA-4 mAbs were diluted in 10 mM sodium phosphate+150 mM NaCl adjusted to pH 4.0, 5.0, 6.0, or 7.0 then added to the plate for 1 hour. Unbound antibodies were washed away with PBST and bound antibodies detected with 0.5 μg/mL HRP-conjugated goat anti-human constant kappa (2060-05, Southern Biotech, Birmingham, Ala., USA) diluted in PBSTB. Plate was washed with PBST once more, then TMB substrate (1-Step™ Ultra TMB-ELISA Substrate Solution, Thermo Fisher Scientific, Waltham, Mass., USA) was added and developed for approximately 1 minute before stopping the reaction with 1 N HCl. Absorbance at 450 nm was measured using a spectrophotometer (i3x, Molecular Devices, San Jose, Calif., USA) and plotted using Prism (GraphPad, San Diego, Calif., USA).

### Generation of Cell Pools Stably Expressing GIGA-564

GIGA-564 was expressed from a variant of the pCDNA5/FRT mammalian expression vector (Thermo Fisher Scientific; Waltham, Mass., USA). The vector has a promoter for glutamine synthetase selection and uses an EF1a promoter to drive light chain expression, followed by a BGH polyA sequence and a CMV promoter to drive expression of the heavy chain, followed by a second BGH polyA sequence. The antibody expression construct was built using GeneBlocks (Integrated DNA Technologies, Coralville, Iowa, USA) and NEBuilder® HiFi DNA Assembly Master Mix (New England BioLabs, Ipswich, Mass., USA). The construct was amplified in NEB 10-beta E. coli and purified with the ZymoPURE™ Plasmid Maxiprep Kit (Zymo Research, Irvine, Calif., USA). It was then linearized for transfection by digesting 150 μg of DNA with 3,000 units PvuI-HF restriction enzyme (New England Biolabs, Ipswich, Mass., USA), precipitated, and washed.

Sigma Aldrich's CHOZN cells were cultured in EX-CELL CD CHO Fusion medium (MilliporeSigma, Burlington, Mass., USA) supplemented with 6 mM GlutaMAX (Gibco, Waltham, Mass., USA), shaking at 37° C., 5% CO₂, 125 RPM (25 mm throw) for 7 days prior to electroporation. The day before electroporation, suspension cells were seeded at 0.5×10⁶ viable cells/mL (vc/mL). Linearized plasmid was transfected and pulsed using CM-150 on the Amaxa 4D Nucleofector (Lonza, Basel, Switzerland) and SE Cell Line 4D Nucleofector X kit (Lonza, Basel, Switzerland). Each cuvette had 4 μg of DNA and 10⁷ CHOZN cells concentrated in SE cell solution. Post-electroporation, the transfected cells were transferred to T-75 flasks at a 2×10⁶ vc/mL density for a 1-day recovery in EX-CELL CD CHO Fusion medium (MilliporeSigma, Burlington, Mass., USA) with 6 mM GlutaMAX (Gibco; Waltham, Mass., USA). After 1-day recovery, transfected cells were pelleted and resuspended into 80% EX-CELL CD CHO Cloning medium (MilliporeSigma, Burlington, Mass., USA) and 20% EX-CELL CD CHO Fusion medium (MilliporeSigma, Burlington, Mass., USA) and plated into 96-well plates (non-TC treated, flat bottom, Greiner One-Bio, Kremsmünster, Austria) at 5,000 cell per well and incubated 37° C., 5% CO₂ with humidity.

After 4-5 weeks, confluent transfected wells (minipools) were screened for antibody concentration using the Gator bio-layer interferometry system and Protein-A biosensors (GatorBio, Palo Alto, Calif., USA). Minipools with the highest antibody titer were scaled-up using EX-CELL CD CHO Fusion medium in static 24-well TC-treated plates incubated at 37° C., 5% CO₂ with humidity. Minipools continued to expand and shaker adapted to shaking 6-well plates (non-TC treated, flat bottom, Greiner One-Bio, Kremsmünster, Austria) on a 19 mm shaking platform set at 145 RPM, 37° C., 5% CO₂, with humidity. Material from an 8-day terminal batch in 24-well plates (TC treated, flat bottom, Corning, Corning, NY, USA) was screened using the Gator system as a second method to screen minipools, in order to identify the high producers. Cultures were expanded into shaking 50 mL conical tubes (Midsci, St. Louis, Mo., USA) incubated at 37° C., 5% CO₂, 80% humidity, shaking at 225 RPM with 25 mm throw. Top minipools were combined to generate enriched pools (EPs).

EPs were cultured in shake flasks at 125 RPM, 25 mm throw, seeded at 0.2-0.4×10⁶ viable cell density (VCD)/mL for 3-4 days. EPs were then inoculated in EX-CELL Advanced Fed Batch medium at 0.4×10⁶ VCD/mL in 1 L shake flasks (Corning, Corning, NY, USA) with a vent cap and working volume of 200 mL. Incubator conditions were held constant throughout fed-batch at 37° C., 5% CO₂, 80% humidity, and shaking at 125 RPM (25 mm throw). Beginning day 3 of fed-batch to harvest, growth, viability, and metabolite concentrations were measured offline. Glucose was supplemented when concentration dropped below 4 g/L, up to 6 g/L using 45% glucose stock (Corning, Corning, NY, USA). Cultures were fed days 3, 5, 7, 9, and 11 with EX-CELL Advanced CHO Feed 1 (MilliporeSigma, Burlington, Mass., USA) (4% of working culture volume) and CellVento 4Feed COMP (MilliporeSigma, Burlington, Mass., USA) (2% of working culture volume). Cultures were harvested when viability dropped below 80% and cell culture supernatant was clarified by centrifugation and filtered. Protein was purified using Protein A eluted with 100 mM acetate pH 3. One EP was used for subsequent analysis (EP-1).

### Glycan Analysis

This analysis was performed by the CRO Bionova Scientific (Fremont, Calif., USA). 150 μg of antibody was digested with PNGase F (Prozyme. Hayward, Calif., USA) to liberate glycans. Glycans were isolated and labeled with a fluorescent molecule using a GlykoPrep InstantAB labeling kit (Prozyme, Hayward, Calif., USA) following the manufacturer's instructions. Labeled glycans were injected over a 3.5 μm, 2.1×150 mm XBridge Amide column (Waters, Milford, Mass., USA) on a UPLC (Dionex Ultimate 3000, Waters, Milford, Mass., USA) with a fluorescent detector. Peaks were identified based on known glycans from the Glyko InstantAB biantennary and high-mannose partitioned library standards (Prozyme, Hayward, Calif., USA), with the total percent of each glycoform reported as the integrated area under the identified peak divided by the sum of the AUC (area under the curve) for all peaks.

### Skin Inflammation, Colonic Epithelial and Heart Toxicity Scoring

Hematoxylin and eosin (H&E)-stained sections of skin from near the whisker region was scored as follows 1: 1-3 small foci of lymphocyte aggregates per section, 2: 4-10 small foci or 1-3 intermediate foci, 3: 4 or more intermediate or the presence of large foci, 4: marked interstitial fibrosis in parenchyma and large foci of lymphocyte aggregates.

The colonic epithelial score was determined by assessment of H&E-stained colon rolls. To determine this score, after reviewing the whole slide the four regions in which damage was most severe were selected for scoring, and the score from all four areas was added together to determine the cumulative score. To determine the score for individual areas, both the epithelial structures affected and the consistency of the damage were taken into account. For this the damage to epithelial structures was scored as 0: no lesions, 1: mucosa damage, 2: submucosa damage, 3: muscularis/serosa damage. The consistency of damage in each area was scored as 1: focal, 2: patchy, 3: diffuse. Both scores were then multiplied together to determine the area score.

For the heart pathology score, lymphocyte infiltration on H&E-stained heart sections in pericardium, right or left atrium, base of aorta, and left or right ventricle each count for 1 point each. The number of CD45⁺ cells were counted on heart formalin-fixed paraffin-embedded (FFPE) sections stained with CD45. The visual fields for inflammatory cell infiltration analysis were randomly selected, and the average score of three fields for each sample was determined.

### Kidney Pathology Staining and Scoring

Tissues were collected and were placed in 10% buffered neutral formalin and then paraffin embedded. Sectioning, staining, and scoring was performed by Allele (San Diego, Calif., USA). Briefly, blocks were cut in 5 μm sections that were placed on glass slides for anti-IgG (UltraPolymer Goat anti-Mouse heavy and light chain IgG-HRP, Cell IDx, San Diego, Calif., USA) or anti-C3 (EPR19394, Abcam, Cambridge, UK) immunohistochemistry (IHC) staining. Stained slides were prepared as digital images.

A board-certified veterinary pathologist with experience in laboratory animals and toxicologic pathology, who was blinded to the study, evaluated the anti-IgG slides for location, intensity, and percent of positive staining. Findings were scored on a scale of 1 to 4 for intensity (0: negative, 1: minimal or slightly positive, 4: very dark), and as a percent of the positive cells in the glomeruli (after reviewing at least 5 glomeruli). This experiment is also described in Example 6.

### Statistical Analysis

Comparison of changes in tumor volume measured longitudinally in multiple groups was determined using a linear mixed effects model with treatment group and day as fixed effects and animal identifier as a random effect, to account for the dependence of repeated measures. All tests were two-sided without adjustments to type I error rates. These analyses were conducted using R version 3.6.2.

For the re-challenge study, tumor volume (in mm³) was analyzed using a linear mixed effects model including treatment group and day as fixed effects and animal identifier as a random effect, to account for repeated measures. Statistical comparisons were made using the Wald test against the isotype control group for the initial challenge and against the naïve control group for the re-challenge portion of the study. All tests were two-sided with an alpha level of 0.05.

Adjusted p-values for comparison of skin inflammation, colonic epithelial damage, CD45⁺ cell infiltration into the heart, heart pathology score, colon length, spleen weight, and kidney Ig or C3 deposition were calculated using the Benjamini-Hochberg step-down procedure to account for multiple comparisons. Analyses were performed using R version 3.6.2. To determine if there was any statistical difference in the percent body weight induced by pembrolizumab or pembrolizumab plus a CTLA-4 mAb in comparison to control, a mixed effect model on change in body weight including day and treatment group as fixed effects and animal identifier as a random effect, to account for repeated measurements within animal, was used.

For flow cytometry data, the Wilcoxon rank sum test was used to determine statistical significance, with an alpha level of 0.05.

### Results

This study presented evidence that checkpoint inhibition was not a primary mechanism of action for efficacy of anti-CTLA-4 antibodies. Instead, the primary mechanism for efficacy is FcR-mediated Treg depletion in the tumor microenvironment. The study identified a monoclonal antibody (mAb) (GIGA-564) that binds to CTLA-4 at an epitope that differs from ipilimumab's by only a few amino acids, yet has limited checkpoint inhibitor activity. Surprisingly, the weak checkpoint inhibitor had superior anti-tumor activity compared to ipilimumab in a murine model. The weak checkpoint inhibitor also induced less Treg proliferation and has increased ability to induce in vitro FcR signaling and in vivo depletion of intratumoral Tregs. Further experiments showed that the enhanced FcR activity of the weak checkpoint inhibitor likely contributes to its enhanced anti-tumor activity. The results of the present study showed that weak checkpoint inhibition was associated with lower toxicity in murine models.

The results herein showed that compared to ipilimumab, the weak B7 ligand blocking anti-CTLA-4 antibody GIGA-564 induced less peripheral Treg proliferation, more efficient intratumoral Treg depletion, superior anti-tumor efficacy, and less toxicity in human CTLA4 knock-in mouse models. This work suggests a translational path forward to improve outcomes for cancer patients.

The results of the study showed that the efficacy of anti-CTLA-4 drugs is due to depletion of Tregs in the tumor microenvironment. Checkpoint inhibition by anti-CTLA-4 is not necessary and may cause toxicities.

The inventors surprisingly found that GIGA-564 is:
(1) an anti-CTLA-4 with limited checkpoint inhibitor activity, GIGA-564 has superior efficacy to ipilimumab in mouse models;
(2) induced less Treg proliferation than ipilimumab in tumor bearing mice;
(3) had superior Treg depletion in the tumor microenvironment and more efficiently depleted intratumoral CTLA-4^{Hi};
(4) improved cell signaling via interactions between CTLA-4-bound GIGA-564 and FcR; and
(5) induced less toxicity than ipilimumab in murine models expressing human CTLA-4.

### In Vitro Characterization of a Diverse Panel of Anti-CTLA-4 Antibodies

A library of natively paired single chain variable fragments (scFvs) specific to CTLA-4 were generated by immunizing Trianni Mouse® animals, which produce antibodies with fully-human variable domains and mouse constant regions; then used a proprietary microfluidics platform and yeast scFv display to select for CTLA-4 binders. Fourteen of the antibodies were cloned as full-length human IgG1 antibodies, produced in Chinese hamster ovary (CHO) cells, and were shown to bind to recombinant human CTLA-4 by surface plasmon resonance (SPR) with low nanomolar affinity (average equilibrium dissociation constant, KD: 8.7 nM), similar to that of ipilimumab (FIG. 20A-20B and Table 23).

TABLE 23
In vitro characterization of scFvs reformatted as full-length antibodies.

| | | koff | KD | MFI of | MFI of | | Cell-based | Cell-based | Cell- |
|---|---|---|---|---|---|---|---|---|---|
| | kon human | human | human | CTLA-4 + | CD27 + | KD cyno | blocking | blocking | based |
| | CTLA-4 | CTLA-4 | CTLA-4 | CHO | CHO | CTLA-4 | activity EC50 | activity max | assay |
| Antibody | (M−1 s−1) | (s−1) | (nM) | cells | cells | (nM) | (μg/mL) | signal (RLU) | plate |
| Ipilimumab | 6.50E+04 | 3.20E−04 | 4.9 | 860 | 26.6 | 1.3 | 0.34 | 307 | A |
| analog | | | | | | | | | |
| aCTLA-4.1 | 8.50E+04 | 3.90E−04 | 4.6 | 1008 | 18.6 | 1.0 | 0.10 | 349 | A |
| aCTLA-4.2 | 1.90E+05 | 2.80E−04 | 1.5 | 1050 | 18.2 | 1.2 | 0.18 | 273 | C |
| aCTLA-4.3 | 6.40E+04 | 3.60E−04 | 5.7 | 854 | 15.3 | 0.8 | 0.17 | 63 | C |
| aCTLA-4.4 | 1.00E+05 | 5.20E−04 | 5.1 | 1123 | 11.7 | 3.1 | 0.12 | 413 | A |
| aCTLA-4.9 | 2.10E+05 | 1.60E−04 | 0.77 | 1328 | 18.5 | 1.0 | 0.13 | 237 | C |
| aCTLA-4.11 | 4.20E+03 | 2.00E−04 | 48 | 219 | 14.8 | no | 0.89 | 47 | D |
| | | | | | | binding | | | |
| aCTLA-4.12 | 5.10E+04 | 2.70E−04 | 5.3 | 1013 | 17.1 | 4.5 | 2.17 | 13 | D |
| aCTLA-4.14 | 1.30E+05 | 4.20E−04 | 3.2 | 1024 | 17 | 9.6 | 0.09 | 73 | A |
| aCTLA-4.15 | 5.40E+04 | 5.20E−04 | 9.8 | 717 | 15.9 | 23.6 | 0.09 | 33 | A |
| (GIGA-564) | | | | | | | | | |
| aCTLA-4.22 | 2.30E+04 | 5.10E−04 | 22 | 641 | 15.2 | 3.1 | 0.26 | 134 | A |
| aCTLA-4.24 | 6.30E+04 | 4.30E−04 | 6.8 | 934 | 26 | 0.9 | 0.19 | 333 | B |
| aCTLA-4.26 | 6.10E+04 | 3.50E−04 | 5.7 | 939 | 17.8 | 1.6 | 0.18 | 338 | B |
| aCTLA-4.28 | 9.00E+04 | 4.20E−04 | 4.6 | 1033 | 17.2 | 2.2 | 0.18 | 548 | B |
| aCTLA-4.29 | 7.60E+04 | 2.30E−04 | 3 | 999 | 16.3 | 1.2 | 0.15 | 444 | B |

All 14 mAbs bound CHO cells stably expressing human CTLA-4, with no off-target binding to CHO cells expressing the irrelevant target CD27 (FIG. 20C and Table 23). Next, the ability of these mAbs to block interaction of CTLA-4 with its endogenous ligands (the B7 proteins CD80 and CD86) was assessed by a cell-based assay. The mAbs exhibited a variety of half maximal effective concentration (EC50, μg/mL; average: 0.34; range: 0.1-2.17), and a broad range of maximum signals (average: 240; range: 13-548), where lower values indicated less blocking (FIG. 20D and Table 23). The correlations between affinity KD and blocking EC50 or between affinity KD and blocking maximum signal were not significant (linear regression; R²=0.06 and R²=0.16, respectively; p>0.05, FIG. 20E). For this set of 14 antibodies, it was speculated that B7 ligand blocking is a function of anti-CTLA-4 binding epitope and not CTLA-4 binding affinity. Of interest, aCTLA-4.15, subsequently re-named GIGA-564, showed a combination of high affinity and low blocking maximum signal.

### FcR Effector Function is Required for the Anti-Tumor Efficacy of Ipilimumab in a Murine Model

The role of Treg depletion in the efficacy of CTLA-4 mAbs has remained controversial. Additionally, the role of the Fc effector function in the anti-tumor efficacy of ipilimumab has not been reported. Thus, the anti-tumor effect of conventional anti-CTLA-4 mAbs, including ipilimumab, was investigated in the presence and absence of Fc effector functions.

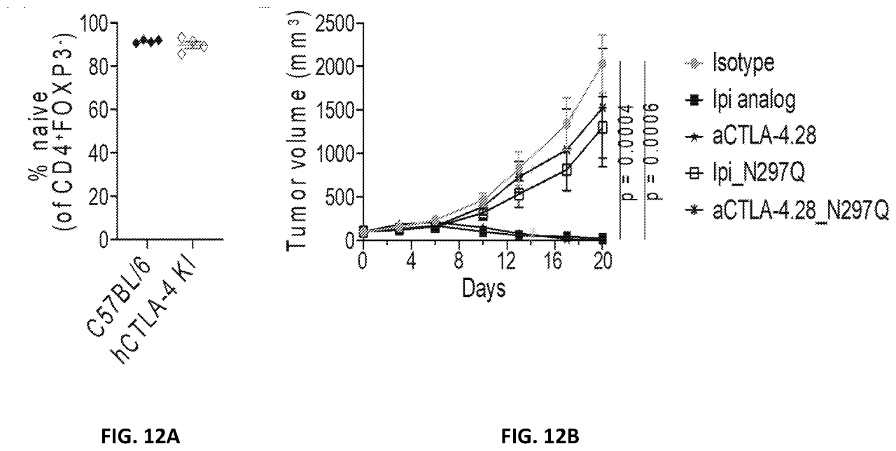

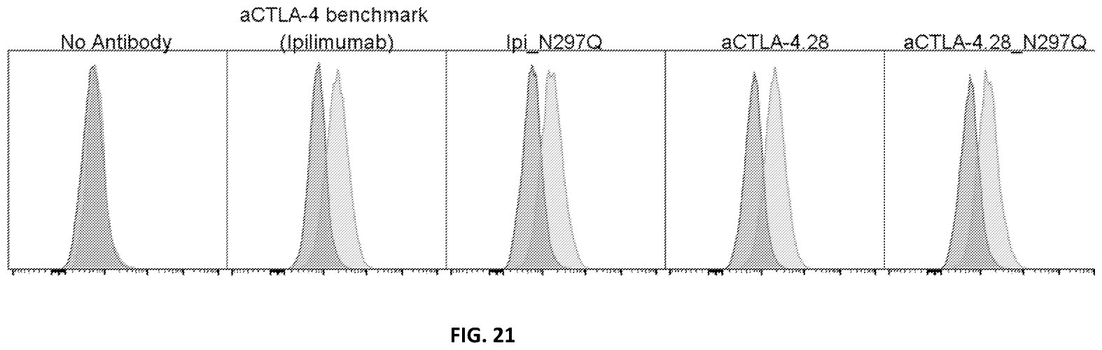

First, wild-type human IgG1 and human IgG1 N297Q mutant Fc variants of ipilimumab and another strong blocker, aCTLA-4.28 (FIG. 20 and Table 23) were generated. The N297Q mutation eliminates Fc glycosylation and the ability to bind FcRs, thus abrogating Fc effector functions such as ADCC. The CTLA-4 mAbs with the N297Q mutation were still able to bind CTLA-4⁺ CHO cells (FIG. 21). As ipilimumab does not bind murine CTLA-4, a human CTLA4 knock-in (hCTLA-4 KI) mouse model, where the human CTLA4 coding region is knocked-in to the murine Ctla4 locus, was used for in vivo efficacy studies. The majority (89.9% and 91.5%, respectively) of CD4 conventional T cells (CD4⁺FOXP3⁻) in the lymph node of hCTLA-4 KI and wildtype mice were naïve (CD44^{Lo}CD62L⁺) (FIG. 12A), suggesting that CTLA-4 is functional in hCTLA-4 KI mice. Both the ipilimumab analog and aCTLA-4.28 with a wildtype human IgG1 Fc led to strong regression of MC38 tumors (colon cancer model) in hCTLA-4 KI mice (FIG. 12B). However, in mice treated with ipilimumab_N297Q or aCTLA-4.28_N297, tumor growth was not significantly different from isotype control treated mice (FIG. 12B). These data indicated that anti-CTLA-4 mAbs, including ipilimumab, require FcR binding for anti-tumor activity and that blocking the binding of B7 ligands to CTLA-4 is not sufficient for the anti-tumor activity of anti-CTLA-4 mAbs.

GIGA-564 has Weak Ability to Block CTLA-4 from Binding to CD80/CD86

To confirm the weaker blocking ability of GIGA-564, ELISAs were performed to separately determine the ability of CD80 or CD86 to bind CTLA-4 in the presence of CTLA-4 mAbs (FIG. 13A). If binding of the mAb to CTLA-4 blocked CTLA-4 from binding to the ligand, the amount of ligand detected would decrease with increasing mAb concentration. GIGA-564 had much weaker blocking efficiency for both CD80 and CD86 than ipilimumab or aCTLA-4.28, as the curves were strongly shifted to the right (FIG. 13B and Table 24). Additionally, GIGA-564 was unable to fully block either ligand, even at the highest concentrations (FIG. 13B). This is consistent with its inability to induce full CD28 signaling in the cell-based signaling assay (FIG. 20D), which required the mAbs to strongly block binding of CTLA-4 to CD80 and CD86.

TABLE 24
GIGA-564 has minimal ability to block
CD80 or CD86 from binding CTLA-4

| | CD80 | | | CD86 | | |
|---|---|---|---|---|---|---|
| | | | IC50 | | | IC50 |
| Antibody | Max | Min | (μg/mL) | Max | Min | (μg/mL) |
| Ipilimumab | 0.96 | 0.04 | 0.07 | 1.04 | 0.09 | 0.07 |
| GIGA-564 | 0.97 | 0.18 | 2.23 | 1.07 | 0.36 | 0.30 |
| aCTLA-4.28 | 1.09 | 0.04 | 0.12 | 1.05 | 0.12 | 0.12 |

Max: maximum absorbance at 450 nm, normalized to prembrolizumab
Min: minimum absorbance at 450 nm, normalized to pembrolizumab

The key amino acids in the CTLA-4 epitope for GIGA-564 and ipilimumab were determined using the shotgun mutagenesis epitope mapping method. Epitope mapping revealed that GIGA-564 and ipilimumab have overlapping but distinct epitopes. In particular K130, Y139, L141, and I143 are key amino acids that are part of the epitope for both GIGA-564 and ipilimumab (FIG. 13C-13D). However, R70 is a key amino acid for the epitope of ipilimumab but not GIGA-564 (FIG. 13C-13D). Importantly, R70 is also important for CD80 and CD86 binding to CTLA-4 (FIG. 13D). It was speculated that R70 on CTLA-4 may be more available to bind CD80 and CD86 when CTLA-4 is bound by GIGA-564 compared to ipilimumab, explaining why GIGA-564 only minimally blocks CD80/CD86 binding to CTLA-4.

### GIGA-564 Treatment Induces Anti-Tumor Responses in Murine Models

The anti-tumor efficacy of GIGA-564 was tested. Both GIGA-564 and commercial ipilimumab led to nearly complete control of MC38 tumors in hCTLA-4 KI mice when they were dosed at 5 mg/kg twice a week (FIG. 14A). Similarly, in hCTLA-4 KI mice bearing RM-1 tumors (prostate cancer model), which are more resistant to anti-CTLA-4, GIGA-564 led to similar tumor growth inhibition to commercial ipilimumab when dosed at 5 mg/kg on days 0, 3, and 6 (FIG. 14B). Thus, it was found that anti-CTLA-4 anti-tumor activity is independent of checkpoint inhibition via CTLA-4 blocking in multiple tumor models.

Highly efficacious dosing of anti-CTLA-4 mAbs at 5 mg/kg made it difficult to identify any potential difference in the anti-tumor efficacy of ipilimumab and GIGA-564. Therefore, the ability of 0.3 mg/kg of ipilimumab or GIGA-564 to control progression of MC38 tumors in hCTLA-4 KI mice was assessed. GIGA-564 was more effective in limiting tumor progression than commercial ipilimumab at this low dose (FIG. 14C).

### GIGA-564 Induces Less Peripheral Treg Proliferation and Efficiently Depletes Intratumoral Tregs

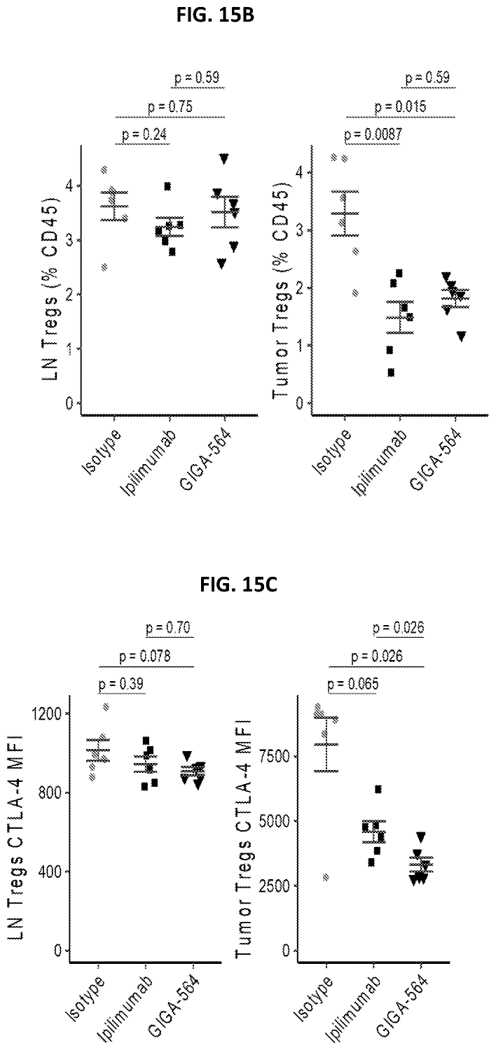

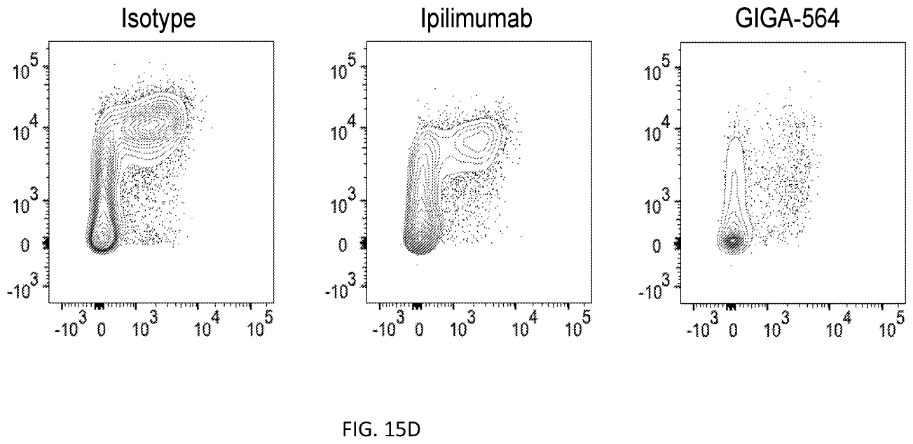

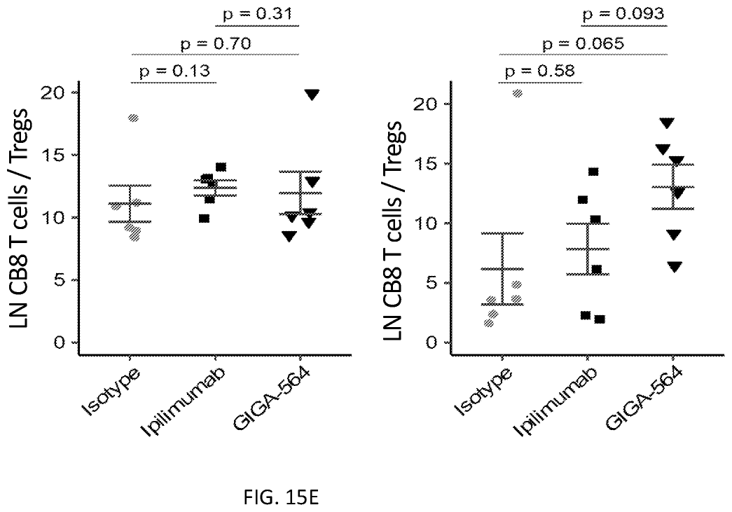

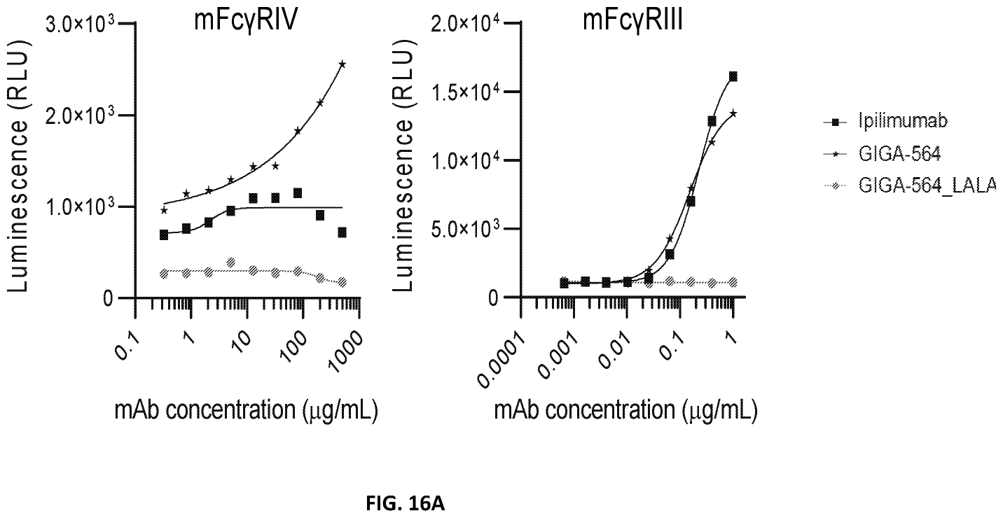

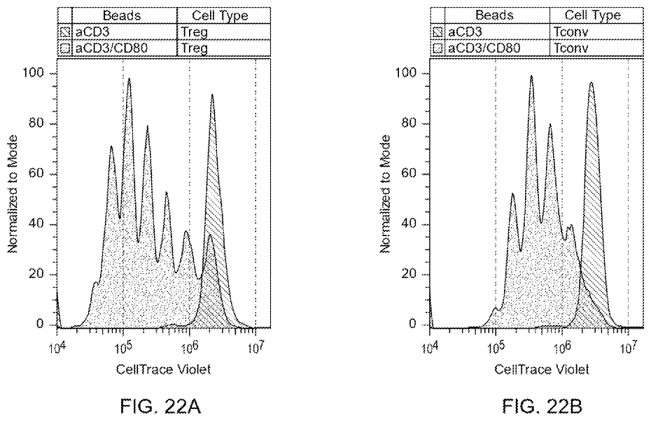

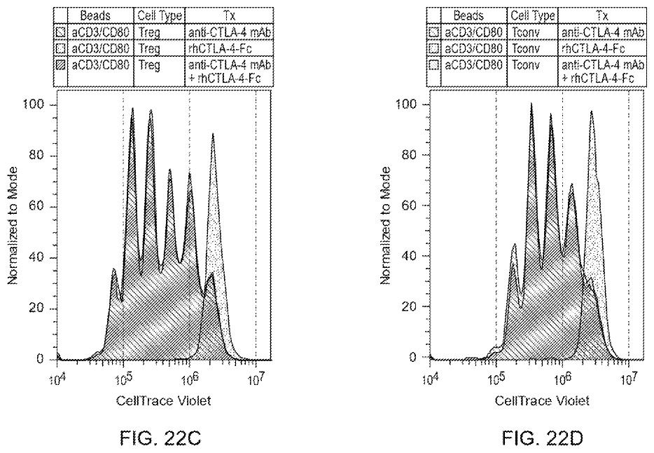

Tregs stimulated via CD3/CD80 proliferate more than Tregs stimulated via CD3 alone, and CTLA-4 blockade overcomes inhibition by exogenous CTLA-4-Fc (FIG. 22). In agreement with this, blocking or acute loss of CTLA-4 enhances Treg proliferation in vivo. Thus, it was hypothesized that GIGA-564 would induce less Treg proliferation than ipilimumab. To test this possibility, hCTLA-4 KI mice bearing established MC38 tumors were treated with mAbs at 5 mg/kg on days 0, 3, and 6 and analyzed the proliferation status of peripheral T cell subsets from a non-draining lymph node on day 7 (FIG. 15A). This analysis revealed that ipilimumab increased the proliferation of Tregs more than it increased the proliferation of conventional T cells (Tconv) or CD8 T cells (FIG. 15A). Furthermore, GIGA-564 induced significantly less peripheral Treg proliferation than ipilimumab (FIG. 15A; Wilcoxon rank sum test, p=<0.05). These data are consistent with the in vitro data that showed that GIGA-564 only weakly blocks the interaction of CTLA-4 with its B7 ligands (FIG. 13B, FIG. 20D, and Table 23).

Intratumoral Tregs express more surface CTLA-4 than effector T cells or even peripheral Tregs. Thus, as FcR effector function activity of antibodies is dependent on the amount of antibody that binds to the cell surface, CTLA-4 mAbs selectively deplete intratumoral Tregs. Furthermore, antibody-mediated depletion of CTLA-4^{Hi} Tregs in the tumor microenvironment (TME) has been shown to begin within the first 24 hours after antibody administration. Thus, to determine the ability of GIGA-564 to deplete intratumoral Tregs hCTLA-4 KI mice bearing established MC38 tumors were treated with 5 mg/kg of isotype, ipilimumab, or GIGA-564 and then analyzed Treg populations in the periphery and tumor by flow cytometry 1 day later. It was found that both anti-CTLA-4 antibodies depleted intratumoral Tregs (Wilcoxon rank sum test, p<0.05) but not peripheral Tregs (FIG. 15B; Wilcoxon rank sum test, p>0.05). It was also found that for animals administered GIGA-564, the population of remaining intratumoral Tregs had a lower CTLA-4 mean fluorescence intensity (MFI) than analogous populations from animals administered ipilimumab (FIG. 15C-15D; Wilcoxon rank sum test, p<0.05). Additionally, GIGA-564 weakly enhanced the intratumoral CD8/Treg ratio compared to ipilimumab (FIG. 15E). GIGA-564 was more effective at depleting CTLA-4^{Hi} intratumoral Tregs, which in addition to the reduced Treg proliferation induced by GIGA-564, may explain why GIGA-564 is superior at controlling tumor growth at low doses than ipilimumab. The results show that GIGA-564 eliminates intratumoral Tregs. Human CTLA-4 knock-in mice bearing MC38 tumors were treated with 5 mpk of ipilimumab or GIGA-564 and sacrified 1 day later. Flow cytometry showed that GIGA-564 is more efficient than ipilumab at depleting CTLA-4+ Tregs in the tumor microenvironment.

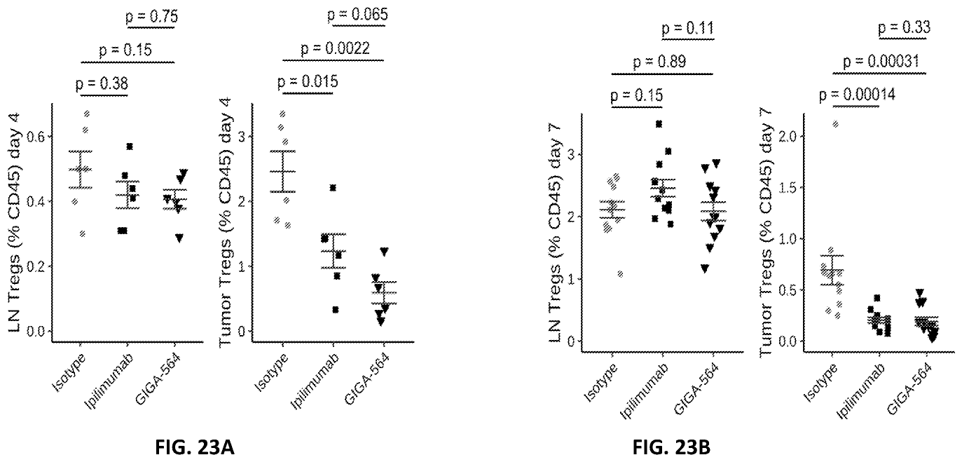

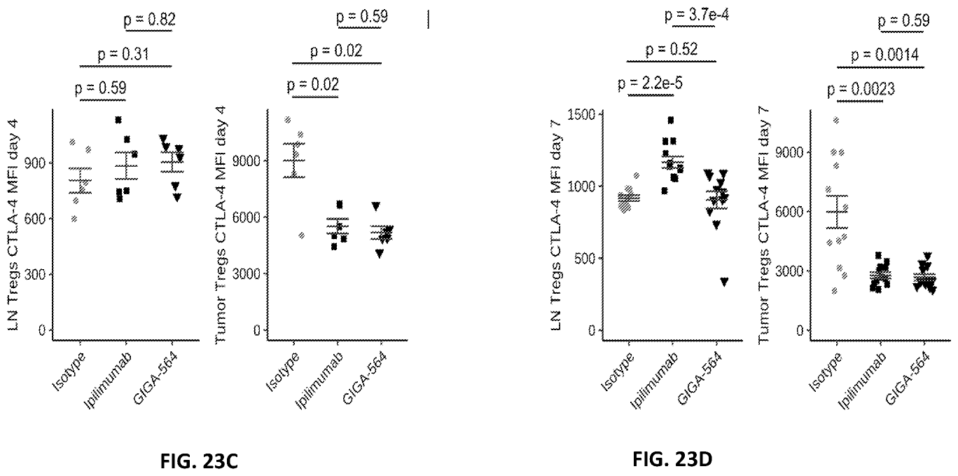

Additional flow analysis of the TME or peripheral lymph node of hCTLA-4 KI mice with established MC38 tumors treated with 2 or 3 doses of GIGA-564 or ipilimumab was performed (harvesting on day 4 or 7, respectively). Even with additional treatments, both GIGA-564 and ipilimumab were able to specifically deplete intratumoral Tregs but not peripheral Tregs (FIG. 23).

GIGA-564 Induces More FcR Signaling than Ipilimumab

The increased efficacy of low dose GIGA-564 compared to ipilimumab (FIG. 14C) may be in part due to the enhanced ability by which GIGA-564 depletes CTLA-4^{Hi} intratumoral Tregs (FIG. 15C-15E). To investigate this, the ability of GIGA-564 and ipilimumab to induce mouse FcR signal transduction were compared. It was found that GIGA-564 resulted in more FcR signaling than ipilimumab in Jurkat effector cells expressing murine FcγRIV, but not FcγRIII, when co-cultured with CHO cells stably expressing CTLA-4 (FIG. 16A and Table 25). This increased FcγRIV signaling induced by GIGA-564 may explain why GIGA-564 more efficiently depletes intratumoral Tregs than ipilimumab.

TABLE 25
Fc receptor signaling of GIGA-564 and ipilimumab

| | Maximum luminescence (RLU) | | | EC50 (μg/mL) | |
|---|---|---|---|---|---|
| | | GIGA- | GIGA- | | GIGA- |
| FcR assay | Ipilimumab | 564 | 564_LALAPG | Ipilimumab | 564 |
| mFcyRIV | 990 | 2,557 | 297 | 2.21 | not determined* |
| mFcYRIII | 17,496 | 14,311 | 1,163 | 0.22 | 0.15 |
| hFcyRIIIa-V | 59,205 | 87,184 | 4,255 | 0.04 | 0.02 |
| hFcyRIIIa-F | 6,880 | 15,818 | 2,434 | 0.09 | 0.04 |
| hFcyRIIa-H | 973 | 802 | 210 | 0.05 | 0.04 |
| hFcyRIIa-R | 2,641 | 2,560 | 1,572 | 0.21 | 0.08 |
| hFcyRIIb | 5,801 | 5,173 | 978 | 0.17 | 0.11 |

*GIGA-564 EC50 not determined due to poor curve fit

As the increased murine FcγRIV signaling induced by GIGA-564 may contribute to the enhanced depletion of intratumoral Tregs and efficacy induced by GIGA-564 in hCTLA-4 KI MC38 tumor-bearing mice, it was important to determine if GIGA-564 also induces more human FcR signal transduction. It was found that GIGA-564 induced more human FcγRIIIa signaling than ipilimumab (FIG. 16B and Table 25). However, the increased FcR signaling induced by GIGA-564 was FcR-specific (FIG. 16C-16D and Table 25). From a translational perspective, GIGA-564 may have increased ability to induce FcR signaling and thus FcR effector functions in patients, especially if expression of FcγRIIIa is used as a biomarker.

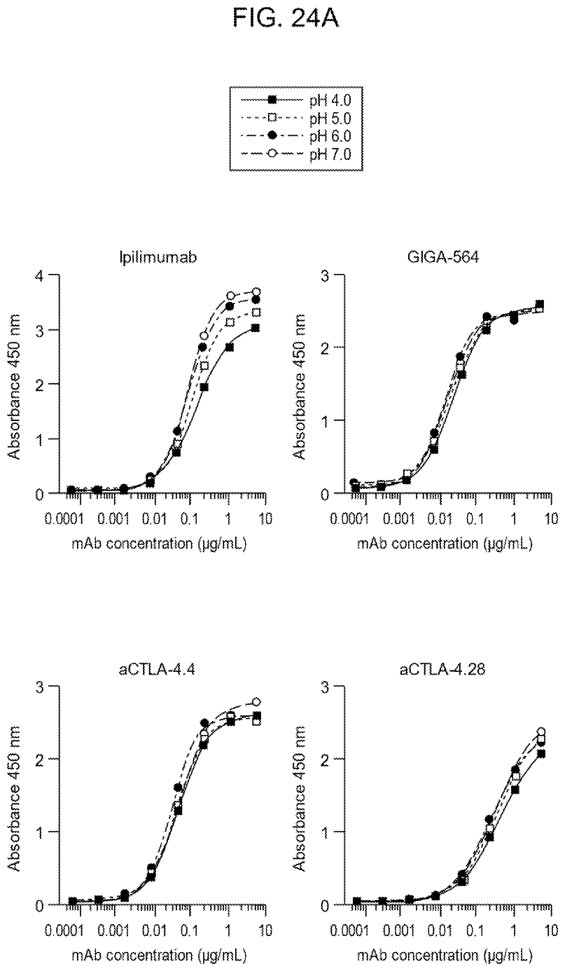

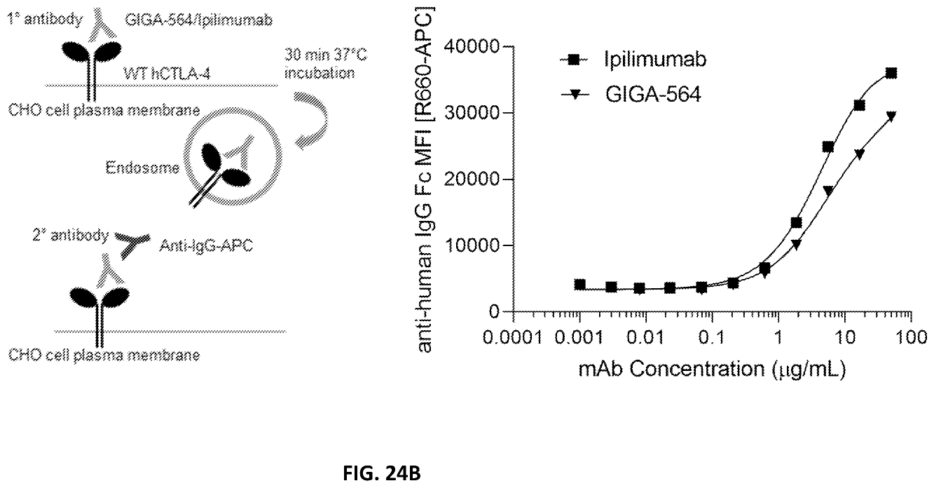

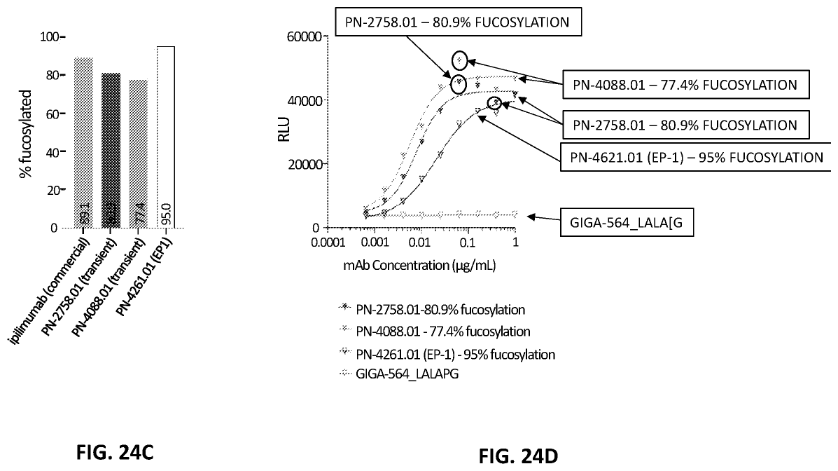

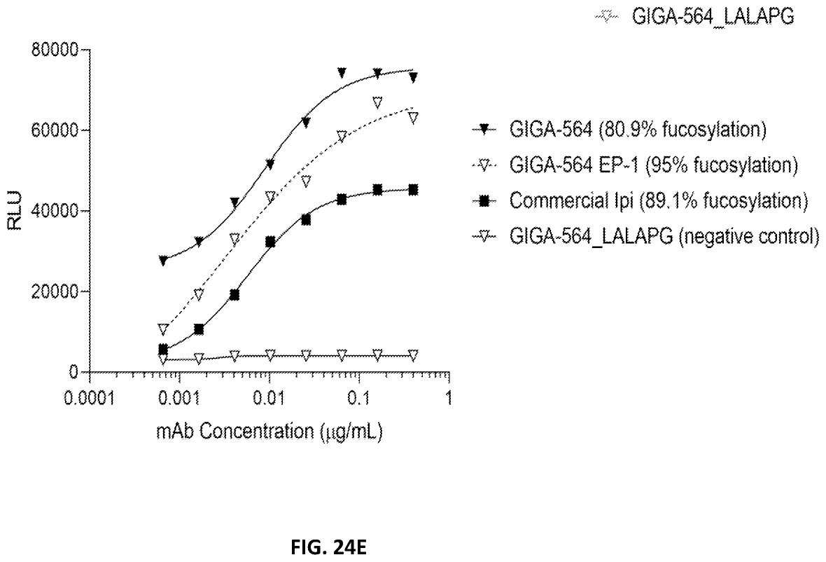

It has previously been suggested that CTLA-4 mAbs with reduced binding at lower pH have enhanced ability to accumulate on the surface of Tregs because they dissociate from CTLA-4 in the early endosome. The hypothesis is that reduced binding at low pH allows for more CTLA-4 recycling, and thus more total CTLA-4 surface expression. A CTLA-4 mAb with reduced binding at low pH would selectively bind CTLA-4 in the periphery over CTLA-4 in the TME. Since the purpose of this study was to generate a CTLA-4 mAb that would target intratumoral Tregs, it was determined the pH sensitivity of several CTLA-4 mAbs and found that GIGA-564 similarly bound to CTLA-4 at all tested pHs, including the at lower pHs often present in the TME (FIG. 24A).

FcR signaling can also be affected by several other factors including the amount of antibody that accumulates on the cell surface and the affinity of the Fc portion of mAb to FcRs. No evidence was found that more GIGA-564 accumulates on the surface of CTLA-4⁺ cells than ipilimumab (FIG. 24B). The affinity of an Fc for FcγRIIIa is in part controlled by Fc glycosylation, specifically Fc fucosylation, because afucosylation enhances the affinity of the Fc for FcγRIIIa and thus ADCC. It was found that GIGA-564 transiently expressed in ExpiCHO cells was less fucosylated than ipilimumab, while a CHO cell pool stably expressing GIGA-564 produced material that was 95% fucosylated (FIG. 24C). Human FcγRIIIa assays revealed that the amount of GIGA-564 afucosylation in each preparation correlated with FcγRIIIa signaling (FIG. 24D). Accordingly, an afucosylated form of GIGA-2328 has been generated as described in Example 17.

Further investigation revealed that GIGA-564 still demonstrates higher FcR activity than ipilimumab, even in the context of decreased afucosylation (FIG. 24E). The increased FcR activity of GIGA-564 likely contributes to its enhanced anti-tumor efficacy, and this may translate well to patients as GIGA-564 also induces enhanced human FcγRIIIa signaling.

### GIGA-564 Induces Durable Anti-Tumor Immune Responses

While first generation anti-CTLA-4s led to durable anti-tumor immunity, little is known about the ability of an anti-CTLA-4 with minimal blocking activity to induce a durable anti-tumor immune response. To investigate this translationally important question, hCTLA-4 KI mice bearing established MC38 tumors were treated on days 0, 3, and 6 with control (isotype), ipilimumab, or GIGA-564 at 1 mg/kg, an FDA-approved dose of ipilimumab that induced a durable complete response in approximately 50% of animals (5/8 ipilimumab treated animals and 5/9 GIGA-564 treated animals) (FIG. 6A). Animals with a complete response on day 43 were then re-challenged with MC38 tumor cells on the opposite flank from the original tumor site. All durable complete responders in the group previously treated with commercial ipilimumab and 4/5 of the durable complete responders in the group previously treated with GIGA-564 did not develop tumors at the re-challenge site (FIG. 17B). Thus, GIGA-564, an anti-CTLA-4 with minimal blocking activity, leads to durable anti-tumor immunity by preventing tumor development at a distant site.

GIGA-564 Induces Less Toxicity than Ipilimumab in Murine Models

In the clinic, CTLA-4 antibodies are most frequently used in combination with PD-1 antibodies, but this combination can induce relatively high frequency of severe adverse events. Thus, a CTLA-4 mAb with enhanced efficacy and reduced toxicity would be beneficial to patients. Several lines of evidence reveal that a significant portion of the toxicities induced by conventional CTLA-4 mAbs likely arises through blocking CTLA-4 from binding CD80 and CD86. This suggests that a CTLA-4 mAb with minimal blocking activity would not only have enhanced anti-tumor efficacy but may also result in less toxicity. Accordingly, the toxicity induced by ipilimumab and GIGA-564 was compared in the presence of anti-PD-1.

hCTLA-4 KI mice treated with a CTLA-4 mAb have been shown to recapitulate some of the toxicity induced by CTLA-4 mAbs in patients, and repeated treatment of BALB/c mice with anti-mouse CTLA-4 antibodies was shown to induce intestinal inflammation.

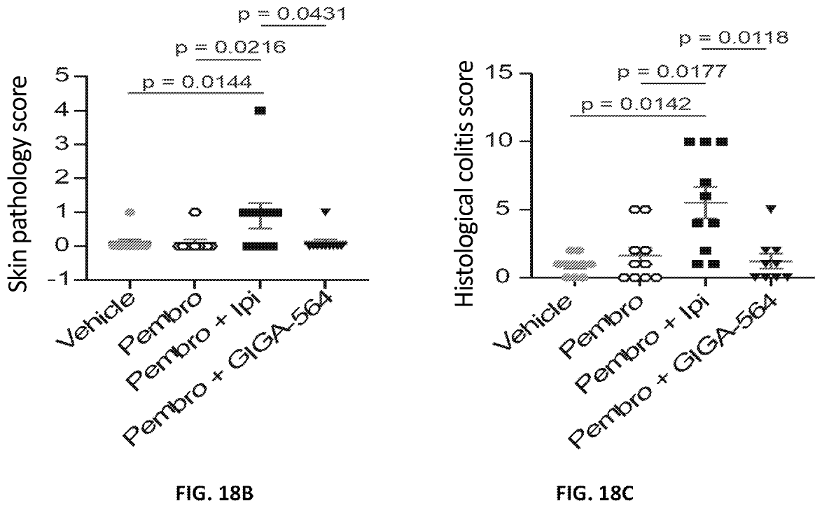

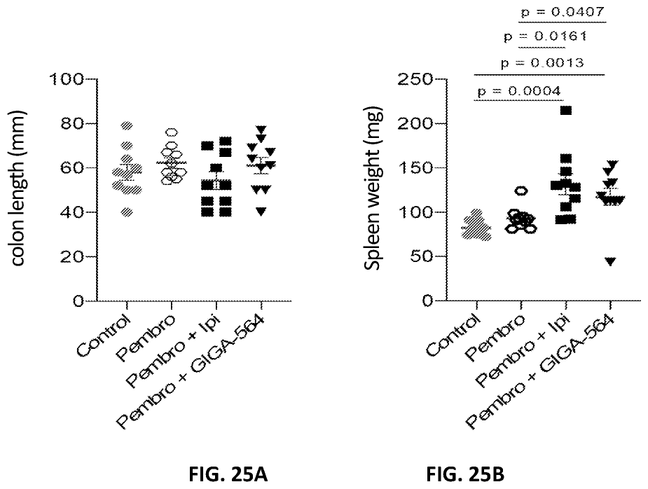

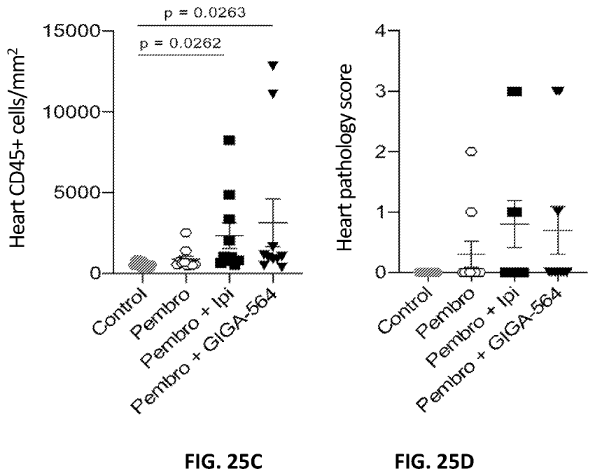

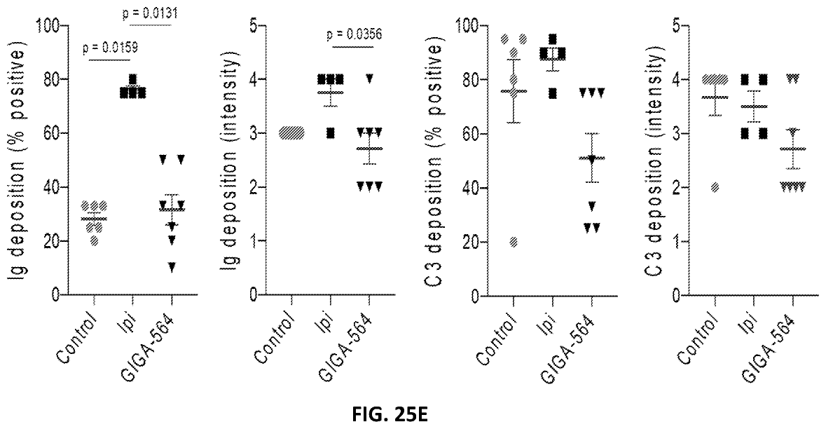

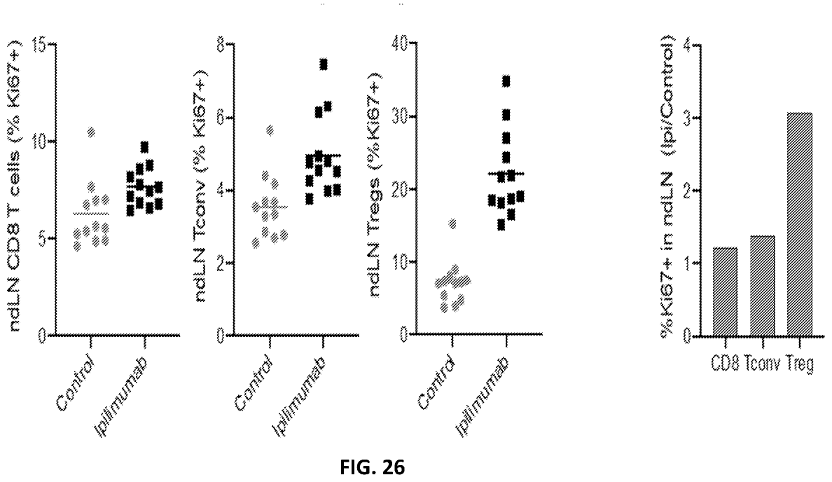

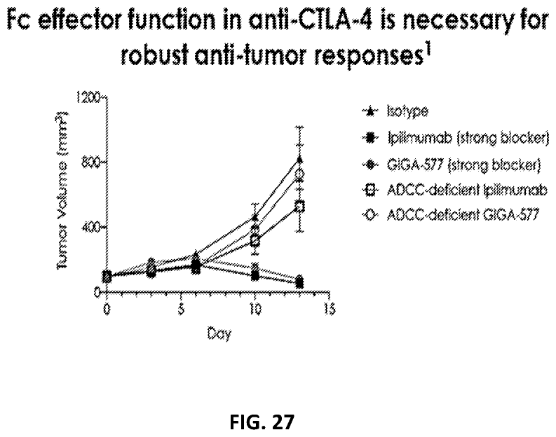

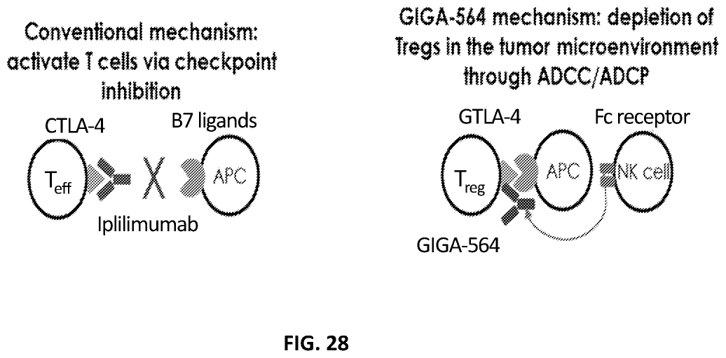

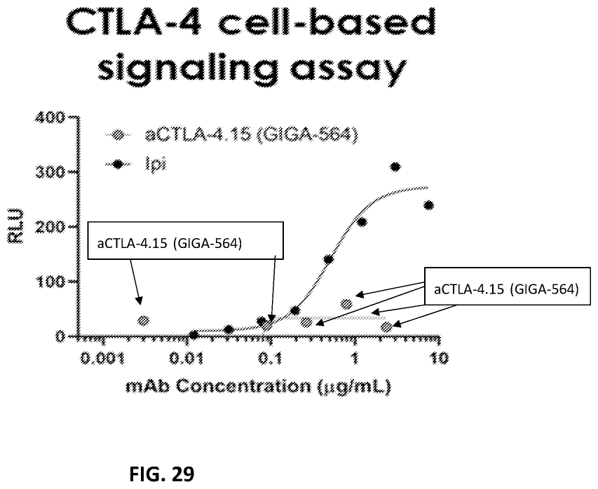

Thus, in the present study, hCTLA-4/hPD-1 double knock-in mice on the BALB/c background were treated with vehicle, pembrolizumab, pembrolizumab plus ipilimumab, or pembrolizumab plus GIGA-564 every 3 days for 9 doses (FIG. 18A). One week after the last dose, mice were euthanized and tissues were collected for pathology analysis (FIG. 18B-18C and FIG. 25A-25B). Histopathological analysis revealed that pembrolizumab plus ipilimumab, but not pembrolizumab plus GIGA-564, induced more colonic epithelial damage and skin inflammation than vehicle treated mice or mice treated with pembrolizumab alone (FIG. 18B-18C), while both combination treatments led to a minor increase in immune cell infiltration to the heart (FIG. 25C-25D). As CTLA-4 mAbs have been shown to induce kidney deposition in hCTLA-4 KI mice on the C57BL/6 background, kidneys were collected on day 20 post initiation of treatment from hCTLA-4 KI mice bearing MC38 tumors treated with vehicle, 5 mg/kg ipilimumab, or 5 mg/kg GIGA-564 twice a week for 5 doses (FIG. 14A). Kidneys were processed into formalin fixed paraffin embedded (FFPE) blocks. FFPE sections were stained for anti-mouse immunoglobin and scored by a board-certified veterinary pathologist blinded from the study. Ipilimumab, but not GIGA-564, significantly increased the percent of glomeruli with murine Ig deposition (FIG. 25E), suggesting that ipilimumab induces more kidney damage than GIGA-564 in hCTLA-4 KI mice. Thus, GIGA-564 induces less toxicity than ipilimumab in these murine models.

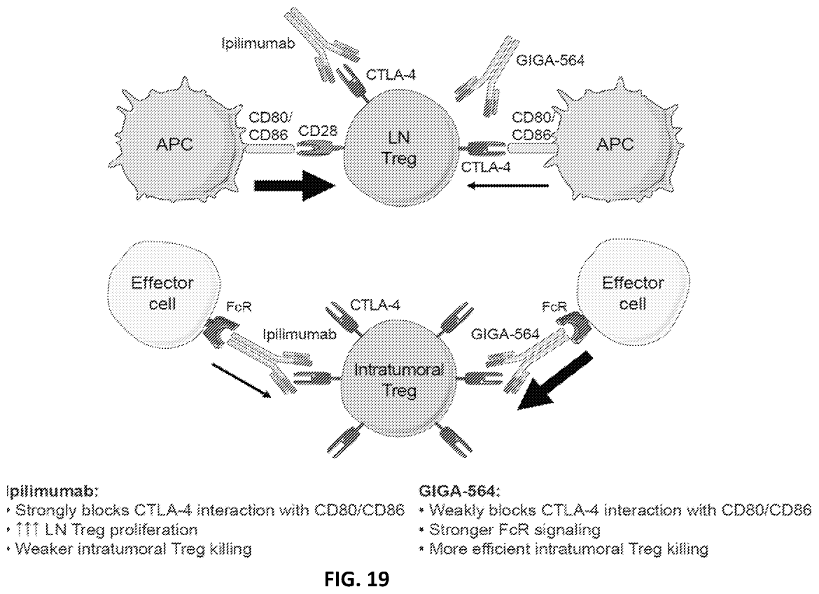

In conclusion, GIGA-564 is a novel CTLA-4 mAb with minimal B7 ligand blocking activity resulting in enhanced efficacy but reduced toxicity in multiple murine models (FIG. 19), suggesting that reducing CTLA-4 checkpoint inhibition yields translationally relevant benefits for both efficacy and safety.

It was shown herein that GIGA-564, an anti-CTLA-4 with minimal ability to block CTLA-4 binding to its CD80/CD86 ligands and enhanced FcR activity, had superior anti-tumor activity and reduced toxicity compared to ipilimumab in murine models expressing human CTLA-4. It was found that in hCTLA-4 KI mice, ipilimumab enhanced Treg proliferation more than proliferation of CD4⁺FOXP3⁻ or CD8 T cells. GIGA-564 leads to less Treg proliferation and increased anti-tumor activity in a murine model when compared to the first generation anti-CTLA-4 mAb ipilimumab.

It has recently been suggested that anti-CTLA-4-mediated depletion of intratumoral Tregs may be transient, explaining why intratumoral Treg depletion is not obvious at all timepoints following anti-CTLA-4 therapy. Thus, one possibility is that anti-CTLA-4 mediated expansion of peripheral Tregs provides a reservoir that reseeds the intratumoral Treg niche. Additionally, while anti-CTLA-4 mAbs efficiently induce depletion of intratumoral Tregs in the MC38 model primarily used here, the ability of anti-CTLA-4s to deplete intratumoral Tregs in patients is expected to be variable. This is because the expression of CTLA-4 by intratumoral Tregs varies among and within patients as can the affinity of FcRs, which are able to mediate antibody-dependent killing of CTLA-4^{Hi} Tregs. Furthermore, GIGA-564 may synergize well with anti-PD-1s, because it would be expected to deplete the intratumoral Tregs an anti-PD-1 could induce.

Several lines of evidence suggest that blocking the ability of CTLA-4 to bind CD80/CD86 may strongly contribute to the severe adverse events induced by first generation CTLA-4 mAbs. First, CTLA4 mutations in humans that decrease binding of CTLA-4 to its B7 ligands are associated with immune infiltration into the brain, gastrointestinal tract, and lung, and can result in diarrhea, colitis, lymphocytic interstitial lung disease, and occasionally autoimmune thyroiditis. Clinically it has also been reported that the gastrointestinal biopsies of CHAI patients (CTLA-4 haploinsufficiency with autoimmune infiltration) are reminiscent of patients treated with anti-CTLA-4 blocking antibodies. Other relevant clinical data has been reported after treatment with abatacept (CTLA-4-Ig), which strongly binds the B7 proteins CD80 and CD86. This study found that pembrolizumab plus GIGA-564 induced less colonic epithelial damage and skin pathology than pembrolizumab plus ipilimumab in human CTLA-4/PD-1 double knock-in mice on the BALB/c background. Gastrointestinal and dermatological adverse events in patients are among the most common severe adverse events associated with ipilimumab or ipilimumab plus anti-PD-1 therapy.

Thus, the data shown herein suggested that clinical translation of GIGA-564 may result in reduced rates of these adverse events commonly associated with conventional CTLA-4 mAbs and thus benefit patients.

Surprisingly, it was found that GIGA-564 has enhanced FcR activity in vitro and in vivo. As next-generation anti-CTLA-4s with enhanced FcR activity have improved anti-tumor efficacy, it is likely that the enhanced FcR activity of GIGA-564 contributes to the improved anti-tumor efficacy of this mAb. The data presented herein indicate that the enhanced FcR function of transiently produced GIGA-564 can be due to the partially afucosylated status of this material; however, further investigation revealed that GIGA-564 still demonstrated higher human FcγRIIIa activity even in the context of decreased afucosylation, suggesting that our results will translate to patients. Of note, the GIGA-564 material used in the murine toxicity study had relatively high levels of afucosylation (80.9%) and Fc effector function, further emphasizing the role of checkpoint inhibition in the induction of adverse events induced by conventional anti-CTLA-4 mAbs.

Another strategy that has been reported to enhance FcR function of CTLA-4 mAbs is selection of mAbs that dissociate from CTLA-4 at acidic pHs, leading to enhanced recycling of CTLA-4 and thus accumulation of the anti-CTLA-4 mAb on the cell surface. However, the tumor microenvironment is often acidic, and thus anti-CTLA-4s that preferentially bind at neutral pH will be less able to bind CTLA-4 in the tumor microenvironment, precisely where it was aimed to selectively deplete Tregs. Importantly, GIGA-564 was able to bind to CTLA-4 efficiently at low pH and can thus target intratumoral Tregs.

It has been argued that the FcR-dependent activity of anti-CTLA-4s may result from FcRs holding CTLA-4 away from the immune synapse and thus serves to further functionally inhibit the CTLA-4/B7 immune checkpoint. As CTLA-4 is most highly expressed by Tregs and co-stimulation is also important for Treg proliferation, anti-CTLA-4s that block the ability of CTLA-4 to bind its B7 ligands induce significant Treg proliferation. Thus, the fact that GIGA-564 only weakly blocks the ability of CTLA-4 to bind its B7 ligands and has enhanced FcR activity but induces less Treg proliferation than ipilimumab, suggests that the Fc activity of anti-CTLA-4s play little role in the inhibiting the ability of anti-CTLA-4s to bind its B7 ligands.

In conclusion, the inventors of the present application showed that the weak blocking CTLA-4 mAb GIGA-564 reduced proliferation of peripheral Tregs, more efficiently depleted intratumoral CTLA-4^{Hi}, and had superior anti-tumor activity while inducing less toxicity than ipilimumab in murine models expressing human CTLA-4.

### 8.15. Example 14: Development and Validation of CHO Cells Expressing Cyno CTLA-4 on the Cell Surface

To determine the ability of GIGA-564 vs ipilimumab (Ipi) to bind cynomolgus CTLA-4 on the cell surface, CHO cells stably expressing cynomolgus CTLA-4 (cyno CTLA-4) or human CTLA-4 (hCTLA-4) were generated.

The ability of PE-labeled anti-CTLA-4 antibodies, clones L3D10 or BNI3, to bind to cyno CTLA-4 or hCTLA-4+ CHO cells was tested to validate the expression of cyno CTLA-4 or hCTLA-4 on the surface of these cells.

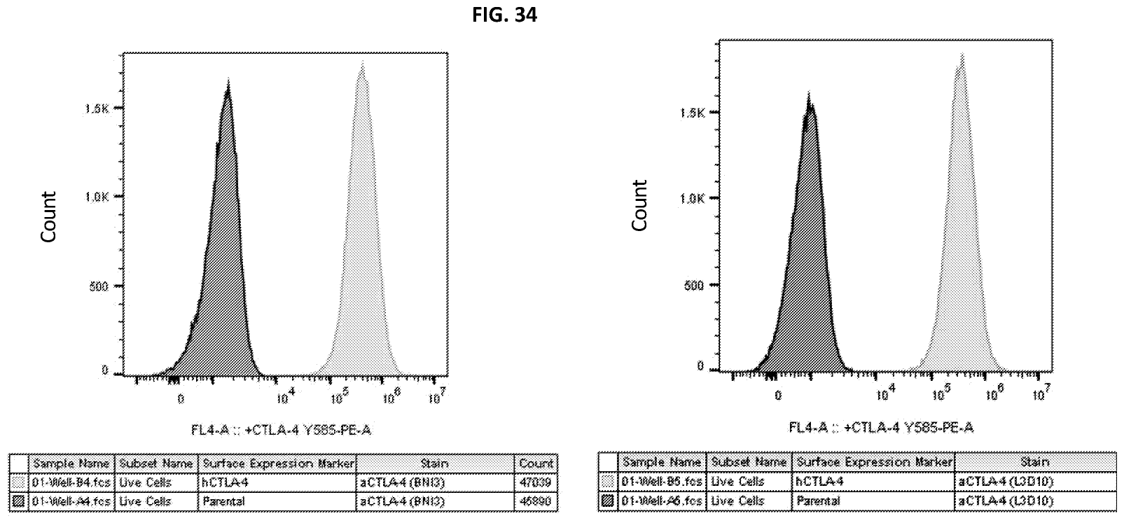

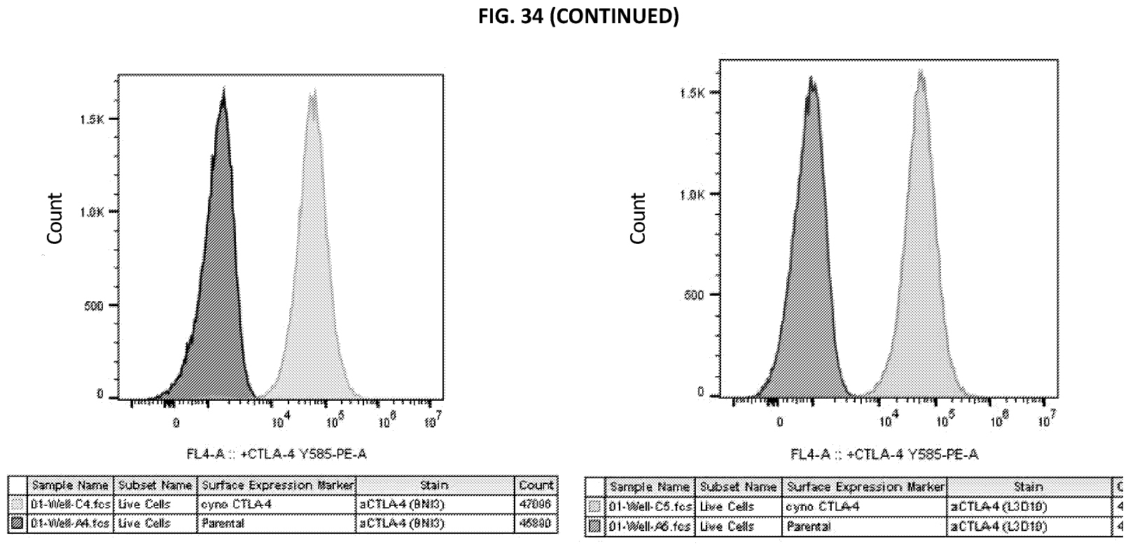

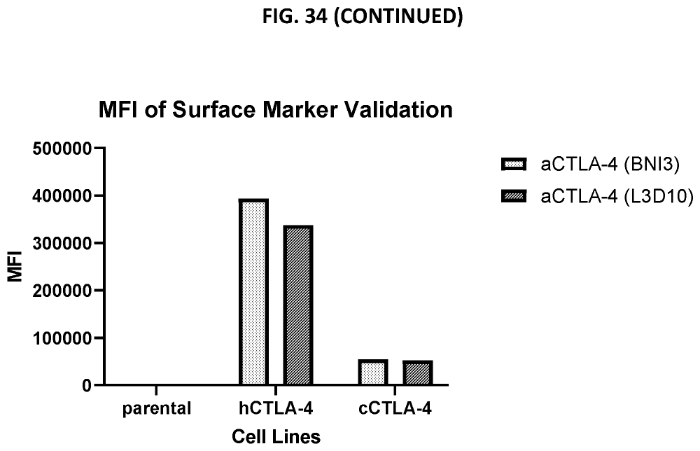

In this experiment, parental CHO cells and cyno CTLA-4 CHO cells or hCTLA-4 CHO cells (1E6 cells each cell line) were first washed with MACS buffer (DPBS+0.5% BSA+2 mM EDTA) and then incubated with either PE-labeled anti-CTLA-4 clones L3D10 or BNI3. Afterwards, the cells were washed twice with MACS buffer to remove excess, unbound antibody. Then, binding of mAbs to cyno or human CTLA-4 on the cell surface was analyzed by flow cytometry (FIG. 34). Flow histograms of FIG. 34 show that the cyno CTLA-4 and hCTLA-4 CHO cell lines express antigen on the cell surface bound by anti-CTLA-4 clone L3D10 or BNI3, this validates expression of cyno or human CTLA-4 on the surface of these cell lines, respectively.

### 8.16. Example 15: GIGA-564 has Reduced Ability to Bind Cyno CTLA-4 Compared to Ipilimumab

To determine the ability of GIGA-564 to bind to cynomolgus (cyno) and human CTLA-4 expressed on the surface of mammalian cells, parental CHO cells and cyno CTLA-4 CHO cells or hCTLA-4 CHO cells (1E6 cells each cell line) were first washed with MACS buffer (DPBS+0.5% BSA+2 mM EDTA) and then incubated with 10 ug/mL of Ipilimumab, Atezolizumab (negative control), or GG-564 at 4° C.

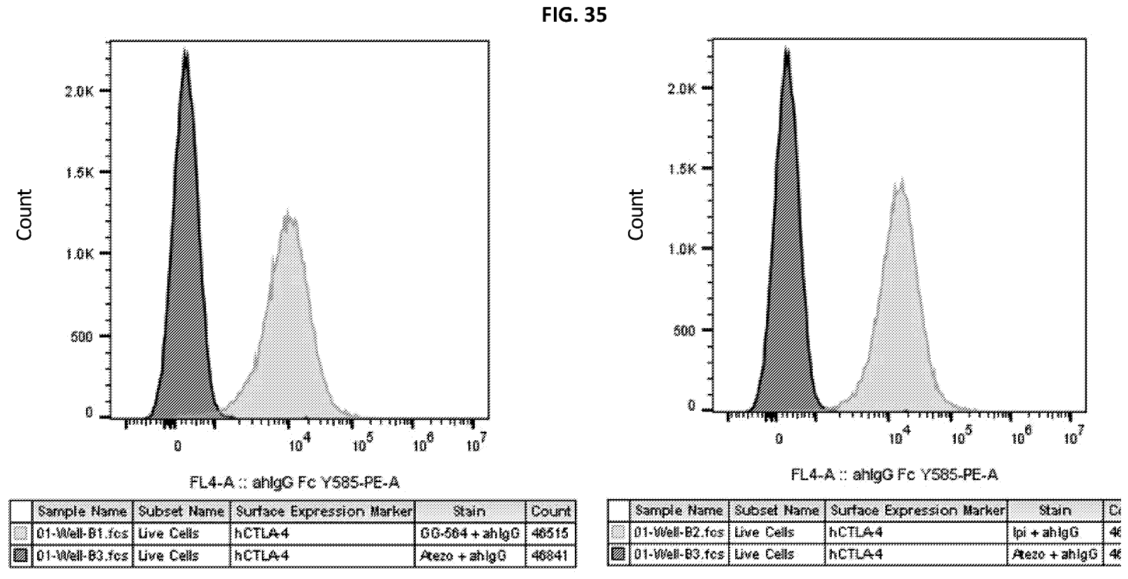

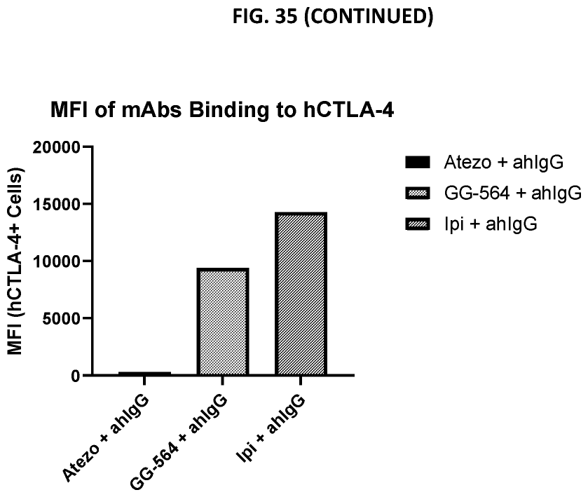

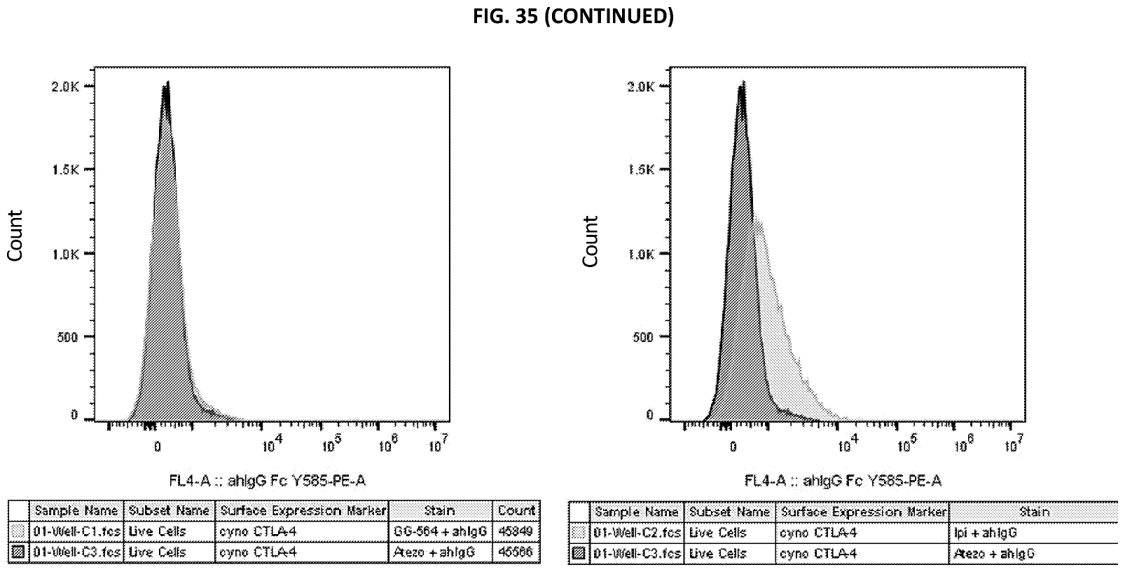

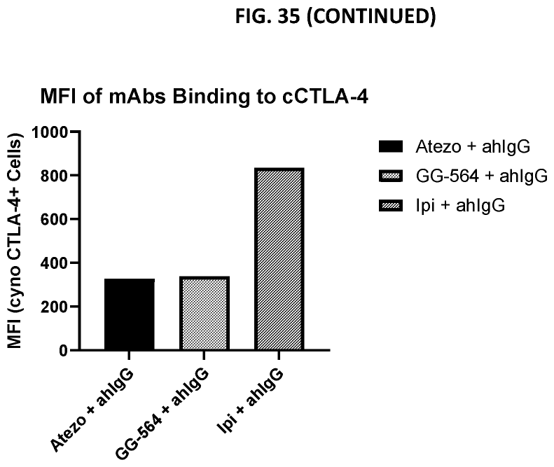

Afterwards, the cells were washed twice with MACS buffer to remove excess, unbound antibodies. The cells were then stained with PE-labeled rat anti-human IgG Fc antibody to detect bound human antibodies. After staining, the cells were washed twice with MACS buffer to remove excess, unbound PE-conjugated anti-human antibodies. Then, binding of mAbs to cyno or human CTLA-4+ CHO cells was analyzed by flow cytometry. FIG. 35 shows that while GIGA-564 and Ipilimumab (Ipi) have relatively similar ability to bind hCTLA-4; compared to Ipi, GIGA-564 has a greatly reduced ability to bind to cyno CTLA-4 expressed on the cell surface. The results suggest that Ipi and GIGA-564 bind to different CTLA-4 epitopes.

Next, the ability of GIGA-564 to bind recombinant cynomolgus monkey CTLA-4 (rcmCTLA-4) was tested by ELISA.

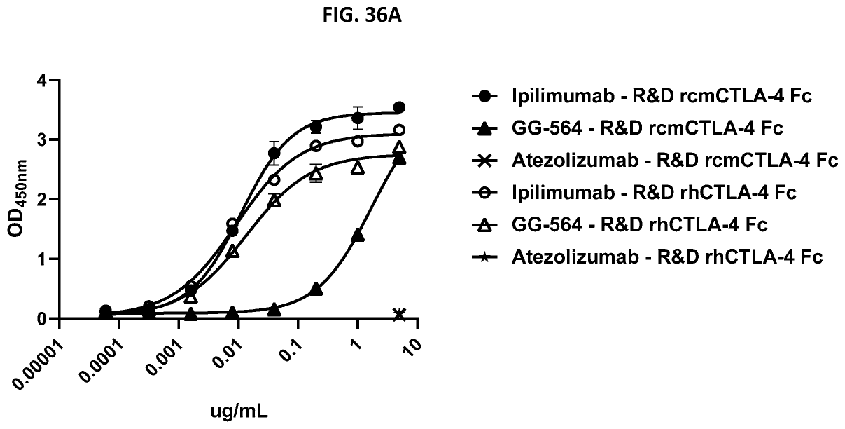

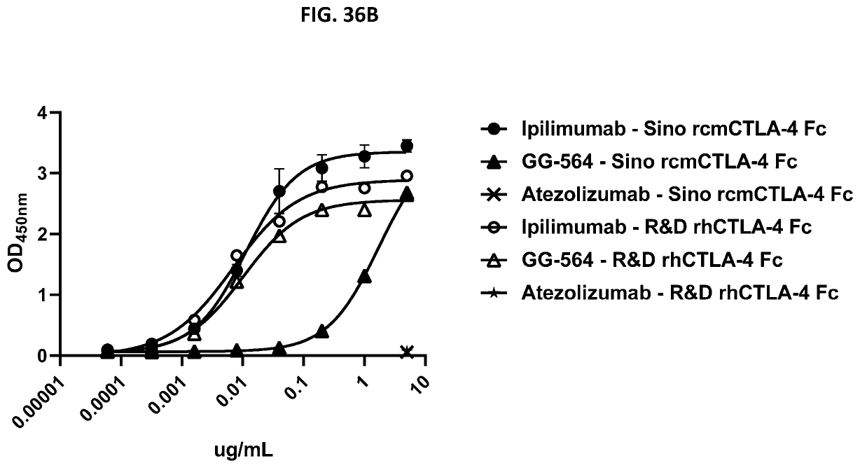

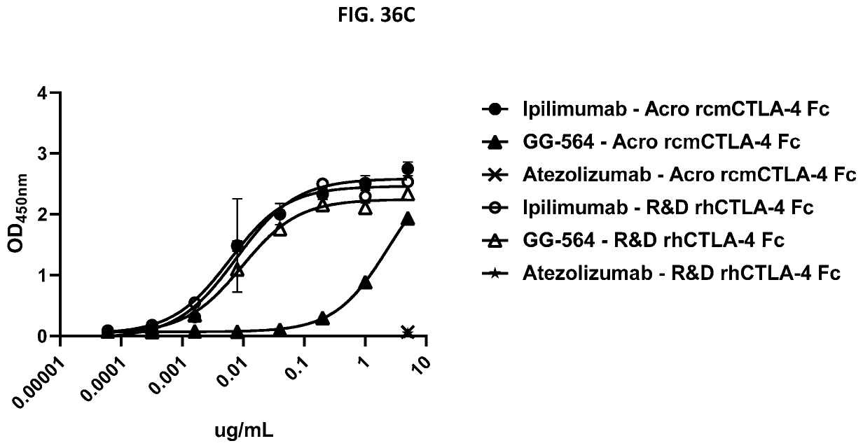

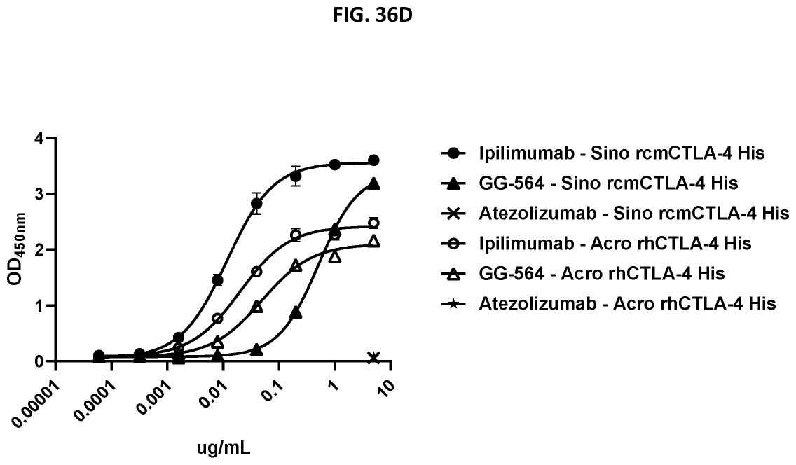

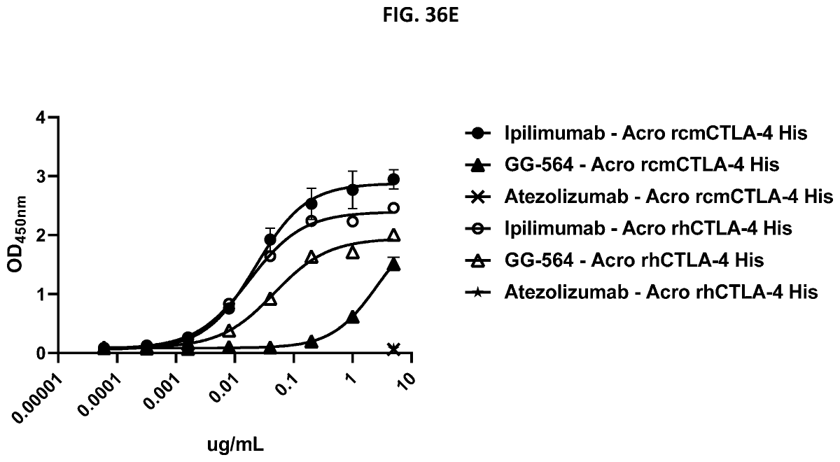

In these ELISAs binding of GIGA-564 to various CTLA-4 proteins sourced from multiple manufacturers, including Fc chimera and his-tagged formats, was tested. To test the binding of GIGA-564 to Fc chimera CTLA-4 proteins, rcmCTLA-4-Fc from R&D systems (9336-CT-200, FIG. 36A), Sino Biological (90213-C02H, FIG. 36B), or ACROBiosystems (CT4-C5256, FIG. 36C) were coated at 1 ug/mL on one half of separate ELISA plates. Recombinant human CTLA-4-Fc (rhCTLA-4) from R&D Systems (7268-CT-100, FIGS. 36A-36C) was coated at 1 ug/mL, on the other half of each of the plates. Similarly, to test the binding of GIGA-564 to his-tagged CTLA-4, rcmCTLA-4-His from Sino Biological (90213-C08H, FIG. 36D) or ACROBiosystems (CT4-C5227, FIG. 36E) were coated at 1 ug/mL on one half of separate ELISA plates. RhCTLA-4-Fc (ACROBiosystems, CT4-H5229, FIGS. 36D-E) was coated at 1 ug/mL, on the other half of each of these plates. After coating, the plates were incubated overnight at 4° C.

The following day, the plates were blocked with 5% milk in PBST for 1 hour on a plate shaker at room temperature. Titration series of Ipilimumab, Atezolizumab (negative control), and GG-564 (starting at 5 ug/mL) were added to the plates and incubated on a plate shaker for 1 hour at room temperature to allow mAb binding. Excess, unbound mAbs were removed by washing with PBST. Bound mAbs were then detected with an HRP-conjugated anti-kappa light chain antibody (0.5 ug/ml, Southern Biotech 2060-50). After incubation on a plate shaker for 1 hour at room temperature and washing, the plates were developed with TMB substrate. After sufficient signal was achieved, 1 N hydrochloric acid was added to stop development. Absorbance at 450 nm was read using a Spectramax i3x plate reader (Molecular Devices). EC50 values were calculated by plotting absorbance vs. the log of concentration using Prism (GraphPad). As Ipi has similar binding to human and cyno CTLA-4, in most cases, GIGA-564 had reduced ability to bind cyno CTLA-4 compared to human CTLA-4. These results suggest that the Ipi and GIGA-564 epitopes are different in practice.

Table 28 shows the EC50 values and fold change for each antibody tested in the ELISA assays.

TABLE 28

| | | | Fold |
|---|---|---|---|
| mAb | Antigen | EC50 | Change* |
| GG-564 | R&D rcmCTLA-4 Fc | 1.71** | 126 |
| GG-564 | R&D rhCTLA-4 Fc | 0.0136 | |
| Ipilimumab | R&D rcmCTLA-4 Fc | 0.0111 | 1.2 |
| Ipilimumab | R&D rhCTLA-4 Fc | 0.0092 | |
| GG-564 | Sino Biological rcmCTLA-4 Fc | 1.7290** | 170 |
| GG-564 | R&D rhCTLA-4 Fc | 0.0102 | |
| Ipilimumab | Sino Biological rcmCTLA-4 Fc | 0.0112 | 1.6 |
| Ipilimumab | R&D rhCTLA-4 Fc | 0.0070 | |
| GG-564 | ACROBiosystems rcmCTLA-4 Fc | 2.43** | 254 |
| GG-564 | R&D rhCTLA-4 Fc | 0.0096 | |
| Ipilimumab | ACROBiosystems cCTLA-4 Fc | 0.0077 | 1.3 |
| Ipilimumab | R&D rhCTLA-4 Fc | 0.0060 | |
| GG-564 | Sino Biological rcmCTLA-4 His | 0.5117 | 10 |
| GG-564 | ACROBiosystems rhCTLA-4 His | 0.0496 | |
| Ipilimumab | Sino Biological rcmCTLA-4 His | 0.0118 | 0.6 |
| Ipilimumab | ACROBiosystems rhCTLA-4 His | 0.0203 | |
| GG-564 | ACROBiosystems rcmCTLA-4 His | 2.65** | 57 |
| GG-564 | ACROBiosystems rhCTLA-4 His | 0.0468 | |
| Ipilimumab | ACROBiosystems rcmCTLA-4 His | 0.0226 | 1.3 |
| Ipilimumab | ACROBiosystems rhCTLA-4 His | 0.0176 | |

*fold change: EC50 of cyno divided by EC50 of human
**did not reach signal saturation

### 8.17. Example 16: Fucosylation Analysis of GIGA-564, GIGA-2328, and Ipilimumab

Table 30 shows a fucosylation analysis of GIGA-564 and Ipiliumumab produced with unmanipulated fucosylation, and Table 31 shows a fucosylation analysis of GIGA-564 and Ipiliumumab produced with conditions to promote afucosylation. Each antibody was digested with PNGase F to liberate glycans. Glycans were isolated and labeled with a fluorescent molecule using a GlykoPrep InstantAB labeling kit (Prozyme) following the manufacturer's instructions. Labeled glycans were injected over a XBridge Amide column on an Ultra Performance Liquid Chromatography (UPLC) system with a fluorescent detector. Peaks were identified based on known glycans from the Glyko InstantAB biantennary and high-mannose partitioned library standards (Prozyme), with the total percent of each glycoform reported as the integrated area under the identified peak divided by the sum of the area under the curve for all peaks. Each value represents and independent measurement of the indicated lot. If more than one measurement was made, the minimum, maximum, and mean values are also provided.

TABLE 30
Fucosylation analysis of GIGA-564 and Ipiliumumab produced with unmanipulated fucosylation

| GIGA-564 (transient | | GIGA-564 (stable | | Ipilimumab (transient | | Ipilimumab (stable | | | |
|---|---|---|---|---|---|---|---|---|---|
| expression) | | expression) | | expression) | | expression) | | Ipilimumab (Yervoy) | |
| | % fucosylated | | % fucosylated | | % fucosylated | | % fucosylated | | % fucosylated |
| min | 77.4 | min | 81.6 | min | 82.7 | min | 88.0 | min | 88.4 |
| max | 80.9 | max | 95.0 | max | 83.5 | max | 88.3 | max | 89.7 |
| average | 79.4 | axerage | 90.3 | average | 83.1 | average | 88.2 | average | 89.1 |
| Lot | % fucosylated | Lot | % fucosylated | Lot | % fucosylated | Lot | % fucosylated | Lot | % fucosylated |
| PN- | 80.9 | PN- | 95.0 | PN- | 82.7 | PN- | 88.3 | PN- | 89.1 |
| 2758.01 | | 4261.01 | | 4265.01 | | 4489 | | 4094 | |
| PN- | 77.4 | PN- | 94.3 | PN- | 83.5 | PN- | 88.0 | PN- | 89.4 |
| 4088.01 | | 4261.02 | | 4265.01 | | 4532.01 | | 2167 | |
| PN- | 79.8 | PN- | 89.9 | | | | | PN- | 88.8 |
| 2758.01 | | 4262.01 | | | | | | 2753 | |
| | | PN- | 92.0 | | | | | PN- | 88.4 |
| | | 4324.01 | | | | | | 4094 | |
| | | PN- | 91.6 | | | | | PN- | 89.7 |
| | | 4324.01 | | | | | | 4094 | |
| | | PN- | 85.5 | | | | | | |
| | | 4554.01 | | | | | | | |
| | | PN- | 81.6 | | | | | | |
| | | 4555.01 | | | | | | | |
| | | PN- | 91.2 | | | | | | |
| | | 4324.01 | | | | | | | |
| | | PN- | 91.4 | | | | | | |
| | | 4324.02 | | | | | | | |

Some of the data shown in Table 30 is also provided in Example 13.

TABLE 31
Fucosylation analysis of GIGA-564 and Ipiliumumab
produced with conditions to promote afucosylation

| GIGA-564 (transient | | GIGA-564 (stable | | Ipilimumab (transient | | Ipilimumab (stable | |
|---|---|---|---|---|---|---|---|
| expression) | | expression) | | expression) | | expression) | |
| Lot | % fucosylated | Lot | % fucosylated | | % fucosylated | | % fucosylated |
| PN- | 5.2 | PN- | 25.1 | min | 5.2 | min | 3.3 |
| 2328.01 | | 4534.01 | | | | | |
| | | | | max | 6.3 | max | 3.9 |
| | | | | average | 5.9 | average | 3.6 |
| | | | | Lot | % fucosylated | Lot | % fucosylated |
| | | | | PN- | 5.2 | PN- | 3.3 |
| | | | | 2326.01 | | 4491.01 | |
| | | | | PN- | 6.3 | PN- | 3.9 |
| | | | | 4266.01 | | 4533.01 | |
| | | | | PN- | 6.2 | | |
| | | | | 4266.01 | | | |

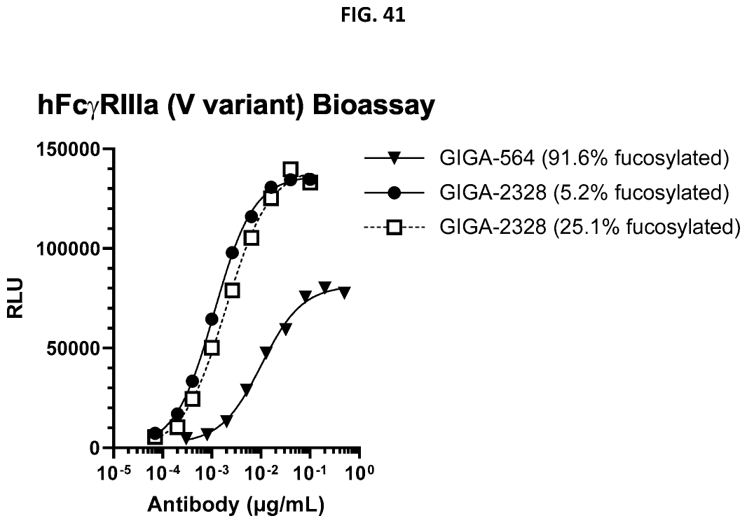

GIGA-2328 was designed to have low fucosylation for the purpose of enhancing FcγRIIIa signaling. As shown in FIG. 41, an ADCC reporter bioassay for human FcγRIIIa, V variant (G7011) from Promega Corporation was carried out following the manufacturer's instructions. Briefly, CHO target cells stably expressing human CTLA-4 with the Y201G mutation to enhance cell surface expression were suspended in RPMI 1640+4% FBS media with the indicated antibody and incubated at 37° C. for 30 minutes. Jurkat/NFAT-Luc effector cells expressing human FcγRIIIa, V variant were added to each well at an effector:target ratio of 5:1 and incubated at 37° C. for 6 hours. Luciferase activity was measured by using the included Bio-Glo Luciferase Assay Reagent with the SpectraMax i3x reader. Luciferase activity measured in relative luminescence units (RLU) were plotted against the concentration of antibody.

TABLE 34
GIGA-2328 has lower overall fucosylation than GIGA-564.

| Glycoform | GIGA-564 | GIGA-2328¹ | GIGA-2328² |
|---|---|---|---|
| fucosylated | 91.6 | 5.2 | 25.1 |
| afucosylated | 7.2 | 91.6 | 74.0 |
| G0-GlcNac | 0.4 | 7.5 | 1.4 |
| G0F-GlcNac | 0.6 | 0.6 | 0.3 |
| G0 | 3.9 | 55.5 | 60.2 |
| G0F | 64.1 | 2.9 | 19.4 |
| Man5/G1-1* | 2.8 | 11.7 | 8.3 |
| G1-2 | | | 3.5 |
| G1F-1 | 17.7 | 1.0 | 3.5 |
| G1F-2 | 7.0 | 0.7 | 1.5 |
| G2 | | | 0.6 |
| Man6 | 0.1 | 6.3 | |
| G2F | 2.2 | | 0.4 |
| Man7 | | 4.2 | |
| Man8 | | 4.3 | |
| Man9 | | 2.0 | |
| Unknown | 1.1 | 3.2 | 0.9. |

¹antibody was expressed by transient transfection in ExpiCHO cells (Thermo Fisher)
²antibody was stably expressed in the CHOZN cell line (Sigma Aldrich)
*in some cases separate values for Man5/G1-1 were summed to calculate this value.
Some data in Table 34 includes repeated data shown in Table 30 and Table 31.

Each antibody was digested with PNGase F to liberate glycans. Glycans were isolated and labeled with a fluorescent molecule using a GlykoPrep InstantAB labeling kit (Prozyme) following the manufacturer's instructions. Labeled glycans were injected over a XBridge Amide column on an Ultra Performance Liquid Chromatography (UPLC) system UPLC with a fluorescent detector. Peaks were identified based on known glycans from the Glyko InstantAB biantennary and high-mannose partitioned library standards (Prozyme), with the total percent of each glycoform reported as the integrated area under the identified peak divided by the sum of the area under the curve for all peaks

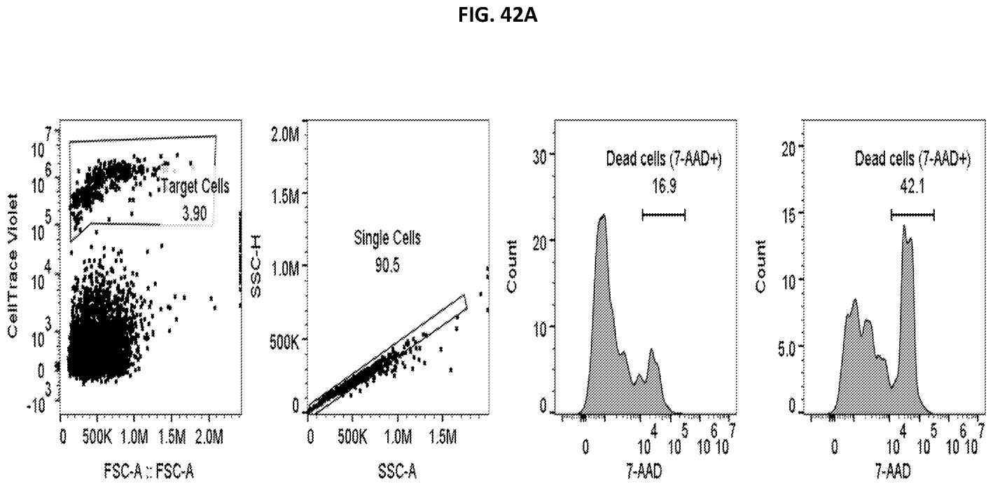

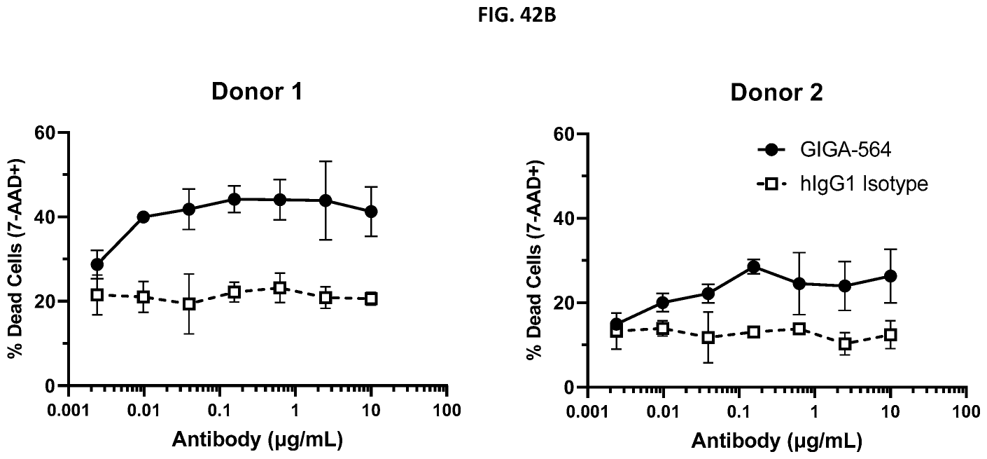

FIGS. 42A-42B. GIGA-564 induces antibody-dependent cellular cytotoxicity (ADCC) and/or antibody-dependent cellular phagocytosis (ADCP) by human PBMCs against a target cell line expressing CTLA-4. Cryopreserved human PBMCs from two donors were thawed and recovered overnight in RMPI media containing 100 U/mL IL-2 and 10% FBS. CHO target cells stably expressing human CTLA-4 with the Y201G mutation to enhance cell surface expression were stained with CellTrace Violet (Thermo Fisher), then incubated with either GIGA-564 or a human IgG1 isotype control at the indicated concentrations at 37° C. for 30 minutes. PMBC effector cells were then added at a 20:1 effector to target ratio and the samples were incubated at 37° C. for 4 hours to allow for ADCC. The samples were then washed with MACS buffer, stained with the viability dye 7-Aminoactinomycin D (7-AAD) to label dead cells, and analyzed by flow cytometry. (FIG. 42A) Representative gating strategy to determine ADCC. Samples were first gated for target cells (CellTrace Violet+), then single cells, then dead (7-AAD+) cells. (FIG. 42B) Plots of percent dead cells at each concentration of GIGA-564 and human IgG1 isotype. GIGA-564 leads to higher percent dead CTLA-4+ target cells than the isotype control.

### 8.18. Example 17: Effect of Different IgG1 Allotypes on FcγRIIIa Signaling

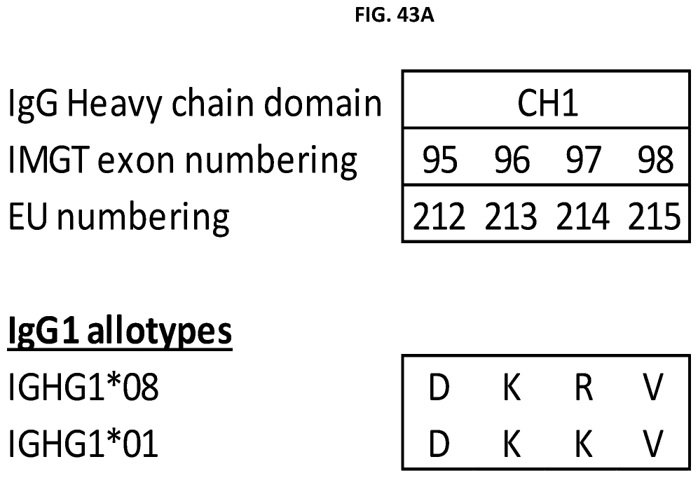

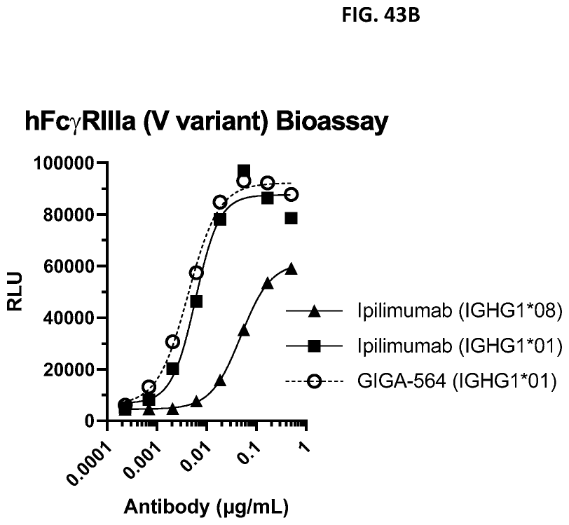

IgG1 allotype may impact signaling via the FcγRIIIa receptor. FIG. 43A shows sequence information of IgG1 allotype IGHG1*08 and IGHG1*01, which differ by one amino acid in the CH1 domain. The differing residue between these two allotypes is highlighted, and surrounding residues are provided for reference. Position given in both IMGT and EU numbering.

FIG. 43B shows clinical ipilimumab (Yervoy) uses the IGHG1*08 allotype, and results in lower FcγRIIIa signaling than Ipilimumab and GIGA-564 produced by GigaGen, which use the IGHG1*01. These results suggest that allotype may impact FcγRIIIa signaling. ADCC reporter bioassays for human FcγRIIIa, V variant (G7011) were purchased from Promega Corporation. The assays were carried out following the manufacturer's instructions. Briefly, CHO target cells stably expressing human CTLA-4 with the Y201G mutation to enhance cell surface expression were suspended in RPMI 1640+4% FBS media with the indicated antibody and incubated at 37° C. for 30 minutes. Jurkat/NFAT-Luc effector cells expressing human FcγRIIIa, V variant were added to each well at an effector:target ratio of 5:1 and incubated at 37° C. for 6 hours. Luciferase activity was measured by using the included Bio-Glo Luciferase Assay Reagent with the SpectraMax i3x reader. Luciferase activity measured in relative luminescence units (RLU) were plotted against the concentration of antibody.

### 8.19. Example 16: Formulation of ABPs for Clinical Use

The study was performed to determine a formulation with optimal protein stability and ease of administration and to have a tonicity (near isotonic, 290 mOsm/kg) for simpler administration.

GIGA-564 was formulated at 20 mg/mL in a variety of buffers, then measured using differential scanning fluorimetry to look for conformation and unfolding.
Range of formulations tested: from pH 5 to pH 7.6
Buffer: The buffer was either citrate or phosphate (of varying concentrations) as was appropriate for the pH
Salt Concentration: The salt concentration ranged from 0-200 mM NaCl
Sucrose: Sucrose was tested at a range from 0-10% (weight to volume), 10% is about 290 nM

Samples run on differential scanning fluorimetry over 25-95° C. and scattering was reported as mAU. Measurement output was the ratio of fluorescence at 350 nm to 330 nm. The 350 signal doesn't change much, however as the protein unfolds the 330 signal decreases significantly (driven by tryptophan becoming more accessible).

Tonset is the temperature at which the signal starts to increase.

Tm-1-2,-3 etc. are unfolding temperatures of different domains, defined by an inflection point in the 350/330 data and local maximum in the first derivative.

For an antibody, there are not always 3 distinct Tm, but generally: Tm1=CH₂; TM2=fab; Tm3=CH₃.

Tagg=the temperature at which the scattering increases, indicating an increase in size of the particles.

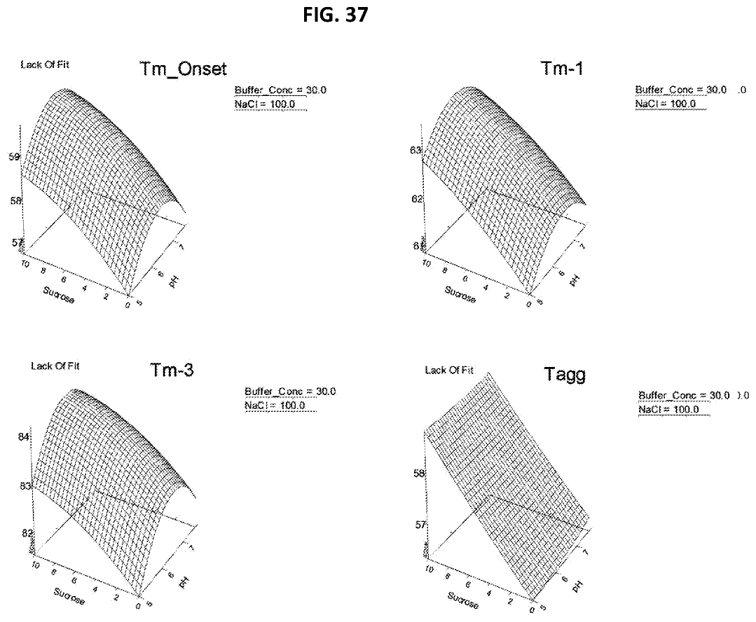

The 350/330 ratio at 25° C. indicates the conformation—lower is more folded. FIG. 37 provides response plots that show the impact of pH and sucrose concentration when NaCl and buffer concentration are fixed at 100 mM and 30 mM, respectively. The results show that (i) higher Tms and Tagg are better; (ii) higher [sucrose] improves results; (iii) best pH in the middle (˜6.3); (iv) pH has little effect on Tagg.

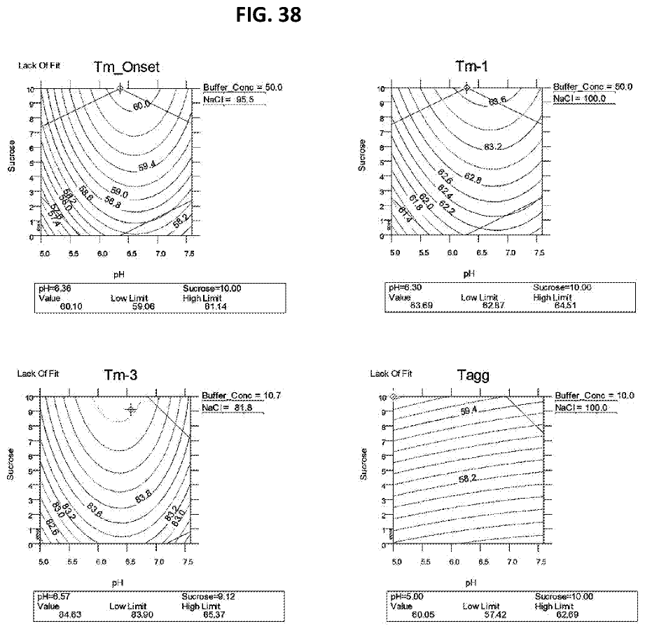

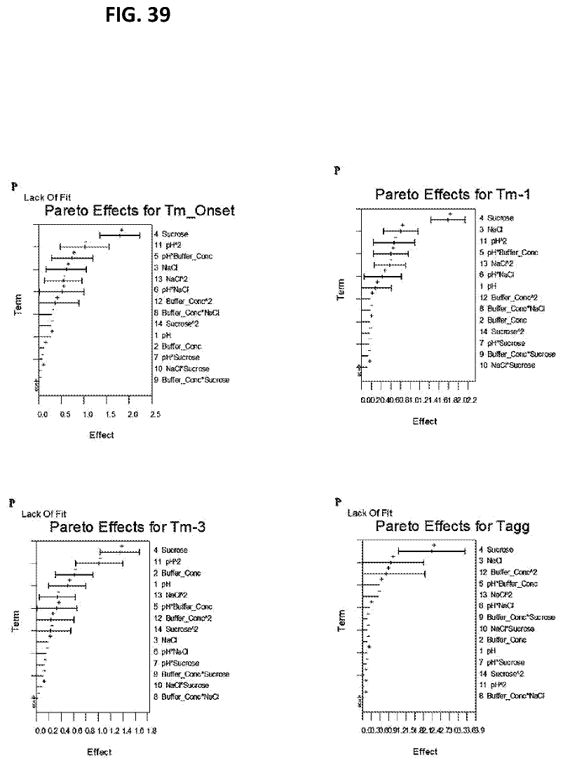

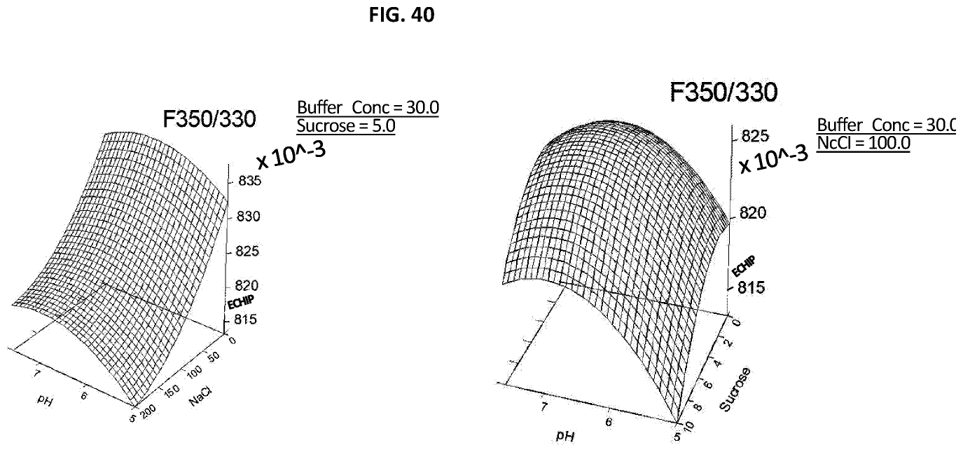

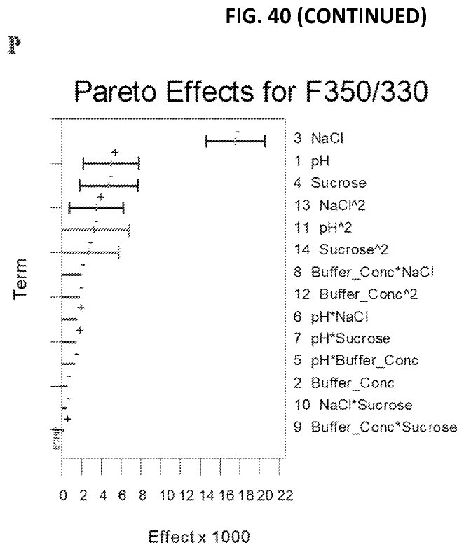

Next, optimal conditions for the formulation were calculated. Theoretical response plots as shown in FIG. 38 were generated to determine the buffer concentration and NaCl amount expected to result in the highest Tm as shown. The results show that (i) best buffer concentration varies from 10-50 mM; and (ii) the calculated ideal [NaCl] for all parameters is high, from 80-100 mM. Next, a pareto analysis was performed to determine which variables had the most impact on the formulation. Data in FIG. 39 suggested (i) for all, sucrose concentration was the most important variable; and (ii) pH was moderately important for Tm-onset, TM-3. Additionally, FIG. 40 shows (i) high NaCl was the most important for conformation; (ii) pH had some effect, with lower pH being better; and (iii) sucrose had some effect, with higher being better.

Based on these findings, the below formulations are generated and tested.

| form. | mg/ml | pH | buffer | type | [NaCl] | [sucrose] | PS-20 | Osmo |
|---|---|---|---|---|---|---|---|---|
| 1 | 20 | 6.5 | 20 mM | histidine | — | 270 mM | 0.02% | 290 mOsm/kg |
| 2 | 20 | 6.5 | 20 mM | histidine | 50 mM | 270 mM | 0.02% | 390 mOsm/kg |
| 3 | 20 | 6.5 | 20 mM | histidine | 50 mM | 170 mM | 0.02% | 290 mOsm/kg |
| 4 | 20 | 6 | 20 mM | histidine | — | 270 mM | 0.02% | 290 mOsm/kg |
| 5 | 20 | 6 | 20 mM | histidine | 50 mM | 270 mM | 0.02% | 390 mOsm/kg |
| 6 | 20 | 6 | 20 mM | histidine | 50 mM | 170 mM | 0.02% | 290 mQsm/kg |
| 7 | 20 | 5.5 | 20 mM | citrate | — | 270 mM | 0.02% | 290 mOsm/kg |
| 8 | 20 | 5.5 | 20 mM | citrate | 50 mM | 270 mM | 0.02% | 390 mOsm/kg |
| 9 | 20 | 5.5 | 20 mM | citrate | 50 mM | 170 mM | 0.02% | 290 mOsm/kg |

TABLE 30
Phase II rationale: comparison to commercial IO mabs

| | pH | buffer | mM | sugar | mM | salt | mM | other | mM | surfactant | % |
|---|---|---|---|---|---|---|---|---|---|---|---|
| atezolizumab | 5.8 | histidine | 20 | sucrose | 120 | none | 0 | | | PS-20 | 0.040% |
| ipilimumab | 7.0 | tris | 26 | mannitol | 55 | NaCl | 100 | | | PS-80 | 0.010% |
| nivolumab | 6.0 | citrate | 20 | mannilol | 165 | NaCl | 50 | | | PS-80 | 0.020% |
| pembrolizumab | 5.5 | histidine | 2.5 | sucrose | 51 | none | 0 | | | PS-80 | 0.005% |
| durvalumab | 6.0 | histidine | 2.5 | trehalose | 28 | none | 0 | | | PS-80 | 0.002% |
| ovelumab | 5.0-5.6 | none | 0 | mannitol | 28 | none | 0 | | | PS-20 | 0.005% |
| cemiplimab | 6.0 | histidine | 10 | sucrose | 146 | none | 0 | proline | 130 | PS-80 | 0.200% |

Alternative or additional components of the formulation can include Trehalose instead of sucrose, and at appropriate pHs, histidine or succinate could be used instead of citrate/phosphate.

### 8.1. Example 17: Manufacturing of ABP, GIGA-564 and GIGA-2328

GIGA-564 can be made in CHO cells. In some variations it can be expressed transiently, for example using ExpiCHO cells, Expi293 cells, or other cell lines commonly used for transient transfection. In some variations it can be expressed from a cell line modified to stably express the heavy and light chain sequences of GIGA-564 using a cell line commonly used for stable expression, such as DG44, CHOZN GS, or others. The stable expression construct may be integrated using methods such as targeted integration, random integration, transposase-mediated integration, lentiviral transduction, or others. In some variations an enhancer element of some kind may be used to enhance transcription or translation and thus titers, for example a 2G UNic element, UCOE element, or others.

GIGA-2328 (aCTLA-4.15 with low fucosylation) is produced in cells selected due to their ability to produce proteins with reduced core fucosylation. In some variations GIGA-2328 will be produced in CHO cells expressing the bacterial protein RMD (GDP-6-deoxy-D-lyxo-4-hexulose reductase), which prevents the production of UDP-Fucose from UDP-Mannose. In some variations, fucosylated GIGA-564 can also be produced using cells expressing the bacterial protein RMD (GDP-6-deoxy-D-lyxo-4-hexulose reductase) if an external source of fucose is given to the cells. In some variations, GIGA-2328 may be produced in CHO cells lacking or having reduced expression of Fut8. In some variations, GIGA-2328 may be produced by adding the fucosylation inhibitor 2-Fluorfucose (2FF) to the media the cells are grown in. In some variations, GIGA-2328 may be produced in cells overexpressing the glycosyltransferase (GnTIII). Additionally, GIGA-2328 can be produced by other CHO cells selected or modified to have reduced fucosylation levels or by another cell line selected for its ability to produce proteins with reduced fucosylation.

To manufacture GIGA-564 cells are thawed from storage in liquid nitrogen and expanded to reach a sufficient density for inoculation of a vessel for bioproduction (including but not limited batch, fed batch or perfusion culture in a shake flask, TPP or bioreactor). In one variation, the fed batch culture is grown for approximately 14 days with addition of glucose and feeds, as appropriate. After the fed batch culture is complete the culture is harvested by methods that may include cell settling, centrifugation, and/or filtration.

GIGA-564 is purified from the harvested cell culture fluid using one or more of several chromatographic steps. The first step is Protein A or similar affinity chromatography, using a resin such as MabSelect Sure, MabSelect PrismA, CaptivA, or others. The affinity chromatography step may be followed by low pH viral inactivation, followed subsequently by cation exchange chromatography in bind and elute mode, using a resin such as POROS XS, CaptoS ImpaAct or others. Anion exchange filtration in flow-through mode is performed using a filter such as Natrix Q, SartobindQ, Mustang Anionic Exchange (Q) or others, and nanofiltration for virus removal is performed using filters such as Planova BioEX, Viresolve, Pegasus or others. Finally, GIGA-564 is subjected to ultrafiltration/diafiltration to concentrate and buffer exchange to the final formulation conditions. A similar approach for manufacturing and purifying can be used for manufacturing and purification of GIGA-2328.

### 9. INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes.

### 10. EQUIVALENTS

While various specific embodiments have been illustrated and described, the above specification is not restrictive. It will be appreciated that various changes can be made without departing from the spirit and scope of the present disclosure(s). Many variations will become apparent to those skilled in the art upon review of this specification.

### Table 19 Provides Sequences for Antibody Light Chains, Heavy Chains, CDRs, and Human CTLA4.

TABLE 19

| SEQ | | |
|---|---|---|
| ID | | Chain |
| NO | Sequence | (Antibody) |
| 1 | EIVLTQSPGTLSLSPGEGATLSCRASQSFSSNYLAWYQQKPGQAPR | L1 (A1) |
| | LLIYGASSRATGIPDRFSGSGSGTDFTLTISRLEPEDFAVYYCQQYG | “aCTLA-4.9” |
| | TSPFTFGPGTKVDIK | |
| 2 | EIVLTQSPGTLSLSPGERATLSCRASQSVSSSYLAWYQQKPGQAPR | L2 (A2) |
| | LLIYGASSRATGIPDRFSGSGSGTDFTLTISRLEPEDFAVYYCQQYG | “aCTLA-4.4” |
| | RSPFTFGPGTKVDIK | |
| 3 | EIVLTQSPGTLSLSPGERATLSCRASQSVSSSYLAWYQQKPGQAPR | L3 (A3) |
| | LLIYGASSRATGIPDRFSGSGSGTDFTLTISRLEPEDFAVYYCQQYG | “aCTLA-4.2” |
| | SSPFTFGPGTKVDIK | |
| 4 | EIVLTQSPGTLSLSPGDRATLSCRASQSGSSSYLAWYQQKPGQAPR | L4 (A4) |
| | LLIYGASSRATGIPDRFSGSGSGTDFTLTISRLEPEDFAVYYCQQYG | “aCTLA-4.29” |
| | TSPFTSGPGTKVDIK | |
| 5 | EIVLTQSPGTLSLSPGDRATLSCRASQSGSSSYLAWYQQKPGQAPR | L5 (A5) |
| | LLIYGASSRATGIPDRFSGSGSGTDFTLTISRLEPEDFAVYYCQQYG | “aCTLA-4.28” |
| | TSPFTSGPGTKVDIK | |
| 6 | DIQMTQSPSSLSASVGDRVTITCRASQSISSYLNWYQQKPGKAPKL | L6 (A6) |
| | LIYAASSLQSGVPSRFSGSGSGTDFTLTISSLQPEDFATYYCQQSYS | “aCTLA-4.26” |
| | TPFTFGPGTKVDIK | |
| 7 | EIVLTQSPGTLSLSPGERATLSCRASQSVSSSYLAWYQQKPGQAPR | L7 (A7) |
| | LLIYGASSRATGIPDRFSGSGSGTDFTLTISRLEPEDFAVYYCQQYG | “aCTLA-4.3” |
| | SSPFTFGPGTKVDIK | |
| 8 | EIVLTQSPGTLSLSPGERATLSCRASQSVSYLAWYQQKPGQAPRLL | L8 (A8) |
| | IYGASSRATGIPDRFSGSGSGTDFTLTISRLEPEDFAVYYCQQYGSS | “aCTLA-4.1” |
| | PWTFGQGTKVEIK | |
| 9 | DIQMTQSPSSLSASVGDRVTITCRASQSISSYLNWYQQKPGKAPKL | L9 (A9) |
| | LIYAASSLQSGVPSRFSGSGSGTDFTLTISSLQPEDFATYYCQQSYS | “aCTLA-4.24” |
| | TPFTFGPGTKVDIK | |
| 10 | EIVLTQSPGTLSLSPGERATLSCRASQSVSSSYLAWYQQKPGQAPR | L10 (A10) |
| | LLIYGASSRATGIPDRFSGSGSGTDFTLTISRLEPEDFAVYYCQQYG | “aCTLA-4.22” |
| | SSPFTFGPGTKVDIK | |
| 11 | DIQLTQSPSFLSASVGDRVTITCRASQGISSYLAWYQQKPGKAPKL | L11 (A11) |
| | LIYAASTLQSGVPSRFSGSGSGTEFTLTISSLQPEDFATYYCQQLNS | “aCTLA-4.31” |
| | YPPTFGQGTKVEIK | |
| 12 | EIVLTQSPGTLSLSPGERATLSCRASQSVSSSYLAWYQQKPGQAPR | L12 (A12) |
| | LLIYGASSRATGIPDRFSGSGSGTDFTLTISRLEPEDFAVYYCQQYG | “aCTLA-4.12” |
| | TSPWTFGQGTKVEIK | |
| 13 | EIVLTQSPGTLSLSPGERATLSCRASQSVSSSYLAWYQQKPGQAPR | L13 (A13) |
| | LLIYGASSRATGIPDRFSGSGSGTDFTLTISRLEPEDFAVYYCQQYG | “aCTLA-4.14” |
| | SSPFTFGPGTKVDIK | |
| 14 | EIVLTQSPGTLSLSPGERATLSCRASQSVSSSYLAWYQQKPGQAPR | L14 (A14), |
| | LLIYGASSRATGIPDRFSGSGSGTDFTLTISRLEPEDFAVYYCQQYG | “aCTLA-4.15” |
| | SSPWTSGQGTKVEIK | |
| 15 | EIVLTQSPGTLSLSPGERATLSCRASQSVSSSYLAWYQQKPGQAPR | L15 (A15) |
| | LLIYGASSRATGIPDRFSGSGSGTDFTLTISRLEPEDFAVYYCQQYG | “aCTLA-4.18” |
| | SSPFTFGPGTKVDIK | |
| 16 | DIQMTQSPSSLSASVGDRVTITCRASQGISNYLAWYQQKPGKVPK | L16 (A16) |
| | LLIYAASTLQSGVPSRFSGSGSGTDFTLTISSLQPEDVATYYCQNYN | “aCTLA-4.5” |
| | SAPWTFGQGTKVEIK | |
| 17 | DIQMTQSPSSLSASVGDRVTITCRASQAIRNDLGWYQQKPGKAPK | L17 (A17) |
| | RLIYAASSLQSGVPPRFSGSGSGTEFTLTISSLQPEDFATYYCLQHN | “aCTLA-4.17” |
| | SYPLTFGGGTKVEIK | |
| 18 | EIVLTQSPGTLSLSPGERATLSCRASQSVSYLAWYQQKPGQAPRLL | L18 (A18) |
| | IYGASSRATGIPDRFSGSGSGTDFTLTISRLEPEDFAVYYCQQYGSS | “aCTLA-4.11” |
| | PWTFGQGTKVEIK | |
| 19 | EIVLTQSPGTLSLSPGERATLSCRASQSVSSSYLAWYQQKPGQAPR | L19 (A19) |
| | LLIYGASSRATGIPDRFSGSGSGTDFTLTISRLEPEDFAVYYCQQYG | “aCTLA-4.7” |
| | SSSMYTFGQGTKLEIK | |
| 20 | EIVLTQSPGTLSLSPGERATLSCRASQSVSSSYLAWYQQKPGQAPR | L20 (A20) |
| | LLIYGASSRATGIPDRFSGSGSGTDFTLTISRLEPEDFAVYYCQQYG | “aCTLA-4.25” |
| | RSPFTFGPGTKVDIK | |
| 21 | DIVMTQSPLSLPVTPGEPASISCRSSQSLLHSNGYNYLDWYLQKPG | L21 (A21) |
| | QSPQLLIYLGSNRASGVPDRFSGSGSGTDFTLKISRVEAEDVGVYY | “aCTLA-4.10” |
| | CMQTLQTPLTFGGGTKVEIK | |
| 22 | DIQMTQSPSSLSASVGDRVTITCRASQAIRNDLGWYQQKPGKAPK | L22 (A22) |
| | RLIYAASSLQSGVPPRFSGSGSGTEFTLTISSLQPEDFATYYCLQHN | “aCTLA-4.21” |
| | SYPLTFGGGTKVEIK | |
| 23 | EIVLTQSPGTLSLSPGERATLSCRASQSVSSSYLAWYQQKPGQAPR | L23 (A23) |
| | LLIYGASSRATGIPDRFSGSGSGTDFTLTISRLEPEDFAVYYCQQYG | “aCTLA-4.23” |
| | PSPWTFGQGTKVEIK | |
| 24 | DIQMTQSPSSLSASVGDRVTITCRASQSISSYLNWYQQKPGKAPKL | L24 (A24) |
| | LIYAASSLQSGVPSRFSGSGSGTDFTLTISSLQPEDFATYYCQQSYR | “aCTLA-4.27” |
| | TPFTFGPGTKVDIK | |
| 25 | EIVMTQSPATLSLSPGERATLSCRASQSVSSSYLSWYQQKPGQAPR | L25 (A25) |
| | LLIYGASTRATGIPARFSGSGSGTDFTLTISNLQPEDFAVYYCQQGY | “aCTLA-4.32” |
| | NLPFTAGPGTKVDIK | |
| 26 | DVVMTQSPLSLPVTLGQPASISCRSSQSLVYSDGNTYLNWFQQRP | L26 (A26) |
| | GQSPRRLIYKVSNRDSGVPDRFSGSGSGTDFTLKISRVEAEDVGVY | “aCTLA-4.20” |
| | YCMQGTHWPITSGQGTRLEIK | |
| 27 | EIVLTQSPGTLSLSPGERATLSCRASQSVSSSYLAWYQQKPGQAPR | L27 (A27) |
| | LLIYGASSRATGIPDRFSGSGSGTDFTLTISRLEPEDFAVYHCQQYG | “aCTLA-4.8” |
| | RSPWTLGQGTKVEIK | |
| 28 | EIVLTQSPGTLSLSPGERATLSCRASQSVSSSYLAWYQQKPGQAPR | L28 (A28) |
| | LLIYGASSRATGIPDRFSGSGSGTDFTLTISRLEPEDFAVYYCQQYG | “aCTLA-4.19” |
| | SSPWTSGQGTKVEIK | |
| 101 | QVQLVESGGGVVQPGRSLRLSCAASGFTFSSFGMHWVRQAPGKG | H1 (A1) |
| | LEWVAVIWYDGRNKYYVDSVKGRFTISRDNSNNTLYLQMNSLRA | “aCTLA-4.9” |
| | EDTAVYYCARGEFFGEFFDYWGQGTLVTVSSA | |
| 102 | QVQLVESGGGVVQPGRSLRLSCAASGFTFSNYGMNWVRQAPGK | H2 (A2) |
| | GLEWVAVIWYDGRNKHY ADSVKGRFTISRDNSKNTLYLQMNSLR | “aCTLA-4.4” |
| | AEDTAVYYCARGGDWGPYFDYWGQGTLVTVSSA | |
| 103 | QVQLVESGGGVVQPGRSLRLSCIASGFTFSSYGMHWVRQAPGKG | H3 (A3) |
| | LEWVAVNWYDGSNKHYADSVKGRFTISRDNSKNTLYLQMNSLR | “aCTLA-4.2” |
| | AEDTAVYYCARGGVWGPYFDYWGQGTLVTVSSA | |
| 104 | QVQLVESGGGVVQPGRSLRISCAASGFTFSSYGIHWVRQAPGKGL | H4 (A4) |
| | QWVAVIWYDGRNKYYADSVKGRFTISRDNSKNTLYLQMNSLRA | “aCTLA-4.29” |
| | EDTAVYYCSRSGSFGAFDIWGQGTMVTVSSA | |
| 105 | QVQLVESGGGVVQPGRSLRLSCAASGFTFSSYGMHWVRQAPGKG | H5 (A5) |
| | LEWVAVIWYDGNNKYYADSVKGRFTISRDNSKNTLYLQMYSLRA | “aCTLA-4.28” |
| | EDTAVYYCARGGILAAGIFDYWGQGTLVTVSSA | |
| 106 | QVQLVESGGGVVQPGRSLRLSCAASGFTFSSYGMHWVRQAPGKG | H6 (A6) |
| | LEWVAVIWYDGSYKYYADSVKGRFTISRDDSKNTLYLQMSSLRA | “aCTLA-4.26” |
| | EDTAVYYCARAPHYAILTGYYEDYWGQGTLVTVSSA | |
| 107 | QVQLVESGGGVVQPGRSLRLSCAASGFTLSSFGMHWVRQAPGKG | H7 (A7) |
| | LEWVAVIWYDGSNKYYADSVKGRFIISRDNSKTALYLQMNSLRA | “aCTLA-4.3” |
| | EDTAVYYCARAHYFGAFDIWGQGTMVTVSSA | |
| 108 | QVQLVESGGGVVQPGRSLRLSCAASGFTFSRYGMHWVRQAPGKG | H8 (A8) |
| | LEWVAVIWYDGRNKYYADSVKGRFTISRDNSKNTLYLQMNSLRA | “aCTLA-4.1” |
| | EDTALYSCARAGELGPFDYWGQGTLVTVSSA | |
| 109 | QVQLVESGGGVVQPGRSLRLSCAASGFTFSSHGMHWVRQAPGKG | H9 (A9) |
| | LEWVAVIWYDGSNKHYADSVKGRFTISRDNSKNTLYLQMNSLRA | “aCTLA-4.24” |
| | EDTAVYYCARGDILTGYYGYWGQGTLVTVSSA | |
| 110 | QVQLVESGGGVVQPGRSLRLSCVASGFTLSSYGMHWVRQAPGKG | H10 (A10) |
| | LEWVAVIWYDGSNKHYADSVKGRFTISRDNSKNTLSLQMNSLRA | “aCTLA-4.22” |
| | EDTAVYYCARGGQLGPFDYWGQGTLVTVSSA | |
| 111 | QVQLVESGGGVVQPGRSLRLSCAASGFTFSSYGMHWVRQAPGKG | H11 (A11) |
| | LEWVAVIWYDVGNKYYIDSVKGRFTISRDNSKNTLYLQMNSLRA | “aCTLA-4.3]” |
| | EDTAVYYCARDYYGSGSPRHFDYWGQGTLVTVSSA | |
| 112 | QVQLVESGGGVVQPGRSLRLSCAASGFTFSSYGMHWVRQAPGKG | H12 (A12) |
| | LEWVAVIWYDGSNKYYADSVKGRFTFSRDNSKNTLYLQMNSLR | “aCTLA-4.12” |
| | AEDTAVYYCARGGLMGAFDYWGQGTLVTVSSA | |
| 113 | QVQLVESGGGVVQPGRSLRLSCAASGFTFSSYGMHWVRQAPGKG | H13 (A13) |
| | LEWVAVIWYDGRNKDYADSVKGRITISRDNSKNTLYLQMNSLRA | “aCTLA-4.14” |
| | EDTAVYYCARGGLLGPYFDYWGQGTLVTVSSA | |
| 114 | QVQLVESGGGVVQPGRSLRLSCAASGFTFSSYGMHWVRQAPGTG | H14 (A14) |
| | LEWVAVIWYEGRNKYYADPVKGRFTISRDNSKNTLYLQMNSLRD | “aCTLA-4.15” |
| | DDTAVYYCARAGDLGAFDIWGQGTMVTVSSA | |
| 115 | QVQLVESGGGVVQPGRSLRLSCAASGFTFRSYGMHWVRQAPGKG | H15 (A15) |
| | LEWVAVIWYDGSNKHYADSVKGRFTISRDNSKNTLYLQMNSLRA | “aCTLA-4.18” |
| | EDTAVYYCARNGLIGAFDIWGQGTMVTVSSA | |
| 116 | QVQLVESGGGVVQPGRSLRLSCAASGFTFSSYGIHWVRQAPGKGL | H16 (A16) |
| | EWVAVIWYDGSNKYYADSVKGRFTISRDNSKNTLYLQMNSLRAE | “aCTLA-4.5” |
| | DTAVYYCARGSLLGPFDYWGQGTLVTVSSA | |
| 117 | EVQLVESGGGLVQPGGSLRLSCAASGFTFSNYAMSWVRQAPGKG | H17 (A17) |
| | LEWVSAISGGGLSTYYADSVKGRFTISRDNSKNTLYLQMNSLRAE | “aCTLA-4.17” |
| | DTAVYYCAKDLLWLGFDYWGQGTLVTVSSA | |
| 118 | QVQLVESGGGVVQPGRSLRLSCAASGFTFSSYGMHWVRQAPGKG | H18 (A18) |
| | LEWVAVIWYDGSNKFYADSVKGRFTISSDNSKNTLYLQMNSLRA | “aCTLA-4.11” |
| | EDTAVYYCARGGHLGSFDYWGQGTLVTVSSA | |
| 119 | QVQLVQSGAEVKKPGASVKVSCKASGYTFTSYYMHWVRQAPGQ | H19 (A19) |
| | GLEWMGIINPSVGSTSYAQKFQGRVTMTRDTSTSTVYMELSSLRS | “aCTLA-4.7” |
| | EDTAVYYCAREVRVRGVIIPFFDYWDQGTLVTVSSA | |
| 120 | QVQLVQSGAEVKKPGASVKVSCKASGYTFTSYGISWVRQAPGQG | H20 (A20) |
| | LEWMGWISAYNGNTNYAQKFQGRVTMTTDTSTSTAYMELRSLRS | “aCTLA-4.25” |
| | DDTAVYYCAKVSGYFDYWGQGTLVTVSSA | |
| 121 | QVQLVESGGGVVQPGRSLRLSCAASGFTFSSYGMHWVRQAPGKG | H21 (A21) |
| | LEWVAVIWYDGNNKYYADSVKGRFTISRDNSKNTLYLHMNSLRA | “aCTLA-4.10” |
| | DDTAVYYCARMLRGAPYYYGMDVWGQGTTVTVSSA | |
| 122 | EVOLVESGGGLVQPGGSLRLSCAASGFTFSSYAMSWVRQAPGKG | H22 (A22) |
| | LEWVSGISGSGGSTYYADSVKGRFTISRDNSKNTLYLQMNSLRAE | “aCTLA-4.2]” |
| | DTAVYYCAKLGIAWYFDVWGRGTLVTVSSA | |
| 123 | QVQLVQSGAEVKKPGASVKVSCKASGYTFTSYGISWVRQAPGQG | H23 (A23) |
| | LEWMGWISAYNGNTYYAQKFQGRVTMTTDTSTSTAYVELRSLRS | “aCTLA-4.23” |
| | DDTAVYYCARVTGRDAFDIWGQGTMVTVSSA | |
| 124 | QVQLVQSGAEVKKPGASVKVSCKASGYTFTSYGISWVRQAPGQG | H24 (A24) |
| | LECMGWISAYNGNTNYAQKFQGRVTMITDTSTSTAYMELRSLRS | “aCTLA-4.27” |
| | DDTAVYYCARVGPINLDYWGQGTLVTVSSA | |
| 125 | QVQLVQSGAEVKKPGASVKVSCKASGYTFTNYGISWVRQAPGQG | H25 (A25) |
| | LEWMGWISVYNGNTNYAQKFQGRVTMTTDTSTSTAYMELRSLIS | “aCTLA-4.32” |
| | DDTAVYYCARLGKGLFDYWGQGTLVTVSSA | |
| 126 | QVQLVQSGAEVKKPGASVKVSCKASDYTFTYYGISWVRQAPGQG | H26 (A26) |
| | LEWMGWISAYNGNTNYAQKLQGRVTMTTDTSTNTAYLELRSLRS | “aCTLA-4.20” |
| | DDTAVYYCARDYYDSSGYFDYWGQGTLVTVSSA | |
| 127 | QVQLVQSGAEVKKPGASVKVSCKASGYTFTSYGISWVRQAPGQG | H27 (A27) |
| | LEWMGWISAYNGNTNYAQKLQGRVTMTTDTSTSTAYMELRSLR | “aCTLA-4.8” |
| | SDDTAVYYCGRWVRGVEYWGQGTLVTVSSA | |
| 128 | QVQLVESGGGVVQPGRSLGLSCAASGFTFSTYGMHWVRQAPGKG | H28 (A28) |
| | LEWVAVTLYDGSNKYYADSVKGRFTISRDNSKNTLYLQMNSLRA | “aCTLA-4.19” |
| | EDTAVYYCARASLTGSFDYWGQGTLVTVSSA | |
| 1001 | QSFSSNY | CDR1-L1 (A1) |
| 1002 | QSVSSSY | CDR1-L2 (A2) |
| 1003 | QSVSSSY | CDR1-L3 (A3) |
| 1004 | QSGSSSY | CDR1-L4 (A4) |
| 1005 | QSGSSSY | CDR1-L5 (A5) |
| 1006 | QSISSY | CDR1-L6 (A6) |
| 1007 | QSVSSSY | CDR1-L7 (A7) |
| 1008 | QSVSY | CDR1-L8 (A8) |
| 1009 | QSISSY | CDR1-L9 (A9) |
| 1010 | QSVSSSY | CDR1-L10 |
| | | (A10) |
| 1011 | QGISSY | CDR1-L11 |
| | | (A11) |
| 1012 | QSVSSSY | CDR1-L12 |
| | | (A12) |
| 1013 | QSVSSSY | CDR1-L13 |
| | | (A13) |
| 1014 | QSVSSSY | CDR1-L14 |
| | | (A14) |
| 1015 | QSVSSSY | CDR1-L15 |
| | | (A15) |
| 1016 | QGISNY | CDR1-L16 |
| | | (A16) |
| 1017 | QAIRND | CDR1-L17 |
| | | (A17) |
| 1018 | QSVSY | CDR1-L18 |
| | | (A18) |
| 1019 | QSVSSSY | CDR1-L19 |
| | | (A19) |
| 1020 | QSVSSSY | CDR1-L20 |
| | | (A20) |
| 1021 | QSLLHSNGYNY | CDR1-L21 |
| | | (A21) |
| 1022 | QAIRND | CDR1-L22 |
| | | (A22) |
| 1023 | QSVSSSY | CDR1-L23 |
| | | (A23) |
| 1024 | QSISSY | CDR1-L24 |
| | | (A24) |
| 1025 | QSVSSSY | CDR1-L25 |
| | | (A25) |
| 1026 | QSLVYSDGNTY | CDR1-L26 |
| | | (A26) |
| 1027 | QSVSSSY | CDR1-L27 |
| | | (A27) |
| 1028 | QSVSSSY | CDR1-L28 |
| | | (A28) |
| 2001 | GAS | CDR2-L1 (A1) |
| 2002 | GAS | CDR2-L2 (A2) |
| 2003 | GAS | CDR2-L3 (A3) |
| 2004 | GAS | CDR2-L4 (A4) |
| 2005 | GAS | CDR2-L5 (A5) |
| 2006 | AAS | CDR2-L6 (A6) |
| 2007 | GAS | CDR2-L7 (A7) |
| 2008 | GAS | CDR2-L8 (A8) |
| 2009 | AAS | CDR2-L9 (A9) |
| 2010 | GAS | CDR2-L10 |
| | | (A10) |
| 2011 | AAS | CDR2-L11 |
| | | (A11) |
| 2012 | GAS | CDR2-L12 |
| | | (A12) |
| 2013 | GAS | CDR2-L13 |
| | | (A13) |
| 2014 | GAS | CDR2-L14 |
| | | (A14) |
| 2015 | GAS | CDR2-L15 |
| | | (A15) |
| 2016 | AAS | CDR2-L16 |
| | | (A16) |
| 2017 | AAS | CDR2-L17 |
| | | (A17) |
| 2018 | GAS | CDR2-L18 |
| | | (A18) |
| 2019 | GAS | CDR2-L19 |
| | | (A19) |
| 2020 | GAS | CDR2-L20 |
| | | (A20) |
| 2021 | LGS | CDR2-L21 |
| | | (A21) |
| 2022 | AAS | CDR2-L22 |
| | | (A22) |
| 2023 | GAS | CDR2-L23 |
| | | (A23) |
| 2024 | AAS | CDR2-L24 |
| | | (A24) |
| 2025 | GAS | CDR2-L25 |
| | | (A25) |
| 2026 | KVS | CDR2-L26 |
| | | (A26) |
| 2027 | GAS | CDR2-L27 |
| | | (A27) |
| 2028 | GAS | CDR2-L28 |
| | | (A28) |
| 3001 | QQYGTSPFT | CDR3-L1 (A1) |
| 3002 | QQYGRSPFT | CDR3-L2 (A2) |
| 3003 | QQYGSSPFT | CDR3-L3 (A3) |
| 3004 | QQYGTSPFT | CDR3-L4 (A4) |
| 3005 | QQYGTSPFT | CDR3-L5 (A5) |
| 3006 | QQSYSTPFT | CDR3-L6 (A6) |
| 3007 | QQYGSSPFT | CDR3-L7 (A7) |
| 3008 | QQYGSSPWT | CDR3-L8 (A8) |
| 3009 | QQSYSTPFT | CDR3-L9 (A9) |
| 3010 | QQYGSSPFT | CDR3-L10 |
| | | (A10) |
| 3011 | QQLNSYPPT | CDR3-L11 |
| | | (A11) |
| 3012 | QQYGTSPWT | CDR3-L12 |
| | | (A12) |
| 3013 | QQYGSSPFT | CDR3-L13 |
| | | (A13) |
| 3014 | QQYGSSPWT | CDR3-L14 |
| | | (A14) |
| 3015 | QQYGSSPFT | CDR3-L15 |
| | | (A15) |
| 3016 | QNYNSAPWT | CDR3-L16 |
| | | (A16) |
| 3017 | LOHNSYPLT | CDR3-L17 |
| | | (A17) |
| 3018 | QQYGSSPWT | CDR3-L18 |
| | | (A18) |
| 3019 | QQYGSSSMYT | CDR3-L19 |
| | | (A19) |
| 3020 | QQYGRSPFT | CDR3-L20 |
| | | (A20) |
| 3021 | MQTLQTPLT | CDR3-L21 |
| | | (A21) |
| 3022 | LOHNSYPLT | CDR3-L22 |
| | | (A22) |
| 3023 | QQYGPSPWT | CDR3-L23 |
| | | (A23) |
| 3024 | QQSYRTPFT | CDR3-L24 |
| | | (A24) |
| 3025 | QQGYNLPFT | CDR3-L25 |
| | | (A25) |
| 3026 | MQGTHWPIT | CDR3-L26 |
| | | (A26) |
| 3027 | QQYGRSPWT | CDR3-L27 |
| | | (A27) |
| 3028 | QQYGSSPWT | CDR3-L28 |
| | | (A28) |
| 4001 | GFTFSSFG | CDR1-H1 |
| | | (A1) |
| 4002 | GFTFSNYG | CDR1-H2 |
| | | (A2) |
| 4003 | GFTFSSYG | CDR1-H3 |
| | | (A3) |
| 4004 | GFTFSSYG | CDR1-H4 |
| | | (A4) |
| 4005 | GFTFSSYG | CDR1-H5 |
| | | (A5) |
| 4006 | GFTFSSYG | CDR1-H6 |
| | | (A6) |
| 4007 | GFTLSSFG | CDR1-H7 |
| | | (A7) |
| 4008 | GFTFSRYG | CDR1-H8 |
| | | (A8) |
| 4009 | GFTFSSHG | CDR1-H9 |
| | | (A9) |
| 4010 | GFTLSSYG | CDR1-H10 |
| | | (A10) |
| 4011 | GFTFSSYG | CDR1-H11 |
| | | (A11) |
| 4012 | GFTFSSYG | CDR1-H12 |
| | | (A12) |
| 4013 | GFTFSSYG | CDR1-H13 |
| | | (A13) |
| 4014 | GFTFSSYG | CDR1-H14 |
| | | (A14) |
| 4015 | GFTFRSYG | CDR1-H15 |
| | | (A15) |
| 4016 | GFTFSSYG | CDR1-H16 |
| | | (A16) |
| 4017 | GFTFSNYA | CDR1-H17 |
| | | (A17) |
| 4018 | GFTFSSYG | CDR1-H18 |
| | | (A18) |
| 4019 | GYTFTSYY | CDR1-H19 |
| | | (A19) |
| 4020 | GYTFTSYG | CDR1-H20 |
| | | (A20) |
| 4021 | GFTFSSYG | CDR1-H21 |
| | | (A21) |
| 4022 | GFTFSSYA | CDR1-H22 |
| | | (A22) |
| 4023 | GYTFTSYG | CDR1-H23 |
| | | (A23) |
| 4024 | GYTFTSYG | CDR1-H24 |
| | | (A24) |
| 4025 | GYTFTNYG | CDR1-H25 |
| | | (A25) |
| 4026 | DYTFTYYG | CDR1-H26 |
| | | (A26) |
| 4027 | GYTFTSYG | CDR1-H27 |
| | | (A27) |
| 4028 | GFTFSTYG | CDR1-H28 |
| | | (A28) |
| 5001 | IWYDGRNK | CDR2-H1 |
| | | (A1) |
| 5002 | IWYDGRNK | CDR2-H2 |
| | | (A2) |
| 5003 | NWYDGSNK | CDR2-H3 |
| | | (A3) |
| 5004 | IWYDGRNK | CDR2-H4 |
| | | (A4) |
| 5005 | IWYDGNNK | CDR2-H5 |
| | | (A5) |
| 5006 | IWYDGSYK | CDR2-H6 |
| | | (A6) |
| 5007 | IWYDGSNK | CDR2-H7 |
| | | (A7) |
| 5008 | IWYDGRNK | CDR2-H8 |
| | | (A8) |
| 5009 | IWYDGSNK | CDR2-H9 |
| | | (A9) |
| 5010 | IWYDGSNK | CDR2-H10 |
| | | (A10) |
| 5011 | IWYDVGNK | CDR2-H11 |
| | | (A11) |
| 5012 | IWYDGSNK | CDR2-H12 |
| | | (A12) |
| 5013 | IWYDGRNK | CDR2-H13 |
| | | (A13 |
| 5014 | IWYEGRNK | CDR2-H14 |
| | | (A14) |
| 5015 | IWYDGSNK | CDR2-H15 |
| | | (A15) |
| 5016 | IWYDGSNK | CDR2-H16 |
| | | (A16) |
| 5017 | ISGGGLST | CDR2-H17 |
| | | (A17) |
| 5018 | IWYDGSNK | CDR2-H18 |
| | | (A18) |
| 5019 | INPSVGST | CDR2-H19 |
| | | (A19) |
| 5020 | ISAYNGNT | CDR2-H20 |
| | | (A20) |
| 5021 | IWYDGNNK | CDR2-H21 |
| | | (A21) |
| 5022 | ISGSGGST | CDR2-H22 |
| | | (A22) |
| 5023 | ISAYNGNT | CDR2-H23 |
| | | (A23) |
| 5024 | ISAYNGNT | CDR2-H24 |
| | | (A24) |
| 5025 | ISVYNGNT | CDR2-H25 |
| | | (A25) |
| 5026 | ISAYNGNT | CDR2-H26 |
| | | (A26) |
| 5027 | ISAYNGNT | CDR2-H27 |
| | | (A27) |
| 5028 | TLYDGSNK | CDR2-H28 |
| | | (A28) |
| 6001 | ARGEFFGEFFDY | CDR3-H1 |
| | | (A1) |
| 6002 | ARGGDWGPYFDY | CDR3-H2 |
| | | (A2) |
| 6003 | ARGGVWGPYFDY | CDR3-H3 |
| | | (A3) |
| 6004 | SRSGSFGAFDI | CDR3-H4 |
| | | (A4) |
| 6005 | ARGGILAAGIFDY | CDR3-H5 |
| | | (A5) |
| 6006 | ARAPHYAILTGYYEDY | CDR3-H6 |
| | | (A6) |
| 6007 | ARAHYFGAFDI | CDR3-H7 |
| | | (A7) |
| 6008 | ARAGELGPFDY | CDR3-H8 |
| | | (A8) |
| 6009 | ARGDILTGYYGY | CDR3-H9 |
| | | (A9) |
| 6010 | ARGGQLGPFDY | CDR3-H10 |
| | | (A10) |
| 6011 | ARDYYGSGSPRHFDY | CDR3-H11 |
| | | (A11) |
| 6012 | ARGGLMGAFDY | CDR3-H12 |
| | | (A12) |
| 6013 | ARGGLLGPYFDY | CDR3-H13 |
| | | (A13) |
| 6014 | ARAGDLGAFDI | CDR3-H14 |
| | | (A14) |
| 6015 | ARNGLIGAFDI | CDR3-H15 |
| | | (A15) |
| 6016 | ARGSLLGPFDY | CDR3-H16 |
| | | (A16) |
| 6017 | AKDLLWLGFDY | CDR3-H17 |
| | | (A17) |
| 6018 | ARGGHLGSFDY | CDR3-H18 |
| | | (A18) |
| 6019 | AREVRVRGVIIPFFDY | CDR3-H19 |
| | | (A19) |
| 6020 | AKVSGYFDY | CDR3-H20 |
| | | (A20) |
| 6021 | ARMLRGAPYYYGMDV | CDR3-H21 |
| | | (A21) |
| 6022 | AKLGIAWYFDV | CDR3-H22 |
| | | (A22) |
| 6023 | ARVTGRDAFDI | CDR3-H23 |
| | | (A23) |
| 6024 | ARVGPINLDY | CDR3-H24 |
| | | (A24) |
| 6025 | ARLGKGLFDY | CDR3-H25 |
| | | (A25) |
| 6026 | ARDYYDSSGYFDY | CDR3-H26 |
| | | (A26) |
| 6027 | GRWVRGVEY | CDR3-H27 |
| | | (A27) |
| 6028 | ARASLTGSFDY | CDR3-H28 |
| | | (A28) |
| 7001 | MACLGFQRHKAQLNLAARTWPCTLLFFLLFIPVFCKAMHVAQPA | Human |
| | VVLASSRGIASFVCEYASPGKATEVRVTVLRQADSQVTEVCAATY | CTLA4 |
| | MMGNELTFLDDSICTGTSSGNQVNLTIQGLRAMDTGLYICKVELM | |
| | YPPPYYLGIGNGTQIYVIDPEPCPDSDFLLWILAAVSSGLFFYSFLLT | |
| | AVSLSKMLKKRSPLTTGVYVKMPPTEPECEKQFQPYFIPIN | |

### Table 20 Provides the Sequence Identifiers for the Light Chain, Heavy Chain, and CDRs of the Indicated Clones

TABLE 20

| Antibody | SEQ ID NO | | | | | | | |
|---|---|---|---|---|---|---|---|---|
| Clone | Light | Heavy | CDR1 | CDR2 | CDR3 | CDR1 | CDR2 | CDR3 |
| Number | Chain | Chain | (Light) | (Light) | (Light) | (Heavy) | (Heavy) | (Heavy) |
| 1 | 8000 | 8496 | 8992 | 9488 | 9984 | 10480 | 10976 | 11472 |
| 2 | 8001 | 8497 | 8993 | 9489 | 9985 | 10481 | 10977 | 11473 |
| 3 | 8002 | 8498 | 8994 | 9490 | 9986 | 10482 | 10978 | 11474 |
| 4 | 8003 | 8499 | 8995 | 9491 | 9987 | 10483 | 10979 | 11475 |
| 5 | 8004 | 8500 | 8996 | 9492 | 9988 | 10484 | 10980 | 11476 |
| 6 | 8005 | 8501 | 8997 | 9493 | 9989 | 10485 | 10981 | 11477 |
| 7 | 8006 | 8502 | 8998 | 9494 | 9990 | 10486 | 10982 | 11478 |
| 8 | 8007 | 8503 | 8999 | 9495 | 9991 | 10487 | 10983 | 11479 |
| 9 | 8008 | 8504 | 9000 | 9496 | 9992 | 10488 | 10984 | 11480 |
| 10 | 8009 | 8505 | 9001 | 9497 | 9993 | 10489 | 10985 | 11481 |
| 11 | 8010 | 8506 | 9002 | 9498 | 9994 | 10490 | 10986 | 11482 |
| 12 | 8011 | 8507 | 9003 | 9499 | 9995 | 10491 | 10987 | 11483 |
| 13 | 8012 | 8508 | 9004 | 9500 | 9996 | 10492 | 10988 | 11484 |
| 14 | 8013 | 8509 | 9005 | 9501 | 9997 | 10493 | 10989 | 11485 |
| 15 | 8014 | 8510 | 9006 | 9502 | 9998 | 10494 | 10990 | 11486 |
| 16 | 8015 | 8511 | 9007 | 9503 | 9999 | 10495 | 10991 | 11487 |
| 17 | 8016 | 8512 | 9008 | 9504 | 10000 | 10496 | 10992 | 11488 |
| 18 | 8017 | 8513 | 9009 | 9505 | 10001 | 10497 | 10993 | 11489 |
| 19 | 8018 | 8514 | 9010 | 9506 | 10002 | 10498 | 10994 | 11490 |
| 20 | 8019 | 8515 | 9011 | 9507 | 10003 | 10499 | 10995 | 11491 |
| 21 | 8020 | 8516 | 9012 | 9508 | 10004 | 10500 | 10996 | 11492 |
| 22 | 8021 | 8517 | 9013 | 9509 | 10005 | 10501 | 10997 | 11493 |
| 23 | 8022 | 8518 | 9014 | 9510 | 10006 | 10502 | 10998 | 11494 |
| 24 | 8023 | 8519 | 9015 | 9511 | 10007 | 10503 | 10999 | 11495 |
| 25 | 8024 | 8520 | 9016 | 9512 | 10008 | 10504 | 11000 | 11496 |
| 26 | 8025 | 8521 | 9017 | 9513 | 10009 | 10505 | 11001 | 11497 |
| 27 | 8026 | 8522 | 9018 | 9514 | 10010 | 10506 | 11002 | 11498 |
| 28 | 8027 | 8523 | 9019 | 9515 | 10011 | 10507 | 11003 | 11499 |
| 29 | 8028 | 8524 | 9020 | 9516 | 10012 | 10508 | 11004 | 11500 |
| 30 | 8029 | 8525 | 9021 | 9517 | 10013 | 10509 | 11005 | 11501 |
| 31 | 8030 | 8526 | 9022 | 9518 | 10014 | 10510 | 11006 | 11502 |
| 32 | 8031 | 8527 | 9023 | 9519 | 10015 | 10511 | 11007 | 11503 |
| 33 | 8032 | 8528 | 9024 | 9520 | 10016 | 10512 | 11008 | 11504 |
| 34 | 8033 | 8529 | 9025 | 9521 | 10017 | 10513 | 11009 | 11505 |
| 35 | 8034 | 8530 | 9026 | 9522 | 10018 | 10514 | 11010 | 11506 |
| 36 | 8035 | 8531 | 9027 | 9523 | 10019 | 10515 | 11011 | 11507 |
| 37 | 8036 | 8532 | 9028 | 9524 | 10020 | 10516 | 11012 | 11508 |
| 38 | 8037 | 8533 | 9029 | 9525 | 10021 | 10517 | 11013 | 11509 |
| 39 | 8038 | 8534 | 9030 | 9526 | 10022 | 10518 | 11014 | 11510 |
| 40 | 8039 | 8535 | 9031 | 9527 | 10023 | 10519 | 11015 | 11511 |
| 41 | 8040 | 8536 | 9032 | 9528 | 10024 | 10520 | 11016 | 11512 |
| 42 | 8041 | 8537 | 9033 | 9529 | 10025 | 10521 | 11017 | 11513 |
| 43 | 8042 | 8538 | 9034 | 9530 | 10026 | 10522 | 11018 | 11514 |
| 44 | 8043 | 8539 | 9035 | 9531 | 10027 | 10523 | 11019 | 11515 |
| 45 | 8044 | 8540 | 9036 | 9532 | 10028 | 10524 | 11020 | 11516 |
| 46 | 8045 | 8541 | 9037 | 9533 | 10029 | 10525 | 11021 | 11517 |
| 47 | 8046 | 8542 | 9038 | 9534 | 10030 | 10526 | 11022 | 11518 |
| 48 | 8047 | 8543 | 9039 | 9535 | 10031 | 10527 | 11023 | 11519 |
| 49 | 8048 | 8544 | 9040 | 9536 | 10032 | 10528 | 11024 | 11520 |
| 50 | 8049 | 8545 | 9041 | 9537 | 10033 | 10529 | 11025 | 11521 |
| 51 | 8050 | 8546 | 9042 | 9538 | 10034 | 10530 | 11026 | 11522 |
| 52 | 8051 | 8547 | 9043 | 9539 | 10035 | 10531 | 11027 | 11523 |
| 53 | 8052 | 8548 | 9044 | 9540 | 10036 | 10532 | 11028 | 11524 |
| 54 | 8053 | 8549 | 9045 | 9541 | 10037 | 10533 | 11029 | 11525 |
| 55 | 8054 | 8550 | 9046 | 9542 | 10038 | 10534 | 11030 | 11526 |
| 56 | 8055 | 8551 | 9047 | 9543 | 10039 | 10535 | 11031 | 11527 |
| 57 | 8056 | 8552 | 9048 | 9544 | 10040 | 10536 | 11032 | 11528 |
| 58 | 8057 | 8553 | 9049 | 9545 | 10041 | 10537 | 11033 | 11529 |
| 59 | 8058 | 8554 | 9050 | 9546 | 10042 | 10538 | 11034 | 11530 |
| 60 | 8059 | 8555 | 9051 | 9547 | 10043 | 10539 | 11035 | 11531 |
| 61 | 8060 | 8556 | 9052 | 9548 | 10044 | 10540 | 11036 | 11532 |
| 62 | 8061 | 8557 | 9053 | 9549 | 10045 | 10541 | 11037 | 11533 |
| 63 | 8062 | 8558 | 9054 | 9550 | 10046 | 10542 | 11038 | 11534 |
| 64 | 8063 | 8559 | 9055 | 9551 | 10047 | 10543 | 11039 | 11535 |
| 65 | 8064 | 8560 | 9056 | 9552 | 10048 | 10544 | 11040 | 11536 |
| 66 | 8065 | 8561 | 9057 | 9553 | 10049 | 10545 | 11041 | 11537 |
| 67 | 8066 | 8562 | 9058 | 9554 | 10050 | 10546 | 11042 | 11538 |
| 68 | 8067 | 8563 | 9059 | 9555 | 10051 | 10547 | 11043 | 11539 |
| 69 | 8068 | 8564 | 9060 | 9556 | 10052 | 10548 | 11044 | 11540 |
| 70 | 8069 | 8565 | 9061 | 9557 | 10053 | 10549 | 11045 | 11541 |
| 71 | 8070 | 8566 | 9062 | 9558 | 10054 | 10550 | 11046 | 11542 |
| 72 | 8071 | 8567 | 9063 | 9559 | 10055 | 10551 | 11047 | 11543 |
| 73 | 8072 | 8568 | 9064 | 9560 | 10056 | 10552 | 11048 | 11544 |
| 74 | 8073 | 8569 | 9065 | 9561 | 10057 | 10553 | 11049 | 11545 |
| 75 | 8074 | 8570 | 9066 | 9562 | 10058 | 10554 | 11050 | 11546 |
| 76 | 8075 | 8571 | 9067 | 9563 | 10059 | 10555 | 11051 | 11547 |
| 77 | 8076 | 8572 | 9068 | 9564 | 10060 | 10556 | 11052 | 11548 |
| 78 | 8077 | 8573 | 9069 | 9565 | 10061 | 10557 | 11053 | 11549 |
| 79 | 8078 | 8574 | 9070 | 9566 | 10062 | 10558 | 11054 | 11550 |
| 80 | 8079 | 8575 | 9071 | 9567 | 10063 | 10559 | 11055 | 11551 |
| 81 | 8080 | 8576 | 9072 | 9568 | 10064 | 10560 | 11056 | 11552 |
| 82 | 8081 | 8577 | 9073 | 9569 | 10065 | 10561 | 11057 | 11553 |
| 83 | 8082 | 8578 | 9074 | 9570 | 10066 | 10562 | 11058 | 11554 |
| 84 | 8083 | 8579 | 9075 | 9571 | 10067 | 10563 | 11059 | 11555 |
| 85 | 8084 | 8580 | 9076 | 9572 | 10068 | 10564 | 11060 | 11556 |
| 86 | 8085 | 8581 | 9077 | 9573 | 10069 | 10565 | 11061 | 11557 |
| 87 | 8086 | 8582 | 9078 | 9574 | 10070 | 10566 | 11062 | 11558 |
| 88 | 8087 | 8583 | 9079 | 9575 | 10071 | 10567 | 11063 | 11559 |
| 89 | 8088 | 8584 | 9080 | 9576 | 10072 | 10568 | 11064 | 11560 |
| 90 | 8089 | 8585 | 9081 | 9577 | 10073 | 10569 | 11065 | 11561 |
| 91 | 8090 | 8586 | 9082 | 9578 | 10074 | 10570 | 11066 | 11562 |
| 92 | 8091 | 8587 | 9083 | 9579 | 10075 | 10571 | 11067 | 11563 |
| 93 | 8092 | 8588 | 9084 | 9580 | 10076 | 10572 | 11068 | 11564 |
| 94 | 8093 | 8589 | 9085 | 9581 | 10077 | 10573 | 11069 | 11565 |
| 95 | 8094 | 8590 | 9086 | 9582 | 10078 | 10574 | 11070 | 11566 |
| 96 | 8095 | 8591 | 9087 | 9583 | 10079 | 10575 | 11071 | 11567 |
| 97 | 8096 | 8592 | 9088 | 9584 | 10080 | 10576 | 11072 | 11568 |
| 98 | 8097 | 8593 | 9089 | 9585 | 10081 | 10577 | 11073 | 11569 |
| 99 | 8098 | 8594 | 9090 | 9586 | 10082 | 10578 | 11074 | 11570 |
| 100 | 8099 | 8595 | 9091 | 9587 | 10083 | 10579 | 11075 | 11571 |
| 101 | 8100 | 8596 | 9092 | 9588 | 10084 | 10580 | 11076 | 11572 |
| 102 | 8101 | 8597 | 9093 | 9589 | 10085 | 10581 | 11077 | 11573 |
| 103 | 8102 | 8598 | 9094 | 9590 | 10086 | 10582 | 11078 | 11574 |
| 104 | 8103 | 8599 | 9095 | 9591 | 10087 | 10583 | 11079 | 11575 |
| 105 | 8104 | 8600 | 9096 | 9592 | 10088 | 10584 | 11080 | 11576 |
| 106 | 8105 | 8601 | 9097 | 9593 | 10089 | 10585 | 11081 | 11577 |
| 107 | 8106 | 8602 | 9098 | 9594 | 10090 | 10586 | 11082 | 11578 |
| 108 | 8107 | 8603 | 9099 | 9595 | 10091 | 10587 | 11083 | 11579 |
| 109 | 8108 | 8604 | 9100 | 9596 | 10092 | 10588 | 11084 | 11580 |
| 110 | 8109 | 8605 | 9101 | 9597 | 10093 | 10589 | 11085 | 11581 |
| 111 | 8110 | 8606 | 9102 | 9598 | 10094 | 10590 | 11086 | 11582 |
| 112 | 8111 | 8607 | 9103 | 9599 | 10095 | 10591 | 11087 | 11583 |
| 113 | 8112 | 8608 | 9104 | 9600 | 10096 | 10592 | 11088 | 11584 |
| 114 | 8113 | 8609 | 9105 | 9601 | 10097 | 10593 | 11089 | 11585 |
| 115 | 8114 | 8610 | 9106 | 9602 | 10098 | 10594 | 11090 | 11586 |
| 116 | 8115 | 8611 | 9107 | 9603 | 10099 | 10595 | 11091 | 11587 |
| 117 | 8116 | 8612 | 9108 | 9604 | 10100 | 10596 | 11092 | 11588 |
| 118 | 8117 | 8613 | 9109 | 9605 | 10101 | 10597 | 11093 | 11589 |
| 119 | 8118 | 8614 | 9110 | 9606 | 10102 | 10598 | 11094 | 11590 |
| 120 | 8119 | 8615 | 9111 | 9607 | 10103 | 10599 | 11095 | 11591 |
| 121 | 8120 | 8616 | 9112 | 9608 | 10104 | 10600 | 11096 | 11592 |
| 122 | 8121 | 8617 | 9113 | 9609 | 10105 | 10601 | 11097 | 11593 |
| 123 | 8122 | 8618 | 9114 | 9610 | 10106 | 10602 | 11098 | 11594 |
| 124 | 8123 | 8619 | 9115 | 9611 | 10107 | 10603 | 11099 | 11595 |
| 125 | 8124 | 8620 | 9116 | 9612 | 10108 | 10604 | 11100 | 11596 |
| 126 | 8125 | 8621 | 9117 | 9613 | 10109 | 10605 | 11101 | 11597 |
| 127 | 8126 | 8622 | 9118 | 9614 | 10110 | 10606 | 11102 | 11598 |
| 128 | 8127 | 8623 | 9119 | 9615 | 10111 | 10607 | 11103 | 11599 |
| 129 | 8128 | 8624 | 9120 | 9616 | 10112 | 10608 | 11104 | 11600 |
| 130 | 8129 | 8625 | 9121 | 9617 | 10113 | 10609 | 11105 | 11601 |
| 131 | 8130 | 8626 | 9122 | 9618 | 10114 | 10610 | 11106 | 11602 |
| 132 | 8131 | 8627 | 9123 | 9619 | 10115 | 10611 | 11107 | 11603 |
| 133 | 8132 | 8628 | 9124 | 9620 | 10116 | 10612 | 11108 | 11604 |
| 134 | 8133 | 8629 | 9125 | 9621 | 10117 | 10613 | 11109 | 11605 |
| 135 | 8134 | 8630 | 9126 | 9622 | 10118 | 10614 | 11110 | 11606 |
| 136 | 8135 | 8631 | 9127 | 9623 | 10119 | 10615 | 11111 | 11607 |
| 137 | 8136 | 8632 | 9128 | 9624 | 10120 | 10616 | 11112 | 11608 |
| 138 | 8137 | 8633 | 9129 | 9625 | 10121 | 10617 | 11113 | 11609 |
| 139 | 8138 | 8634 | 9130 | 9626 | 10122 | 10618 | 11114 | 11610 |
| 140 | 8139 | 8635 | 9131 | 9627 | 10123 | 10619 | 11115 | 11611 |
| 141 | 8140 | 8636 | 9132 | 9628 | 10124 | 10620 | 11116 | 11612 |
| 142 | 8141 | 8637 | 9133 | 9629 | 10125 | 10621 | 11117 | 11613 |
| 143 | 8142 | 8638 | 9134 | 9630 | 10126 | 10622 | 11118 | 11614 |
| 144 | 8143 | 8639 | 9135 | 9631 | 10127 | 10623 | 11119 | 11615 |
| 145 | 8144 | 8640 | 9136 | 9632 | 10128 | 10624 | 11120 | 11616 |
| 146 | 8145 | 8641 | 9137 | 9633 | 10129 | 10625 | 11121 | 11617 |
| 147 | 8146 | 8642 | 9138 | 9634 | 10130 | 10626 | 11122 | 11618 |
| 148 | 8147 | 8643 | 9139 | 9635 | 10131 | 10627 | 11123 | 11619 |
| 149 | 8148 | 8644 | 9140 | 9636 | 10132 | 10628 | 11124 | 11620 |
| 150 | 8149 | 8645 | 9141 | 9637 | 10133 | 10629 | 11125 | 11621 |
| 151 | 8150 | 8646 | 9142 | 9638 | 10134 | 10630 | 11126 | 11622 |
| 152 | 8151 | 8647 | 9143 | 9639 | 10135 | 10631 | 11127 | 11623 |
| 153 | 8152 | 8648 | 9144 | 9640 | 10136 | 10632 | 11128 | 11624 |
| 154 | 8153 | 8649 | 9145 | 9641 | 10137 | 10633 | 11129 | 11625 |
| 155 | 8154 | 8650 | 9146 | 9642 | 10138 | 10634 | 11130 | 11626 |
| 156 | 8155 | 8651 | 9147 | 9643 | 10139 | 10635 | 11131 | 11627 |
| 157 | 8156 | 8652 | 9148 | 9644 | 10140 | 10636 | 11132 | 11628 |
| 158 | 8157 | 8653 | 9149 | 9645 | 10141 | 10637 | 11133 | 11629 |
| 159 | 8158 | 8654 | 9150 | 9646 | 10142 | 10638 | 11134 | 11630 |
| 160 | 8159 | 8655 | 9151 | 9647 | 10143 | 10639 | 11135 | 11631 |
| 161 | 8160 | 8656 | 9152 | 9648 | 10144 | 10640 | 11136 | 11632 |
| 162 | 8161 | 8657 | 9153 | 9649 | 10145 | 10641 | 11137 | 11633 |
| 163 | 8162 | 8658 | 9154 | 9650 | 10146 | 10642 | 11138 | 11634 |
| 164 | 8163 | 8659 | 9155 | 9651 | 10147 | 10643 | 11139 | 11635 |
| 165 | 8164 | 8660 | 9156 | 9652 | 10148 | 10644 | 11140 | 11636 |
| 166 | 8165 | 8661 | 9157 | 9653 | 10149 | 10645 | 11141 | 11637 |
| 167 | 8166 | 8662 | 9158 | 9654 | 10150 | 10646 | 11142 | 11638 |
| 168 | 8167 | 8663 | 9159 | 9655 | 10151 | 10647 | 11143 | 11639 |
| 169 | 8168 | 8664 | 9160 | 9656 | 10152 | 10648 | 11144 | 11640 |
| 170 | 8169 | 8665 | 9161 | 9657 | 10153 | 10649 | 11145 | 11641 |
| 171 | 8170 | 8666 | 9162 | 9658 | 10154 | 10650 | 11146 | 11642 |
| 172 | 8171 | 8667 | 9163 | 9659 | 10155 | 10651 | 11147 | 11643 |
| 173 | 8172 | 8668 | 9164 | 9660 | 10156 | 10652 | 11148 | 11644 |
| 174 | 8173 | 8669 | 9165 | 9661 | 10157 | 10653 | 11149 | 11645 |
| 175 | 8174 | 8670 | 9166 | 9662 | 10158 | 10654 | 11150 | 11646 |
| 176 | 8175 | 8671 | 9167 | 9663 | 10159 | 10655 | 11151 | 11647 |
| 177 | 8176 | 8672 | 9168 | 9664 | 10160 | 10656 | 11152 | 11648 |
| 178 | 8177 | 8673 | 9169 | 9665 | 10161 | 10657 | 11153 | 11649 |
| 179 | 8178 | 8674 | 9170 | 9666 | 10162 | 10658 | 11154 | 11650 |
| 180 | 8179 | 8675 | 9171 | 9667 | 10163 | 10659 | 11155 | 11651 |
| 181 | 8180 | 8676 | 9172 | 9668 | 10164 | 10660 | 11156 | 11652 |
| 182 | 8181 | 8677 | 9173 | 9669 | 10165 | 10661 | 11157 | 11653 |
| 183 | 8182 | 8678 | 9174 | 9670 | 10166 | 10662 | 11158 | 11654 |
| 184 | 8183 | 8679 | 9175 | 9671 | 10167 | 10663 | 11159 | 11655 |
| 185 | 8184 | 8680 | 9176 | 9672 | 10168 | 10664 | 11160 | 11656 |
| 186 | 8185 | 8681 | 9177 | 9673 | 10169 | 10665 | 11161 | 11657 |
| 187 | 8186 | 8682 | 9178 | 9674 | 10170 | 10666 | 11162 | 11658 |
| 188 | 8187 | 8683 | 9179 | 9675 | 10171 | 10667 | 11163 | 11659 |
| 189 | 8188 | 8684 | 9180 | 9676 | 10172 | 10668 | 11164 | 11660 |
| 190 | 8189 | 8685 | 9181 | 9677 | 10173 | 10669 | 11165 | 11661 |
| 191 | 8190 | 8686 | 9182 | 9678 | 10174 | 10670 | 11166 | 11662 |
| 192 | 8191 | 8687 | 9183 | 9679 | 10175 | 10671 | 11167 | 11663 |
| 193 | 8192 | 8688 | 9184 | 9680 | 10176 | 10672 | 11168 | 11664 |
| 194 | 8193 | 8689 | 9185 | 9681 | 10177 | 10673 | 11169 | 11665 |
| 195 | 8194 | 8690 | 9186 | 9682 | 10178 | 10674 | 11170 | 11666 |
| 196 | 8195 | 8691 | 9187 | 9683 | 10179 | 10675 | 11171 | 11667 |
| 197 | 8196 | 8692 | 9188 | 9684 | 10180 | 10676 | 11172 | 11668 |
| 198 | 8197 | 8693 | 9189 | 9685 | 10181 | 10677 | 11173 | 11669 |
| 199 | 8198 | 8694 | 9190 | 9686 | 10182 | 10678 | 11174 | 11670 |
| 200 | 8199 | 8695 | 9191 | 9687 | 10183 | 10679 | 11175 | 11671 |
| 201 | 8200 | 8696 | 9192 | 9688 | 10184 | 10680 | 11176 | 11672 |
| 202 | 8201 | 8697 | 9193 | 9689 | 10185 | 10681 | 11177 | 11673 |
| 203 | 8202 | 8698 | 9194 | 9690 | 10186 | 10682 | 11178 | 11674 |
| 204 | 8203 | 8699 | 9195 | 9691 | 10187 | 10683 | 11179 | 11675 |
| 205 | 8204 | 8700 | 9196 | 9692 | 10188 | 10684 | 11180 | 11676 |
| 206 | 8205 | 8701 | 9197 | 9693 | 10189 | 10685 | 11181 | 11677 |
| 207 | 8206 | 8702 | 9198 | 9694 | 10190 | 10686 | 11182 | 11678 |
| 208 | 8207 | 8703 | 9199 | 9695 | 10191 | 10687 | 11183 | 11679 |
| 209 | 8208 | 8704 | 9200 | 9696 | 10192 | 10688 | 11184 | 11680 |
| 210 | 8209 | 8705 | 9201 | 9697 | 10193 | 10689 | 11185 | 11681 |
| 211 | 8210 | 8706 | 9202 | 9698 | 10194 | 10690 | 11186 | 11682 |
| 212 | 8211 | 8707 | 9203 | 9699 | 10195 | 10691 | 11187 | 11683 |
| 213 | 8212 | 8708 | 9204 | 9700 | 10196 | 10692 | 11188 | 11684 |
| 214 | 8213 | 8709 | 9205 | 9701 | 10197 | 10693 | 11189 | 11685 |
| 215 | 8214 | 8710 | 9206 | 9702 | 10198 | 10694 | 11190 | 11686 |
| 216 | 8215 | 8711 | 9207 | 9703 | 10199 | 10695 | 11191 | 11687 |
| 217 | 8216 | 8712 | 9208 | 9704 | 10200 | 10696 | 11192 | 11688 |
| 218 | 8217 | 8713 | 9209 | 9705 | 10201 | 10697 | 11193 | 11689 |
| 219 | 8218 | 8714 | 9210 | 9706 | 10202 | 10698 | 11194 | 11690 |
| 220 | 8219 | 8715 | 9211 | 9707 | 10203 | 10699 | 11195 | 11691 |
| 221 | 8220 | 8716 | 9212 | 9708 | 10204 | 10700 | 11196 | 11692 |
| 222 | 8221 | 8717 | 9213 | 9709 | 10205 | 10701 | 11197 | 11693 |
| 223 | 8222 | 8718 | 9214 | 9710 | 10206 | 10702 | 11198 | 11694 |
| 224 | 8223 | 8719 | 9215 | 9711 | 10207 | 10703 | 11199 | 11695 |
| 225 | 8224 | 8720 | 9216 | 9712 | 10208 | 10704 | 11200 | 11696 |
| 226 | 8225 | 8721 | 9217 | 9713 | 10209 | 10705 | 11201 | 11697 |
| 227 | 8226 | 8722 | 9218 | 9714 | 10210 | 10706 | 11202 | 11698 |
| 228 | 8227 | 8723 | 9219 | 9715 | 10211 | 10707 | 11203 | 11699 |
| 229 | 8228 | 8724 | 9220 | 9716 | 10212 | 10708 | 11204 | 11700 |
| 230 | 8229 | 8725 | 9221 | 9717 | 10213 | 10709 | 11205 | 11701 |
| 231 | 8230 | 8726 | 9222 | 9718 | 10214 | 10710 | 11206 | 11702 |
| 232 | 8231 | 8727 | 9223 | 9719 | 10215 | 10711 | 11207 | 11703 |
| 233 | 8232 | 8728 | 9224 | 9720 | 10216 | 10712 | 11208 | 11704 |
| 234 | 8233 | 8729 | 9225 | 9721 | 10217 | 10713 | 11209 | 11705 |
| 235 | 8234 | 8730 | 9226 | 9722 | 10218 | 10714 | 11210 | 11706 |
| 236 | 8235 | 8731 | 9227 | 9723 | 10219 | 10715 | 11211 | 11707 |
| 237 | 8236 | 8732 | 9228 | 9724 | 10220 | 10716 | 11212 | 11708 |
| 238 | 8237 | 8733 | 9229 | 9725 | 10221 | 10717 | 11213 | 11709 |
| 239 | 8238 | 8734 | 9230 | 9726 | 10222 | 10718 | 11214 | 11710 |
| 240 | 8239 | 8735 | 9231 | 9727 | 10223 | 10719 | 11215 | 11711 |
| 241 | 8240 | 8736 | 9232 | 9728 | 10224 | 10720 | 11216 | 11712 |
| 242 | 8241 | 8737 | 9233 | 9729 | 10225 | 10721 | 11217 | 11713 |
| 243 | 8242 | 8738 | 9234 | 9730 | 10226 | 10722 | 11218 | 11714 |
| 244 | 8243 | 8739 | 9235 | 9731 | 10227 | 10723 | 11219 | 11715 |
| 245 | 8244 | 8740 | 9236 | 9732 | 10228 | 10724 | 11220 | 11716 |
| 246 | 8245 | 8741 | 9237 | 9733 | 10229 | 10725 | 11221 | 11717 |
| 247 | 8246 | 8742 | 9238 | 9734 | 10230 | 10726 | 11222 | 11718 |
| 248 | 8247 | 8743 | 9239 | 9735 | 10231 | 10727 | 11223 | 11719 |
| 249 | 8248 | 8744 | 9240 | 9736 | 10232 | 10728 | 11224 | 11720 |
| 250 | 8249 | 8745 | 9241 | 9737 | 10233 | 10729 | 11225 | 11721 |
| 251 | 8250 | 8746 | 9242 | 9738 | 10234 | 10730 | 11226 | 11722 |
| 252 | 8251 | 8747 | 9243 | 9739 | 10235 | 10731 | 11227 | 11723 |
| 253 | 8252 | 8748 | 9244 | 9740 | 10236 | 10732 | 11228 | 11724 |
| 254 | 8253 | 8749 | 9245 | 9741 | 10237 | 10733 | 11229 | 11725 |
| 255 | 8254 | 8750 | 9246 | 9742 | 10238 | 10734 | 11230 | 11726 |
| 256 | 8255 | 8751 | 9247 | 9743 | 10239 | 10735 | 11231 | 11727 |
| 257 | 8256 | 8752 | 9248 | 9744 | 10240 | 10736 | 11232 | 11728 |
| 258 | 8257 | 8753 | 9249 | 9745 | 10241 | 10737 | 11233 | 11729 |
| 259 | 8258 | 8754 | 9250 | 9746 | 10242 | 10738 | 11234 | 11730 |
| 260 | 8259 | 8755 | 9251 | 9747 | 10243 | 10739 | 11235 | 11731 |
| 261 | 8260 | 8756 | 9252 | 9748 | 10244 | 10740 | 11236 | 11732 |
| 262 | 8261 | 8757 | 9253 | 9749 | 10245 | 10741 | 11237 | 11733 |
| 263 | 8262 | 8758 | 9254 | 9750 | 10246 | 10742 | 11238 | 11734 |
| 264 | 8263 | 8759 | 9255 | 9751 | 10247 | 10743 | 11239 | 11735 |
| 265 | 8264 | 8760 | 9256 | 9752 | 10248 | 10744 | 11240 | 11736 |
| 266 | 8265 | 8761 | 9257 | 9753 | 10249 | 10745 | 11241 | 11737 |
| 267 | 8266 | 8762 | 9258 | 9754 | 10250 | 10746 | 11242 | 11738 |
| 268 | 8267 | 8763 | 9259 | 9755 | 10251 | 10747 | 11243 | 11739 |
| 269 | 8268 | 8764 | 9260 | 9756 | 10252 | 10748 | 11244 | 11740 |
| 270 | 8269 | 8765 | 9261 | 9757 | 10253 | 10749 | 11245 | 11741 |
| 271 | 8270 | 8766 | 9262 | 9758 | 10254 | 10750 | 11246 | 11742 |
| 272 | 8271 | 8767 | 9263 | 9759 | 10255 | 10751 | 11247 | 11743 |
| 273 | 8272 | 8768 | 9264 | 9760 | 10256 | 10752 | 11248 | 11744 |
| 274 | 8273 | 8769 | 9265 | 9761 | 10257 | 10753 | 11249 | 11745 |
| 275 | 8274 | 8770 | 9266 | 9762 | 10258 | 10754 | 11250 | 11746 |
| 276 | 8275 | 8771 | 9267 | 9763 | 10259 | 10755 | 11251 | 11747 |
| 277 | 8276 | 8772 | 9268 | 9764 | 10260 | 10756 | 11252 | 11748 |
| 278 | 8277 | 8773 | 9269 | 9765 | 10261 | 10757 | 11253 | 11749 |
| 279 | 8278 | 8774 | 9270 | 9766 | 10262 | 10758 | 11254 | 11750 |
| 280 | 8279 | 8775 | 9271 | 9767 | 10263 | 10759 | 11255 | 11751 |
| 281 | 8280 | 8776 | 9272 | 9768 | 10264 | 10760 | 11256 | 11752 |
| 282 | 8281 | 8777 | 9273 | 9769 | 10265 | 10761 | 11257 | 11753 |
| 283 | 8282 | 8778 | 9274 | 9770 | 10266 | 10762 | 11258 | 11754 |
| 284 | 8283 | 8779 | 9275 | 9771 | 10267 | 10763 | 11259 | 11755 |
| 285 | 8284 | 8780 | 9276 | 9772 | 10268 | 10764 | 11260 | 11756 |
| 286 | 8285 | 8781 | 9277 | 9773 | 10269 | 10765 | 11261 | 11757 |
| 287 | 8286 | 8782 | 9278 | 9774 | 10270 | 10766 | 11262 | 11758 |
| 288 | 8287 | 8783 | 9279 | 9775 | 10271 | 10767 | 11263 | 11759 |
| 289 | 8288 | 8784 | 9280 | 9776 | 10272 | 10768 | 11264 | 11760 |
| 290 | 8289 | 8785 | 9281 | 9777 | 10273 | 10769 | 11265 | 11761 |
| 291 | 8290 | 8786 | 9282 | 9778 | 10274 | 10770 | 11266 | 11762 |
| 292 | 8291 | 8787 | 9283 | 9779 | 10275 | 10771 | 11267 | 11763 |
| 293 | 8292 | 8788 | 9284 | 9780 | 10276 | 10772 | 11268 | 11764 |
| 294 | 8293 | 8789 | 9285 | 9781 | 10277 | 10773 | 11269 | 11765 |
| 295 | 8294 | 8790 | 9286 | 9782 | 10278 | 10774 | 11270 | 11766 |
| 296 | 8295 | 8791 | 9287 | 9783 | 10279 | 10775 | 11271 | 11767 |
| 297 | 8296 | 8792 | 9288 | 9784 | 10280 | 10776 | 11272 | 11768 |
| 298 | 8297 | 8793 | 9289 | 9785 | 10281 | 10777 | 11273 | 11769 |
| 299 | 8298 | 8794 | 9290 | 9786 | 10282 | 10778 | 11274 | 11770 |
| 300 | 8299 | 8795 | 9291 | 9787 | 10283 | 10779 | 11275 | 11771 |
| 301 | 8300 | 8796 | 9292 | 9788 | 10284 | 10780 | 11276 | 11772 |
| 302 | 8301 | 8797 | 9293 | 9789 | 10285 | 10781 | 11277 | 11773 |
| 303 | 8302 | 8798 | 9294 | 9790 | 10286 | 10782 | 11278 | 11774 |
| 304 | 8303 | 8799 | 9295 | 9791 | 10287 | 10783 | 11279 | 11775 |
| 305 | 8304 | 8800 | 9296 | 9792 | 10288 | 10784 | 11280 | 11776 |
| 306 | 8305 | 8801 | 9297 | 9793 | 10289 | 10785 | 11281 | 11777 |
| 307 | 8306 | 8802 | 9298 | 9794 | 10290 | 10786 | 11282 | 11778 |
| 308 | 8307 | 8803 | 9299 | 9795 | 10291 | 10787 | 11283 | 11779 |
| 309 | 8308 | 8804 | 9300 | 9796 | 10292 | 10788 | 11284 | 11780 |
| 310 | 8309 | 8805 | 9301 | 9797 | 10293 | 10789 | 11285 | 11781 |
| 311 | 8310 | 8806 | 9302 | 9798 | 10294 | 10790 | 11286 | 11782 |
| 312 | 8311 | 8807 | 9303 | 9799 | 10295 | 10791 | 11287 | 11783 |
| 313 | 8312 | 8808 | 9304 | 9800 | 10296 | 10792 | 11288 | 11784 |
| 314 | 8313 | 8809 | 9305 | 9801 | 10297 | 10793 | 11289 | 11785 |
| 315 | 8314 | 8810 | 9306 | 9802 | 10298 | 10794 | 11290 | 11786 |
| 316 | 8315 | 8811 | 9307 | 9803 | 10299 | 10795 | 11291 | 11787 |
| 317 | 8316 | 8812 | 9308 | 9804 | 10300 | 10796 | 11292 | 11788 |
| 318 | 8317 | 8813 | 9309 | 9805 | 10301 | 10797 | 11293 | 11789 |
| 319 | 8318 | 8814 | 9310 | 9806 | 10302 | 10798 | 11294 | 11790 |
| 320 | 8319 | 8815 | 9311 | 9807 | 10303 | 10799 | 11295 | 11791 |
| 321 | 8320 | 8816 | 9312 | 9808 | 10304 | 10800 | 11296 | 11792 |
| 322 | 8321 | 8817 | 9313 | 9809 | 10305 | 10801 | 11297 | 11793 |
| 323 | 8322 | 8818 | 9314 | 9810 | 10306 | 10802 | 11298 | 11794 |
| 324 | 8323 | 8819 | 9315 | 9811 | 10307 | 10803 | 11299 | 11795 |
| 325 | 8324 | 8820 | 9316 | 9812 | 10308 | 10804 | 11300 | 11796 |
| 326 | 8325 | 8821 | 9317 | 9813 | 10309 | 10805 | 11301 | 11797 |
| 327 | 8326 | 8822 | 9318 | 9814 | 10310 | 10806 | 11302 | 11798 |
| 328 | 8327 | 8823 | 9319 | 9815 | 10311 | 10807 | 11303 | 11799 |
| 329 | 8328 | 8824 | 9320 | 9816 | 10312 | 10808 | 11304 | 11800 |
| 330 | 8329 | 8825 | 9321 | 9817 | 10313 | 10809 | 11305 | 11801 |
| 331 | 8330 | 8826 | 9322 | 9818 | 10314 | 10810 | 11306 | 11802 |
| 332 | 8331 | 8827 | 9323 | 9819 | 10315 | 10811 | 11307 | 11803 |
| 333 | 8332 | 8828 | 9324 | 9820 | 10316 | 10812 | 11308 | 11804 |
| 334 | 8333 | 8829 | 9325 | 9821 | 10317 | 10813 | 11309 | 11805 |
| 335 | 8334 | 8830 | 9326 | 9822 | 10318 | 10814 | 11310 | 11806 |
| 336 | 8335 | 8831 | 9327 | 9823 | 10319 | 10815 | 11311 | 11807 |
| 337 | 8336 | 8832 | 9328 | 9824 | 10320 | 10816 | 11312 | 11808 |
| 338 | 8337 | 8833 | 9329 | 9825 | 10321 | 10817 | 11313 | 11809 |
| 339 | 8338 | 8834 | 9330 | 9826 | 10322 | 10818 | 11314 | 11810 |
| 340 | 8339 | 8835 | 9331 | 9827 | 10323 | 10819 | 11315 | 11811 |
| 341 | 8340 | 8836 | 9332 | 9828 | 10324 | 10820 | 11316 | 11812 |
| 342 | 8341 | 8837 | 9333 | 9829 | 10325 | 10821 | 11317 | 11813 |
| 343 | 8342 | 8838 | 9334 | 9830 | 10326 | 10822 | 11318 | 11814 |
| 344 | 8343 | 8839 | 9335 | 9831 | 10327 | 10823 | 11319 | 11815 |
| 345 | 8344 | 8840 | 9336 | 9832 | 10328 | 10824 | 11320 | 11816 |
| 346 | 8345 | 8841 | 9337 | 9833 | 10329 | 10825 | 11321 | 11817 |
| 347 | 8346 | 8842 | 9338 | 9834 | 10330 | 10826 | 11322 | 11818 |
| 348 | 8347 | 8843 | 9339 | 9835 | 10331 | 10827 | 11323 | 11819 |
| 349 | 8348 | 8844 | 9340 | 9836 | 10332 | 10828 | 11324 | 11820 |
| 350 | 8349 | 8845 | 9341 | 9837 | 10333 | 10829 | 11325 | 11821 |
| 351 | 8350 | 8846 | 9342 | 9838 | 10334 | 10830 | 11326 | 11822 |
| 352 | 8351 | 8847 | 9343 | 9839 | 10335 | 10831 | 11327 | 11823 |
| 353 | 8352 | 8848 | 9344 | 9840 | 10336 | 10832 | 11328 | 11824 |
| 354 | 8353 | 8849 | 9345 | 9841 | 10337 | 10833 | 11329 | 11825 |
| 355 | 8354 | 8850 | 9346 | 9842 | 10338 | 10834 | 11330 | 11826 |
| 356 | 8355 | 8851 | 9347 | 9843 | 10339 | 10835 | 11331 | 11827 |
| 357 | 8356 | 8852 | 9348 | 9844 | 10340 | 10836 | 11332 | 11828 |
| 358 | 8357 | 8853 | 9349 | 9845 | 10341 | 10837 | 11333 | 11829 |
| 359 | 8358 | 8854 | 9350 | 9846 | 10342 | 10838 | 11334 | 11830 |
| 360 | 8359 | 8855 | 9351 | 9847 | 10343 | 10839 | 11335 | 11831 |
| 361 | 8360 | 8856 | 9352 | 9848 | 10344 | 10840 | 11336 | 11832 |
| 362 | 8361 | 8857 | 9353 | 9849 | 10345 | 10841 | 11337 | 11833 |
| 363 | 8362 | 8858 | 9354 | 9850 | 10346 | 10842 | 11338 | 11834 |
| 364 | 8363 | 8859 | 9355 | 9851 | 10347 | 10843 | 11339 | 11835 |
| 365 | 8364 | 8860 | 9356 | 9852 | 10348 | 10844 | 11340 | 11836 |
| 366 | 8365 | 8861 | 9357 | 9853 | 10349 | 10845 | 11341 | 11837 |
| 367 | 8366 | 8862 | 9358 | 9854 | 10350 | 10846 | 11342 | 11838 |
| 368 | 8367 | 8863 | 9359 | 9855 | 10351 | 10847 | 11343 | 11839 |
| 369 | 8368 | 8864 | 9360 | 9856 | 10352 | 10848 | 11344 | 11840 |
| 370 | 8369 | 8865 | 9361 | 9857 | 10353 | 10849 | 11345 | 11841 |
| 371 | 8370 | 8866 | 9362 | 9858 | 10354 | 10850 | 11346 | 11842 |
| 372 | 8371 | 8867 | 9363 | 9859 | 10355 | 10851 | 11347 | 11843 |
| 373 | 8372 | 8868 | 9364 | 9860 | 10356 | 10852 | 11348 | 11844 |
| 374 | 8373 | 8869 | 9365 | 9861 | 10357 | 10853 | 11349 | 11845 |
| 375 | 8374 | 8870 | 9366 | 9862 | 10358 | 10854 | 11350 | 11846 |
| 376 | 8375 | 8871 | 9367 | 9863 | 10359 | 10855 | 11351 | 11847 |
| 377 | 8376 | 8872 | 9368 | 9864 | 10360 | 10856 | 11352 | 11848 |
| 378 | 8377 | 8873 | 9369 | 9865 | 10361 | 10857 | 11353 | 11849 |
| 379 | 8378 | 8874 | 9370 | 9866 | 10362 | 10858 | 11354 | 11850 |
| 380 | 8379 | 8875 | 9371 | 9867 | 10363 | 10859 | 11355 | 11851 |
| 381 | 8380 | 8876 | 9372 | 9868 | 10364 | 10860 | 11356 | 11852 |
| 382 | 8381 | 8877 | 9373 | 9869 | 10365 | 10861 | 11357 | 11853 |
| 383 | 8382 | 8878 | 9374 | 9870 | 10366 | 10862 | 11358 | 11854 |
| 384 | 8383 | 8879 | 9375 | 9871 | 10367 | 10863 | 11359 | 11855 |
| 385 | 8384 | 8880 | 9376 | 9872 | 10368 | 10864 | 11360 | 11856 |
| 386 | 8385 | 8881 | 9377 | 9873 | 10369 | 10865 | 11361 | 11857 |
| 387 | 8386 | 8882 | 9378 | 9874 | 10370 | 10866 | 11362 | 11858 |
| 388 | 8387 | 8883 | 9379 | 9875 | 10371 | 10867 | 11363 | 11859 |
| 389 | 8388 | 8884 | 9380 | 9876 | 10372 | 10868 | 11364 | 11860 |
| 390 | 8389 | 8885 | 9381 | 9877 | 10373 | 10869 | 11365 | 11861 |
| 391 | 8390 | 8886 | 9382 | 9878 | 10374 | 10870 | 11366 | 11862 |
| 392 | 8391 | 8887 | 9383 | 9879 | 10375 | 10871 | 11367 | 11863 |
| 393 | 8392 | 8888 | 9384 | 9880 | 10376 | 10872 | 11368 | 11864 |
| 394 | 8393 | 8889 | 9385 | 9881 | 10377 | 10873 | 11369 | 11865 |
| 395 | 8394 | 8890 | 9386 | 9882 | 10378 | 10874 | 11370 | 11866 |
| 396 | 8395 | 8891 | 9387 | 9883 | 10379 | 10875 | 11371 | 11867 |
| 397 | 8396 | 8892 | 9388 | 9884 | 10380 | 10876 | 11372 | 11868 |
| 398 | 8397 | 8893 | 9389 | 9885 | 10381 | 10877 | 11373 | 11869 |
| 399 | 8398 | 8894 | 9390 | 9886 | 10382 | 10878 | 11374 | 11870 |
| 400 | 8399 | 8895 | 9391 | 9887 | 10383 | 10879 | 11375 | 11871 |
| 401 | 8400 | 8896 | 9392 | 9888 | 10384 | 10880 | 11376 | 11872 |
| 402 | 8401 | 8897 | 9393 | 9889 | 10385 | 10881 | 11377 | 11873 |
| 403 | 8402 | 8898 | 9394 | 9890 | 10386 | 10882 | 11378 | 11874 |
| 404 | 8403 | 8899 | 9395 | 9891 | 10387 | 10883 | 11379 | 11875 |
| 405 | 8404 | 8900 | 9396 | 9892 | 10388 | 10884 | 11380 | 11876 |
| 406 | 8405 | 8901 | 9397 | 9893 | 10389 | 10885 | 11381 | 11877 |
| 407 | 8406 | 8902 | 9398 | 9894 | 10390 | 10886 | 11382 | 11878 |
| 408 | 8407 | 8903 | 9399 | 9895 | 10391 | 10887 | 11383 | 11879 |
| 409 | 8408 | 8904 | 9400 | 9896 | 10392 | 10888 | 11384 | 11880 |
| 410 | 8409 | 8905 | 9401 | 9897 | 10393 | 10889 | 11385 | 11881 |
| 411 | 8410 | 8906 | 9402 | 9898 | 10394 | 10890 | 11386 | 11882 |
| 412 | 8411 | 8907 | 9403 | 9899 | 10395 | 10891 | 11387 | 11883 |
| 413 | 8412 | 8908 | 9404 | 9900 | 10396 | 10892 | 11388 | 11884 |
| 414 | 8413 | 8909 | 9405 | 9901 | 10397 | 10893 | 11389 | 11885 |
| 415 | 8414 | 8910 | 9406 | 9902 | 10398 | 10894 | 11390 | 11886 |
| 416 | 8415 | 8911 | 9407 | 9903 | 10399 | 10895 | 11391 | 11887 |
| 417 | 8416 | 8912 | 9408 | 9904 | 10400 | 10896 | 11392 | 11888 |
| 418 | 8417 | 8913 | 9409 | 9905 | 10401 | 10897 | 11393 | 11889 |
| 419 | 8418 | 8914 | 9410 | 9906 | 10402 | 10898 | 11394 | 11890 |
| 420 | 8419 | 8915 | 9411 | 9907 | 10403 | 10899 | 11395 | 11891 |
| 421 | 8420 | 8916 | 9412 | 9908 | 10404 | 10900 | 11396 | 11892 |
| 422 | 8421 | 8917 | 9413 | 9909 | 10405 | 10901 | 11397 | 11893 |
| 423 | 8422 | 8918 | 9414 | 9910 | 10406 | 10902 | 11398 | 11894 |
| 424 | 8423 | 8919 | 9415 | 9911 | 10407 | 10903 | 11399 | 11895 |
| 425 | 8424 | 8920 | 9416 | 9912 | 10408 | 10904 | 11400 | 11896 |
| 426 | 8425 | 8921 | 9417 | 9913 | 10409 | 10905 | 11401 | 11897 |
| 427 | 8426 | 8922 | 9418 | 9914 | 10410 | 10906 | 11402 | 11898 |
| 428 | 8427 | 8923 | 9419 | 9915 | 10411 | 10907 | 11403 | 11899 |
| 429 | 8428 | 8924 | 9420 | 9916 | 10412 | 10908 | 11404 | 11900 |
| 430 | 8429 | 8925 | 9421 | 9917 | 10413 | 10909 | 11405 | 11901 |
| 431 | 8430 | 8926 | 9422 | 9918 | 10414 | 10910 | 11406 | 11902 |
| 432 | 8431 | 8927 | 9423 | 9919 | 10415 | 10911 | 11407 | 11903 |
| 433 | 8432 | 8928 | 9424 | 9920 | 10416 | 10912 | 11408 | 11904 |
| 434 | 8433 | 8929 | 9425 | 9921 | 10417 | 10913 | 11409 | 11905 |
| 435 | 8434 | 8930 | 9426 | 9922 | 10418 | 10914 | 11410 | 11906 |
| 436 | 8435 | 8931 | 9427 | 9923 | 10419 | 10915 | 11411 | 11907 |
| 437 | 8436 | 8932 | 9428 | 9924 | 10420 | 10916 | 11412 | 11908 |
| 438 | 8437 | 8933 | 9429 | 9925 | 10421 | 10917 | 11413 | 11909 |
| 439 | 8438 | 8934 | 9430 | 9926 | 10422 | 10918 | 11414 | 11910 |
| 440 | 8439 | 8935 | 9431 | 9927 | 10423 | 10919 | 11415 | 11911 |
| 441 | 8440 | 8936 | 9432 | 9928 | 10424 | 10920 | 11416 | 11912 |
| 442 | 8441 | 8937 | 9433 | 9929 | 10425 | 10921 | 11417 | 11913 |
| 443 | 8442 | 8938 | 9434 | 9930 | 10426 | 10922 | 11418 | 11914 |
| 444 | 8443 | 8939 | 9435 | 9931 | 10427 | 10923 | 11419 | 11915 |
| 445 | 8444 | 8940 | 9436 | 9932 | 10428 | 10924 | 11420 | 11916 |
| 446 | 8445 | 8941 | 9437 | 9933 | 10429 | 10925 | 11421 | 11917 |
| 447 | 8446 | 8942 | 9438 | 9934 | 10430 | 10926 | 11422 | 11918 |
| 448 | 8447 | 8943 | 9439 | 9935 | 10431 | 10927 | 11423 | 11919 |
| 449 | 8448 | 8944 | 9440 | 9936 | 10432 | 10928 | 11424 | 11920 |
| 450 | 8449 | 8945 | 9441 | 9937 | 10433 | 10929 | 11425 | 11921 |
| 451 | 8450 | 8946 | 9442 | 9938 | 10434 | 10930 | 11426 | 11922 |
| 452 | 8451 | 8947 | 9443 | 9939 | 10435 | 10931 | 11427 | 11923 |
| 453 | 8452 | 8948 | 9444 | 9940 | 10436 | 10932 | 11428 | 11924 |
| 454 | 8453 | 8949 | 9445 | 9941 | 10437 | 10933 | 11429 | 11925 |
| 455 | 8454 | 8950 | 9446 | 9942 | 10438 | 10934 | 11430 | 11926 |
| 456 | 8455 | 8951 | 9447 | 9943 | 10439 | 10935 | 11431 | 11927 |
| 457 | 8456 | 8952 | 9448 | 9944 | 10440 | 10936 | 11432 | 11928 |
| 458 | 8457 | 8953 | 9449 | 9945 | 10441 | 10937 | 11433 | 11929 |
| 459 | 8458 | 8954 | 9450 | 9946 | 10442 | 10938 | 11434 | 11930 |
| 460 | 8459 | 8955 | 9451 | 9947 | 10443 | 10939 | 11435 | 11931 |
| 461 | 8460 | 8956 | 9452 | 9948 | 10444 | 10940 | 11436 | 11932 |
| 462 | 8461 | 8957 | 9453 | 9949 | 10445 | 10941 | 11437 | 11933 |
| 463 | 8462 | 8958 | 9454 | 9950 | 10446 | 10942 | 11438 | 11934 |
| 464 | 8463 | 8959 | 9455 | 9951 | 10447 | 10943 | 11439 | 11935 |
| 465 | 8464 | 8960 | 9456 | 9952 | 10448 | 10944 | 11440 | 11936 |
| 466 | 8465 | 8961 | 9457 | 9953 | 10449 | 10945 | 11441 | 11937 |
| 467 | 8466 | 8962 | 9458 | 9954 | 10450 | 10946 | 11442 | 11938 |
| 468 | 8467 | 8963 | 9459 | 9955 | 10451 | 10947 | 11443 | 11939 |
| 469 | 8468 | 8964 | 9460 | 9956 | 10452 | 10948 | 11444 | 11940 |
| 470 | 8469 | 8965 | 9461 | 9957 | 10453 | 10949 | 11445 | 11941 |
| 471 | 8470 | 8966 | 9462 | 9958 | 10454 | 10950 | 11446 | 11942 |
| 472 | 8471 | 8967 | 9463 | 9959 | 10455 | 10951 | 11447 | 11943 |
| 473 | 8472 | 8968 | 9464 | 9960 | 10456 | 10952 | 11448 | 11944 |
| 474 | 8473 | 8969 | 9465 | 9961 | 10457 | 10953 | 11449 | 11945 |
| 475 | 8474 | 8970 | 9466 | 9962 | 10458 | 10954 | 11450 | 11946 |
| 476 | 8475 | 8971 | 9467 | 9963 | 10459 | 10955 | 11451 | 11947 |
| 477 | 8476 | 8972 | 9468 | 9964 | 10460 | 10956 | 11452 | 11948 |
| 478 | 8477 | 8973 | 9469 | 9965 | 10461 | 10957 | 11453 | 11949 |
| 479 | 8478 | 8974 | 9470 | 9966 | 10462 | 10958 | 11454 | 11950 |
| 480 | 8479 | 8975 | 9471 | 9967 | 10463 | 10959 | 11455 | 11951 |
| 481 | 8480 | 8976 | 9472 | 9968 | 10464 | 10960 | 11456 | 11952 |
| 482 | 8481 | 8977 | 9473 | 9969 | 10465 | 10961 | 11457 | 11953 |
| 483 | 8482 | 8978 | 9474 | 9970 | 10466 | 10962 | 11458 | 11954 |
| 484 | 8483 | 8979 | 9475 | 9971 | 10467 | 10963 | 11459 | 11955 |
| 485 | 8484 | 8980 | 9476 | 9972 | 10468 | 10964 | 11460 | 11956 |
| 486 | 8485 | 8981 | 9477 | 9973 | 10469 | 10965 | 11461 | 11957 |
| 487 | 8486 | 8982 | 9478 | 9974 | 10470 | 10966 | 11462 | 11958 |
| 488 | 8487 | 8983 | 9479 | 9975 | 10471 | 10967 | 11463 | 11959 |
| 489 | 8488 | 8984 | 9480 | 9976 | 10472 | 10968 | 11464 | 11960 |
| 490 | 8489 | 8985 | 9481 | 9977 | 10473 | 10969 | 11465 | 11961 |
| 491 | 8490 | 8986 | 9482 | 9978 | 10474 | 10970 | 11466 | 11962 |
| 492 | 8491 | 8987 | 9483 | 9979 | 10475 | 10971 | 11467 | 11963 |
| 493 | 8492 | 8988 | 9484 | 9980 | 10476 | 10972 | 11468 | 11964 |
| 494 | 8493 | 8989 | 9485 | 9981 | 10477 | 10973 | 11469 | 11965 |
| 495 | 8494 | 8990 | 9486 | 9982 | 10478 | 10974 | 11470 | 11966 |
| 496 | 8495 | 8991 | 9487 | 9983 | 10479 | 10975 | 11471 | 11967 |

TABLE 32
A14 Light Chain CDR sequences for GIGA-564 identified by different algorithms
(Antibody A14):

| Region | Definition | Sequence Fragment | Residues | Length |
|---|---|---|---|---|
| LFR1 | Chothia | EIVLTQSPGTLSLSPGERATLSC------ | 1-23 | 23 |
| | | (SEQ ID NO: 11999) | | |
| | AbM | EIVLTQSPGTLSLSPGERATLSC------ | 1-23 | 23 |
| | | (SEQ ID NO: 12000) | | |
| | Kabat | EIVLTQSPGTLSLSPGERATLSC------ | 1-23 | 23 |
| | | (SEQ ID NO: 12001) | | |
| | Contact | EIVLTQSPGTLSLSPGERATLSCRASQSV | 1-29 | 29 |
| | | (SEQ ID NO: 12002) | | |
| | IMGT | EIVLTQSPGTLSLSPGERATLSCRAS--- | 1-26 | 26 |
| | | (SEQ ID NO: 12003) | | |
| CDR-L1 | Chothia | RASQSVSSSYLA- | 24-35 | 12 |
| | | (SEQ ID NO: 12004) | | |
| | AbM | RASQSVSSSYLA- | 24-35 | 12 |
| | | (SEQ ID NO: 12005) | | |
| | Kabat | RASQSVSSSYLA- | 24-35 | 12 |
| | | (SEQ ID NO: 12006) | | |
| | Contact | ------SSSYLAWY (SEQ ID NO: 12007) | 30-37 | 8 |
| | IMGT | ---QSVSSSY---- (SEQ ID NO: 12008) | 27-33 | 7 |
| LFR2 | Chothia | --WYQQKPGQAPRLLIY (SEQ ID NO: 12009) | 36-50 | 15 |
| | AbM | --WYQQKPGQAPRLLIY (SEQ ID NO: 12010) | 36-50 | 15 |
| | Kabat | --WYQQKPGQAPRLLIY (SEQ ID NO: 12011) | 36-50 | 15 |
| | Contact | ----QQKPGQAPR---- (SEQ ID NO: 12012) | 38-46 | 9 |
| | IMGT | LAWYQQKPGQAPRLLIY (SEQ ID NO: 12013) | 34-50 | 17 |
| CDR-L2 | Chothia | ----GASSRAT (SEQ ID NO: 12014) | 51-57 | 7 |
| | AbM | ----GASSRAT (SEQ ID NO: 12015) | 51-57 | 7 |
| | Kabat | ----GASSRAT (SEQ ID NO: 12016) | 51-57 | 7 |
| | Contact | LLIYGASSRA- (SEQ ID NO: 12017) | 47-56 | 10 |
| | IMGT | ----GA----- (SEQ ID NO: 12018) | 51-52 | 2 |
| LFR3 | Chothia | -----GIPDRESGSGSGTDFTLTISRLEPEDFAVYYC | 58-89 | 32 |
| | | (SEQ ID NO: 12019) | | |
| | AbM | -----GIPDRFSGSGSGTDFTLTISRLEPEDFAVYYC | 58-89 | 32 |
| | | (SEQ ID NO: 12020) | | |
| | Kabat | -----GIPDRFSGSGSGTDFTLTISRLEPEDFAVYYC | 58-89 | 32 |
| | | (SEQ ID NO: 12021) | | |
| | Contact | ----TGIPDRFSGSGSGTDFTLTISRLEPEDFAVYYC | 57-89 | 33 |
| | | (SEQ ID NO: 12022) | | |
| | IMGT | SSRATGIPDRFSGSGSGTDFTLTISRLEPEDFAVYYC | 53-89 | 37 |
| | | (SEQ ID NO: 12023) | | |
| CDR-L3 | Chothia | QQYGSSPWT (SEQ ID NO: 12024) | 90-98 | 9 |
| | AbM | QQYGSSPWT (SEQ ID NO: 12025) | 90-98 | 9 |
| | Kabat | QQYGSSPWT (SEQ ID NO: 12026) | 90-98 | 9 |
| | Contact | QQYGSSPW- (SEQ ID NO: 12027) | 90-97 | 8 |
| | IMGT | QQYGSSPWT (SEQ ID NO: 12028) | 90-98 | 9 |
| LFR4 | Chothia | -SGQGTKVEIK (SEQ ID NO: 12029) | 99-108 | 10 |
| | AbM | -SGQGTKVEIK (SEQ ID NO: 12030) | 99-108 | 10 |
| | Kabat | -SGQGTKVEIK (SEQ ID NO: 12031) | 99-108 | 10 |
| | Contact | TSGQGTKVEIK (SEQ ID NO: 12032) | 98-108 | 11 |
| | IMGT | -SGQGTKVEIK (SEQ ID NO: 12033) | 99-108 | 10 |

TABLE 33
A14 Heavy Chain CDR sequences for GIGA-564 identified by different algorithms
(Antibody A14):

| Region | Definition | Sequence Fragment | Residues | Length |
|---|---|---|---|---|
| HFR1 | Chothia | QVQLVESGGGVVQPGRSLRLSCAAS----- (SEQ ID | 1-25 | 25 |
| | | NO: 12034) | | |
| | AbM | QVQLVESGGGVVQPGRSLRLSCAAS----- (SEQ ID | 1-25 | 25 |
| | | NO: 12035) | | |
| | Kabat | QVQLVESGGGVVQPGRSLRLSCAASGFTFS (SEQ ID | 1-30 | 30 |
| | | NO: 12036) | | |
| | Contact | QVQLVESGGGVVQPGRSLRLSCAASGFTF- (SEQ ID | 1-29 | 29 |
| | | NO: 12037) | | |
| | IMGT | QVQLVESGGGVVQPGRSLRLSCAAS----- (SEQ ID | 1-25 | 25 |
| | | NO: 12038) | | |
| CDR-H1 | Chothia | GFTFSSY--- (SEQ ID NO: 12039) | 26-32 | 7 |
| | AbM | GFTFSSYGMH (SEQ ID NO: 12040) | 26-35 | 10 |
| | Kabat | -----SYGMH (SEQ ID NO: 12041) | 31-35 | 5 |
| | Contact | ----SSYGMH (SEQ ID NO: 12042) | 30-35 | 6 |
| | IMGT | GFTFSSYG-- (SEQ ID NO: 12043) | 26-33 | 8 |
| HFR2 | Chothia | GMHWVRQAPGTGLEWVAVI (SEQ ID NO: 12044) | 33-51 | 19 |
| | AbM | ---WVRQAPGTGLEWVA-- (SEQ ID NO: 12045) | 36-49 | 14 |
| | Kabat | ---WVRQAPGTGLEWVA-- (SEQ ID NO: 12046) | 36-49 | 14 |
| | Contact | ---WVROAPGTGLE----- (SEQ ID NO: 12047) | 36-46 | 11 |
| | IMGT | -MHWVRQAPGTGLEWVAV- (SEQ ID NO: 12048) | 34-50 | 17 |
| CDR-H2 | Chothia | -----WYEGRN--------- (SEQ ID NO: 12049) | 52-57 | 6 |
| | AbM | ---VIWYEGRNKY------- (SEQ ID NO: 12050) | 50-59 | 10 |
| | Kabat | ---VIWYEGRNKYYADPVKG (SEQ ID NO: 12051) | 50-66 | 17 |
| | Contact | WVAVIWYEGRNKY------- (SEQ ID NO: 12052) | 47-59 | 13 |
| | IMGT | ----IWYEGRNK-------- (SEQ ID NO: 12053) | 51-58 | 8 |
| HFR3 | Chothia | KYYADPVKGRFTISRDNSKNTLYLQMNSLRDDDTAVYYCAR | 58-98 | 11 |
| | | (SEQ ID NO: 12054) | | |
| | AbM | --YADPVKGRFTISRDNSKNTLYLQMNSLRDDDTAVYYCAR | 60-98 | 39 |
| | | (SEQ ID NO: 12055) | | |
| | Kabat | ---------RFTISRDNSKNTLYLQMNSLRDDDTAVYYCAR | 67-98 | 32 |
| | | (SEQ ID NO: 12056) | | |
| | Contact | ---YADPVKGRFTISRDNSKNTLYLQMNSLRDDDTAVYYC- | 60-96 | 37 |
| | | (SEQ ID NO: 12057) | | |
| | IMGT | --YYADPVKGRFTISRDNSKNTLYLQMNSLRDDDTAVYYC- | 59-96 | 38 |
| | | (SEQ ID NO: 12058) | | |
| CDR-H3 | Chothia | --AGDLGAFDI | 99-107 | 9 |
| | | (SEQ ID NO: 12059) | | |
| | AbM | --AGDLGAFDI (SEQ ID NO: 12060) | 99-107 | 9 |
| | Kabat | --AGDLGAFDI (SEQ ID NO: 12061) | 99-107 | 9 |
| | Contact | ARAGDLGAFD- (SEQ ID NO: 12062) | 97-106 | 10 |
| | IMGT | ARAGDLGAFDI (SEQ ID NO: 12063) | 97-107 | 11 |
| HFR4 | Chothia | -WGQGTMVTVSS (SEQ ID NO: 12064) | 108-118 | 11 |
| | AbM | -WGQGTMVTVSS (SEQ ID NO: 12065) | 108-118 | 11 |
| | Kabat | -WGQGTMVTVSS (SEQ ID NO: 12066) | 108-118 | 11 |
| | Contact | IWGQGTMVTVSS (SEQ ID NO: 12067) | 107-118 | 12 |
| | IMGT | -WGQGTMVTVSS (SEQ ID NO: 12068) | 108-118 | 11 |

TABLE 35
Light Chain sequences, light chain CDR sequences, heavy chain sequences,
and heavy chain CDR sequences of Ipilimumab.

| Ipilimumab | Amino Acid Sequence | SEQ ID NO: |
|---|---|---|
| Variable Heavy Chain (HC) | QVQLVESGGGVVQPGRSLRLSCAA | 12087 |
| | SGFTFSSYTMHWVRQAPGKGLEW | |
| | VTFISYDGNNKYYADSVKGRFTISRD | |
| | NSKNTLYLQMNSLRAEDTAIYYCAR | |
| | TGWLGPFDYWGQGTLVTVSSA | |
| Variable Light Chain (LC) | EIVLTQSPGTLSLSPGERATLSCRAS | 12088 |
| | QSVGSSYLAWYQQKPGQAPRLLIY | |
| | GAFSRATGIPDRFSGSGSGTDFTLTI | |
| | SRLEPEDFAVYYCQQYGSSPWTFG | |
| | QGTKVEIK | |
| LC CDR1 | QSVGSSY | 12081 |
| LC CDR2 | GAF | 12082 |
| LC CDR3 | QQYGSSPWT | 12083 |
| HC CDR1 | GFTFSSYT | 12084 |
| HC CDR2 | ISYDGNNK | 12085 |
| HC CDR3 | ARTGWLGPFDY | 12086 |

What is claimed is:

1. An isolated antigen binding protein (ABP) that specifically binds a human cytotoxic T-lymphocyte associated protein 4 (CTLA-4), wherein
the ABP contacts amino acids K130, Y139, L141, I143 but does not contact amino acid R70 of the CTLA-4; and/or
the CTLA-4 can associate with CD80/CD86 when bound to the ABP; and/or
an interaction between the ABP and amino acid L74A and/or E68 of the CTLA-4 is greater than an interaction between Ipilimumab and amino acid L74A of CTLA-4.

2. The ABP of claim 1, wherein the ABP comprises a CDR1-L consisting of SEQ ID NO:12078 or SEQ ID NO: 1014, a CDR2-L consisting of SEQ ID NO:12079 or SEQ ID NO: 2014, a CDR3-L consisting of SEQ ID NO:12080 or SEQ ID NO: 3014, a CDR1-H consisting of SEQ ID NO:12075 or SEQ ID NO: 4014, a CDR2-H consisting of SEQ ID NO:12076 or SEQ ID NO: 5014 and a CDR3-H consisting of SEQ ID NO:12077 or SEQ ID NO: 6014.

3. The ABP of claim 1, wherein the ABP comprises a CDR1-L consisting of SEQ ID NO: 12004, a CDR2-L consisting of SEQ ID NO: 12014, a CDR3-L consisting of SEQ ID NO: 12024, a CDR1-H consisting of SEQ ID NO: 12039, a CDR2-H consisting of SEQ ID NO: 12049, and a CDR3-H consisting of SEQ ID NO: 12059.

4. The ABP of claim 1, wherein the ABP comprises a CDR1-L consisting of SEQ ID NO: 12005, a CDR2-L consisting of SEQ ID NO: 12015, a CDR3-L consisting of SEQ ID NO: 12025, a CDR1-H consisting of SEQ ID NO: 12040, a CDR2-H consisting of SEQ ID NO: 12050, and a CDR3-H consisting of SEQ ID NO: 12060.

5. The ABP of claim 1, wherein the ABP comprises a CDR1-L consisting of SEQ ID NO: 12006, a CDR2-L consisting of SEQ ID NO: 12016, a CDR3-L consisting of SEQ ID NO: 12026, a CDR1-H consisting of SEQ ID NO: 12041, a CDR2-H consisting of SEQ ID NO: 12051, and a CDR3-H consisting of SEQ ID NO: 12061.

6. The ABP of claim 1, wherein the ABP comprises a CDR1-L consisting of SEQ ID NO: 12007, a CDR2-L consisting of SEQ ID NO: 12017, a CDR3-L consisting of SEQ ID NO: 12027, a CDR1-H consisting of SEQ ID NO: 12042, a CDR2-H consisting of SEQ ID NO: 12052, and a CDR3-H consisting of SEQ ID NO: 12062.

7. The ABP of claim 1, wherein the ABP comprises a CDR1-L consisting of SEQ ID NO: 12008, a CDR2-L consisting of SEQ ID NO: 12018, a CDR3-L consisting of SEQ ID NO: 12028, a CDR1-H consisting of SEQ ID NO: 12043, a CDR2-H consisting of SEQ ID NO: 12053, and a CDR3-H consisting of SEQ ID NO: 12063.

8. The ABP of claim 1 or 2, wherein the ABP comprises
a variable light chain (V_{L}) comprising a sequence at least 97% identical to SEQ ID NO:14 and a variable heavy chain (V_{H}) comprising a sequence at least 97% identical to SEQ ID NO:114.

9. The ABP of any one of claims 1-8, wherein the ABP comprises an scFv or a full length monoclonal antibody.

10. The ABP of any one of claims 1-9, wherein the ABP comprises an immunoglobulin constant region.

11. The ABP of any one of claims 1-10, wherein
the ABP binds human CTLA-4 with a K_{D} of less than 500 nM, as measured by surface plasmon resonance; or
the ABP binds human CTLA-4 with a K_{D} of less than 200 nM, as measured by surface plasmon resonance; or
the ABP binds human CTLA-4 with a K_{D} of less than 25 nM, as measured by surface plasmon resonance; or
the ABP binds to human CTLA-4 on a cell surface with a K_{D} of less than 25 nM.

12. The ABP of any one of claims 1-11, wherein the ABP is a IgG1 ABP.

13. The ABP of any one of claims 1-12, wherein the ABP comprises an IGHG1*01 human heavy chain constant region gene segment.

14. The ABP of any one of claims 1-13, wherein the ABP comprises a lysine at amino acid position 97 (R97) according to IMGT exon numbering.

15. The ABP of any one of claims 1-13, wherein the ABP comprises a lysine at amino acid position 97 (R214) according to EU numbering.

16. The ABP of any one of claims 1-15, comprising an afucosylated Fc region.

17. The ABP of any one of claims 1-16, produced from a cell comprising a bacterial protein RMD (GDP-6-deoxy-D-lyxo-4-hexulose reductase) or a modification thereof.

18. The ABP of claim 13, wherein the cell is cultured in the absence of fucose.

19. The ABP of any one of claims 1-18, produced from a cell lacking or reduced expression of Fut8.

20. The ABP of any one of claims 1-19, produced from a cell cultured in the presence of a fucosylation inhibitor, 2-Fluorfucose (2FF).

21. The ABP of any one of claims 1-20, produced from a cell overexpressing glycosyltransferase (GnTIII).

22. The ABP of any one of claims 1-21, having been isolated based on its fucosylation status.

23. The ABP of any one of claims 1-22, comprising an Fc region lacking core fucosylation of the N-glycan of the Fc portion.

24. The ABP of any one of claims 1-23, wherein the ABP is an afucosylated monoclonal antibody.

25. A pharmaceutical composition comprising the ABP of any one of claims 1-24 and a pharmaceutically acceptable excipient.

26. The pharmaceutical composition of claim 25, wherein less than 50% of the ABP is fucosylated.

27. The pharmaceutical composition of claim 26, wherein less than 40% of the ABP is fucosylated.

28. The pharmaceutical composition of claim 27, wherein less than 30% of the ABP is fucosylated.

29. The pharmaceutical composition of claim 28, wherein less than 20% of the ABP is fucosylated.

30. The pharmaceutical composition of claim 29, wherein less than 10% of the ABP is fucosylated.

31. The pharmaceutical composition of claim 25, wherein more than 30% of the ABP is fucosylated.

32. The pharmaceutical composition of claim 31, wherein more than 40% of the ABP is fucosylated.

33. The pharmaceutical composition of claim 32, wherein more than 50% of the ABP is fucosylated.

34. The pharmaceutical composition of claim 33, wherein more than 60% of the ABP is fucosylated.

35. The pharmaceutical composition of claim 34, wherein more than 70% of the ABP is fucosylated.

36. The pharmaceutical composition of claim 35, wherein more than 80% of the ABP is fucosylated.

37. The pharmaceutical composition of claim 36, wherein more than 90% of the ABP is fucosylated.

38. The pharmaceutical composition of any one of claims 25-37, having a pH from 5.0 to 6.5.

39. The pharmaceutical composition of any one of claims 25-38, comprising 20 mM of histidine or citrate buffer.

40. The pharmaceutical composition of any one of claims 25-39, comprising 50 mM of NaCl.

41. The pharmaceutical composition of any one of claims 25-40, comprising sucrose at a concentration from 170 mM to 270 mM.

42. The pharmaceutical composition of claim 41, comprising 170 mM or 270 mM of sucrose.

43. The pharmaceutical composition of any one of claims 25-42, comprising 5 mg/mL to 20 mg/mL of the ABP.

44. The pharmaceutical composition of any one of claims 26-43, comprising 20 mg/mL of the ABP.

45. The pharmaceutical composition of any one of claims 26-44, comprising 5 mg/mL of the ABP.

46. A method of treating a disease comprising the step of administering to a subject in need thereof an effective amount of the ABP of any of claims 1-24 or the pharmaceutical composition of any one of claims 25-45.

47. The method of claim 46, wherein the disease is selected from the group consisting of cancer, AIDS, Alzheimer's disease and viral or bacterial infection.

48. The method of claim 46 or claim 47, further comprising the step of administering one or more additional therapeutic agents to the subject.

49. The method of claim 48, wherein the additional therapeutic agent is selected from an anti-PD-L1, an anti-PD1, a LAG-3 inhibitor, aCD47 inhibitor, a TIGIT inhibitor, a chemotherapy agent, an immune-stimulatory agent, radiation, a BRAF inhibitor, a MEK inhibitor, a PI3K inhibitor, a cytokine, a polynucleotide encoding a cytokine, an oncolytic virus encoding a cytokine, and a combination thereof.

50. An isolated polynucleotide encoding the ABP of any of claims 1-24.

51. A vector comprising the isolated polynucleotide of claim 50.

52. A host cell comprising the isolated polynucleotide of claim 50 or the vector of claim 51.

53. The host cell of claim 52, wherein the host cell further comprises a bacterial protein RMD (GDP-6-deoxy-D-lyxo-4-hexulose reductase).

54. The host cell of any one of claims 52-53, wherein the host cell is cultured in the absence of fucose.

55. The host cell of any one of claims 52-54, lacking or having reduced expression of Fut8.

56. The host cell of any one of claims 52-55, cultured in the presence of a fucosylation inhibitor 2-Fluorfucose (2FF).

57. The host cell of any one of claims 52-56, overexpressing glycosyltransferase (GnTIII).

58. A method of producing an isolated antigen binding protein (ABP) that specifically binds human CTLA-4, comprising inducing expression of the ABP in the host cell of any one of claims 52-57, and isolating the ABP.

59. The method of claim 58, further comprising the step of isolating the ABP based on its fucosylation status.

60. The method of any one of claims 58-59, wherein the host cell is cultured in a cultured medium comprising a fucosylation inhibitor.

61. The method of claim 60, wherein the fucosylation inhibitor is 2-Fluorfucose (2FF).

62. A method of reducing CTLA-4^{HI} Tregs in a subject with limited proliferation of remaining Tregs comprising administering an effective dose of
the ABP of any one of claims 1-24 or the pharmaceutical composition of any one of claims 25-45 to the subject.

63. The method of claim 62, wherein the subject is a human subject, optionally, a human subject with RCC (renal cell cancer), NSCLC (non-small cell lung cancer), Merkel cell carcinoma, cSCC, mesothelioma, MSI colorectal cancer, ovarian cancer, or cervical cancer.

64. The method of claim 62 or claim 63, further comprising the step of administering one or more additional therapeutic agents to the subject.

65. The method of claim 64, wherein the additional therapeutic agent is an anti-PD-L1 or an anti-PD1 or a combination thereof.

66. An isolated antigen binding protein (ABP) that specifically binds a human cytotoxic T-lymphocyte associated protein 4 (CTLA-4), wherein the ABP comprises:
(a) a CDR1-L consisting of SEQ ID NO:12078, a CDR2-L consisting of SEQ ID NO:12079, a CDR3-L consisting of SEQ ID NO: 12080, a CDR1-H consisting of SEQ ID NO:12075, a CDR2-H consisting of SEQ ID NO:12076 and a CDR3-H consisting of SEQ ID NO:12077;
(b) a CDR1-L consisting of SEQ ID NO: 1014, a CDR2-L consisting of SEQ ID NO: 2014, a CDR3-L consisting of SEQ ID NO: 3014, a CDR1-H consisting of SEQ ID NO: 4014, a CDR2-H consisting of SEQ ID NO: 5014 and a CDR3-H consisting of SEQ ID NO: 6014;
(c) a CDR1-L consisting of SEQ ID NO: 12004, a CDR2-L consisting of SEQ ID NO: 12014, a CDR3-L consisting of SEQ ID NO: 12024, a CDR1-H consisting of SEQ ID NO: 12039, a CDR2-H consisting of SEQ ID NO: 12049, and a CDR3-H consisting of SEQ ID NO: 12059;
(d) a CDR1-L consisting of SEQ ID NO: 12005, a CDR2-L consisting of SEQ ID NO: 12015, a CDR3-L consisting of SEQ ID NO: 12025, a CDR1-H consisting of SEQ ID NO: 12040, a CDR2-H consisting of SEQ ID NO: 12050, and a CDR3-H consisting of SEQ ID NO: 12060;
(e) a CDR1-L consisting of SEQ ID NO: 12006, a CDR2-L consisting of SEQ ID NO: 12016, a CDR3-L consisting of SEQ ID NO: 12026, a CDR1-H consisting of SEQ ID NO: 12041, a CDR2-H consisting of SEQ ID NO: 12051, and a CDR3-H consisting of SEQ ID NO: 12061;
(f) a CDR1-L consisting of SEQ ID NO: 12007, a CDR2-L consisting of SEQ ID NO: 12017, a CDR3-L consisting of SEQ ID NO: 12027, a CDR1-H consisting of SEQ ID NO: 12042, a CDR2-H consisting of SEQ ID NO: 12052, and a CDR3-H consisting of SEQ ID NO: 12062; or
(g) a CDR1-L consisting of SEQ ID NO: 12008, a CDR2-L consisting of SEQ ID NO: 12018, a CDR3-L consisting of SEQ ID NO: 12028, a CDR1-H consisting of SEQ ID NO: 12043, a CDR2-H consisting of SEQ ID NO: 12053, and a CDR3-H consisting of SEQ ID NO: 12063.

67. The ABP of claim 66, wherein the ABP comprises
a variable light chain (V_{L}) comprising a sequence at least 97% identical to SEQ ID NO:14 and a variable heavy chain (V_{H}) comprising a sequence at least 97% identical to SEQ ID NO:114.

68. The ABP of any one of claims 66-67, wherein the ABP comprises an scFv or a full length monoclonal antibody.

69. The ABP of any one of claims 66-68, wherein the ABP comprises an immunoglobulin constant region.

70. The ABP of any one of claims 66-69, wherein
the ABP binds human CTLA-4 with a K_{D} of less than 500 nM, as measured by surface plasmon resonance; or
the ABP binds human CTLA-4 with a K_{D} of less than 200 nM, as measured by surface plasmon resonance; or
the ABP binds human CTLA-4 with a K_{D} of less than 25 nM, as measured by surface plasmon resonance; or
the ABP binds to human CTLA-4 on a cell surface with a K_{D} of less than 25 nM.

71. The ABP of any one of claims 66-70, wherein the ABP is a IgG1 ABP.

72. The ABP of any one of claims 66-71, wherein the ABP comprises an IGHG1*01 human heavy chain constant region gene segment.

73. The ABP of any one of claims 66-72, wherein the ABP comprises a lysine at amino acid position 97 (R97) according to IMGT exon numbering.

74. The ABP of any one of claims 66-73, wherein the ABP comprises a lysine at amino acid position 97 (R214) according to EU numbering.

75. The ABP of any one of claims 66-74, comprising an afucosylated Fc region.

76. The ABP of any one of claims 66-75, produced from a cell comprising a bacterial protein RMD (GDP-6-deoxy-D-lyxo-4-hexulose reductase) or a modification thereof.

77. The ABP of claim 76, wherein the cell is cultured in the absence of fucose.

78. The ABP of any one of claims 66-77, produced from a cell lacking or reduced expression of Fut8.

79. The ABP of any one of claims 66-78, produced from a cell cultured in the presence of a fucosylation inhibitor, 2-Fluorfucose (2FF).

80. The ABP of any one of claims 66-79, produced from a cell overexpressing glycosyltransferase (GnTIII).

81. The ABP of any one of claims 66-80, having been isolated based on its fucosylation status.

82. The ABP of any one of claims 58-81, comprising an Fc region lacking core fucosylation of the N-glycan of the Fc portion.

83. The ABP of any one of claims 66-82, wherein the ABP is an afucosylated monoclonal antibody.

84. The ABP of claim 83, wherein the afucosylated Fc region has less than 30% fucosylation, and wherein less than 30% fucosylation enhances Fc Gamma Receptor IIIa (FcgRIIIa) signaling or a protein encoded by protein encoded by FcgR3a.

85. A pharmaceutical composition comprising the ABP of any one of claims 66-84, and a pharmaceutically acceptable excipient.

86. The pharmaceutical composition of claim 85, wherein less than 50% of the ABP is fucosylated.

87. The pharmaceutical composition of claim 86, wherein less than 40% of the ABP is fucosylated.

88. The pharmaceutical composition of claim 87, wherein less than 30% of the ABP is fucosylated.

89. The pharmaceutical composition of claim 88, wherein less than 20% of the ABP is fucosylated.

90. The pharmaceutical composition of claim 89, wherein less than 10% of the ABP is fucosylated.

91. The pharmaceutical composition of claim 85, wherein more than 30% of the ABP is fucosylated.

92. The pharmaceutical composition of claim 91, wherein more than 40% of the ABP is fucosylated.

93. The pharmaceutical composition of claim 92, wherein more than 50% of the ABP is fucosylated.

94. The pharmaceutical composition of claim 93, wherein more than 60% of the ABP is fucosylated.

95. The pharmaceutical composition of claim 94, wherein more than 70% of the ABP is fucosylated.

96. The pharmaceutical composition of claim 95, wherein more than 80% of the ABP is fucosylated.

97. The pharmaceutical composition of claim 96, wherein more than 90% of the ABP is fucosylated.

98. The pharmaceutical composition of any one of claims 85-97, having a pH from 5.0 to 6.5.

99. The pharmaceutical composition of any one of claims 85-98, comprising 20 mM of histidine or citrate buffer.

100. The pharmaceutical composition of any one of claims 85-99, comprising 50 mM of NaCl.

101. The pharmaceutical composition of any one of claims 85-100, comprising sucrose at a concentration from 170 mM to 270 mM.

102. The pharmaceutical composition of claim 101, comprising 170 mM or 270 mM of sucrose.

103. The pharmaceutical composition of any one of claims 85-102, comprising 20 mg/mL of the ABP.

104. The pharmaceutical composition of any one of claims 85-103, comprising 5 mg/mL of the ABP.

105. A method of treating cancer comprising the step of:
administering to a subject in need thereof an effective amount of the ABP of any of claims 66-84 or the pharmaceutical composition of any one of claims 85-104.

106. The method of claim 105, wherein the subject has a malignant tumor.

107. The method of claim 105, wherein the ABP, when administered, comprises increased Fc receptor (FcR) signaling as compared to ipilimumab, and wherein said administering reduces the amount of CTLA-4^{HI} Tregs in the subject.

108. The method of claim 107, wherein said administering reduces proliferation of peripheral Tregs in the subject as compared to ipilimumab.

109. The method of any one of claims 105-108, further comprising the step of administering one or more additional therapeutic agents to the subject.

110. The method of claim 109, wherein the additional therapeutic agent is selected from an anti-PD-L1, an anti-PD1, a TIGIT inhibitor, a LAG-3 inhibitor, a CD47 inhibitor, a BRAF inhibitor, a MEK inhibitor, a PI3K inhibitor, a chemotherapy agent, an immune-stimulatory agent, radiation, a cytokine, a polynucleotide encoding a cytokine, an oncolytic virus encoding a cytokine, and a combination thereof.

111. The method of any one of claims 105-110, wherein the ABP comprises an afucosylated Fc region that has less than 30% fucosylation, and wherein less than 30% fucosylation enhances Fc Gamma Receptor IIIa (FcgRIIIa) signaling or a protein encoded by protein encoded by FcgR3a.

112. The method of any one of claims 105-110, wherein the ABP comprises a fucosylated Fc region that has more than 70% fucosylation.

113. An isolated polynucleotide encoding the ABP of any of claims 66-84.

114. A vector comprising the isolated polynucleotide of claim 113.

115. A host cell comprising the isolated polynucleotide of claim 113 or the vector of claim 114.

116. The host cell of claim 115, wherein the host cell is engineered to have reduced fucosylation as compared to a host cell that is not engineered.

117. The host cell of claim 116, wherein the host cell further comprises a bacterial protein RMD (GDP-6-deoxy-D-lyxo-4-hexulose reductase).

118. The host cell of any one of claims 115-117, wherein the host cell is cultured in the absence of fucose.

119. The host cell of any one of claims 115-118, lacking or having reduced expression of Fut8.

120. The host cell of any one of claims 115-119, cultured in the presence of a fucosylation inhibitor 2-Fluorfucose (2FF).

121. The host cell of any one of claims 115-120, overexpressing glycosyltransferase (GnTIII).

122. A method of producing an isolated antigen binding protein (ABP) that specifically binds human CTLA-4, comprising inducing expression of the ABP in the host cell of any one of claims 115-121, and isolating the ABP, wherein the ABP comprises an afucosylated Fc.

123. The method of claim 122, further comprising the step of isolating the ABP based on its fucosylation status.

124. The method of any one of claims 115-123, wherein the host cell is cultured in a cultured medium comprising a fucosylation inhibitor.

125. The method of claim 124, wherein the fucosylation inhibitor is 2-Fluorfucose (2FF).

126. An isolated antigen binding protein (ABP) that specifically binds an antigen, comprising an IGHG1*01 human heavy chain constant region gene segment, optionally wherein the antigen is a human cytotoxic T-lymphocyte associated protein 4 (CTLA-4).

127. The ABP of claim 126, wherein the ABP comprises:
(a) a CDR1-L consisting of SEQ ID NO:12078, a CDR2-L consisting of SEQ ID NO:12079, a CDR3-L consisting of SEQ ID NO: 12080, a CDR1-H consisting of SEQ ID NO:12075, a CDR2-H consisting of SEQ ID NO:12076 and a CDR3-H consisting of SEQ ID NO:12077;
(b) a CDR1-L consisting of SEQ ID NO: 1014, a CDR2-L consisting of SEQ ID NO: 2014, a CDR3-L consisting of SEQ ID NO: 3014, a CDR1-H consisting of SEQ ID NO: 4014, a CDR2-H consisting of SEQ ID NO: 5014 and a CDR3-H consisting of SEQ ID NO: 6014;
(c) a CDR1-L consisting of SEQ ID NO: 12004, a CDR2-L consisting of SEQ ID NO: 12014, a CDR3-L consisting of SEQ ID NO: 12024, a CDR1-H consisting of SEQ ID NO: 12039, a CDR2-H consisting of SEQ ID NO: 12049, and a CDR3-H consisting of SEQ ID NO: 12059;
(d) a CDR1-L consisting of SEQ ID NO: 12005, a CDR2-L consisting of SEQ ID NO: 12015, a CDR3-L consisting of SEQ ID NO: 12025, a CDR1-H consisting of SEQ ID NO: 12040, a CDR2-H consisting of SEQ ID NO: 12050, and a CDR3-H consisting of SEQ ID NO: 12060;
(e) a CDR1-L consisting of SEQ ID NO: 12006, a CDR2-L consisting of SEQ ID NO: 12016, a CDR3-L consisting of SEQ ID NO: 12026, a CDR1-H consisting of SEQ ID NO: 12041, a CDR2-H consisting of SEQ ID NO: 12051, and a CDR3-H consisting of SEQ ID NO: 12061;
(f) a CDR1-L consisting of SEQ ID NO: 12007, a CDR2-L consisting of SEQ ID NO: 12017, a CDR3-L consisting of SEQ ID NO: 12027, a CDR1-H consisting of SEQ ID NO: 12042, a CDR2-H consisting of SEQ ID NO: 12052, and a CDR3-H consisting of SEQ ID NO: 12062; or
(g) a CDR1-L consisting of SEQ ID NO: 12008, a CDR2-L consisting of SEQ ID NO: 12018, a CDR3-L consisting of SEQ ID NO: 12028, a CDR1-H consisting of SEQ ID NO: 12043, a CDR2-H consisting of SEQ ID NO: 12053, and a CDR3-H consisting of SEQ ID NO: 12063.

128. The ABP of claim 127, wherein the ABP comprises
a variable light chain (V_{L}) comprising a sequence at least 97% identical to SEQ ID NO:14 and a variable heavy chain (V_{H}) comprising a sequence at least 97% identical to SEQ ID NO:114.

129. The ABP of claim 126, wherein the ABP comprises:
(a) a CDR1-L consisting of any one of SEQ ID NOs:, a CDR2-L consisting of any one of SEQ ID NOs: 1001-1028, a CDR3-L consisting of any one of SEQ ID NOs: 2001-2028, a CDR1-H consisting of any one of SEQ ID NOs:, a CDR2-H consisting of consisting of any one of SEQ ID NOs: 3001-3028.

130. The ABP of claim 127, wherein the ABP comprises
a variable light chain (V_{L}) comprising a sequence at least 97% identical to any one of SEQ ID NOs:1-28 and a variable heavy chain (V_{H}) comprising a sequence at least 97% identical to SEQ ID NO:1-128.

131. The ABP of claim 126, wherein the ABP comprises a lysine at amino acid position 97 (R97) according to IMGT exon numbering.

132. The ABP of claim 126, wherein the ABP comprises a lysine at amino acid position 97 (R214) according to EU numbering.

133. The ABP of any one of claims 126-132, comprising an afucosylated Fc region.

134. The ABP of any one of claims 126-133, produced from a cell comprising a bacterial protein RMD (GDP-6-deoxy-D-lyxo-4-hexulose reductase) or a modification thereof.

135. The ABP of claim 134, wherein the cell is cultured in the absence of fucose.

136. The ABP of any one of claims 126-135, produced from a cell lacking or reduced expression of Fut8.

137. The ABP of any one of claims 126-136, produced from a cell cultured in the presence of a fucosylation inhibitor, 2-Fluorfucose (2FF).

138. The ABP of any one of claims 126-137, produced from a cell overexpressing glycosyltransferase (GnTIII).

139. The ABP of any one of claims 126-138, having been isolated based on its fucosylation status.

140. The ABP of any one of claims 126-139, comprising an Fc region lacking core fucosylation of the N-glycan of the Fc portion.

141. The ABP of any one of claims 126-140, wherein the ABP is an afucosylated monoclonal antibody.

142. The ABP of any one of claims 131-141, wherein the ABP is selected from an anti-CTLA-4 antibody or antigen-binding fragment thereof, anti-PD-L1 antibody or antigen-binding fragment thereof, an anti-PD1 antibody or antigen-binding fragment thereof, a TIGIT antibody or antigen-binding fragment thereof, a LAG-3 antibody or antigen-binding fragment thereof, a CD47 antibody or antigen-binding fragment thereof, a BRAF antibody or antigen-binding fragment thereof, a MEK antibody or antigen-binding fragment thereof, and a PI3K antibody or antigen-binding fragment thereof.

143. The ABP any one of claims 131-141, wherein the ABP comprises:
(a) a CDR1-L consisting of SEQ ID NO:12078, a CDR2-L consisting of SEQ ID NO:12079, a CDR3-L consisting of SEQ ID NO:12080, a CDR1-H consisting of SEQ ID NO:12075, a CDR2-H consisting of SEQ ID NO:12076 and a CDR3-H consisting of SEQ ID NO:12077;
(b) a CDR1-L consisting of SEQ ID NO: 1014, a CDR2-L consisting of SEQ ID NO: 2014, a CDR3-L consisting of SEQ ID NO: 3014, a CDR1-H consisting of SEQ ID NO: 4014, a CDR2-H consisting of SEQ ID NO: 5014 and a CDR3-H consisting of SEQ ID NO: 6014;
(c) a CDR1-L consisting of SEQ ID NO: 12004, a CDR2-L consisting of SEQ ID NO: 12014, a CDR3-L consisting of SEQ ID NO: 12024, a CDR1-H consisting of SEQ ID NO: 12039, a CDR2-H consisting of SEQ ID NO: 12049, and a CDR3-H consisting of SEQ ID NO: 12059;
(d) a CDR1-L consisting of SEQ ID NO: 12005, a CDR2-L consisting of SEQ ID NO: 12015, a CDR3-L consisting of SEQ ID NO: 12025, a CDR1-H consisting of SEQ ID NO: 12040, a CDR2-H consisting of SEQ ID NO: 12050, and a CDR3-H consisting of SEQ ID NO: 12060;
(e) a CDR1-L consisting of SEQ ID NO: 12006, a CDR2-L consisting of SEQ ID NO: 12016, a CDR3-L consisting of SEQ ID NO: 12026, a CDR1-H consisting of SEQ ID NO: 12041, a CDR2-H consisting of SEQ ID NO: 12051, and a CDR3-H consisting of SEQ ID NO: 12061;
(f) a CDR1-L consisting of SEQ ID NO: 12007, a CDR2-L consisting of SEQ ID NO: 12017, a CDR3-L consisting of SEQ ID NO: 12027, a CDR1-H consisting of SEQ ID NO: 12042, a CDR2-H consisting of SEQ ID NO: 12052, and a CDR3-H consisting of SEQ ID NO: 12062; or
(g) a CDR1-L consisting of SEQ ID NO: 12008, a CDR2-L consisting of SEQ ID NO: 12018, a CDR3-L consisting of SEQ ID NO: 12028, a CDR1-H consisting of SEQ ID NO: 12043, a CDR2-H consisting of SEQ ID NO: 12053, and a CDR3-H consisting of SEQ ID NO: 12063.

144. The ABP of any one of claims 131-141, wherein the ABP comprises
a variable light chain (V_{L}) comprising a sequence at least 97% identical to SEQ ID NO:14 and a variable heavy chain (V_{H}) comprising a sequence at least 97% identical to SEQ ID NO:114.

145. The ABP of any one of claims 131-141, wherein the ABP comprises:
(a) a CDR1-L consisting of any one of SEQ ID NO: 12081: a CDR2-L consisting of SEQ ID NO: 12082, a CDR3-L consisting of SEQ ID NO: 12083, a CDR1-H consisting of SEQ ID NO: 12084, a CDR2-H consisting of consisting of SEQ ID NO: 12085, and a CDR3-H consisting of SEQ ID NO: 12086.

146. The ABP of any one of claims 131-141, wherein the ABP comprises
a variable light chain (V_{L}) comprising a sequence at least 97% identical to SEQ ID NO: 12088 and a variable heavy chain (V_{H}) comprising a sequence at least 97% identical to SEQ ID NO: 12087.

147. A pharmaceutical composition comprising the ABP of any one of claims 126-146 and a pharmaceutically acceptable excipient.

148. The pharmaceutical composition of claim 147, wherein less than 50% of the ABP is fucosylated.

149. The pharmaceutical composition of claim 148, wherein less than 40% of the ABP is fucosylated.

150. The pharmaceutical composition of claim 149, wherein less than 30% of the ABP is fucosylated.

151. The pharmaceutical composition of claim 150, wherein less than 20% of the ABP is fucosylated.

152. The pharmaceutical composition of claim 151, wherein less than 10% of the ABP is fucosylated.

153. The pharmaceutical composition of claim 147, wherein more than 30% of the ABP is fucosylated.

154. The pharmaceutical composition of claim 153, wherein more than 40% of the ABP is fucosylated.

155. The pharmaceutical composition of claim 154, wherein more than 50% of the ABP is fucosylated.

156. The pharmaceutical composition of claim 155, wherein more than 60% of the ABP is fucosylated.

157. The pharmaceutical composition of claim 156, wherein more than 70% of the ABP is fucosylated.

158. The pharmaceutical composition of claim 157, wherein more than 80% of the ABP is fucosylated.

159. The pharmaceutical composition of claim 158, wherein more than 90% of the ABP is fucosylated.

160. The pharmaceutical composition of any one of claims 147-159, having a pH from 5.0 to 6.5.

161. The pharmaceutical composition of any one of claims 147-160, comprising 20 mM of histidine or citrate buffer.

162. The pharmaceutical composition of any one of claims 147-161, comprising 50 mM of NaCl.

163. The pharmaceutical composition of any one of claims 162-162, comprising sucrose at a concentration from 170 mM to 270 mM.

164. The pharmaceutical composition of claim 163, comprising 170 mM or 270 mM of sucrose.

165. The pharmaceutical composition of any one of claims 147-164, comprising 20 mg/mL of the ABP.

166. A method of treating a disease comprising the step of
administering to a subject in need thereof an effective amount of the ABP of any of claims 125-145 or the pharmaceutical composition of any one of claims 147-165.

167. The method of claim 166, wherein the disease is selected from the group consisting of cancer, AIDS, Alzheimer's disease and viral or bacterial infection.

168. The method of claim 166 or claim 167, further comprising the step of administering one or more additional therapeutic agents to the subject.

169. The method of claim 168, wherein the additional therapeutic agent is selected from an anti-PD-L1, an anti-PD1, a TIGIT inhibitor, a LAG-3 inhibitor, a CD47 inhibitor, a BRAF inhibitor, a MEK inhibitor, a PI3K inhibitor, a chemotherapy agent, an immune-stimulatory agent, radiation, a cytokine, a polynucleotide encoding a cytokine, an oncolytic virus encoding a cytokine, and a combination thereof.

170. An isolated polynucleotide encoding the ABP of any of claims 126-146.

171. A vector comprising the isolated polynucleotide of claim 170.

172. A host cell comprising the isolated polynucleotide of claim 170 or the vector of claim 170.

173. The host cell of claim 172, wherein the host cell further comprises a bacterial protein RMD (GDP-6-deoxy-D-lyxo-4-hexulose reductase).

174. The host cell of any one of claims 172-173, wherein the host cell is cultured in the absence of fucose.

175. The host cell of any one of claims 172-174, lacking or having reduced expression of Fut8.

176. The host cell of any one of claims 172-175, cultured in the presence of a fucosylation inhibitor 2-Fluorfucose (2FF).

177. The host cell of any one of claims 172-176, overexpressing glycosyltransferase (GnTIII).

178. A method of producing an isolated antigen binding protein (ABP) that specifically binds human CTLA-4, comprising inducing expression of the ABP in the host cell of any one of claims 171-177, and isolating the ABP.

179. The method of claim 178, further comprising the step of isolating the ABP based on its fucosylation status.

180. The method of any one of claims 178-179, wherein the host cell is cultured in a cultured medium comprising a fucosylation inhibitor.

181. The method of claim 180, wherein the fucosylation inhibitor is 2-Fluorfucose (2FF).

182. A method of reducing CTLA-4^{HI} Tregs in a subject with limited proliferation of remaining Tregs comprising administering an effective dose of
the ABP of any one of claims 126-146 or the pharmaceutical composition of any one of claims 141-164.

183. The method of claim 182, wherein the subject is a human subject, optionally, a human subject with melanoma, RCC (renal cell cancer), NSCLC (non-small cell lung cancer), Merkel cell carcinoma, cSCC, mesothelioma, MSI colorectal cancer, ovarian cancer, or cervical cancer.

184. The method of claim 182 or claim 183, further comprising the step of administering one or more additional therapeutic agents to the subject.

185. The method of claim 184, wherein the additional therapeutic agent is an anti-PD-L1 or an anti-PD1 or a combination thereof.

186. The method of claim 182, wherein the subject has a tumor with high levels of Tregs, high levels of CTLA-4, high levels of NK cells, or high levels of activating FcRs.

187. A method of reducing CTLA-4^{HI} Tregs in a subject with limited proliferation of remaining Tregs comprising administering an effective dose of
the ABP of any one of claims 126-146 or the pharmaceutical composition of any one of claims 146-164.

188. The method of claim 187, wherein the subject is a human subject, optionally, a human subject with melanoma, RCC (renal cell cancer), NSCLC (non-small cell lung cancer), Merkel cell carcinoma, cSCC, mesothelioma, MSI colorectal cancer, ovarian cancer, or cervical cancer.

189. The method of claim 187 or claim 188, further comprising the step of administering one or more additional therapeutic agents to the subject.

190. The method of claim 187, wherein the subject has a tumor with high levels of Tregs, high levels of CTLA-4, high levels of NK cells, or high levels of activating FcRs.

191. A method of reducing CTLA-4^{HI} Tregs in a subject with limited proliferation of remaining Tregs comprising administering to the subject an effective dose of an antigen binding protein (ABP) that specifically binds a human cytotoxic T-lymphocyte associated protein 4 (CTLA-4).

192. The method of claim 191, wherein the subject is a human subject, optionally, a human subject with melanoma, RCC (renal cell cancer), NSCLC (non-small cell lung cancer), Merkel cell carcinoma, cSCC, mesothelioma, MSI colorectal cancer, ovarian cancer, or cervical cancer.

193. The method of claim 191 or claim 192, further comprising the step of administering one or more additional therapeutic agents to the subject.

194. The ABP of claim 191, wherein the ABP comprises
a variable light chain (VL) comprising a sequence at least 97% identical to SEQ ID NO:14 and a variable heavy chain (VH) comprising a sequence at least 97% identical to SEQ ID NO:114.

195. The ABP of claim 191, wherein the ABP comprises:
(a) a CDR1-L consisting of any one of SEQ ID NOs:, a CDR2-L consisting of any one of SEQ ID NOs: 1001-1028, a CDR3-L consisting of any one of SEQ ID NOs: 3001-3028, a CDR1-H consisting of any one of SEQ ID NOs:, a CDR2-H consisting of consisting of any one of SEQ ID NOs: 3001-3028.

196. The ABP of claim 191, wherein the ABP comprises
a variable light chain (VL) comprising a sequence at least 97% identical to any one of SEQ ID NOs:1-28 and a variable heavy chain (VH) comprising a sequence at least 97% identical to SEQ ID NO:101-128.

197. The ABP of claim 191, wherein the ABP comprises:
(a) a CDR1-L consisting of any one of SEQ ID NO: 12081:, a CDR2-L consisting of SEQ ID NO: 12082, a CDR3-L consisting of SEQ ID NO: 12083, a CDR1-H consisting of SEQ ID NO: 12084, a CDR2-H consisting of consisting of SEQ ID NO: 12085, and a CDR3-H consisting of SEQ ID NO: 12086.

198. The ABP of claim 191, wherein the ABP comprises
a variable light chain (V_{L}) comprising a sequence at least 97% identical to SEQ ID NO: 12088 and a variable heavy chain (V_{H}) comprising a sequence at least 97% identical to SEQ ID NO: 12087.
